SEQUENCE LISTING (264 sequences)

SEQ ID NO: 1 - protein, 5 aa, synthetic construct
LPXTG

SEQ ID NO: 2 - protein, 55 aa, synthetic construct
KNRKAKAKPVTRGAGAGGRQRGQNKERPPPVPNPDYEPIRKGQRDLYSGLNQRRI

SEQ ID NO: 3 - protein, 25 aa, synthetic construct
KTNIRSSTRDWKDHKFKWRKDPQDK

SEQ ID NO: 4 - protein, 21 aa, synthetic construct
MALPVTALLLPLALLLHAARP

SEQ ID NO: 5 - protein, 20 aa, synthetic construct
METDTLLLWVLLLWVPGSTG

SEQ ID NO: 6 - protein, 22 aa, synthetic construct
MLLLVTSLLLCELPHPAFLLIP

SEQ ID NO: 7 - protein, 45 aa, synthetic construct
TTTPAPRPPTPAPTIASQPLSLRPEACRPAAGGAVHTRGLDFACD

SEQ ID NO: 8 - protein, 39 aa, synthetic construct
IEVMYPPPYLDNEKSNGTIIHVKGKHLCPSPLFPGPSKP

SEQ ID NO: 9 - protein, 12 aa, synthetic construct
ESKYGPPCPPCP

SEQ ID NO: 10 - protein, 12 aa, synthetic construct
ESKYGPPCPSCP

SEQ ID NO: 11 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 24 aa, synthetic construct
IYIWAPLAGTCGVLLLSLVITLYC

SEQ ID NO: 13 - protein, 27 aa, synthetic construct
FWVLVVVGGVLACYSLLVTVAFIIFWV

SEQ ID NO: 14 - protein, 42 aa, synthetic construct
KRGRKKLLYIFKQPFMRPVQTTQEEDGCSCRFPEEEEGGCEL

SEQ ID NO: 15 - protein, 41 aa, synthetic construct
RSKRSRLLHSDYMNMTPRRPGPTRKHYQPYAPPRDFAAYRS

SEQ ID NO: 16 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 502 aa, synthetic construct
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 485 aa, synthetic construct
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 677 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 489 aa, synthetic construct
MALPVTALLLPLALLLHAARPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 720 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSLDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 481 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 464 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 657 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 468 aa, synthetic construct
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 700 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSLDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 29 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 30 - protein, 6 aa, synthetic construct
QDISKY

SEQ ID NO: 31 - skipped

SEQ ID NO: 32 - protein, 9 aa, synthetic construct
QQGNTLPYT

SEQ ID NO: 33 - protein, 18 aa, synthetic construct
GSTSGSGKPGSGEGSTKG

SEQ ID NO: 34 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 35 - protein, 8 aa, synthetic construct
GVSLPDYG

SEQ ID NO: 36 - protein, 7 aa, synthetic construct
IWGSETT

SEQ ID NO: 37 - protein, 14 aa, synthetic construct
AKHYYYGGSYAMDY

SEQ ID NO: 38 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 39 - protein, 15 aa, synthetic construct
GGGGSGGGGSGGGGS

SEQ ID NO: 40 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 41 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - DNA, 1383 bp, synthetic construct
  source 1..1383: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 43 - protein, 461 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 1467 bp, synthetic construct
  source 1..1467: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 45 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 245 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 47 - protein, 55 aa, synthetic construct
FVPVFLPAKPTTTPAPRPPTPAPTIASQPLSLRPEACRPAAGGAVHTRGLDFACD

SEQ ID NO: 48 - protein, 28 aa, synthetic construct
IYIWAPLAGTCGVLLLSLVITLYCNHRN

SEQ ID NO: 49 - protein, 246 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 50 - protein, 106 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 51 - protein, 10 aa, synthetic construct
RASSSVNYMD

SEQ ID NO: 52 - protein, 7 aa, synthetic construct
ATSNLAS

SEQ ID NO: 53 - protein, 9 aa, synthetic construct
QQWSFNPPT

SEQ ID NO: 54 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 55 - protein, 5 aa, synthetic construct
SYNMH

SEQ ID NO: 56 - protein, 17 aa, synthetic construct
AIYPGNGDTSYNQKFKG

SEQ ID NO: 57 - protein, 246 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 58 - protein, 111 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 59 - protein, 15 aa, synthetic construct
RASESVSVIGAHLIH

SEQ ID NO: 60 - protein, 7 aa, synthetic construct
LASNLET

SEQ ID NO: 61 - protein, 9 aa, synthetic construct
LQSRIFPRT

SEQ ID NO: 62 - protein, 117 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 63 - protein, 5 aa, synthetic construct
DYSIN

SEQ ID NO: 64 - protein, 17 aa, synthetic construct
WINTETREPAYAYDFRG

SEQ ID NO: 65 - protein, 8 aa, synthetic construct
DYSYAMDY

SEQ ID NO: 66 - protein, 246 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 67 - protein, 111 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 68 - protein, 15 aa, synthetic construct
RASESVTILGSHLIY

SEQ ID NO: 69 - protein, 7 aa, synthetic construct
LASNVQT

SEQ ID NO: 70 - protein, 9 aa, synthetic construct
LQSRTIPRT

SEQ ID NO: 71 - protein, 117 aa, synthetic construct
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 72 - protein, 5 aa, synthetic construct
HYSMN

SEQ ID NO: 73 - protein, 17 aa, synthetic construct
RINTESGVPIYADDFKG

SEQ ID NO: 74 - protein, 8 aa, synthetic construct
DYLYSLDF

SEQ ID NO: 75 - protein, 121 aa, synthetic construct
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 76 - protein, 8 aa, synthetic construct
GFTFSSYA

SEQ ID NO: 77 - protein, 8 aa, synthetic construct
ISGSGDYI

SEQ ID NO: 78 - protein, 14 aa, synthetic construct
AKEGTGANSSLADY

SEQ ID NO: 79 - protein, 243 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 80 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 81 - protein, 6 aa, synthetic construct
QSISSY

SEQ ID NO: 82 - skipped

SEQ ID NO: 83 - protein, 8 aa, synthetic construct
QQKYDLLT

SEQ ID NO: 84 - protein, 119 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 85 - protein, 10 aa, synthetic construct
GGSISSSSYY

SEQ ID NO: 86 - protein, 7 aa, synthetic construct
ISYSGST

SEQ ID NO: 87 - protein, 11 aa, synthetic construct
ARDRGDTILDV

SEQ ID NO: 88 - protein, 23 aa, synthetic construct
MALPVTALLLPLALLLHAARPGS

SEQ ID NO: 89 - protein, 243 aa, synthetic construct
QVQLQQPGAELVKPGASVKLSCKASGYTITSYSLHWVKQRPGQGLEWIGEINPANGDHNFSEKFEIKATLTVDSSSNTAFMQLSRLTSEDSAVYYCTRLDDSRFHWYFDVWGAGTTVTVSSGGGGSGGGGSGGGGSQIVLTQSPALMSASPGEKVTMTCTASSSVSYMYWYQQKPRSSPKPWIFLTSNLASGVPARFSGRGSGTSFSLTISSMEAEDAATYYCQQWSGYPPITFGSGTKLEIK

SEQ ID NO: 90 - protein, 47 aa, synthetic construct
SGTTTPAPRPPTPAPTIASQPLSLRPEACRPAAGGAVHTRGLDFACD

SEQ ID NO: 91 - protein, 243 aa, synthetic construct
QIVLTQSPALMSASPGEKVTMTCTASSSVSYMYWYQQKPRSSPKPWIFLTSNLASGVPARFSGRGSGTSFSLTISSMEAEDAATYYCQQWSGYPPITFGSGTKLEIKGGGGSGGGGSGGGGSQVQLQQPGAELVKPGASVKLSCKASGYTITSYSLHWVKQRPGQGLEWIGEINPANGDHNFSEKFEIKATLTVDSSSNTAFMQLSRLTSEDSAVYYCTRLDDSRFHWYFDVWGAGTTVTVSS

SEQ ID NO: 92 - skipped

SEQ ID NO: 93 - protein, 677 aa, synthetic construct; identical to SEQ ID NO: 20

SEQ ID NO: 94-95 - skipped (2 entries)

SEQ ID NO: 96 - protein, 13 aa, synthetic construct
SNYYGSSYWFFDV

SEQ ID NO: 97 - protein, 246 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 98 - protein, 18 aa, synthetic construct
GSTSGGGSGGGSGGGGSS

SEQ ID NO: 99 - protein, 249 aa, synthetic construct
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSS

SEQ ID NO: 100 - protein, 112 aa, synthetic construct
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIK

SEQ ID NO: 101 - protein, 16 aa, synthetic construct
RSSQSLVSRDGNTYLS

SEQ ID NO: 102 - protein, 7 aa, synthetic construct
KISNRFS

SEQ ID NO: 103 - protein, 9 aa, synthetic construct
MQATQFPLT

SEQ ID NO: 104 - protein, 122 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSS

SEQ ID NO: 105 - protein, 5 aa, synthetic construct
SYWIG

SEQ ID NO: 106 - protein, 17 aa, synthetic construct
IIYPGDSDTRYSPSFQG

SEQ ID NO: 107 - protein, 13 aa, synthetic construct
QGDFWSGYGGMDV

SEQ ID NO: 108 - protein, 121 aa, synthetic construct
EVQLQQSGPELVKPGASVKISCKASRYTFTDYNLHWVKLSHEKSLEWIGFIYPYNGGTGYNQKFKNKAKLTVDYSSSTAYMELRSLTSVDAAVYYCARDHRYNEGVSFDYWGQGTTLTVSS

SEQ ID NO: 109 - protein, 10 aa, synthetic construct
RYTFTDYNLH

SEQ ID NO: 110 - protein, 10 aa, synthetic construct
FIYPYNGGTG

SEQ ID NO: 111 - protein, 12 aa, synthetic construct
DHRYNEGVSFDY

SEQ ID NO: 112 - protein, 111 aa, synthetic construct
NIVLTQSPASLAVSLGQRATISCRASESVDGFGNSFMNWYQQKPGQSPKLLIYLASNLESGVPARFSGSGSRTDFTLTIDPVEADDAATYYCQQNNEDPYTFGGGTKLEIK

SEQ ID NO: 113 - protein, 15 aa, synthetic construct
RASESVDGFGNSFMN

SEQ ID NO: 114 - protein, 7 aa, synthetic construct
LASNLES

SEQ ID NO: 115 - protein, 9 aa, synthetic construct
QQNNEDPYT

SEQ ID NO: 116 - protein, 109 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARWGQGTTVTVSS

SEQ ID NO: 117 - protein, 121 aa, synthetic construct
QVQLVQSGAEVKKPGASVKVSCKASRYTFTDYNLHWVRQAPGQGLEWMGFIYPYNGGTGYAQKFQGRVTMTRDTSTSTVYMELSSLRSEDTAVYYCARDHRYNEGVSFDYWGQGTTVTVSS

SEQ ID NO: 118 - protein, 121 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSS

SEQ ID NO: 119 - protein, 121 aa, synthetic construct
QVQLVQSGAEVKKPGASVKISCKASRYTFTDYNLHWVRQAPGQGLEWIGFIYPYNGGTGYAQKFQGRATLTVDTSTSTAYMELSSLRSEDTAVYYCARDHRYNEGVSFDYWGQGTTLTVSS

SEQ ID NO: 120 - protein, 121 aa, synthetic construct
QVQLVQSGAEVKKPGASVKVSCKASRYTFTDYNLHWVRQAPGQGLEWMGFIYPYNGGTGYAQKFQGRVTMTVDYSTSTAYMELSSLRSEDTAVYYCARDHRYNEGVSFDYWGQGTTVTVSS

SEQ ID NO: 121 - protein, 121 aa, synthetic construct
QVQLVQSGAEVKKPGASVKISCKASRYTFTDYNLHWVRQAPGQGLEWIGFIYPYNGGTGYAQKFQGRATLTVDYSTSTAYMELSSLRSEDTAVYYCARDHRYNEGVSFDYWGQGTTLTVSS

SEQ ID NO: 122 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPFGQGTKLEIK

SEQ ID NO: 123 - protein, 111 aa, synthetic construct
DIQMTQSPSSLSASVGDRVTITCRASESVDGFGNSFMNWYQQKPGKAPKLLIYLASNLESGVPSRFSGSGSGTDFTLTISSLQPEDFATYYCQQNNEDPYTFGQGTKLEIK

SEQ ID NO: 124 - protein, 111 aa, synthetic construct
DIQLTQSPSSLSASVGDRATITCRASESVDGFGNSFMNWYQQKPGKAPKLLIYLASNLESGVPSRFSGSGSGTDFTLTISSVQPEDFATYYCQQNNEDPYTFGQGTKLEIK

SEQ ID NO: 125 - protein, 111 aa, synthetic construct
DIQLTQSPSSLSASVGDRATITCRASESVDGFGNSFMNWYQQKPGKAPKLLIYLASNLESGVPSRFSGSGSRTDFTLTISSVQPEDFATYYCQQNNEDPYTFGQGTKLEIK

SEQ ID NO: 126 - protein, 118 aa, synthetic construct [sequence omitted]

SEQ ID NO: 127 - protein, 107 aa, synthetic construct
DVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIK

SEQ ID NO: 128 - protein, 118 aa, synthetic construct
EVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSS

SEQ ID NO: 129 - protein, 107 aa, synthetic construct
DVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELK

SEQ ID NO: 130 - protein, 121 aa, synthetic construct
QVQLVQSGAEVKKPGASVKVSCKASRYTFTDYNLHWVRQAPGQGLEWMGFIYPYSGGTGYAQKFQGRVTMTVDTSTSTAYMELSSLRSEDTAVYYCARDHRYNEGVSFDYWGQGTTVTVSS

SEQ ID NO: 131 - protein, 121 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSS

SEQ ID NO: 132 - protein, 121 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSS

SEQ ID NO: 133 - protein, 121 aa, synthetic construct
QVQLVQSGAEVKKPGASVKVSCKASRYTFTDYNLHWVRQAPGQGLEWMGFIYPYDGGTGYAQKFQGRVTMTVDTSTSTAYMELSSLRSEDTAVYYCARDHRYNEGVSFDYWGQGTTVTVSS

SEQ ID NO: 134 - protein, 108 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARWGQGTLVTVS

SEQ ID NO: 135 - protein, 121 aa, synthetic construct
QVQLVQSGAEVKKPGASVKVSCKASRYTFTDYNLHWVRQAPGQGLEWMGFIYPYNGGTNYAQKFQGRVTITADTSTSTAYMELSSLRSEDTAVYYCARDHRYNEGVSFDYWGQGTLVTVSS

SEQ ID NO: 136 - protein, 121 aa, synthetic construct
QVQLVQSGAEVKKPGASVKVSCKASRYTFTDYNLHWVRQAPGQGLEWIGFIYPYNGGTGYAQKFQGRVTITADTSTSTAYMELSSLRSEDTAVYYCARDHRYNEGVSFDYWGQGTLVTVSS

SEQ ID NO: 137 - protein, 121 aa, synthetic construct
QVQLVQSGAEVKKPGASVKVSCKASRYTFTDYNLHWVRQAPGQGLEWIGFIYPYNGGTGYAQKFKGRVTITADTSTSTAYMELSSLRSEDTAVYYCARDHRYNEGVSFDYWGQGTLVTVSS

SEQ ID NO: 138 - protein, 121 aa, synthetic construct
QVQLVQSGAEVKKPGASVKVSCKASRYTFTDYNLHWVRQAPGQGLEWMGFIYPYNGGTGYAQKFQGRVTLTVDYSTSTAYMELSSLRSEDTAVYYCARDHRYNEGVSFDYWGQGTLVTVSS

SEQ ID NO: 139 - protein, 121 aa, synthetic construct
QVQLVQSGAEVKKPGASVKVSCKASRYTFTDYNLHWVRQAPGQGLEWIGFIYPYNGGTGYAQKFQGRVTLTVDYSTSTAYMELSSLRSEDTAVYYCARDHRYNEGVSFDYWGQGTLVTVSS

SEQ ID NO: 140 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 141 - protein, 111 aa, synthetic construct
EIVLTQSPGTLSLSPGERATLSCRASESVDGFGNSFMNWYQQKPGQAPRLLIYLASNLESGIPARFSGSGSGTDFTLTISRLEPEDFAVYYCQQNNEDPYTFGQGTKVEIK

SEQ ID NO: 142 - protein, 111 aa, synthetic construct
EIVLTQSPGTLSLSPGERATLSCRASESVDGFGNSFMNWYQQKPGQAPRLLIYLASNLESGVPARFSGSGSGTDFTLTISRLEPEDFAVYYCQQNNEDPYTFGQGTKVEIK

SEQ ID NO: 143 - protein, 111 aa, synthetic construct
EIVLTQSPGTLSLSPGERATLSCRASESVDGFGNSFMNWYQQKPGQAPRLLIYLASNLESGVPARFSGSGSRTDFTLTISRLEPEDFAVYYCQQNNEDPYTFGQGTKVEIK

SEQ ID NO: 144 - protein, 107 aa, synthetic construct [sequence omitted]

SEQ ID NO: 145 - protein, 330 aa, synthetic construct [sequence omitted]

SEQ ID NO: 146 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 147 - protein, 330 aa, synthetic construct [sequence omitted]

SEQ ID NO: 148 - protein, 10 aa, synthetic construct
  VARIANT 8: note = X is N, S, Q, or A
RYTFTDYXLH

SEQ ID NO: 149 - protein, 10 aa, synthetic construct
  VARIANT 6: note = X is N, S, Q, or A
FIYPYXGGTG

SEQ ID NO: 150 - protein, 10 aa, synthetic construct
  VARIANT 6: note = X is N, Q, D, S, or A
FIYPYXGGTG

SEQ ID NO: 151 - protein, 12 aa, synthetic construct
  VARIANT 5: note = X is N, Q, S, or A
DHRYXEGVSFDY

SEQ ID NO: 152 - protein, 15 aa, synthetic construct
  VARIANT 7: note = X is D, E, S, or A
  VARIANT 11: note = X is N, S, Q, or A
RASESVXGFGXSFMN

SEQ ID NO: 153 - protein, 7 aa, synthetic construct
  VARIANT 4: note = X is N, Q, D, S, or A
LASXLES

SEQ ID NO: 154 - protein, 9 aa, synthetic construct
  VARIANT 3: note = X is N, Q, D, S, or A
  VARIANT 6: note = X is D, E, S, or A
  VARIANT 4: note = X is N, Q, D, S, or A
QQXXEXPYT

SEQ ID NO: 155 - protein, 121 aa, synthetic construct
  VARIANT 33: note = X is N, S, Q, or A
  VARIANT 55: note = X is N, S, Q, or A
  VARIANT 103: note = X is N, S, Q, or A
QVQLVQSGAEVKKPGASVKVSCKASRYTFTDYXLHWVRQAPGQGLEWMGFIYPYXGGTGYAQKFQGRVTMTRDTSTSTVYMELSSLRSEDTAVYYCARDHRYXEGVSFDYWGQGTTVTVSS

SEQ ID NO: 156 - protein, 121 aa, synthetic construct; identical to SEQ ID NO: 155
  VARIANT 33: note = N is N, S, Q, or A
  VARIANT 55: note = X is N, Q, D, S, or A
  VARIANT 103: note = X is N, S, Q, or A

SEQ ID NO: 157 - protein, 111 aa, synthetic construct
  VARIANT 30: note = X is D, E, S, or A
  VARIANT 34: note = X is N, S, Q, or A
  VARIANT 95: note = X is N, Q, D, S, or A
  VARIANT 98: note = X is D, E, S, or A
  VARIANT 96: note = X is N, Q, D, S, or A
DIQMTQSPSSLSASVGDRVTITCRASESVXGFGXSFMNWYQQKPGKAPKLLIYLASXLESGVPSRFSGSGSGTDFTLTISSLQPEDFATYYCQQXXEXPYTFGQGTKLEIK

SEQ ID NO: 158 - protein, 120 aa, synthetic construct
  VARIANT 33: note = X is N, S, Q, or A
  VARIANT 55: note = X is N, S, Q, or A
  VARIANT 103: note = X is N, S, Q, or A
QVQLVQSGAEVKKPGASVKVSCKASRYTFTDYXLHWVRQAPGQGLEWMGFIYPYXGGTGYAQKFQGRVTMTRDTSTSTVYMELSSLRSEDTAVYYCARDHRYXEGVSFDYWGQGTLVTVS

SEQ ID NO: 159 - protein, 120 aa, synthetic construct; identical to SEQ ID NO: 158
  VARIANT 33: note = X is N, S, Q, or A
  VARIANT 55: note = X is N, Q, D, S, or A
  VARIANT 103: note = X isN, S, Q, or A

SEQ ID NO: 160 - protein, 111 aa, synthetic construct
  VARIANT 30: note = X is D, E, S, or A
  VARIANT 34: note = X is N, S, Q, or A
  VARIANT 57: note = X is N, Q, D, S, or A
  VARIANT 95: note = X is N, Q, D, S, or A
  VARIANT 98: note = X is D, E, S, or A
  VARIANT 96: note = X is N, Q, D, S, or A
EIVLTQSPGTLSLSPGERATLSCRASESVXGFGXSFMNWYQQKPGQAPRLLIYLASXLESGIPDRFSGSGSGTDFTLTISRLEPEDFAVYYCQQXXEXPYTFGQGTKVEIK

SEQ ID NO: 161 - protein, 10 aa, synthetic construct
FIYPYSGGTG

SEQ ID NO: 162 - protein, 10 aa, synthetic construct
FIYPYQGGTG

SEQ ID NO: 163 - protein, 10 aa, synthetic construct
FIYPYAGGTG

SEQ ID NO: 164 - protein, 450 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 218 aa, synthetic construct
DIQLTQSPSSLSASVGDRATITCRASESVDGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 111 aa, synthetic construct
DIQLTQSPSSLSASVGDRATITCRASESVEGFGNSFMNWYQQKPGKAPKLLIYLASNLESGVPSRFSGSGSGTDFTLTISSVQPEDFATYYCQQNNEDPYTFGQGTKLEIK

SEQ ID NO: 167 - protein, 111 aa, synthetic construct
DIQLTQSPSSLSASVGDRATITCRASESVSGFGNSFMNWYQQKPGKAPKLLIYLASNLESGVPSRFSGSGSGTDFTLTISSVQPEDFATYYCQQNNEDPYTFGQGTKLEIK

SEQ ID NO: 168 - protein, 111 aa, synthetic construct
DIQLTQSPSSLSASVGDRATITCRASESVAGFGNSFMNWYQQKPGKAPKLLIYLASNLESGVPSRFSGSGSGTDFTLTISSVQPEDFATYYCQQNNEDPYTFGQGTKLEIK

SEQ ID NO: 169 - protein, 121 aa, synthetic construct
QVQLVQSGAEVKKPGASVKVSCKASRYTFTDYNLHWVRQAPGQGLEWMGFIYPYSGGTGYAQKFQGRVTMTVDYSTSTAYMELSSLRSEDTAVYYCARDHRYNEGVSFDYWGQGTTVTVSS

SEQ ID NO: 170 - protein, 121 aa, synthetic construct
QVQLVQSGAEVKKPGASVKVSCKASRYTFTDYNLHWVRQAPGQGLEWMGFIYPYQGGTGYAQKFQGRVTMTVDYSTSTAYMELSSLRSEDTAVYYCARDHRYNEGVSFDYWGQGTTVTVSS

SEQ ID NO: 171 - protein, 121 aa, synthetic construct
QVQLVQSGAEVKKPGASVKVSCKASRYTFTDYNLHWVRQAPGQGLEWMGFIYPYAGGTGYAQKFQGRVTMTVDYSTSTAYMELSSLRSEDTAVYYCARDHRYNEGVSFDYWGQGTTVTVSS

SEQ ID NO: 172 - protein, 450 aa, synthetic construct
QVQLVQSGAEVKKPGASVKISCKASRYTFTDYNLHWVRQAPGQGLEWIGFIYPYNGGTGYAQKFQGRATLTVDTSTSTAYMELSSLRSEDTAVYYCARDHRYNEGVSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 450 aa, synthetic construct
QVQLVQSGAEVKKPGASVKISCKASRYTFTDYNLHWVRQAPGQGLEWIGFIYPYNGGTGYAQKFQGRATLTVDYSTSTAYMELSSLRSEDTAVYYCARDHRYNEGVSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 174 - protein, 218 aa, synthetic construct
DIQLTQSPSSLSASVGDRATITCRASESVDGFGNSFMNWYQQKPGKAPKLLIYLASNLESGVPSRFSGSGSRTDFTLTISSVQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 450 aa, synthetic construct
QVQLVQSGAEVKKPGASVKVSCKASRYTFTDYNLHWVRQAPGQGLEWMGFIYPYSGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 176 - protein, 450 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - protein, 450 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 178 - protein, 450 aa, synthetic construct
QVQLVQSGAEVKKPGASVKVSCKASRYTFTDYNLHWVRQAPGQGLEWMGFIYPYNGGTGYAQKFQGRVTMTVDYSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - protein, 111 aa, synthetic construct; identical to SEQ ID NO: 167

SEQ ID NO: 180 - protein, 107 aa, synthetic construct
RTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGES

SEQ ID NO: 181 - protein, 111 aa, synthetic construct
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 182 - protein, 218 aa, synthetic construct
DIQLTQSPSSLSASVGDRATITCRASESVDGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGES

SEQ ID NO: 183 - protein, 232 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 184 - protein, 110 aa, synthetic construct
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCP

SEQ ID NO: 185 - protein, 231 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCP

SEQ ID NO: 186 - protein, 108 aa, synthetic construct
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSCDKTHT

SEQ ID NO: 187 - protein, 229 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSCDKTHT

SEQ ID NO: 188 - protein, 232 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 189 - protein, 232 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 190 - protein, 218 aa, synthetic construct
DIQLTQSPSSLSASVGDRATITCRASESVSGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGES

SEQ ID NO: 191 - protein, 231 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCP

SEQ ID NO: 192 - protein, 231 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCP

SEQ ID NO: 193 - protein, 229 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSCDKTHT

SEQ ID NO: 194 - protein, 229 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSCDKTHT

SEQ ID NO: 195 - protein, 111 aa, synthetic construct; identical to SEQ ID NO: 124

SEQ ID NO: 196 - protein, 121 aa, synthetic construct; identical to SEQ ID NO: 118

SEQ ID NO: 197 - protein, 121 aa, synthetic construct; identical to SEQ ID NO: 131

SEQ ID NO: 198 - protein, 121 aa, synthetic construct; identical to SEQ ID NO: 132

SEQ ID NO: 199 - RNA, 1770 nt, synthetic construct
  source 1..1770: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgagactactgttatttgaccaatgaattgacttctaataaaatctagtggtgatgctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 200 - RNA, 1771 nt, synthetic construct
  source 1..1771: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 201 - RNA, 1771 nt, synthetic construct; identical to SEQ ID NO: 200
  source 1..1771: mol_type = other RNA

SEQ ID NO: 202 - RNA, 1891 nt, synthetic construct
  source 1..1891: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 203 - RNA, 1950 nt, synthetic construct
  source 1..1950: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtgcagctggtgcagtctggggctgaggtgaagaagcctgggtcctcggtgaaggtctcctgcaaggattctggaggcaccttcagcagctatgctatcagctgggtgcgacaggcccctggacaagggcttgagtggatgggagggatcatccctatctttggtacaacaaactacgcacagcagttccagggcagagtcacgattaccgcggacgaatccacgagcacagcctacatggagctgagcagcctgagatctgaggacacggccgtgtattactgtgcgagagaggccgtggccgccgactggctggacccctggggccagggaaccctggtcaccgtctcctcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 204 - RNA, 1822 nt, synthetic construct
  source 1..1822: mol_type = other RNA
agcataaaagactcactatttgttttcgcgcccagttgcaaaaagtgtcgtaattgactaagaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 205 - RNA, 1822 nt, synthetic construct
  source 1..1822: mol_type = other RNA
agcataaaaggcttctagcaataggctataattcttcatcgtgttgtaacaagcgaactctgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 206 - RNA, 1774 nt, synthetic construct
  source 1..1774: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 207 - RNA, 1761 nt, synthetic construct
  source 1..1761: mol_type = other RNA
agcataaaagctagcttttctcttctgtcaaccccacacgcctttggcacagaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 208 - RNA, 1840 nt, synthetic construct
  source 1..1840: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 209 - RNA, 1899 nt, synthetic construct
  source 1..1899: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtgcagctggtgcagtctggggctgaggtgaagaagcctgggtcctcggtgaaggtctcctgcaaggattctggaggcaccttcagcagctatgctatcagctgggtgcgacaggcccctggacaagggcttgagtggatgggagggatcatccctatctttggtacaacaaactacgcacagcagttccagggcagagtcacgattaccgcggacgaatccacgagcacagcctacatggagctgagcagcctgagatctgaggacacggccgtgtattactgtgcgagagaggccgtggccgccgactggctggacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 210 - RNA, 1822 nt, synthetic construct
  source 1..1822: mol_type = other RNA
agcataaaagggctgctccttgctatagaagacctgggacagaggactgctgtctgccctctctggtcaccctgcctagctagaggatctgtgaccccagccgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgcttgcagaaaaagaaaaatgagctcaaaatttgctttgagagctacagggaattgctattactcctgtaccttctgctcaatttcctttcctcatctcaaataaatgccttgttacaagatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 211 - RNA, 1771 nt, synthetic construct
  source 1..1771: mol_type = other RNA
agcataaaagactcactatttgttttcgcgcccagttgcaaaaagtgtcgtaattgactaagaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 212 - RNA, 1776 nt, synthetic construct
  source 1..1776: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 213 - RNA, 1774 nt, synthetic construct
  source 1..1774: mol_type = other RNA
agcataaaagtcacatttgcttctgacatagttgtgttgactcacaaccccagaaacagacatcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 214 - RNA, 1822 nt, synthetic construct
  source 1..1822: mol_type = other RNA
agcataaaagggctgctccttgctatagaagacctgggacagaggactgctgtctgccctctctggtcaccctgcctagctagaggatctgtgaccccagccgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgcttgcagaaaaagaaaaatgagctcaaaatttgctttgagagctacagggaattgctattactcctgtaccttctgctcaatttcctttcctcatctcaaataaatgccttgttacaagatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 215 - RNA, 1771 nt, synthetic construct
  source 1..1771: mol_type = other RNA
agcataaaagactcactatttgttttcgcgcccagttgcaaaaagtgtcgtaattgactaagaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 216 - RNA, 1771 nt, synthetic construct
  source 1..1771: mol_type = other RNA
agcataaaaggcttctagcaataggctataattcttcatcgtgttgtaacaagcgaactctgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 217 - RNA, 1720 nt, synthetic construct
  source 1..1720: mol_type = other RNA
agcataaaagtcacatttgcttctgacatagttgtgttgactcacaaccccagaaacagacatcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 218 - RNA, 1776 nt, synthetic construct
  source 1..1776: mol_type = other RNA
agcataaaagtcacatttgcttctgacatagttgtgttgactcacaaccccagaaacagacatcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 219 - RNA, 2352 nt, synthetic construct
  source 1..2352: mol_type = other RNA
agcataaaagtcacatttgcttctgacatagttgtgttgactcacaaccccagaaacagacatcgaattcgctgccaccatggagacagacaccctcctgctgtgggtgctgctcctgtgggtgcccggcagcaccggcgacatcgtgctgacccagagccccgccatcctgagcgccagccccggcgagaaggtgaccatgacctgccgggcctccagcagcgtgaactacatggactggtaccagaagaagcccggcagcagccccaagccctggatctacgccaccagcaacctggccagcggcgtgcccgcccggttcagcgggagcggcagcggcaccagctacagcctgaccatcagccgggtggaggccgaggacgccgccacctactactgccagcagtggagcttcaacccacccaccttcggaggcggcaccaagctggagatcaagggcagcaccagcggaggcggcagcggcggagggagcggaggcggaggcagcagcgaggtgcagctgcagcagagcggcgccgagctggtgaagcccggcgccagcgtgaagatgagctgcaaggccagcggctacaccttcaccagctacaacatgcactgggtgaagcagaccccaggccagggcctggagtggatcggcgccatctaccccggcaacggcgacacaagctacaaccagaagttcaagggcaaggccaccctgaccgccgacaagtccagcagcaccgcctacatgcagctgagcagcctgaccagcgaggacagcgccgattactactgcgcccggagcaactactacggcagcagctactggttcttcgacgtctggggcgccggcaccaccgtgaccgtgagcagcgagagcaagtacggcccaccctgcccgccctgccccgccccagagttcgacggcggccccagcgtgttcctgttcccacccaagcccaaggacaccctgatgatcagccggaccccagaggtgacctgcgtcgtggtggacgtgagccaggaggaccccgaggtgcagttcaactggtacgtggacggcgtggaggtgcacaacgccaagaccaagccccgggaggagcagttccagagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtctacaccctgccacccagccaggaggagatgaccaagaaccaggtgagcctgacctgcctggtgaagggcttctaccccagcgacatcgccgtggagtgggagagcaacggccagcccgagaacaactacaagaccaccccaccagtgctggacagcgacggcagcttcttcctctacagccggctgaccgtggacaagagccggtggcaggagggcaacgtcttcagctgcagcgtgatgcacgaggccctgcacaaccactacacccagaaaagcctgagcctcagcctgggcaagatgttctgggtgctggtcgtggtgggcggcgtgctggcctgctacagcctgctggtgaccgtggccttcatcatcttctgggtgaagcggggccggaagaagctgctctacatcttcaagcagcccttcatgcggcccgtgcagaccacccaggaggaagacggctgcagctgccggttccccgaggaggaagagggcggctgcgagctgcgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggaggaggggcaagggccacgacggcctgtaccagggcctgagcaccgccaccaaggacacctacgacgccctgcacatgcaggccctgccaccacggtgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 220 - RNA, 2481 nt, synthetic construct
  source 1..2481: mol_type = other RNA
agcataaaagtcacatttgcttctgacatagttgtgttgactcacaaccccagaaacagacatcgaattcgctgccaccatggagacagacaccctcctgctgtgggtgctgctcctgtgggtgcccggcagcaccggcgacatcgtgctgacccagagccccgccatcctgagcgccagccccggcgagaaggtgaccatgacctgccgggcctccagcagcgtgaactacatggactggtaccagaagaagcccggcagcagccccaagccctggatctacgccaccagcaacctggccagcggcgtgcccgcccggttcagcgggagcggcagcggcaccagctacagcctgaccatcagccgggtggaggccgaggacgccgccacctactactgccagcagtggagcttcaacccacccaccttcggaggcggcaccaagctggagatcaagggcagcaccagcggaggcggcagcggcggagggagcggaggcggaggcagcagcgaggtgcagctgcagcagagcggcgccgagctggtgaagcccggcgccagcgtgaagatgagctgcaaggccagcggctacaccttcaccagctacaacatgcactgggtgaagcagaccccaggccagggcctggagtggatcggcgccatctaccccggcaacggcgacacaagctacaaccagaagttcaagggcaaggccaccctgaccgccgacaagtccagcagcaccgcctacatgcagctgagcagcctgaccagcgaggacagcgccgattactactgcgcccggagcaactactacggcagcagctactggttcttcgacgtctggggcgccggcaccaccgtgaccgtgagcagcctggacgagagcaagtacggcccaccctgcccgccctgccccgccccagagttcctgggcggccccagcgtgttcctgttcccacccaagcccaaggacaccctgatgatcagccggaccccagaggtgacctgcgtcgtggtggacgtgagccaggaggaccccgaggtgcagttcaactggtacgtggacggcgtggaggtgcacaacgccaagaccaagccccgggaggagcagttcaacagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtctacaccctgccacccagccaggaggagatgaccaagaaccaggtgagcctgacctgcctggtgaagggcttctaccccagcgacatcgccgtggagtgggagagcaacggccagcccgagaacaactacaagaccaccccaccagtgctggacagcgacggcagcttcttcctctacagccggctgaccgtggacaagagccggtggcaggagggcaacgtcttcagctgcagcgtgatgcacgaggccctgcacaaccactacacccagaaaagcctgagcctcagcctgggcaagatgttctgggtgctggtcgtggtgggcggcgtgctggcctgctacagcctgctggtgaccgtggccttcatcatcttctgggtgcggagcaagcggagccggggcggccacagcgactacatgaacatgaccccacggcggcccggccccacccggaagcactaccagccctacgccccaccacgggacttcgccgcctaccggagcaagaggggccggaagaagctgctctacatcttcaagcagcccttcatgcggcccgtgcagaccacccaggaggaagacggctgcagctgccggttccccgaggaggaagagggcggctgcgagctgcgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggaggaggggcaagggccacgacggcctgtaccagggcctgagcaccgccaccaaggacacctacgacgccctgcacatgcaggccctgccaccacggtgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 221 - RNA, 2545 nt, synthetic construct
  source 1..2545: mol_type = other RNA
agcataaaagtcacatttgcttctgacatagttgtgttgactcacaaccccagaaacagacatcgaattcgctgccaccatggagacagacaccctcctgctgtgggtgctgctcctgtgggtgcccggcagcaccggcgacatcgtgctgacccagagccccgccatcctgagcgccagccccggcgagaaggtgaccatgacctgccgggcctccagcagcgtgaactacatggactggtaccagaagaagcccggcagcagccccaagccctggatctacgccaccagcaacctggccagcggcgtgcccgcccggttcagcgggagcggcagcggcaccagctacagcctgaccatcagccgggtggaggccgaggacgccgccacctactactgccagcagtggagcttcaacccacccaccttcggaggcggcaccaagctggagatcaagggcagcaccagcggaggcggcagcggcggagggagcggaggcggaggcagcagcgaggtgcagctgcagcagagcggcgccgagctggtgaagcccggcgccagcgtgaagatgagctgcaaggccagcggctacaccttcaccagctacaacatgcactgggtgaagcagaccccaggccagggcctggagtggatcggcgccatctaccccggcaacggcgacacaagctacaaccagaagttcaagggcaaggccaccctgaccgccgacaagtccagcagcaccgcctacatgcagctgagcagcctgaccagcgaggacagcgccgattactactgcgcccggagcaactactacggcagcagctactggttcttcgacgtctggggcgccggcaccaccgtgaccgtgagcagcctggacgagagcaagtacggcccaccctgcccgccctgccccgccccagagttcctgggcggccccagcgtgttcctgttcccacccaagcccaaggacaccctgatgatcagccggaccccagaggtgacctgcgtcgtggtggacgtgagccaggaggaccccgaggtgcagttcaactggtacgtggacggcgtggaggtgcacaacgccaagaccaagccccgggaggagcagttcaacagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtctacaccctgccacccagccaggaggagatgaccaagaaccaggtgagcctgacctgcctggtgaagggcttctaccccagcgacatcgccgtggagtgggagagcaacggccagcccgagaacaactacaagaccaccccaccagtgctggacagcgacggcagcttcttcctctacagccggctgaccgtggacaagagccggtggcaggagggcaacgtcttcagctgcagcgtgatgcacgaggccctgcacaaccactacacccagaaaagcctgagcctcagcctgggcaagatgttctgggtgctggtcgtggtgggcggcgtgctggcctgctacagcctgctggtgaccgtggccttcatcatcttctgggtgcggagcaagcggagccggggcggccacagcgactacatgaacatgaccccacggcggcccggccccacccggaagcactaccagccctacgccccaccacgggacttcgccgcctaccggagcaagaggggccggaagaagctgctctacatcttcaagcagcccttcatgcggcccgtgcagaccacccaggaggaagacggctgcagctgccggttccccgaggaggaagagggcggctgcgagctgcgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggaggaggggcaagggccacgacggcctgtaccagggcctgagcaccgccaccaaggacacctacgacgccctgcacatgcaggccctgccaccacggtgataaaggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 222 - RNA, 2481 nt, synthetic construct
  source 1..2481: mol_type = other RNA
agcataaaagtcacatttgcttctgacatagttgtgttgactcacaaccccagaaacagacatcgaattcgctgccaccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcactggacgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaaaagcctctccctgtctctgggtaaaatgttttgggtgctggtggtggttggtggagtcctggcttgctatagcttgctagtaacagtggcctttattattttctgggtgaggagtaagaggagcaggggcggccacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 223 - RNA, 2545 nt, synthetic construct
  source 1..2545: mol_type = other RNA
agcataaaagtcacatttgcttctgacatagttgtgttgactcacaaccccagaaacagacatcgaattcgctgccaccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcactggacgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaaaagcctctccctgtctctgggtaaaatgttttgggtgctggtggtggttggtggagtcctggcttgctatagcttgctagtaacagtggcctttattattttctgggtgaggagtaagaggagcaggggcggccacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 224 - RNA, 1788 nt, synthetic construct
  source 1..1788: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgacatcgtgatgacccagaccccacacagcagccccgtgaccctgggccagcccgccagcatcagctgccggagcagccagagcctggtgagccgggacggcaacacctacctgagctggctgcagcagcggcccggccagccaccccggctgctgatctacaagatcagcaaccggttcagcggcgtgcccaacaggttcagcggcagcggcgccggcaccgacttcaccctgaagatcagccgggtgaaggccgaggacgtgggcgtgtactactgcatgcaggccacccagttcccactgaccttcggccagggcacccggctggagatcaagggcggaggcggcagcggaggcggcgggagcggcggcggaggcagcgaggtgcagctggtgcagagcggcgccgaggtgaagaagcccggcgagagcctgaaaatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcagggcgacttctggagcggctacggcggcatggacgtctggggccagggcacaaccgtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccacgggacttcgccgcctaccggagcagggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccacggtgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 225 - RNA, 1852 nt, synthetic construct
  source 1..1852: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgacatcgtgatgacccagaccccacacagcagccccgtgaccctgggccagcccgccagcatcagctgccggagcagccagagcctggtgagccgggacggcaacacctacctgagctggctgcagcagcggcccggccagccaccccggctgctgatctacaagatcagcaaccggttcagcggcgtgcccaacaggttcagcggcagcggcgccggcaccgacttcaccctgaagatcagccgggtgaaggccgaggacgtgggcgtgtactactgcatgcaggccacccagttcccactgaccttcggccagggcacccggctggagatcaagggcggaggcggcagcggaggcggcgggagcggcggcggaggcagcgaggtgcagctggtgcagagcggcgccgaggtgaagaagcccggcgagagcctgaaaatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcagggcgacttctggagcggctacggcggcatggacgtctggggccagggcacaaccgtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccacgggacttcgccgcctaccggagcagggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccacggtgataaaggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 226 - RNA, 1788 nt, synthetic construct
  source 1..1788: mol_type = other RNA
agcataaaagtcacatttgcttctgacatagttgtgttgactcacaaccccagaaacagacatcgaattcgctgccaccatggccttaccagtgaccgccttgctcctgccgctggccttgctgctccacgccgccaggccggatattgtgatgacccagactccacactcctcacctgtcacccttggacagccggcctccatatcctgcaggtctagtcaaagcctcgtatccagagatggaaacacctacttgagttggcttcagcagaggccaggccagcctccaagactcctaatttataagatttctaaccggttctctggggtcccaaacagattcagtggcagtggggcagggacagatttcacactgaaaatcagcagggtgaaagctgaggatgtcggggtttattactgcatgcaagctacacagtttcccctcaccttcggccaagggacacgactggagattaaaggcggcggaggatctggcggaggtggtagtggcggaggcggatctgaggtgcagttggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacagggcgatttttggagtggttatgggggtatggacgtctggggccaagggaccacggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 227 - RNA, 1852 nt, synthetic construct
  source 1..1852: mol_type = other RNA
agcataaaagtcacatttgcttctgacatagttgtgttgactcacaaccccagaaacagacatcgaattcgctgccaccatggccttaccagtgaccgccttgctcctgccgctggccttgctgctccacgccgccaggccggatattgtgatgacccagactccacactcctcacctgtcacccttggacagccggcctccatatcctgcaggtctagtcaaagcctcgtatccagagatggaaacacctacttgagttggcttcagcagaggccaggccagcctccaagactcctaatttataagatttctaaccggttctctggggtcccaaacagattcagtggcagtggggcagggacagatttcacactgaaaatcagcagggtgaaagctgaggatgtcggggtttattactgcatgcaagctacacagtttcccctcaccttcggccaagggacacgactggagattaaaggcggcggaggatctggcggaggtggtagtggcggaggcggatctgaggtgcagttggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacagggcgatttttggagtggttatgggggtatggacgtctggggccaagggaccacggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 228 - RNA, 79 nt, Mus musculus
  source 1..79: mol_type = other RNA
agcataaaagtcacatttgcttctgacatagttgtgttgactcacaaccccagaaacagacatcgaattcgctgccacc

SEQ ID NO: 229 - RNA, 97 nt, unidentified
  source 1..97: mol_type = other RNA
agcataaaagctcggtcctgggagggtttaaaagccagggggccgtctcgacctcagtctgaccttccctcccggcagcagcgaattcgctgccacc

SEQ ID NO: 230 - RNA, 117 nt, unidentified
  source 1..117: mol_type = other RNA
agcataaaagggctgctccttgctatagaagacctgggacagaggactgctgtctgccctctctggtcaccctgcctagctagaggatctgtgaccccagccgaattcgctgccacc

SEQ ID NO: 231 - RNA, 66 nt, Homo sapiens
  source 1..66: mol_type = other RNA
agcataaaagctagcttttctcttctgtcaaccccacacgcctttggcacagaattcgctgccacc

SEQ ID NO: 232 - RNA, 65 nt, unidentified
  source 1..65: mol_type = other RNA
agcataaaagactagtattcttctggtccccacagactcagagagaacccgaattcgctgccacc

SEQ ID NO: 233 - RNA, 76 nt, synthetic construct
  source 1..76: mol_type = other RNA
agcataaaagactcactatttgttttcgcgcccagttgcaaaaagtgtcgtaattgactaagaattcgctgccacc

SEQ ID NO: 234 - RNA, 76 nt, synthetic construct
  source 1..76: mol_type = other RNA
agcataaaaggcttctagcaataggctataattcttcatcgtgttgtaacaagcgaactctgaattcgctgccacc

SEQ ID NO: 235 - RNA, 73 nt, synthetic construct
  source 1..73: mol_type = other RNA
agcataaaagaaataagagagaaaagaagagtaagaagaaatataagaccccggcgccgaattcgctgccacc

SEQ ID NO: 236 - RNA, 54 nt, Mus musculus
  source 1..54: mol_type = other RNA
tcacatttgcttctgacatagttgtgttgactcacaaccccagaaacagacatc

SEQ ID NO: 237 - RNA, 72 nt, unidentified
  source 1..72: mol_type = other RNA
ctcggtcctgggagggtttaaaagccagggggccgtctcgacctcagtctgaccttccctcccggcagcagc

SEQ ID NO: 238 - RNA, 92 nt, unidentified
  source 1..92: mol_type = other RNA
ggctgctccttgctatagaagacctgggacagaggactgctgtctgccctctctggtcaccctgcctagctagaggatctgtgaccccagcc

SEQ ID NO: 239 - RNA, 41 nt, Homo sapiens
  source 1..41: mol_type = other RNA
ctagcttttctcttctgtcaaccccacacgcctttggcaca

SEQ ID NO: 240 - RNA, 40 nt, unidentified
  source 1..40: mol_type = other RNA
actagtattcttctggtccccacagactcagagagaaccc

SEQ ID NO: 241 - RNA, 51 nt, synthetic construct
  source 1..51: mol_type = other RNA
actcactatttgttttcgcgcccagttgcaaaaagtgtcgtaattgactaa

SEQ ID NO: 242 - RNA, 51 nt, synthetic construct
  source 1..51: mol_type = other RNA
gcttctagcaataggctataattcttcatcgtgttgtaacaagcgaactct

SEQ ID NO: 243 - RNA, 48 nt, synthetic construct
  source 1..48: mol_type = other RNA
aaataagagagaaaagaagagtaagaagaaatataagaccccggcgcc

SEQ ID NO: 244 - RNA, 69 nt, unidentified
  source 1..69: mol_type = other RNA
aggatccgagactactgttatttgaccaatgaattgacttctaataaaatctagtggtgatgctgtaca

SEQ ID NO: 245 - RNA, 70 nt, unidentified
  source 1..70: mol_type = other RNA
aggatccagttcagacttcaaatagtggcaaataaaaagtgctatttgtgatggtttgcttctgtgtaca

SEQ ID NO: 246 - RNA, 126 nt, unidentified
  source 1..126: mol_type = other RNA
aggatccagttcagacttcaaatagtggcaaataaaaagtgctatttgtgatggtttgcttctggagactactgttatttgaccaatgaattgacttctaataaaatctagtggtgatgctgtaca

SEQ ID NO: 247 - RNA, 126 nt, unidentified
  source 1..126: mol_type = other RNA
aggatccgagactactgttatttgaccaatgaattgacttctaataaaatctagtggtgatgcagttcagacttcaaatagtggcaaataaaaagtgctatttgtgatggtttgcttctgtgtaca

SEQ ID NO: 248 - RNA, 124 nt, Homo sapiens
  source 1..124: mol_type = other RNA
aggatccgctggagcctcggtggccatgcttcttgccccttgggcctccccccagcccctcctccccttcctgcacccgtacccccgtggtctttgaataaagtctgagtgggcggcatgtaca

SEQ ID NO: 249 - RNA, 190 nt, unidentified
  source 1..190: mol_type = other RNA
aggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtaca

SEQ ID NO: 250 - RNA, 109 nt, unidentified
  source 1..109: mol_type = other RNA
aggatccgctgaccctttgacacccttcttgtcctcctctctggccccatccaggcaaccaaataaagtttgagtgtaccaggaacagaatcctggggcttgctgtaca

SEQ ID NO: 251 - RNA, 134 nt, unidentified
  source 1..134: mol_type = other RNA
aggatccgcttgcagaaaaagaaaaatgagctcaaaatttgctttgagagctacagggaattgctattactcctgtaccttctgctcaatttcctttcctcatctcaaataaatgccttgttacaagatgtaca

SEQ ID NO: 252 - RNA, 296 nt, unidentified
  source 1..296: mol_type = other RNA
aggatccctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggatgtaca

SEQ ID NO: 253 - RNA, 56 nt, unidentified
  source 1..56: mol_type = other RNA
gagactactgttatttgaccaatgaattgacttctaataaaatctagtggtgatgc

SEQ ID NO: 254 - RNA, 57 nt, unidentified
  source 1..57: mol_type = other RNA
agttcagacttcaaatagtggcaaataaaaagtgctatttgtgatggtttgcttctg

SEQ ID NO: 255 - RNA, 113 nt, unidentified
  source 1..113: mol_type = other RNA
agttcagacttcaaatagtggcaaataaaaagtgctatttgtgatggtttgcttctggagactactgttatttgaccaatgaattgacttctaataaaatctagtggtgatgc

SEQ ID NO: 256 - RNA, 113 nt, unidentified
  source 1..113: mol_type = other RNA
gagactactgttatttgaccaatgaattgacttctaataaaatctagtggtgatgcagttcagacttcaaatagtggcaaataaaaagtgctatttgtgatggtttgcttctg

SEQ ID NO: 257 - RNA, 111 nt, Homo sapiens
  source 1..111: mol_type = other RNA
gctggagcctcggtggccatgcttcttgccccttgggcctccccccagcccctcctccccttcctgcacccgtacccccgtggtctttgaataaagtctgagtgggcggca

SEQ ID NO: 258 - RNA, 177 nt, unidentified
  source 1..177: mol_type = other RNA
caaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggca

SEQ ID NO: 259 - RNA, 96 nt, unidentified
  source 1..96: mol_type = other RNA
gctgaccctttgacacccttcttgtcctcctctctggccccatccaggcaaccaaataaagtttgagtgtaccaggaacagaatcctggggcttgc

SEQ ID NO: 260 - RNA, 121 nt, unidentified
  source 1..121: mol_type = other RNA
gcttgcagaaaaagaaaaatgagctcaaaatttgctttgagagctacagggaattgctattactcctgtaccttctgctcaatttcctttcctcatctcaaataaatgccttgttacaaga

SEQ ID NO: 261 - RNA, 283 nt, unidentified
  source 1..283: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctgga

SEQ ID NO: 262 - protein, 119 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLG

SEQ ID NO: 263 - protein, 218 aa, synthetic construct; identical to SEQ ID NO: 165

SEQ ID NO: 264 - protein, 240 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLG

(19) United States
(12) Patent Application Publication
(10) Pub. No.: US 2025/0302763 A1
(43) Pub. Date: Oct. 2, 2025

(54) IMMUNE ENGINEERING AMPLIFICATION

(71) Applicant: Capstan Therapeutics, Inc., San Diego, CA (US)

(72) Inventors: Adrian Ion Bot, Beverly Hills, CA (US); Haig Aghajanian, Lafayette Hill, PA (US); Michael Rosenzweig, Newton, MA (US); Yan Zhang, Poway, CA (US); Stuart Aaron Sievers, Van Nuys, CA (US); Ramin Farzaneh-Far, Brookline, MA (US)

(21) Appl. No.: 19/061,978

(22) Filed: Feb. 24, 2025

Related U.S. Application Data
(60) Provisional application No. 63/556,735, filed on Feb. 22, 2024.
(60) Provisional application No. 63/708,513, filed on Oct. 17, 2024.
(60) Provisional application No. 63/721,154, filed on Nov. 15, 2024.

(51) Int. Cl. A61K 9/51; A61K 39/44

(52) U.S. Cl. CPC A61K 9/5123; A61K 39/44; A61K 2039/5158; A61K 2039/545

(57) ABSTRACT

This disclosure provides methods of increasing in vivo transfection efficiency and pharmacologic activity of T cells, by administering multiple small doses within a compact time period of T cell-targeted lipid nanoparticles encapsulating mRNA encoding an antigen receptor that recognizes an antigen of a cell against which immune activity is to be directed. Also provided are methods of depleting B cells, and methods of treating B cell-mediated diseases and disorders by depleting B cells and achieving immunological reset, entailing administration of immune cell-targeted lipid nanoparticles encapsulating mRNA encoding an antigen receptor recognizing a B cell marker as multiple small doses within a compact time period. The antigen receptor can be a T cell receptor or a chimeric antigen receptor.

### CROSS-REFERENCE TO RELATED APPLICATIONS

This application claims priority to U.S. provisional application No. 63/556,735, filed Feb. 22, 2024; U.S. provisional application No. 63/708,513, filed Oct. 17, 2024; and U.S. provisional application No. 63/721,154, filed Nov. 15, 2024; the disclosures of each of which are expressly incorporated by reference herein.

### REFERENCE TO AN ELECTRONIC SEQUENCE LISTING

The instant application contains a Sequence Listing that has been submitted electronically and is hereby incorporated by reference in its entirety. The Sequence Listing was created on Feb. 21, 2025, is named “24-0260-US_Sequence-Listing_ST26.xml”, and is 327,469 bytes in size.

### BACKGROUND OF THE DISCLOSURE

CAR-T therapy (that is, therapy utilizing T cells expressing a chimeric antigen receptor) is a revolutionary and potentially curative therapy for patients with hematologic cancers. To date, there are six FDA approved ex vivo autologous CAR-T cell therapies on the U.S. market for the treatment of various B cell malignancies and hundreds more autologous and allogeneic CAR-T and CAR natural killer (NK) products being tested in clinical trials across the world (Wang et al., Cancers (Basel) 15(4): 1003, 2021). These ex vivo cell therapies have shown remarkable success in providing durable responses to patients with advanced and refractory cancers (Cappell et al., Nat Rev Clin Oncol 20: 359-371, 2023; Melenhorst et al., Nature 602: 503-509, 2022). Unfortunately, access to these therapies has been limited by challenges in manufacturing (costs, time, scaling), geography, number of specialized CAR T centers, the need for lymphodepleting chemotherapy, and safety concerns of an integrative approach (Gajra et al., Pharmaceut Med 36:163-171, 2022).

Because many of the CAR methods involve the use of autologous T cells, it is a significant limitation of the technology that patients may have T cells that are damaged or weakened due to prior chemotherapy or hematopoietic stem-cell transplantation. These compromised T cells may not proliferate well during manufacturing or may produce cells with insufficient potency that cannot be used for patient treatment. This can result in manufacturing failures or poor expansion and activity in patients. Additionally, the individualized nature of autologous manufacturing, together with the variability in patients' T cells, may lead to variable potency of manufactured T cells produced thereby. This variability may cause unpredictable treatment outcomes.

The entire CAR-T cell manufacturing process is dependent on the viability of each patient's T cells and, for the approved CAR-T cell therapies, takes approximately two to four weeks. As a result, up to 31% of intended patients did not receive treatment during the registrational trials for Yescarta and Kymriah (commercial CAR-T products) because of interval complications from underlying disease during manufacturing or due to manufacturing failures. Patients also must undergo lymphodepletion prior to CAR-T cells being administered to facilitate engraftment of the CAR-T cells. However, many potential patients are too ill to undergo the lymphodepletion regimen.

Allogeneic ex vivo CAR-T therapy is an emerging treatment that has attempted to overcome these limitations of access and manufacturing scalability. In contrast to autologous ex vivo CAR-T therapy, which uses a patient's own immune cells, allogeneic approaches use immune cells from a donor, which are then genetically modified to express a CAR protein to attack cancer cells. However, the need to avoid host reaction to the allogeneic cells adds to the complexity of the treatment and the procedure still requires lymphodepletion.

Thus, there remains a need in the art for methods of generating in vivo engineered lymphocytes with greater efficiency, effectiveness, and safety.

### SUMMARY

In certain aspects, this disclosure provides methods of immune engineering amplification and associated methods of treatment in a compact administration regimen wherein plural doses of a T cell-targeted lipid nanoparticle (tLNP) encapsulating mRNA encoding a T cell antigen receptor are administered to a subject so that each subsequent dose after an initial dose is administered within 1 to 5 days after the immediately previous dose. Among other advantages, these compact regimens can make use of smaller individual and/or cumulative dosages than would be needed to achieve similar effects using other administration schedules. Such regimens achieve a greater transfection efficiency, pharmacologic or clinical effect, and/or safety, as compared to an administration regimen using a comparatively larger dose administered as a single dose or multiple doses at an interval of ≥7 days. In some embodiments of the compact regimen all doses of a therapeutic cycle are administered within ≤5, ≤6, ≤7, or ≤8 days of the initial dose.

In further aspects, a compact administration regimen further comprises administration of a conditioning biological response modifier (BRM), such as a γ-chain receptor cytokine, for example IL-2, IL-7 or IL-15, or a pan-activating cytokine such as interleukin-12 (IL-12) or IL-18, or an immune checkpoint inhibitor such as an antagonist of cytotoxic T-lymphocyte associated protein 4 (CTLA-4), programmed cell death protein 1 (PD-1), program cell death ligand 1 (PD-L1), T-cell immunoglobulin and mucin-domain-containing-3 (Tim-3), lymphocyte activation gene 3 (LAG-3) or indoleamine 2,3-dioxygenase (IDO) or agonists of 4-immunoglobulin and BB cell surface glycoprotein (4-1in), OX40 or inducible costimulator (ICOS), in addition to administration of the tLNP. Typically, the BRM is administered 2-5 days prior to administration of the tLNP, although in some embodiments the BRM can also be administered concurrently with the tLNP. In some embodiments, conditioning agent administration enables lower individual and/or total dosages than would otherwise be required to achieve a similar effect. In other embodiments, conditioning agent administration replaces the initial dose of tLNP so that the total number of doses in the compact administration regimen is reduced, in some instances to a single dose of tLNP or single dose per therapeutic cycle. In some embodiments, conditioning agent administration facilitates use of multiple therapeutic cycles. In some embodiments, conditioning agent administration facilitates augmentation of biological and clinical response by co-opting additional effector mechanisms with additive or synergistic effect (such as endogenous immunity—adaptive or innate immune mechanisms).

These and other features, objects, and advantages of this invention will become better understood from the description that follows. In the description, reference was made to the accompanying drawings, which form a part hereof and in which there was shown by way of illustration, not limitation, embodiments of the invention. The description of preferred embodiments was not intended to limit the invention to cover all modifications, equivalents, and alternatives. Reference should therefore be made to the claims recited herein for interpreting the scope of the invention.

### BRIEF DESCRIPTION OF THE DRAWINGS

The patent or patent application file contains at least one drawing in color. Copies of this patent or patent application publication with color drawings will be provided by the Office upon request and payment of the necessary fee.

The disclosure will be better understood and features, aspects, and advantages other than those set forth above will become apparent when consideration was given to the following detailed description thereof. Such detailed description refers to the following drawings.

FIG. 1A depicts the developmental stages of B cell maturation and the expression of three B cell lineage antigens, CD19, CD20, and BCMA (B cell maturation antigen), across the various stages. Any cells expressing such antigens can be referred to generically as B cells. When referring to “B cell depletion” any or all cells in this developmental B cell lineage can be meant. “B cell mediated autoimmunity” and the like refers to a role for any or all non-naïve B cells.

FIG. 1B shows B cell numbers (cells per μL) in cultures of human peripheral blood mononuclear cells (PBMCs) from two donors following treatment with CD8-targeted tLNPs encapsulating mRNA encoding an anti-CD19 (tLNP-98219) or anti-CD20 CAR (tLNP-982520), or mCherry. Data for a 0.6 μg dosage is shown. Donor numbering is arbitrary and does not carry over from one experiment to another.

FIGS. 2A-2B show immunohistochemistry staining with an anti-CD20 antibody of spleen sections 9 days after a first infusion of CD8-targeted tLNPs encapsulating mRNA encoding a pharmacologically active anti-CD20 CAR according to different administration schedules, dosages, and cumulative dosages as indicated.

FIG. 3 depicts the percentage of leukocytes (CD45⁺ cells) that are B cells in bone marrow, liver, lymph node, and spleen, as determined by flow cytometry, after administration of CD8-targeted tLNPs encapsulating mRNA encoding a pharmacologically active anti-CD20 CAR (or PBS control) according to the indicated schedule and dose in two individual cynomolgus macaques for each schedule. X indicates that a sample was not available.

FIG. 4 depicts the enumeration of CAR-expressing CD8⁺ T cells after administration of CD8-targeted tLNPs encapsulating mRNA encoding a pharmacologically active anti-CD20 CAR according to the indicated dosages and administration schedules in a set of cynomolgus macaques (CM01-CM18) as the number of cells per μl (upper panels) or percentage of the total CD8⁺ T cell population (lower panels). The dashed line indicates when the dose was administered.

FIG. 5 depicts time courses of cytokine expression, specifically IL-6, monocyte chemoattractant protein 1 (MCP-1), interferon γ (IFNγ), and tumor necrosis factor α (TNFα), following the first (or only) infusion of CD8-targeted tLNPs encapsulating mRNA encoding a pharmacologically active anti-CD20 CAR (or a control anti-CD19 CAR) to cynomolgus macaques according to the indicated schedule, dose, and number of animals (n).

FIGS. 6A-6B depict CAR expression levels in CD4+ (FIG. 6A) and CD8+ (FIG. 6B) T cells 24 hours after in vitro transfection of human resting T cells from multiple individual donors with CD8-targeted tLNPs encapsulating mRNA encoding an anti-CD19 CAR.

FIG. 7 depicts expression levels of activation markers, specifically 4-1BB, CD25, CD69. PD-1, and TIM3, for the transfected T cells receiving the 0.6 μg dose in FIG. 6A and FIG. 6B as compared to T cells from the same donors transfected with CD8-targeted tLNPs encapsulating mRNA encoding mCherry and untransfected cells that had been co-cultured for 24 hours with NALM6 cells (CD19⁺), Raji cells (CD19⁺), K562 cells (CD19⁻), or no additional cells. Only the cells transfected to express the anti-CD19 CAR and co-cultured with CD19⁺ cells showed above background levels of activation marker expression (denoted by the box surrounding these results). Donor numbers are arbitrarily assigned and do not necessarily carry over from one experiment to another. Each shape represents an individual donor and duplicate samples are plotted

FIG. 8 depicts in vitro transfection efficiency of unactivated and activated-expanded T cells from two human donors transfected with CD8-targeted tLNPs encapsulating mRNA encoding an anti-CD19 CAR as the percentage of cells expressing the CAR.

FIGS. 9A-9C depict B cell repopulation of various B cell subsets in individual cynomolgus macaques following B cell depletion by anti-CD20 CAR-expressing T cells reprogrammed with CD8-targeted tLNPs encapsulating mRNA encoding a pharmacologically active anti-CD20 CAR. FIG. 9A depicts concentration (cells/μL) of different subsets of B cells in the blood before and at various time points after transfection using an immune engineering amplification regimen (1 mg/kg 3xQ72h). FIG. 9B depicts the level of B cell depletion by B cell subset in four tissues, specifically bone marrow, liver, lymph node, and spleen, at 9 days after first infusion of CD8-targeted tLNPs encapsulating mRNA encoding a pharmacologically active anti-CD20 CAR with 0.1, 0.3, or 1 mg/kg, 3xQ72h and repopulation at 63 days after first infusion for the 1 mg/kg dose. In both blood and tissue, these data showed that repopulation was predominantly by naïve B cells. FIG. 9C is an idealized depiction of treatment of autoimmunity with this mode of B cell depletion.

FIG. 10 portrays three schematic protocols for the integration of standard of care treatments of autoimmunity with the herein disclosed compact regimens for administering T cell-targeted tLNP encapsulating mRNA encoding a T cell antigen receptor (such as a CAR, T cell receptor (TCR), or T cell engager (TCE)) with specificity of a B cell surface antigen for improved treatment of autoimmunity.

FIGS. 11A-11E depict phenotyping of PBMCs from autoimmune and healthy donors as determined by staining with fluorescently labeled antibodies and flow cytometry. FIG. 11A are bar graphs depicting surface antigen density for CD8 and CD19 on CD8+ (top panel) and CD19+ (bottom panel) cells, respectively, for PBMCs from DM (dermatomyositis), MS (multiple sclerosis), rheumatoid arthritis (RA), scleroderma, and SLE (systemic lupus erythematosus) and patients and healthy volunteers. Each circle represents an individual donor, either as the average of two replicates or a single data point when there were an insufficient number of cells for a replicate. FIG. 11B depicts average proportions of major lymphocyte subsets from select donors with the indicated disease but excluding donors lacking B cells due to prior treatment with rituximab. The order of cell types represented in the bars in FIG. 11B is, from top to bottom: NKT cells, classical monocytes, intermediate monocytes, non-classical monocytes, dendritic cells, NK cells, B cells, CD4+ T cells, and CD8+ T cells. FIGS. 11C-11E depict the proportions of subsets within the CD4+ (FIG. 11C), CD8+ (FIG. 11D), and monocyte (FIG. 11E) populations from donors with the indicated diseases. The order of cell types represented in the bars in FIG. 11C is, from top to bottom: CD4+ Naïve, CD4+ T central memory (TCM), CD4+ T effector memory (TEM), and CD4+ T effector memory cells re-expressing CD45RA (TEMRA) T cells. FIGS. 11C-11D depict the average proportions of major lymphocyte subsets from select donors with the indicated disease but excluding donors lacking B cells due to prior treatment with rituximab. FIG. 11E depicts the proportions of monocytes in a selection of individual donors as identified in Table 14. The order of cell types represented in the bars in FIG. 11D is, from top to bottom: CD8+ Naïve, CD8+ TCM, CD8+ TEM, CD8+ TEMRA T cells. The order of cell types represented in the bars in FIG. 11E is, from top to bottom: Dendritic cells (DC) {CD14− CD16−}, Non-classical {CD14− CD16+}, Intermediate {CD14+CD16+}, Classical {CD14+CD16−} monocytes. The various donors are identified by disease and donor number (see Table 14). Where a donor number is followed by a letter, the different letters indicate distinct donations. Error bars represent standard deviation.

FIGS. 12A-12C depict the results of transfecting donor PBMCs with CD8-targeted tLNP encapsulating an mRNA encoding an anti-CD19 CAR. FIG. 12A depicts the number of CAR molecules expressed per cell 24 hours post transfection in CD8+ (first panel) and CD4+(second panel) cells, as well as the percentage of CD8+ (third panel) and CD4+ (fourth panel) T cells that were CAR+, grouped by donor disease. FIG. 12B depicts the number of total B cells 24 hours (first panel) and 72 hours (second panel) after transfection with CD8-targeted tLNP encapsulating an mRNA encoding an anti-CD19 CAR for PBMCs from the indicated donors versus non-transfected controls (NTD). tLNP-98219: CD8-targeted tLNP encapsulating an mRNA encoding an anti-CD19 CAR. FIG. 12C depicts the percentage of B cells killed for the same samples as in FIG. 12B. In FIGS. 12A-12C, each circle represents a distinct donor with blood samples collected at a distinct time. Donors lacking B cells due to prior treatment with rituximab were excluded from this analysis.

FIGS. 13A-13B depict expression of T cell activation markers by CD8+ and CD4+ cells and cytokine secretion 24 hours after transfection of PBMC cultures. In FIG. 13A the first panel shows CD25 expression in CD8⁺ cells, and the second panel shows CD69 expression in CD8+ cells. The third panel shows CD25 expression in CD4+ cells, and the fourth panel shows CD69 expression in CD4+ cells. FIG. 13B shows cytokine secretion in the PBMC cultures post transfection with CD8-targeted tLNP encapsulating an mRNA encoding an anti-CD19 CAR. The first panel shows IFN-γ secretion, and the second panel shows TNF-α secretion. Cytokine production is reported in pg/mL of culture supernatant. As in FIG. 12A-FIG. 12C, each circle represents a distinct donor with blood samples collected at a distinct time. Donors lacking B cells due to prior treatment with rituximab were excluded from this analysis.

FIG. 14 illustrates the experimental scheme for PBMC and pan T cell transfected a first time with mRNA encoding either luciferase or an anti-CD19 CAR and a second time with either mCherry or an anti-BCMA CAR.

FIGS. 15A-15B display flow cytometry histograms for expression in CD8+ cells of an anti-BCMA CAR (FIG. 15A) or mCherry (FIG. 15B) in PBMC (left panels) and pan T cells (right panels) from two human donors (upper vs. lower panels) transfected a first time with CD8-targeted tLNP encapsulating mRNA encoding either luciferase or an anti-CD19 CAR and 72 hours later transfected a second time with nothing (NTD), CD5-targeted tLNP encapsulating mRNA encoding mCherry, or CD8-targeted tLNP encapsulating mRNA encoding an anti-BCMA CAR. Each panel is a composite of four histograms traces: luciferase transfection followed by no transfection (Luc+NTD), anti-CD19 CAR transfection followed by no transfection (CD19−CAR+NTD), luciferase transfection followed by anti-BCMA CAR transfection (Luc+BCMA-CAR tLNP), and anti-CD19 CAR transfection followed by anti-BCMA CAR transfection (CD19−CAR+BCMA-CAR tLNP), top to bottom, respectively.

FIGS. 16A-16B display flow cytometry histograms for expression in CD48+ cells of an anti-BCMA CAR (FIG. 16A) or mCherry (FIG. 16B) in PBMC (left panels) and pan T cells (right panels) from two human donors (upper vs. lower panels) transfected a first time with CD8-targeted tLNP encapsulating mRNA encoding either luciferase or an anti-CD19 CAR and 72 hours later transfected a second time with nothing (NTD), CD5-targeted tLNP encapsulating mRNA encoding mCherry, or CD8-targeted tLNP encapsulating mRNA encoding an anti-BCMA CAR. Each panel is a composite of four histograms traces: luciferase transfection followed by no transfection (Luc+NTD), anti-CD19 CAR transfection followed by no transfection (CD19−CAR+NTD), luciferase transfection followed by anti-BCMA CAR transfection (Luc+BCMA-CAR tLNP), and anti-CD19 CAR transfection followed by anti-BCMA CAR transfection (CD19−CAR+BCMA-CAR tLNP), top to bottom, respectively.

FIGS. 17A-17B display tumor cell killing curves for co-cultures of BCMA⁺ CD19⁻ tumor cell lines (RPMI8226 and K562) constitutively expressing both luciferase and GFP with the various transfected cells. Tumor cell growth as total GFP area was monitored for ˜96 hours after the coculture was initiated at 24 hours after the second transfection.

FIGS. 18A-18D depict the results of in vivo transfection of mice administered a mouse CD8-targeted tLNP encapsulating an anti-CD19 CAR as determined by flow cytometry. FIGS. 18A-18C present the percent of cells expressing the CAR in four tissues, spleen (upper right panels), blood (upper left panels), bone marrow (lower left panels), and lymph node (lower right panels) for cytolytic T lymphocytes (CD4⁻ T cells; FIG. 18A), CD4⁺ T cells (FIG. 18B), and B cells (FIG. 18C). FIG. 18D depicts CAR expression level as median fluorescence intensity.

FIGS. 19A-19B show percentages and numbers of B cells in the spleen of NSG immunodeficient mice engrafted with human PBMCs at the indicated time after administration of tLNPs. FIG. 19A shows B cells in spleen as a percentage of human immune cells (CD45+) in mice treated with CD8-targeted tLNP encapsulating either mCherry mRNA or an anti-CD19 CAR mRNA (RM_61416). FIG. 19B shows total B cell counts per microliter of splenic single cell suspension (calculated using counting beads in the same samples). Each dot represents one animal, bar height is mean, and wickers indicate standard deviation. Samples with low viability (less than 5% live cells in the single suspension) were excluded from the analysis.

FIGS. 20A-20F show mCherry and CAR expression in CD8+ T cells in NSG immunodeficient mice engrafted with human PBMCs at the indicated time after administration of tLNPs. FIG. 20A shows frequency of mCherry+ CD8+ and CD4+ T cells in mice treated with CD8-targeted tLNP encapsulating mCherry mRNA. FIG. 20B shows mCherry median fluorescence intensity (MFI) in the mCherry+ CD8+ T cells (n=5 per group). FIG. 20C shows frequency of CAR expression in CD8+ and CD4+ T cells in mice treated with CD8-targeted tLNP encapsulating anti-CD19 CAR mRNA (RM_61416). FIG. 20D shows CAR median fluorescence intensity (MFI) in the CAR+ CD8+ T cells (n=10 per group). FIG. 20E shows quantification of CAR molecules per CAR+ CD8+ T cell based on MESF (n=10/group). FIG. 20F shows the correlation between rising CAR expression level and B cell depletion. All results were obtained from spleen samples. Each dot in FIGS. 20A through 20E represents one animal, bar height is mean, and error bars indicate standard deviation. Mice with less than 5% live human CD45+ cells in the spleen were excluded from this analysis.

FIGS. 21A-21B show B cell frequency and CAR expression after treatment of NCG immunodeficient mice engrafted with human PBMCs with CD8-targeted tLNP encapsulating anti-CD19 CAR mRNA (RM_61416) compared to controls. FIG. 21A shows percentage of B cells in spleen (upper panel) and blood (lower panel) after treatment with CD8-targeted tLNP encapsulating mCherry mRNA (7.5 μg), anti-CD19 CAR mRNA (RM_61416) (indicated doses), or PBS control. Each dot represents one animal. Frequency of B cells was measured by the % of B cells in human CD45+ cells. FIG. 21B shows CAR expression in the blood and spleen in the different dosage and treatment groups. N=5 mice in the mCherry mRNA-treated group and 10 mice in each of the anti-CD19 CAR mRNA (RM_61416)- and PBS-treated groups.

FIGS. 22A-22B show evaluation of B cell depletion and recovery time course across treatments (with CD8-targeting tLNPs encapsulating mRNA encoding anti-CD19 CAR, human anti-CD20 CAR, or mCherry), dose regimens, and time points. The O days post treatment data are from pre-treatment samples. FIG. 22A shows longitudinal measure of B cell frequency within human CD45 cells in the blood of NSG mice engrafted with human CD34+ cells. Individual animals shown for each dose regimen (daily, every 2 days, and every 3 days). FIG. 22B shows frequency of B cells at the terminal time point (Day 21 post final administration) for each dose regimen.

FIG. 23 shows B cell composition based on CD19 and CD20 expression. Each bar graph for the blood, spleen, and bone marrow presents the composition of the human CD45+CD3− fraction on each tissue, composed by four subsets based on CD19 and CD20 expression (in order from top to bottom of the bars): non-B cells (CD19−CD20−), CD19−CD20+B cells, CD19+CD20− B cells, and CD19+CD20+B cells.

FIGS. 24A-24B show the frequency of B cell subsets for each dose regimen (daily, every 2 days, every 3 days) in spleen (FIG. 24A) and bone marrow (FIG. 24B) 21 days after treatment. The percentage of each subset was calculated based on a parent population of CD19⁺ B cells.

FIGS. 25A-25B depict B cell depletion in cynomolgus macaques following treatment with a CD8-targeted tLNP encapsulating an mRNA encoding anti-CD20 CAR (tLNP-982520) that was physiologically active in the monkeys according to the indicated schedules and dosages. FIG. 25A reports the number of B cells per microliter of blood pre- and post-treatment. Blood samples were collected 6 hours after the first (or only) administration and on various days thereafter, as indicated. FIG. 25B reports the percentage of CD45+ cells that were B cells when the animals were sacrificed (i.e., the day the last blood sample was collected) for animals receiving 1 dose of 2.0 mg/kg, 2 doses of 1.0 or 1.5 mg/kg, 2 doses with a step up from 1.0 to 2.0 mg/kg in the 2nd dose, or 3 doses of 1 mg/kg. Each circle represents an individual animal.

FIGS. 25C-25D depict the rapid and transient rise of the T cell activation markers ICOS (FIG. 25C) and PD-1 (FIG. 25D) in CD8+ T cells (and the considerably lesser effect in CD4+ T cells) following 2 or 3 administrations of the indicated dosages of tLNP-982520 as baseline subtracted percent positive cells averaged for the two recovery animals in each dosage group.

FIG. 25E depicts transient expansion of CD8⁺ T cells in contrast to other PBMC subsets following 2 or 3 administrations of the indicated dosages of tLNP-982520. Cell counts were generated by bridging flow cytometry to CBC counts.

FIG. 25F portrays the overall effects of an immune response associated with various T cell engineering modalities. FIG. 25G portrays the roles and effects of the various components of the immune response associated with various T cell engineering modalities.

FIG. 25H depicts B cell recovery by B cell phenotyping of the peripheral blood by flow cytometry, pre-dose (days −12 and −3) through recovery (day 42) of 3 different 2-dose regimens as indicated (n=2 animals/regimen). Doses were administered on days 1 and 4. Each graph shows an individual animal. The height of the bar shows the number of B cells per μl of blood and the fill stack shows the relative contribution of each phenotype as defined by indicated markers (see Legend). The first digit in the animal number corresponds to the Group number in Table 15. The asterisk in the upper left panel indicates a datapoint missing due to a cytometer error. Note the different scales on the y-axes of the individual panels which were adjusted so that the proportions of the different phenotypes was easier to perceive in samples with fewer cells.

FIG. 25I depicts liver enzyme levels for ALT and AST pre-dose through day 10 for a control animal (n=1) and 3 different 2-dose regimens as indicated (n=3 animals/regimen). Each animal is shown as a data point; bar height=mean; error bars=range. Shaded area shows the normal reference range. ● PBS control, ▪ 1.0 mg/kg dose, ▴ 1.5 mg/kg dose, ♦ 1.0 mg/kg 1st dose|2.0 mg/kg 2nd dose depicts B cell recovery by B cell phenotyping of the peripheral blood by flow cytometry, pre-dose (days −12 and −3) through recovery (day 42) of 3 different 2-dose regimens as indicated (n=2 animals/regimen). Doses were administered on days 1 and 4. Each graph shows an individual animal. The height of the bar shows the number of B cells per μl of blood and the fill stack shows the relative contribution of each phenotype as defined by indicated markers (see Legend). The first digit in the animal number corresponds to the Group number in Table 15. The asterisk in the upper left panel indicates a datapoint missing due to a cytometer error. Note the different scales on the y-axes of the individual panels which were adjusted so that the proportions of the different phenotypes was easier to perceive in samples with fewer cells.

FIG. 25J depicts cytokine levels for IL6, INF-γ, MCP-1, and TNF-α 6 hours and 24 hours after each dose administration for a control animal (n=1) or 3 different 2-dose regimens as indicated (n=3 animals/regimen). Each animal is shown as a data point; bar height=mean; error bars=range. Shaded area shows the assay range. ● PBS control, ▪ 1.0 mg/kg dose, ▴ 1.5 mg/kg dose, ♦ 1.0 mg/kg 1st dose|2.0 mg/kg 2nd dose.

FIGS. 26A-26G depict the effects of depleting single PBMC subsets from the transfected cultures. The result of transfection of undepleted PBMC (WT PBMC) with tLNP-981219 (at three doses: 0.01 g, 0.0375 g and 0.6 g of mRNA encoding an anti-CD19 CAR) is compared to the results for PBMC depleted of CD4+ T cells, CD8+ T cells, NK cells, and monocytes from two human donors. FIG. 26A shows the effect on B cell killing. FIG. 26B shows the effect on CD69 expression by CD8+ T cells, an indicator of T cell activation. FIG. 26C-26F shows the effect on secretion IFNγ, granulocyte-macrophage colony-stimulating factor (GM-CSF), IL-6, and MCP-1, respectively. FIG. 26G depicts a schematic model of the interactions of the various components.

FIG. 27A-27B depict the percentage of CAR+ CD8+ T cells (27A) and number of B cells (27B) over time in blood from cynomolgus macaques pre-treated with and without the corticosteroid dexamethasone. The animals were administered tLNP-982520 (anti-CD20 CAR) using either a 60-minute (closed symbols) or 90-minute (open symbols) infusion according to a compact regimen (2×Q72 hrs). Each shape represents an individual animal; numbering is arbitrary and does not carry over to other figures.

FIG. 28 depicts the percentage of CD45+ cells that were B cells (CD20+) at necropsy in spleen, bone marrow, and lymph node from cynomolgus macaques treated with tLNP encapsulating an mRNA encoding an anti-CD20 CAR (tLNP-982520) according to a compact regimen with and without pretreatment with the corticosteroid dexamethasone. Untransfected controls (PBS) are included in each panel.

FIG. 29 depicts cytokine levels for IL6, MCP-1, and INF-γ 6 hours, 24 hours, and 48 hours after each dose of tLNP administration, for animals receiving 60-minute tLNP infusions (n=3, dark shade) or low dose dexamethasone prior to 90-minute tLNP infusion (n=3, light shade). Each animal is shown as a data point; bar height=mean; error bars=range.

FIG. 30 depicts the role of low dose corticosteroids in lowering pro-inflammatory cytokine release and acute phase response that can arise in CAR-T therapy using tLNP. Pre-treatment with low dose corticosteroids does not interfere with the cytotoxicity of this CAR-T therapy.

FIG. 31 depicts the titer of antibody against the anti-CD8 antibody used to target tLNPs in cynomolgus macaques treated with CD8-targeted tLNP encapsulating mRNA encoding either an anti-CD19 (tLNP-98219) or anti-CD20 (tLNP982520), or PBS. Animal numbers (1-8) are arbitrary. ADA: anti-drug antibody response. Animal numbers (1-8) are arbitrary and do not carry over from one experiment to another.

### DETAILED DESCRIPTION OF THE DISCLOSURE

Provided herein are compositions and methods for in vivo generation of CAR-T, or other antigen receptor-bearing T cells, resulting in more as well as more active T cells by using administration of repeated lower doses in a compact regimen or schedule. Antigen receptor bearing NK, NKT, or myeloid cells can also be generated. The use of in vivo engineered cells avoids manufacturing complexities and lymphodepletion-associated toxicities of current ex vivo CAR-T cell treatments. One approach to in vivo generation of exogenous antigen receptor-bearing T cells uses lipid nanoparticles to deliver mRNA encoding the antigen receptor. Both lipid composition of the LNP and decoration of the surface of the LNP with a binding moiety recognizing a T cell or other immune cell surface antigen can contribute to the LNP preferentially being taken up by such cells rather than in non-target tissues such as liver. LNP with such a binding moiety attached to its surface is referred to as a targeted-LNP (tLNP) and is provided herein.

As currently practiced ex vivo CAR-T cell therapies can lead to severe toxicities, although late stage or severe autoimmunity and ongoing inflammation may contribute to such outcomes. In vivo CAR-T therapies such as the compact regimen disclosed herein, allow treatment earlier in the course of disease due in part to greater acceptability to patients due to the absence of immunodepleting chemotherapy. This can have the further benefit of reducing the risk, occurrence, and/or severity of toxicity related to CAR-T therapy.

Using mRNA to reprogram a T cell to express an antigen receptor generates a transiently engineered T cell, as expression levels diminish as the T cell proliferates upon encountering the cognate antigen of the exogenous antigen receptor, diluting the mRNA and as the mRNA is degraded by the normal metabolic activity of the T cell. Experience with ex vivo CAR-T cells does not address how much antigen receptor needs to be expressed and how long that expression needs to last for there to be substantial pharmacologic effect and/or clinical efficacy. It is disclosed herein that multiple smaller doses, each subsequent dose administered within a few days of a preceding dose produces more T cells with greater activity than the same or greater cumulative dose administered once or at weekly intervals. Many embodiments are described in which the antigen receptor is a CAR but it should be understood that there are generally alternative embodiments relating to an antigen receptor generically, a TCR, or a TCE. As will be appreciated by a person of skill in the art, the properties of CARs, TCRs, and TCEs do have different characteristics that can impact the nature of the immune reactivity conferred. For example, TCEs are typically pan-T cell reagents so that even if a TCE mRNA is encapsulated in a CD8-targeted tLNP, the TCE will be secreted and reprogram all types of T cells.

It is to be understood that the particular aspects described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Headings are provided for convenience only and are not to be construed to limit the invention in any manner. Embodiments illustrated under any heading may be combined with embodiments illustrated under any other heading.

All references cited in the disclosure are hereby incorporated by reference in their entirety, as if fully set forth herein. To the extent any materials incorporated herein by reference conflict with the present disclosure, the present disclosure controls.

Prior to setting forth this disclosure in more detail, it may be helpful to provide abbreviations and definitions of certain terms to be used herein. Additional definitions are set forth throughout this disclosure.

### Definitions

Throughout this specification, unless the context specifically indicates otherwise, the terms “comprise” and “include” and variations thereof (e.g., “comprises,” “comprising,” “includes,” and “including”) are understood to indicate the inclusion of a stated component, feature, element, or step or group of components, features, elements or steps but not the exclusion of any other component, feature, element, or step or group of components, features, elements, or steps. Any of the terms “comprising”, “consisting essentially of”, and “consisting of” may be replaced with either of the other two terms, while retaining their ordinary meanings

As used herein, the singular forms “a,” “an,” and “the” include plural referents unless the context clearly indicates otherwise. It should be understood that the terms “a” and “an” as used herein refer to “one or more” of the enumerated components.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

Throughout this disclosure, any concentration range, percentage range, ratio range, or integer range is to be understood to include the value of any integer within the recited range and, when appropriate, fractions thereof (such as one tenth and one hundredth of an integer), unless otherwise indicated. Also, any number range of this disclosure relating to any physical feature, such as polymer subunits, size, or thickness, are to be understood to include any integer within the recited range, unless otherwise indicated. Throughout this disclosure, numerical ranges are inclusive of their recited endpoints, unless specifically stated otherwise.

As used herein and in the drawings, ranges and amounts can be expressed as “about” a particular value or range. The term “about” can also refer to +10% of a given value or range of values. Therefore, about 5% also means 4.5%-5.5%, for example. About also includes the exact amount. For example, “about 5%” means “4.5%-5.5%” and also discloses “⁵%.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

The phrase “at least one of” when followed by a list of items or elements refers to an open-ended set of one or more of the elements in the list, which may, but does not necessarily, include more than one of the elements.

“Derivative,” as used herein, refers to a chemically or biologically modified version of a compound that is structurally similar to a parent compound and (actually or theoretically) derivable from that parent compound. Generally, a “derivative” differs from an “analogue” in that a parent compound can be the starting material to generate a “derivative,” whereas the parent compound is not necessarily be used as the starting material to generate an “analogue.” A derivative can have different chemical or physical properties than the parent compound. For example, a derivative can be more hydrophilic or hydrophobic, or it can have altered reactivity as compared to the parent compound. Although a derivative can be obtained by physical (for example, biological or chemical) modification of the parent compound, a derivative can also be conceptually derived, for example, as when a protein sequence is designed based on one or more known sequences, an encoding nucleic acid is constructed, and the derived protein obtained by expression of the encoding nucleic acid.

“Subject” or “patient” as used herein are used interchangeably and refer to a warm-blooded animal such as a mammal, preferably a human, which is afflicted with, or has the potential to be afflicted with cancer, fibrosis, an autoimmune disease, or rejection of an allogeneic organ or tissue transplant, as described herein.

“Contacting” as used herein includes the physical contact of at least one substance to another substance.

“Express” or “expression” as used herein refers to transcription and/or translation of a nucleic acid coding sequence resulting in production of the encoded polypeptide.

“Pathogenic cell” as used herein refers to cells that are the direct cause of the disease or disorder in question as well as those that contribute to the overall pathogenesis. For example, in the context of cancer both the neoplastic cells themselves and cells of the supporting tumor stroma are pathogenic cells. In another example, in the context of autoimmunity, disease control by B cell depletion therapy can result from B cell immunomodulatory effects rather than a direct effect on the production of autoreactive antibody (see, for example, Hampe C S. B Cell in Autoimmune Diseases. Scientifica (Cairo). 2012; 2012:215308. doi: 10.6064/2012/215308. PMID: 23807906; PMCID: PMC3692299, and Lee, D. S. W., Rojas, O. L. & Gommerman, J. L. B cell depletion therapies in autoimmune disease: advances and mechanistic insights. Nat Rev Drug Discov 20, 179-199 (2021). doi.org/10.1038/s41573-020-00092-2, which are each incorporated by reference for all that they teach regarding the role of B cells in autoimmunity and B cell depletion therapy to the extent that it is not inconsistent with the present disclosure).

As used herein “transfection” or “transfecting” refers to the introduction of nucleic acids into cells by non-viral methods. Transfection can be mediated by calcium phosphate, cationic polymers, magnetic beads, electroporation and lipid-based reagents. In particular embodiments disclosed herein transfection is mediated by solid lipid nanoparticles (LNP) including targeted LNP (tLNP). The term transfection is used in distinction to transduction—transfer of genetic material from cell to cell or virus to cell—and transformation—the uptake of extracellular genetic material by the natural processes of a cell. As used herein, phrases such as “delivering a nucleic acid into a cell” are synonymous with transfection.

“Reprogramming,” as used herein with respect to immune cells, refers to changing the functionality of an immune cell with respect to antigenic specificity by causing expression of an exogenous T cell receptor (TCR), a chimeric antigen receptor (CAR), or an immune cell engager (collectively termed “reprogramming agents”). Generally, T lymphocytes and natural killer (NK) cells can be reprogrammed with a TCR, a CAR, or an immune cell engager while only a CAR or an immune cell engager is used in reprogramming monocytes. In the case of an immune cell engager, the immune cells engaged and redirected against the pursued cell antigen of the immune cell engager are reprogrammed cells whether or not they express the reprogramming agent. Reprogramming can be transient or durable depending on the nature of the engineering agent.

“Engineering agent,” as used herein, refers to agents that confer the expression of a reprogramming agent by an immune cell, particularly a non-B lymphocyte or monocyte. Engineering agents can include nucleic acids, including mRNA that encode the reprogramming agent. Engineering agents can also include nucleic acids that are or encode components of gene editing systems such as RNA-guided nucleases, guide RNA, and nucleic acid templates for knocking-in a reprogramming agent or knocking-out an endogenous antigen receptor. Gene editing systems comprise base-editors, prime-editors or gene-writers. RNA-guided nucleases include CRISPR nucleases such as Cas9, Cas12, Cas13, Cas3, CasMINI, Cas7-11, and CasX. For transient expression of a reprogramming agent, such as a CAR, an mRNA encoding the reprogramming agent can be used as the engineering agent. For durable expression of the reprogramming agent, such as an exogenous, modified, or corrected gene (and its gene product), the engineering agent can comprise mRNA-encoded RNA-directed nucleases, guide RNAs, nucleic acid templates and other components of gene/genome editing systems.

Examples of gene editing components that are encoded by a nucleic acid molecule include an mRNA encoding an RNA-guided nuclease, a gene or base editing protein, a prime editing protein, a Gene Writer protein (e.g., a modified or modularized non-long terminal repeat (LTR) retrotransposon), a retrotransposase, an RNA writer, a zinc finger nuclease (ZFN), a transcription activator-like effector nuclease (TALEN), a meganuclease, a transposase, a retrotransposon, a reverse transcriptase (e.g., M-HLV reverse transcriptase), a nickase or inactive nuclease (e.g., Cas9, nCas9, dCas9), a DNA recombinase, a CRISPR nuclease (e.g., Cas9, Cas12, Cas13, Cas3, CasMINI, Cas7-11, CasX), a DNA nickase, a Cas9 nickase (e.g., D10A or H840A), or any fusion or combination thereof. Other components include a guide RNA (gRNA), a single guide RNA (sgRNA), a prime editing guide RNA (pegRNA), a clustered regularly interspaced short palindromic repeat (CRISPR) RNA (crRNA), a trans-activating clustered regularly interspaced short palindromic repeat (CRISPR) RNA (tracrRNA), or a DNA molecule to be inserted or serve as a template for double-strand break (DSB) repair at a specific genomic locus. Genome-, gene-, and base-editing technology are reviewed in Anzalone et al., Nature Biotechnology 38:824-844, 2020, Sakuma, Gene and Genome Editing 3-4:100017, 2022, and Zhou et al., MedComm 3(3):e155, 2022, each of which is incorporated by reference for all that they teach about the components and uses of this technology to the extent that it does not conflict with the present disclosure.

“Target antigen” or “targeted antigen”, as used herein refers to a surface antigen of an immune cells which is bound by the targeting moiety of a tLNP.

“Pursued antigen”, as used herein, refers to the antigen recognized by the reprogramming agent (such as a TCR, CAR or immune cell engager). It is common in the art to use the term target (or targeted) antigen with reference to any antigen that is bound by an antigen (or other) receptor. This has potential to be confusing where two distinct functional classes of antigen are concerned. In an effort to avoid this confusion, target (or targeted) antigen has been used herein to refer to the antigen bound by the targeting moiety of a nanoparticle and pursued antigen (or cell or tissue or indication, etc.) has been used to refer to an antigen bound by a reprogramming agent. (The substitution is not used in the terms “effector to target ratio,” “target cell, “off-target,” and “on-target” as that would tend to increase potential confusion rather than reduce it.) In the treatment of diseases, the pursued antigen will be expressed by a pathogenic cell but may also be expressed by normal cells.

“Potentiation,” as used herein refers to an effect of increasing the potency of a drug or other therapeutic agent.

“T cell antigen receptor,” as used herein, refers to any protein with an antigen binding domain that upon engaging its cognate antigen can bring about activation of a T cell. T cell antigen receptors include CARs, TCRs, and TCEs or combinations thereof. In some instances, if expressed in or engaged with an NK cell or a monocyte, the T cell antigen receptor can bring about activation of these cells. A T cell antigen receptor can also be referred to as a T cell-activating antigen receptor.

“Conditioning agent,” as used herein, refers to a biological response modifier (BRM) that enhances the efficiency of engineering an immune cell, expands the number of immune cells available to be engineered or the number of engineered cells in a target tissue (for example, a tumor, fibrotic tissue, or tissue undergoing autoimmune attack), promotes activity of the engineered cell in a target tissue, or broadens the range of operative mechanisms contributing to a therapeutic immune reaction. A conditioning agent may be provided by delivering an encoding nucleic acid in a tLNP. Exemplary BRMs include cytokines, such as IL-7, IL-15, or IL-18, and immune checkpoint inhibitors, such antibodies that block the binding of PD-1 and PD-L1 to each other.

Conditioning may be defined by the timing of its administration in relation to administration of an engineering agent, such as pre-treatment conditioning, concurrent conditioning, and post-treatment conditioning. In pre-treatment conditioning, a conditioning agent is administered prior to administration of an engineering agent. In various embodiments, a conditioning agent is administered one to several times in the week prior to administration of an engineering agent. In some embodiments the last pre-conditioning administration is the day before or the day of administration of an engineering agent. Pre-treatment conditioning is typically an activating conditioning. Post-treatment conditioning takes place subsequent to at least an initial dose of the engineering agent and may not itself be initiated until after a final dose of the engineering agent in a cycle of a set number of multiple doses. While pre-treatment conditioning and post-treatment conditioning can take place outside of the time interval in which an engineering agent is administered, concurrent conditioning extends over the same time interval as that over which an engineering agent is administered. Indeed, in some embodiments, an engineering agent and a conditioning agent are packaged in the same nanoparticle. In other embodiments the conditioning and engineering agents are packaged in separate nanoparticles, or a conditioning agent is administered systemically.

Conditioning can also be classified according to its effect. Activating conditioning leads to the expansion of polyfunctional immune effector cells amenable to in vivo engineering and/or the mobilization of immune effector cells resulting in the localization in tumor or other disease-associated tissue. The γ-chain receptor cytokines promote both effects, stimulating both proliferation and migration. Proliferation of immune effector cells will also be stimulated by highly active, pan-activating cytokines, such as IL-12 and IL-18. Activating conditioning is generally carried out prior to administration of the in vivo engineering agent, although it can continue to be given concurrently, especially when the in vivo engineering agent is administered multiple times at intervals of up to several days. Repeated cycles of activating conditioning followed by treatment with the in vivo engineering agent can also be used. Further discussion of activating conditioning can be found in WO2024040195 which is incorporated by reference for all that it teaches about activating conditioning, conditioning agents and their use that is not inconsistent with this disclosure.

The term “immune cell,” as used herein, can refer to any cell of the immune system. However, particular aspects can exclude polymorphonuclear leukocytes and/or B cells, or be limited to non-B lymphocytes such as T cell and/or NK cells, or to monocytes such as dendritic cells and/or macrophages in their various forms.

The term “immunogenic drug,” as used herein in relation to antidrug antibodies (ADA), refers to any component of a pharmaceutical product (therapeutic, prophylactic, diagnostic, and the like) with the potential to induce an antibody response when administered to a patient.

The term “nucleic acid” or “nucleic acid molecule,” as used herein, refers to either an RNA or DNA molecule, especially those encoding an expressible polypeptide, where context does not dictate otherwise. Description of the disclosed embodiments focuses primarily on mRNA molecules having the structure of a canonical mRNA. However, polypeptides can also be encoded in and expressed from circular and self-amplifying (also known as self-replicating) RNA molecules. Accordingly, the sequence of any of the herein disclosed linear mRNA molecules can be incorporated into a circular or self-amplifying/self-replicating RNA molecule. Similarly, each of these RNA molecules can be encoded as a DNA molecule. Each of the disclosed nucleic acid sequences, RNA or DNA, should be understood to disclose the corresponding DNA or RNA sequence, respectively

The terms “treatment,” “treating,” etc., as used herein, refer to the medical management of a patient with the intent to cure, ameliorate, stabilize, or prevent a disease, pathological condition, or disorder. Treatment relates to the provision of care and, without more, does not require any particular effectiveness. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. Various embodiments may specifically include or exclude one or more of these modes of treatment.

As used herein, a “+” (plus sign) or “−” (minus sign) located immediately after a cell surface antigen indicates the presence or absence, respectively, of that antigen on the surface of a cell. The plus or minus sign can be in regular typeface or a superscript. For example, a CD8+ cell means the same thing as a CD8⁺ cell, both of which mean that CD8 is detectably expressed on the surface of the cell. Similarly, a CD8− cell means the same thing as a CD8⁻ cell, both of which mean that CD8 is not detectably expressed on the surface of the cell.

### Immune Engineering Amplification

For in vivo engineering of cells with tLNP-delivered RNA molecules, using an administration schedule comprising multiple (e.g., 2-4 doses, 2-3 doses, or 2 doses) comparatively smaller dosage amounts within a few days from one to the next (e.g., 2-5 days, 2-4 days, or 3 days) of a T cell-targeted tLNP encapsulating mRNA encoding an antigen receptor was more efficacious than comparatively larger doses administered once or a week apart. The greater efficacy of the procedure, referred to as a compact regimen, includes greater efficiency of engineering for the 2^{nd} and subsequent administrations (thus, immune engineering amplification) as well as a more extensive depletion of the cells attacked by the engineered T cells. This was true even when the cumulative dose was substantially less.

The term “immune engineering amplification” refers to a method for increasing the number of reprogrammed or otherwise engineered immune cells via an initial step of increasing the transfection efficiency of the immune cells to be engineered. Immune engineering amplification can be used to generate large numbers of effector cells, for example, T effector cells, which can enable effective treatment even when there is a large burden of cells expressing the pursued antigen.

Immune engineering amplification can offer a variety of advantages. The most basic is an improved efficiency of transfection after the initial activating dose of the compact regimen. This can be accompanied by a higher expression level of the polypeptide encoded by the transfected nucleic acid (such as an RNA, for example, mRNA). While in many embodiments the activating agent and the therapeutic agent (discussed below) are one and the same, this is not necessarily the case. When the activating agent and therapeutic agent are different there is a further advantage of being able to separately modulate the potentiating and therapeutic functions of the method. The lower individual and/or cumulative dosages possible with the compact regimen can provide a greater safety margin or a higher therapeutic index, and that can enable dosage intensification to obtain greater pharmacodynamic or therapeutic effect. These safety effects can be further augmented by administering a low dose corticosteroid prior to the activating and/or therapeutic agents. In some embodiments, therapeutic effect is accomplished through B cell depletion which has the attendant benefit of blunting induction of antidrug antibodies (ADA). By “blunting” it is meant that the response is absent or diminished compared to what could occur without B cell depletion. However, B cell depletion through the compact regimen can also be used to blunt induction of ADA against other therapeutic agents including other tLNP-delivered agents or any other potentially immunogenic therapeutic agent. The ability to separate the potentiating and therapeutic functions by using different compositions as the activating and therapeutic agents enables immune engineering to be applied to direct immune activity (such as cytolytic activity) against cells in which the therapeutically pursued antigen has expression that is in some manner restricted so that the therapeutic agent would not provide a robust potentiating effect. These and other advantages and how they can be exploited are described further below.

Accordingly, in certain aspects, this disclosure provides methods of potentiating or amplifying the in vivo transfection of T cells (or other immune cells) in a subject comprising initially administering an activating agent, for example, a T cell activating agent, and subsequently administering a therapeutic agent according to a compact administration schedule lasting not more than 7 or 8 days for a cycle of treatment. The therapeutic agent comprises an immune cell-targeted tLNP encapsulating a nucleic acid encoding a T cell antigen receptor. This antigen receptor can reprogram the immune cell to specifically bind to an antigen of the cell against which immune activity is to be directed (a pursued antigen). In some embodiments, the immune cell is a T cell, for example a CD8+ T cell. In some embodiments, the antigen receptor is a CAR. In some embodiments, the nucleic acid is an mRNA. In some embodiments, the tLNP is targeted to CD8.

As used herein, the term “therapeutic agent” refers to an agent that generates a therapeutic outcome intended to treat a particular disease. The therapeutic agent can comprise a T cell-targeted tLNP encapsulating a nucleic acid encoding a T cell antigen receptor. In certain embodiments, the therapeutic outcome includes B cell depletion to treat autoimmune disease, activated fibroblast killing to treat fibrosis or cancer, or tumor cell killing to treat cancer. Thus, a population of pursued cells such as B cells, fibrosis-promoting cells, or tumor cells that express the antigen recognized by the T cell antigen receptor will be depleted or killed. In certain embodiments involving an initial depletion of B cells (accomplished by immune engineering amplification) to disrupt ADA induction, the agent accomplishing the B cell depletion is still referred to as a therapeutic agent even if the ultimate therapeutic outcome is mediated by a second therapeutic agent or any other pharmaceutic that has the potential to induce an antibody response.

The term “activating agent” refers to an agent that activates an immune cell (such as T cell, referred to herein as a “T cell activating agent”), thereby increasing transfection efficiency and other metabolic activity. In the context of a compact regimen and immune engineering amplification, an activating agent is administered before a therapeutic agent to increase the number of activated immune cells that can be transfected with the therapeutic agent, realizing that the activating agent and the therapeutic agent can be one and the same. That is, a T cell targeted tLNP encapsulating an RNA encoding a T cell activating antigen receptor serves the function of activating agent and therapeutic agent. According to various aspects, the T cell activating agent can fall into one of three classes which can be used individually or in combinations. 1) In certain aspects, the therapeutic agent also serves as the T cell activating agent (that is, they both comprise the same RNA-encoded T cell antigen receptor). This can be particularly appropriate where the pursued antigen is well-expressed on readily accessible cells (a non-restricted antigen), for example, CD19 or BCMA on B cells. 2) In certain aspects, the T cell activating agent comprises an immune cell-targeted tLNP encapsulating a nucleic acid encoding a T cell antigen receptor that has a different specificity than that of the therapeutic agent. This can be particularly appropriate where expression of the pursued antigen is in some way restricted, for example, the pursued antigen is expressed at a low level, on a small population of cells, and/or on cells that are not readily accessible so that only a limited number of transfected immune cells would engage cognate antigen and become activated. Instead, a first T cell antigen receptor provided by the activating agent can be directed against a non-restricted antigen (such as a readily accessible antigen) for example, CD19 or BCMA on B cells, to generate a large population of activated immune cells that can then be transfected with the therapeutic agent providing a second T cell antigen receptor that bind a pursued antigen, thereby generating a larger number of immune cells directed against the cells expressing the pursued antigen. Thus, in some such embodiments, the tLNP providing a first T cell antigen receptor can serve solely as an activating agent. The use of tLNP providing T cell antigen receptors serving different roles can also be particularly appropriate where the therapeutic agent can induce an undesirable reaction such as anti-drug antibodies (ADA), cytokine release syndrome (CRS), toxicity, and the like. By reducing exposure to the therapeutic agent such undesirable reactions can be avoided or mitigated. 3) Finally, in certain aspects, the T cell activating agent can comprise a BRM, that is, a conditioning agent. The T cell activating agent/conditioning agent can be provided as the protein or as a BRM-encoding RNA encapsulated in a tLNP. This can be particularly appropriate where reducing exposure to the therapeutic agent is desirable such as avoiding or reducing the induction of ADA, CRS, toxicity, and the like.

Regarding the difference between antigens having restricted or non-restricted expression, B cells are widely distributed in the body, easily accessible to T cells, and express CD19 at a level so the antigen can be productively engaged by a T cell antigen receptor, thus providing an objective example of non-restricted expression as the term is used in the context of whether the antigen can support a robust potentiation effect in immune engineering amplification. In contrast, a solid tumor can be surrounded by a fibrotic matrix, the antigen can be unique to the tumor, and/or the tumor may express only few molecules per cell thus providing an objective example of restricted expression. However, the difference between antigens having restricted or non-restricted expression can also be relative. That is, some antigens will produce a robust potentiation effect while others produce a weaker potentiation effect and so to obtain better transfection efficiency, an antigen with relatively non-restricted expression can be pursued in the initial activating stage of immune engineering amplification and the antigen having restricted expression can be pursed in the later stage of immune engineering amplification by the therapeutic agent. It should be kept in mind when choosing an antigen with non-restricted expression solely for its usefulness in potentiation, that the initial immune activity directed against the antigen should not produce adverse effects that are intolerable in the context of the disease or condition to be treated. Antigens found on antigen-presenting cells or in the B cell lineage are attractive choices for antigens with non-restricted expression and include (in addition to CD19) CD20, CD22, CD38, CD123, CD138, DEC205, BCMA, FcRL5, and GPRC5D. Examples of antigens having restricted expression include EGFRvIII, Her2/Neu, PSCA, PSMA, mesothelin, FAP, trop2, DLL3, GPC3, Claudin 18.2, GD2 and as TCR targets HPV E6, E7, NYESO1, PRAME, Melan A, Tyrosinase, PSMA, SSX2, and EBV latent antigen.

In some aspects, immune engineering amplification comprises administering multiple doses of a T cell-targeted tLNP encapsulating an mRNA encoding a T cell antigen receptor in a compact regimen, wherein a population of cells in the subject expresses an antigen recognized the T cell antigen receptor, whereby more T cells express the antigen receptor as a result to a subsequent administration than as a result of the initial administration. (The phrase “comprising administering multiple doses of a T cell-targeted tLNP” may be alternatively stated as “comprising repeatedly administering a T cell-targeted tLNP”). In certain embodiments, the multiple doses of a compact regimen is 2-4 doses, 2-3 doses, 3 doses or 2 doses. In some embodiments, more T cells express the T cell antigen receptor after administration of at least 2-4 doses, 2-3 doses, 3 doses or 2 doses in the compact regimen as compared to the same total dosage administered over a same time interval as a single dose or as multiple doses where each subsequent dose is administered ≥7 days after an immediately preceding dose. In some embodiments of the compact regimen, a same dosage is administered for the initial and subsequent administrations. In other embodiments, a smaller dosage is administered for the initial administration than for the subsequent administration(s). In some embodiments, dosage can be from about 0.05 or 0.075 mg/kg to about 3 mg/kg per administration. When the initial dosage is smaller than the subsequent dosage(s), the dosage for the initial administration can be from about 0.075 to about 1.5 mg/kg, for example, 0.1, 0.5, 1.0 or 1.5 mg/kg or within any range bound by a pair of those values, and the dosage for subsequent administration(s) can be from about 1.0 to about 3.0 mg/kg per administration, for example 1.0, 1.5, 2.0, 2.5, or 3.0 mg/kg or within any range bound by a pair of those values.

The immune engineering amplification effect can also be used to promote durable engineering. In some embodiments, the initial dose comprises an mRNA encoding an immune receptor to recognize its cognate antigen and initiate the potentiation process, but subsequent doses comprise gene editing components to knock-in a reprograming agent which can be the same or different than the one provided by the initial dose. The transfection efficiency, and thus the gene editing efficiency is improved due to the potentiation.

The immune engineering amplification effect can also be used to promote engineering of immune cells, such as T cells, to express any exogenous polypeptide. In some embodiments, the initial dose comprises an mRNA encoding an immune receptor to recognize its cognate antigen and initiate the potentiation process, but subsequent doses comprise RNA encoding a pro-inflammatory cytokine, an anti-inflammatory cytokine, an intracellular signaling protein, an antibody for intracellular expression or secretion including a secreted immune suppressing antibody or an immune checkpoint inhibitor antibody, an antigen to induce an immune reaction, or any other polypeptide that would be useful to be expressed in an immune cell. The transfection efficiency and level of expression is improved by the potentiation and the level of expression as compared to a protocol not producing the immune engineering amplification effect can be increased as well.

At the initial administration, many of the T cells encountered by the tLNP will be resting T cells. As compared to activated T cells, resting T cells are transfected less efficiently. However, those resting T cells that do become transfected and express the antigen receptor encoded by the transfected mRNA (that is, they have been reprogrammed) will become activated upon encountering cells expressing the cognate antigen of the antigen receptor. Accordingly, and without being bound to a particular mechanism, this amplification was due to administering a subsequent dose of the tLNP while the initially transfected cells remained activated so that a greater proportion of T cells were activated and more efficiently transfected. By a week after the initial administration, the amplification no longer occurred indicating that some amplification-associated feature of activation was no longer operating. The compact administration regimen schedule was designed to assure that subsequent dosages were administered while a high proportion of T cells were activated and in an “amplification-permissive” state and thus were more efficiently transfected. As activated T cells were more metabolically active, they also expressed a higher level of the antigen receptor which contributed to the efficacy of the transfected T cell.

As set out above, by a “compact regimen” or “compact schedule” of administration (which are used herein interchangeably) is meant that each subsequent dose is administered within 5 days of the immediately preceding dose. However, internalization of the tLNP also temporarily reduces the presence of the targeted surface antigen (for example, CD8, CD2, CD4, CD5, etc.) on the cell surface. It is therefore advantageous to wait for expression levels of the targeted surface antigen to be restored to gain maximal effect from the subsequent dose of tLNP. Transfected cells can express significant levels of antigen receptor for a day or two, for example, so one can afford to wait to administer the subsequent dose. Thus, in various embodiments, each subsequent dose (e.g., 1, 2 or 3 subsequent doses) is administered 1 to 5 days after the immediately preceding dose such as 1, 2, 3, 4, or 5 days after the immediately preceding dose or the subsequent dose is administered in a time range bound by any pair of those values, for example, 2-5, 2-4, 2-3, or 3-4.

In some embodiments, the interval between doses is always the same, for example, every 2^{nd}, 3^{rd}, or 4^{th} day. The recycling time of the marker targeted by the tLNP can affect what interval between dose administrations is best. For CD8, for example, 2 to 4 days appears best, with 2 days allowing sufficient time for CD8 to reappear on the surface of the cell after internalization connected with internalization of a CD8-targeted tLNP. For targeted markers with a faster recycling time 1 day can be sufficient.

In principle, increased sustained expression and supraphysiological signaling through a CAR construct may lead to T cell exhaustion or antigen-induced cell death (see for example the review Liu et al., Front Immunol. 2021; 12: 748768). It is now broadly accepted that factors such as kinetics of CAR expression may greatly influence the outcome of cognate antigen interaction (activation versus anergy). Since tLNP-mRNA CAR technology can afford very high albeit brief CAR expression levels, it was not evident whether this would lead to activation or exhaustion of T cells in presence of the cognate antigen. Data presented in the Examples below demonstrate that the former rather than the latter ensues.

As pursued antigen expressing cells are depleted, and in some embodiments eliminated, fewer engineered T cells will encounter antigen expressing cells to become activated and the amplification effect will wane. How many administrations can or need to be made before amplification wanes will depend in part on the dosage used. It can be expected that higher dosages will lead to faster depletion of pursued pathogenic cells and thus sooner waning of the amplification effect than lower doses. Accordingly, in some embodiments, 2-3 administrations of the tLNP will be sufficient to substantially deplete the pathogenic cells while lower dosages can lead to longer persistence of greater numbers of pathogenic cells and the amplification will persist longer and 4, 5, 6, or more administrations can be used. To the extent that depletion of the pathogenic cells is not uniform throughout the body, the amplification effect can persist locally where the pathogenic (or other pursued antigen expressing) cells persist even if it is no longer observable systemically. For example, in the depletion of B cells they are observed to quickly disappear from blood with even quite small dosages whereas depletion of B cells in lymph nodes takes longer to be observed and is more extensive with higher dosages. Depletion of B cells from liver, spleen, and bone marrow is intermediate between these extremes.

Further, as long as there are activated T cells from a prior round of engineering, there will be amplification at the subsequent administration, even if at that point there are no antigen-bearing pursued cells remaining at the time of the subsequent administration, as the amplification effect is not dependent on their presence but on the presence of the previously activated T cells. Thus, in some embodiments, the compact administration regimen can be continued indefinitely until the desired degree of depletion (or elimination) of the pathogenic (or other pursued) cells is achieved. However, in certain embodiments, the multiple administrations of the compact regimen consist of 2, 3, 4, 5, or 6 administrations or a range bound by any pair of those values, for example 2 to 3, 2 to 4, 3 to 4, or 2 to 6 administrations. Similarly, repeated cycles of a compact regimen, separated by a break in dosing, can be used to extend treatment over a longer period of time. Each cycle comprises multiple administrations and in certain embodiments each cycle consists of 2, 3, 4, 5, or 6 administrations or a range bound by any pair of those values, for example 2 to 3, 2 to 4, 3 to 4, or 2 to 6 administrations. In various embodiments, the break between cycles can be for a fixed time interval, for example, 1, 2, 3, 4, 5, or 6 weeks, or until after reappearance of normal cells expressing the pursued antigen, or until after the reappearance or re-expansion of the pathogenic cells, or until relapse (that is recurrence of symptomatic disease).

Thus, multiple cycles can be used to ensure depletion/elimination of pathogenic cells before or after recovery of normal cells expressing the pursued antigen, for example, if there is some indication that a single cycle is or was insufficient to achieve the desired degree of depletion, to remediate elimination that was incomplete, or to treat recurrence of the disease.

An activating conditioning agent can be used to augment or extend the amplifying effect of interaction of the reprogrammed T cell with the cognate antigen of its activating antigen receptor(s). In some embodiments, the activating conditioning agent is administered 2 to 5 days before a subsequent dose of tLNP. In some embodiments, the activating conditioning agent is administered 2 to 5 days before the first subsequent dose of tLNP serving primarily to augment the activating and transfectability effects of cognate antigen recognition. In other embodiments, the activating conditioning agent is administered 2 to 5 days before a 2^{nd} or later subsequent dose of tLNP serving primarily to counteract the reduced amplification (and related activation and transfectability) due to reduced presence of cells expressing the pursued (cognate) antigen. As noted above, the activating conditioning agent can be a γ-chain receptor cytokine, such as IL-2, IL-7 or IL-15, or a pan-activating cytokine such as IL-12 or IL-18, or an immune checkpoint inhibitor such as an antagonist of CTLA-4, PD-1, PD-L1, Tim-3, LAG-3 or IDO or agonists of 4-1BB, OX40 or ICOS. In some embodiments, the activating conditioning agent is IL-7. In some embodiments, the activating conditioning agent is IL-18. In some embodiments, the activating conditioning agent is a blocking antibody against CTLA-4, PD-1 or PD-L1. These one or more doses of activating conditioning agent administered 2 to 5 days before administration of tLNP can be, for example, a single dose 2 to 5 days before, two doses 2 and 3 to 5 days before or 3 and 4 to 5 days before, daily doses 2 to 4 or 5 days before, or 3 to 5 days before, each alternative within these ranges a separate embodiment. In some embodiments, an additional dose of activating conditioning agent is administered on the same day as the first administration of tLNP.

In alternative aspects for each of the described variations of a compact administration regimen, the initial dose of tLNP in a compact schedule of administration is replaced with one or more doses of an activating conditioning agent to provide the activating and transfectability effects associated with the initial dose of an engineering agent providing expression of a T cell antigen receptor. Accordingly, while the compact schedule has been described as comprising an initial dose of tLNP and 1 or more subsequent doses of tLNP each administered within 1-5 days of the immediately previous dosage, this alternative aspect can comprise a single administration of tLNP although additional doses of tLNP as described herein can also be administered. By replacing the initial tLNP dose with an activating conditioning agent the cumulative dosage of tLNP (and its payload) can be further reduced thereby reducing the opportunity for toxicity from tLNP components or generation of antibodies to those components (termed anti-drug antibodies). As noted above, the activating conditioning agent can be a γ-chain receptor cytokine, such as IL-2, IL-7 or IL-15, or a pan-activating cytokine such as IL-12 or IL-18, or an immune checkpoint inhibitor such as an antagonist of CTLA-4, PD-1, PD-L1, Tim-3, LAG-3 or IDO or agonists of 4-1BB, OX40 or ICOS. In some embodiments, the activating conditioning agent is IL-7. In some embodiments, the activating conditioning agent is IL-18. In some embodiments, the activating conditioning agent is a blocking antibody against CTLA-4, PD-1 or PD-L1. These one or more doses of activating conditioning agent is administered 2 to 5 days before administration of tLNP, for example, a single dose 2 to 5 days before, two doses 2 and 3 to 5 days before or 3 and 4 to 5 days before, daily doses 2 to 4 or 5 days before, or 3 to 5 days before, each alternative within these ranges a separate embodiment. In some embodiments, an additional dose of activating conditioning agent is administered on the same day as the first administration of tLNP.

As an alternative to being provided as a polypeptide, an activating conditioning agent can also be provided as an encoding RNA encapsulated in a tLNP. In some embodiments, the tLNP encapsulating an RNA encoding an activating conditioning agent and a tLNP encapsulating an RNA encoding a T cell antigen receptor can target the same antigen. In some embodiments, both of these types of tLNP target CD8. Use of an RNA-encoded BRM as an activating conditioning agent can allow for more localized administration, lower overall dosage, and reduced side-effects. An RNA-encoded cell-tethered BRM or cell tethered-cytokine can further augment this localizing effect. The timing of administration can be the same as that described for the polypeptide forms. In some embodiments, the RNA comprises mRNA.

The use of an activating conditioning agent in conjunction with a compact administration schedule as disclosed herein can further reduce the number of doses and/or further reduce individual or cumulative dosages needed and thereby reduce or avoid dose limiting toxicities due to high and/or repeat exposure such as inducing immunogenicity, infusion reactions, or either on- or off-target effects. By reducing the number of doses and or cumulative dosage the use of an activating conditioning agent in conjunction with a compact administration schedule as disclosed herein can also facilitate and expand the safe use of multiple cycles of treatment.

Further details about activating conditioning, as well as other modes of conditioning that can be useful in conjunction with the in vivo reprogramming of T cells, are disclosed in WO2024040194A1 and WO2024040195A1 which are each incorporated by reference herein in their entirety to the extent that they are not inconsistent with the present disclosure.

In some embodiments, the T cell is a cytolytic T cell. In various further embodiments, the T cell, or cytolytic T cell, is a CD8⁺ T cell, a CD4+ T cell, or an NKT cell. In some embodiments, the T cell surface antigen targeted by the LNP targeting moiety is CD2, CD3, CD4, CD5, CD7, CD8, CD28, 4-1BB (CD137), CD166, CTLA-4, OX40, PD-1, GITR, LAG-3, TIM-3, CD25, low affinity IL-2 receptor, IL-7 receptor, IL-12 receptor, IL-15 receptor, IL-18 receptor, IL-21 receptor, CXCR5, or CX3CL1 receptor.

In some embodiments, the T cell antigen receptor is a CAR, a TCR, or a TCE. In some embodiments, a single T cell antigen receptor is to be expressed by T cells and a tLNP encapsulates a single engineering agent conferring expression of the single T cell antigen receptor. In other embodiments plural T cell antigen receptors are expressed, for example, 2 or 3. In some embodiments, the plural T cell antigen receptors have specificity for different epitopes on the same antigen or for different antigens. In some embodiments, the plural T cell antigen receptors are all of the same type, for example all CARs, all TCRs, or all TCEs, while in other embodiments they are of mixed types, for example, CAR and TCR, CAR and TCE, TCR and TCE, or CAR, TCR, and TCE. In some embodiments, an engineering agent for each of the plural T cell antigen receptors is carried in separate tLNPs and the tLNPs can target the same or different T cell surface antigens. In other embodiments, an engineering agent for each of the plural T cell antigen receptors is carried in the same tLNPs. An engineering agent, for example, an mRNA, can be monocistronic or multicistronic. Thus, a tLNP carrying an engineering agent for each of the plural T cell antigen receptors can encapsulate multiple monocistronic engineering agents, one or more multicistronic engineering agents, or a combination thereof. In some embodiments the multicistronic engineering agent is bicistronic. In some embodiments, a T cell antigen receptor has specificity for a single antigen of a pathogenic cell while in other embodiments a T cell antigen receptor has specificity for 2 or more antigens of a pathogenic cell. For embodiments involving use of plural species of tLNP, at typical dosages a substantial majority of targeted T cells are transfected by each of the species of tLNP unless the targeting moieties of the tLNP species are chosen to direct different species to different T cell subsets.

It should be appreciated that a CD8+ CAR-T cell can have different effects than an TCE recognizing the same pursued antigen. The T cell recruiting specificity of an TCE needs to activate the T cell in order to have significant biological effect. For this reason, CD3 has been most often chosen as the specificity for the T cell recruiting specificity of an TCE although other activating pan T cell markers have been utilized. As a pan T cell marker, CD3 is found on both CD8+ and CD4+ T cells so that both types of T cell will be reprogrammed with the TCE and have more diverse, and possibly countervailing, activities than a population of CD8+ CAR T cells. Although in many embodiments tLNP are preferred, in alternative embodiments tropic LNP (that is, LNP which preferentially mediate transfection of T cells or other immune cells based on their lipid content rather than utilizing a targeting moiety) are substituted for tLNP. However, tropic LNP will generally be less specific in their targeting than a tLNP.

### LNP Compositions

The LNP composition contributes to the formation of stable tLNP, efficient encapsulation of a payload mRNA, protection of the mRNA from degradation until it is delivered into a cell, and promotion of endosomal escape of the mRNA into the cytoplasm. These functions are primarily independent of the specificity of the binding moiety (or moieties) serving to direct or bias the tLNP to a particular cell type(s).

In certain embodiments the LNP comprises a lipid composition comprising an ionizable cationic lipid, PEG-lipid comprising functionalized PEG-lipid and non-functionalized PEG-lipid, a phospholipid, and a sterol.

The LNPs and/or tLNPs can include the various components in amounts sufficient to provide a nanoparticle with a desired shape, fluidity, and bio-acceptability as described herein. With respect to LNPs or tLNPs of this disclosure, in some embodiments, the LNP (or tLNP) comprises at least one ionizable cationic lipid (e.g., as described herein) in an amount in the range of from about 35 to about 65 mol %, or any integer bound sub-range thereof, e.g., in an amount of from about 40 to about 65 mol %, about 40 to about 60 mol %, or about 40 molt % to about 62 mol %. In some embodiments, the LNP or tLNP comprises about 58 mol %, about 60 mol %, or 62 mol % ionizable cationic lipid. In some embodiments, the LNP (or tLNP) comprises a phospholipid in an amount in the range of from about 7 to about 30 mol %, or any integer bound sub-range thereof, e.g., in an amount of from about 13 to about 30 mol %. In some embodiments, the LNP or tLNP comprises about 10 mol % phospholipid. In some embodiments, the LNP (or tLNP) comprises a sterol in an amount in the range of from about 20 to about 50 mol % or any integer bound sub-range thereof, e.g., in an amount in the range of from about 20 to about 45 mol %, or about 30 to about 50 mol %, or about 30 to about 45 mol %. In some embodiments, the LNP or tLNP comprises about 30.5, 26.5, or 23.5 mol % sterol. In some embodiments, the LNP (or tLNP) comprises at least one co-lipid in an amount in the range of from about 1 to about 30 mol %. In some embodiments, an LNP or tLNP comprises total PEG-lipid in an amount in the range of from about 1 mol % to about 5 mol % or any integer x 10-1 bound sub-range thereof, e.g., in an amount in the range of from about 1 mol % to about 2 mol % total PEG-lipid. In some embodiments, the LNP (or tLNP) comprises at least one unfunctionalized PEG-lipid in an amount of from O to about 5 mol % or any integer x 101 bound sub-range thereof, e.g., in the range of amount O to about 3 mol %, or about 1 to about 5 mol %, about 0.5 to about 5 mol %, or about 0.5 to about 3 mol %. In some embodiments, the LNP or tLNP comprises about 1.4 mol % unfunctionalized PEG-lipid. In some embodiments, the LNP or tLNP comprises at least one functionalized PEG-lipid in an amount in the range of from about 0.1 to about 5 mol % or any integer x 101 bound sub-range thereof, e.g., in the range of from about 0.1 to 0.3 mol %. In certain embodiments, an LNP or tLNP comprises about 0.1 mol %, about 0.2 mol %, or about 0.3 mol % functionalized PEG-lipid. In some embodiments, the LNP or tLNP comprises about 0.1 mol % functionalized PEG-lipid. In some embodiments, the functionalized PEG-lipid is conjugated to a binding moiety. In certain instances, a tLNP is an LNP that further comprises an antibody (for example, a whole IgG) as the binding moiety which is present at an antibody:mRNA ratio (w/w) of about 0.3 to about 1.0.

In certain aspects, this disclosure provides an LNP or tLNP, wherein the LNP or tLNP comprises about 35 mol % to about 65 mol % of an ionizable cationic lipid, about 0.5 mol % to about 3 mol % of a PEG-lipid (including non-functionalized PEG-lipid and optionally a functionalized PEG-lipid), about 7 mol % to about 13 mol % of a phospholipid, and about 30 mol % to about 50 mol % of a sterol. In some embodiments, an LNP or tLNP comprises a payload with a net negative charge for example, a peptide, a polypeptide, a protein, a small molecule, or a nucleic acid molecule, and combinations thereof. A payload is generally encompassed by or in the interior of an LNP or tLNP. As disclosed herein dosages always refer to the amount of payload being provided. In some embodiments, a payload comprises one or more species of nucleic acid molecule. For tLNP encapsulating mRNA dosages are typically in the range of 0.05 to 5 mg/kg without regard for recipient species. In some embodiments, the dosage is in the range of 0.1 to 1 mg/kg.

With respect to LNPs or tLNPs of this disclosure, in some embodiments, the ratio of total lipid to nucleic acid is about 10:1 to about 50:1 on a weight basis. In some embodiments, the ratio of total lipid to nucleic acid is about 10:1, about 20:1, about 30:1, or about 40:1 to about 50:1, or 10:1 to 20:1, 30:1, 40:1 or 50:1, or any range bound by a pair of these ratios. The ratio of lipid to nucleic acid can also be reported as an N/P ratio, the ratio of positively chargeable lipid amine (N=nitrogen) groups to negatively-charged nucleic acid molecule phosphate (P) groups. In some embodiments, the N/P ratio is from about 3 to about 9, about 3 to about 7, about 3 to about 6, about 4 to about 6, about 5 to about 6, or about 6. In some embodiments, the N/P ratio is from 3 to 9, 3 to 7, 3 to 6, 4 to 6, 5 to 6, or 6. In certain embodiments as described herein, the LNP (or tLNP) comprises a binding moiety, wherein the binding moiety comprises an antigen binding domain of an antibody and wherein the antibody is a whole antibody and the ratio of a lipid to nucleic acid is in the range of from about 0.3 to about 1.0 w/w.

Due to physiologic and manufacturing constraints LNP or tLNP, particles with a hydrodynamic diameter of about 50 to about 150 nm are desirable for in vivo use. Accordingly, in some embodiments, the LNP or tLNP has a hydrodynamic diameter of 50 to 150 nm and in some embodiments the hydrodynamic diameter is ≤120, ≤110, ≤100, or ≤90 nm. Uniformity of particle size is also desirable with a polydispersity index (PDI) of ≤0.2 (on a scale of 0 to 1) being acceptable. Both hydrodynamic diameter and polydispersity index are determined by dynamic light scattering (DLS). Particle diameter as assessed from cryo-transmission electron microscopy (Cryo-TEM) can be smaller than the DLS-determined value.

Particular compositions for precursors to tLNPs and tLNPs are disclosed in U.S. patent application Ser. No. 18/731,223 filed May 31, 2024, as well as PCT Application No. PCT/US23/72426 filed Aug. 17, 2023, each of which is incorporated by reference in its entirety. LNP and tLNP compositions can include those of Table 19. In various embodiments, N/P can be from 3 to 9 or any integer-bound sub-range in that range or about any integer in that range.

### Phospholipids

As described above, in various embodiments, the LNPs and tLNPs include a phospholipid. As would be understood by the person or ordinary skill in the art, phospholipids are amphiphilic molecules. Due to the amphiphilic nature of phospholipids, these molecules are known to form bilayers and by including them in the LNPs and tLNPs, as described herein, they can provide membrane formation, stability, and rigidity. As used herein, phospholipids include a hydrophilic head group, including a functionalized phosphate group, and two hydrophobic tail groups derived from fatty acids. For example, in various embodiments as described herein, the phospholipids include a phosphate group functionalized with ethanolamine, choline, glycerol, serine, or inositol. As described above, the phospholipid includes two hydrophobic tail groups derived from fatty acids. These hydrophobic tail groups can be derived from unsaturated or saturated fatty acids. For example, the hydrophobic tail groups can be derived from a C12-C20 fatty acid.

With respect to LNPs or tLNPs of this disclosure, in various embodiments, the phospholipid comprises dimyristoylphosphatidyl glycerol (DMPG), dimyristoylphosphatidyl choline (DMPC), dipalmitoyl phosphatidylcholine (DPPC), distearoylphosphatidylcholine (DSPC), distearoyl-glycero-phosphate (18:0 PA, DSGP), dioleoylphosphatidyl ethanolamine (DOPE), dioleoyl-glycero-phosphate (18:1 PA, DOGP), or diarachidoylphosphotidylcholine (DAPC), or a combination thereof. In various embodiments, the phospholipid is dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC). In some embodiments, the phospholipid is distearoylphosphatidylcholine (DSPC). Phospholipids can contribute to formation of a membrane, whether monolayer, bilayer, or multi-layer, surrounding the core of the LNP or tLNP. Additionally, phospholipids such as DSPC, DMPC, DPPC, DAPC impart stability and rigidity to membrane structure. Phospholipids, such as DOPE, impart fusogenicity. Further phospholipids, such as DMPG, which attains negative charge at physiologic pH, facilitates charge modulation. Thus, phospholipids constitute means for facilitating membrane formation, means for imparting membrane stability and rigidity, means for imparting fusogenicity, and means for charge modulation. Some embodiments specifically include one or more of the above phospholipids while other embodiments specifically exclude one or more of the above phospholipids.

In some embodiments, an LNP or tLNP has about 7 mol % to about 13 mol % phospholipid, about 7 mol % to about 10 mol % phospholipid, or about 10 mol % to about 13 mol % phospholipid. In certain embodiments, an LNP has about 7 mol %, about 10 mol %, or about 13 mol % phospholipid. In certain instances, the phospholipid is DSPC. In certain instances, the phospholipid is DAPC.

### Sterols

The disclosed LNP and tLNP comprise a sterol. Sterol refers to a subgroup of steroids that contain at least one hydroxyl (OH) group. More specifically, a gonane derivative with an OH group substituted for an H at position 3, or said differently, but equivalently, a steroid with an OH group substituted for an H at position 3. Examples of sterols include, without limitation, cholesterol, ergosterol, P-sitosterol, stigmasterol, stigmastanol, 20-hydroxycholesterol, 22-hydroxycholesterol, and the like. With respect to LNPs or tLNPs of this disclosure, in various embodiments, the sterol is cholesterol, 20-hydroxycholesterol, 20(S)-hydroxycholesterol, 22-hydroxycholesterol, or a phytosterol or combinations thereof. In further embodiments, the phytosterol comprises campesterol, sitosterol, or stigmasterol, or combinations thereof. In certain embodiments, the cholesterol is not animal-sourced but is obtained by synthesis using a plant sterol as a starting point. LNPs incorporating C-24 alkyl (such as methyl or ethyl) phytosterols have been reported to provide enhanced gene transfection. The length of the alkyl tail, the flexibility of the sterol ring, and polarity related to a retained C-3 —OH group are important to obtaining high transfection efficiency. While P-sitosterol and stigmasterol performed well, vitamin D2, D3 and calcipotriol, (analogs lacking intact body of cholesterol) and betulin, lupeol ursolic acid and olenolic acid (comprising a 5th ring) should be avoided. Sterols serve to fill space between other lipids in the LNP or tLNP and influence LNP or tLNP shape. Sterols also control fluidity of lipid compositions, reducing temperature dependence. Thus, sterols such as cholesterol, ergosterol, 20-hydroxycholesterol, 22-hydroxycholesterol, campesterol, fucosterol, P-sitosterol, and stigmasterol constitute means for controlling LNP shape and fluidity or sterol means for increasing transfection efficiency. Some embodiments specifically include one or more of the above sterols while other embodiments specifically exclude one or more of the above sterols. In designing a lipid composition for a LNP or tLNP, in some embodiments, sterol content can be chosen to compensate for different amounts of other types of lipids, for example, ionizable cationic lipid or phospholipid.

In some embodiments, an LNP or tLNP has about 27 mol % or about 30 mol % to about 50 mol % sterol, or about 30 mol % to about 38 mol % sterol. In certain embodiments, an LNP or tLNP has about 30.5 mol %, about 33.5 mol %, or about 37.5 mol % sterol. In certain embodiments, an LNP or tLNP has 27 mol % or 30 mol % to 50 mol % sterol or 30 mol % to 38 mol % sterol. In further embodiments, an LNP or tLNP has 30.5 mol %, 33.5 mol %, or 37.5 mol % sterol. In certain instances, the sterol is cholesterol. In certain embodiments, the sterol is a mixture of sterols, for example, cholesterol and P-sitosterol or cholesterol and 20-hydroxycholesterol. In some instances, the sterol component is about 25 mol % 20-hydroxycholesterol and about 75 mol % cholesterol. In some instances, the sterol component is about 25 mol % P-sitosterol and about 75 mol % cholesterol. In some instances, the sterol component is about 50 mol % P-sitosterol and about 50 mol % cholesterol. In some instances, a sterol component is 25 mol % 20-hydroxycholesterol and 75 mol % cholesterol. In further instances, a sterol component is 25 mol % P-sitosterol and 75 mol % cholesterol. In still further instances, a sterol component is 50 mol % P-sitosterol and 50 mol % cholesterol.

### Co-Lipids

With respect to the LNP or the tLNP, in some embodiments, the co-lipid is absent or comprises an ionizable lipid. In some embodiments the ionizable lipid is cholesterol hemisuccinate (CHEMS). In some embodiments, the co-lipid is a charged lipid, such as a quaternary ammonium headgroup-containing lipid. In some instances, the quaternary ammonium headgroup-containing lipid comprises 1,2-dioleoyl-3-trimethylammonium propane (DOTAP), N-(1-(2,3-dioleyloxy)propyl)-N,N,N-trimethylammonium chloride (DOTMA), or 3β-(N—(N′,N′-Dimethylaminoethane)carbamoyl)cholesterol (DC-Chol), or combinations thereof. In addition to the chloride salts of the quaternary ammonium headgroup containing lipids, further instances include bromide, mesylate, and tosylate salts.

### PEG-Lipids

With respect to a LNP or tLNP of this disclosure, a PEG-lipid is a lipid conjugated to a polyethylene glycol (PEG). In some embodiments as described herein, the PEG-lipid is a C14-C20 lipid conjugated with a PEG. For example, in various embodiments as described herein, the PEG-lipid is a C14-C20 lipid conjugated with a PEG, or a C14-C18 lipid conjugated with a PEG, or a C14-C16 lipid conjugated with a PEG. In certain embodiments as described herein, the PEG-lipid is a fatty acid conjugated with a PEG. The fatty acid of the PEG-lipid can have a variety of chain lengths. For each, in some embodiments, the PEG-lipid is a fatty acid conjugated with PEG, wherein the fatty acid chain length is in the range of C14-C20 (e.g., in the range of C14-C18, or C14-C16). PEG-lipids with fatty acid chain lengths less than C14 are too rapidly lost from the LNP or tLNP while those with chain lengths greater than C20 are prone to difficulties with formulation.

PEG can be made in a large range of sizes. In certain embodiments, the PEG of the disclosed LNP and tLNP is PEG-1000 to PEG-5000. It is to be understood that polyethylene preparations of these sizes are polydisperse and that the nominal size indicates an approximate average molecular weight of the distribution. Taking the molecular weight of an individual repeating unit of (OCH2CH2)n to be 44, a PEG molecule with n=22 would have a molecular weight of 986, with n=45 a molecular weight of 1998, and with n=113 a molecular weight of 4990. n≈22 to 113 is used to represent PEG-lipids incorporating PEG moieties in the range of PEG-1000 to PEG-5000 such as PEG-1000, PEG-1500, PEG-2000, PEG-2500, PEG-3000, PEG-3500, PEG-4000, PEG-4500, and PEG-5000, although some molecules from preparations at the average molecular weight boundaries will have an n outside that range. For individual preparations n≈22 is used to represent PEG-lipids incorporating PEG moieties from PEG-1000, n≈45 is used to represent PEG-lipids incorporating PEG moieties from PEG-2000 n≈67 is used to represent PEG-lipids incorporating PEG moieties from PEG-3000, n≈90 is used to represent PEG-lipids incorporating PEG moieties from PEG-4000, n≈113 is used to represent PEG-lipids incorporating PEG moieties from PEG-5000. Some embodiments incorporate PEG moieties in a range bounded by any pair of the foregoing values of n or average molecular weight. In some embodiments of the PEG-lipid, a PEG is of 500-5000 or 1000-5000 Da molecular weight (MW). For example, in some embodiments, the PEG of the PEG-lipid has a molecular weight in the range of 1500-5000 Da or 2000-5000 Da. In some embodiments as described herein, the PEG-lipid has a molecular weight in the range of 500-4000 Da, or 500-3000 Da, or 1000-4000 Da, or 1000-3000, or 1000-2500, or 1500-4000, or 1500-3000, or 1500-2500 Da. In some embodiments, the PEG moiety is PEG-500, PEG-1000, PEG-1500, PEG-2000, PEG-2500, PEG-3000, PEG-3500, PEG-4000, PEG-4500, and PEG-5000. In some embodiments, the PEG unit has a MW of 2000 Da (sometime abbreviated as PEG(2k)). Some embodiments incorporate PEG moieties of PEG-1000, PEG-2000, or PEG-5000. In some instances, the PEG moiety is PEG-2000. Certain embodiments comprise a DSG-PEG, for example, DSG-PEG-2000. Certain embodiments comprise a DSPE-PEG, for example, DSPE-PEG-2000. Certain embodiments comprise both DSG-PEG-2000 and/or DSPE-PEG2000.

Common PEG-lipids fall into two classes: diacyl glycerols and diacyl phospholipids. Examples of diacyl glycerol PEG-lipids include DMG-PEG (1,2-dimyristoyl-glycero-3-methoxypolyethylene glycol), DPG-PEG (1,2-dipalmitoyl-glycero-3-methoxypolyethylene glycol), DSG-PEG (1,2-distearoyl-glycero-3-methoxypolyethylene glycol), and DOG-PEG (1,2-dioleoyl-glycero-3-methoxypolyethylene glycol). Examples of diacyl phospholipids include DMPE-PEG (1,2-dimyristoyl-glycero-3-phosphoethanolamine-3-methoxypolyethylene glycol), DPPE-PEG (1,2-dipalmitoyl-glycero-3-phosphoethanolamine-3-methoxypolyethylene glycol), DSPE-PEG (1,2-distearoyl-glycero-3-phosphoethanolamine-3-methoxypolyethylene glycol), and DOPE-PEG (1,2-dioleoyl-glycero-3-phosphoethanolamine-3-methoxypolyethylene glycol).

In some embodiments, the MW2000 PEG-lipid (e.g., a PEG-lipid comprising a PEG of a molecular weight of 2000 Da) comprises DMG-PEG2000 (1,2-dimyristoyl-glycero-3-methoxypolyethylene glycol-2000), DPG-PEG2000 (1,2-dipalmitoyl-glycero-3-methoxypolyethylene glycol-2000), DSG-PEG2000 (1,2-distearoyl-glycero-3-methoxypolyethylene glycol-2000), DOG-PEG2000 (1,2-dioleoyl-glycero-3-methoxypolyethylene glycol-2000), DMPE-PEG200 (1,2-dimyristoyl-glycero-3-phosphoethanolamine-3-methoxypolyethylene glycol-2000), DPPE-PEG2000 (1,2-dipalmitoyl-glycero-3-phosphoethanolamine-3-methoxypolyethylene glycol-2000), DSPE-PEG2000 (1,2-distearoyl-glycero-3-phosphoethanolamine-3-methoxypolyethylene glycol-2000), DOPE-PEG2000 (1,2-dioleoyl-glycero-3-phosphoethanolamine-3-methoxypolyethylene glycol-2000), or combinations thereof. In some embodiments, the PEG unit has a MW of 2000 Da. In some embodiments, the MW2000 PEG-lipid comprises DMrG-PEG2000 (1,2-dimyristoyl-rac-glycero-3-methoxypolyethylene glycol-2000), DPrG-PEG2000 (1,2-dipalmitoyl-rac-glycero-3-methoxypolyethylene glycol-2000), DSrG-PEG2000 (1,2-distearoyl-rac-glycero-3-methoxypolyethylene glycol-2000), DorG-PEG2000 (1,2-dioleoyl-glycero-3-methoxypolyethylene-rac-glycol-2000), DMPEr-PEG200 (1,2-dimyristoyl-rac-glycero-3-phosphoethanolamine-3-methoxypolyethylene glycol-2000), DPPEr-PEG2000 (1,2-dipalmitoyl-rac-glycero-3-phosphoethanolamine-3-methoxypolyethylene glycol-2000), DSPEr-PEG2000 (1,2-distearoyl-rac-glycero-3-phosphoethanolamine-3-methoxypolyethylene glycol-2000), DOPEr-PEG2000 (1,2-dioleoyl-rac-glycero-3-phosphoethanolamine-3-methoxypolyethylene glycol-2000), or combinations thereof. The glycerol in these lipids is chiral. Thus, in some embodiments, the PEG-lipid is racemic. Alternatively, optically pure antipodes of the glycerol portion can be employed, that is, the glycerol portion is homochiral. As used herein with respect to glycerol moieties, optically pure means ≥95% of a single enantiomer (D or L). In some embodiments, the enantiomeric excess is >98%. In some embodiments, the enantiomeric excess is >99%. Additional PEG-lipids, including achiral PEG-lipids built on a symmetric dihydroxyacetone scaffold, a symmetric 2-(hydroxymethyl)butane-1,4-diol, or a symmetric glycerol scaffold, are disclosed in U.S. Provisional Application No. 63/362,502, filed on Apr. 5, 2022, and PCT/US2023/017648 application filed on Apr. 5, 2023 (WO 2023/196445), both entitled PEG-Lipids and Lipid Nanoparticles, which are incorporated by reference in their entirety.

The above PEG-lipid examples are presented as methoxypolyethylene glycols, but the terminus need not necessarily be methoxyl. With respect to any of the PEG-lipids that have not been functionalized, in alternative embodiments, the PEG moiety of the PEG lipids can terminate with a methoxyl, a benzyloxyl, a 4-methoxybenzyloxyl, or a hydroxyl group (that is, an alcohol). The terminal hydroxyl facilitates functionalization. The methoxyl, benzyloxyl, and 4-methoxybenzyloxyl groups are advantageously provided for PEG-lipid that will be used as a component of the LNP without functionalization. However, all four of these alternatives are useful as the (non-functionalized) PEG-lipid component of LNPs. The 4-methoxybenzyloxyl group, often used as a protecting group during synthesis of the PEG-lipid, is readily removed to generate the corresponding hydroxyl group. Thus, the 4-methoxybenzyloxyl group offers a convenient path to the alcohol when it is not synthesized directly. The alcohol is useful for being functionalized, prior to incorporation of the PEG-lipid into a LNP, so that a binding moiety can be conjugated to it as a targeting moiety for the LNP (making it a tLNP). As used herein, the terminus of the PEG moiety, and similar constructions, refers to the end of the PEG moiety that is not attached to the lipid.

A PEG-moiety provides a hydrophilic surface on the LNP, inhibiting aggregation or merging of LNP, thus contributing to their stability and reducing polydispersity, i.e. reducing the heterogeneity of a dispersion of LNPs. Additionally, a PEG moiety can impede binding by the LNP, including binding to plasma proteins. These plasma proteins include apoE which is understood to mediate uptake of LNP by the liver so that inhibition of binding can lead to an increase in the proportion of LNP reaching other tissues. These plasma proteins also include opsonins so that inhibition of binding reduces recognition by the reticuloendothelial system. The PEG-moiety can also be functionalized to serve as an attachment point for a targeting moiety. Conjugating a cell- or tissue-specific binding moiety to the PEG-moiety enables a tLNP to avoid the liver and bind to its target tissue or cell type, greatly increasing the proportion of LNP that reaches the targeted tissue or cell type. PEG-lipid can thus serve as means for inhibiting LNP binding, and PEG-lipid conjugated to a binding moiety can serve as means for LNP-targeting.

As used herein, the term “functionalized PEG-lipid” and similar constructions refer generally to both the unreacted and reacted entities. The lipid composition of a LNP can be described referencing the reactive species even after conjugation has taken place (forming a tLNP). For example, a lipid composition can be described as comprising DSPE-PEG-maleimide and can be said to further comprise a binding moiety without explicitly noting that upon reaction to form the conjugate the maleimide will have been converted to a succinimide (or hydrolyzed succinimide). Similarly, if the reactive group is bromomaleimide, after conjugation it will be maleimide. These differences of chemical nomenclature for the unreacted and reacted species are to be implicitly understood even when not explicitly stated. Certain embodiments comprise a DSG-PEG, for example, DSG-PEG-2000. Certain embodiments comprise a functionalized DSPE-PEG, for example, functionalized DSPE-PEG-2000. Certain embodiments comprise both DSG-PEG-2000 and functionalized DSPE-PEG-2000. In some instances, the functionalized PEG-lipid is functionalized with a maleimide moiety, for example, DSPE-PEG-2000-MAL.

In certain aspects, the LNP comprises one or more PEG-lipids and/or functionalized PEG-lipids; when both a functionalized and unfunctionalized PEG-lipid, the PEG-lipid present they can be the same or different; and one or more ionizable cationic lipids; the LNP can further comprise a phospholipid, a sterol, a co-lipid, or any combination thereof. The term “functionalized PEG-lipid” refers to a PEG-lipid in which the PEG moiety has been derivatized with a chemically reactive group that can be used for conjugating a targeting moiety to the PEG-lipid. The functionalized PEG-lipid can be reacted with a binding moiety so that the binding moiety is conjugated to the PEG portion of the lipid. The conjugated binding moiety can thus serve as a targeting moiety for the LNP to constitute a tLNP. In some embodiments, the binding moiety is conjugated to the functionalized PEG-lipid after an LNP comprising the functionalized PEG-lipid is formed. In other embodiments, the binding moiety is conjugated to the PEG-lipid and then the conjugate is inserted into a previously formed LNP.

In certain embodiments, the LNP is a tLNP comprising one or more functionalized PEG-lipids that has been conjugated to a binding moiety. In certain embodiments, the tLNP also comprises PEG-lipids not functionalized or conjugated with a binding moiety. In some embodiments, the functionalization is a maleimide. In some embodiments the functionalization is a bromomaleimide or bromomaleimide amide, alkynylamide, or alkynylimide moiety at the terminal hydroxyl end of the PEG moiety. In some embodiments, the binding moiety comprises an antibody or antigen binding portion thereof. In some embodiments, the binding moiety is a polypeptide comprising a binding domain and an N- or C-terminal extension comprising an accessible thiol group. In some embodiments, the conjugation linkage comprises a reaction product of a thiol in the binding moiety with a functionalized PEG-lipid. In some embodiments, the functionalization is a maleimide, azide, alkyne, dibenzocyclooctyne (DBCO), bromomaleimide or bromomaleimide amide, alkynylamide, or alkynylimide. In some embodiments, the binding moiety comprises an antibody or antigen binding portion thereof. In some embodiments, the binding moiety is a polypeptide comprising a binding domain and an N- or C-terminal extension comprising an accessible thiol group.

In certain embodiments, the PEG-lipid and/or functionalized PEG-lipid comprises a scaffold selected from Formula S1, Formula S2, Formula S3, or Formula S4:

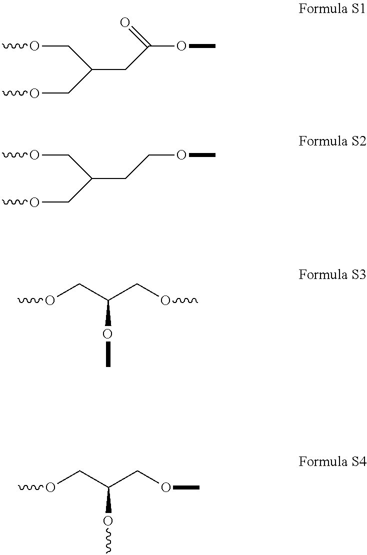

wherein [symbol] represents the points of ester connection with a fatty acid, and [symbol] represents the point of ester (S1) or ether (S2, S3, and S4) formation with the PEG moiety. In some embodiments, the fatty acid esters are C14-C20 straight-chain alkyl fatty acids. In some embodiments, the PEG moiety is functionalized and the fatty acid esters are C16-C20 straight-chain alkyl fatty acids. For example, the straight-chain alkyl fatty acid is C14, C15, C16, C17, C18, C19, or C20. In some embodiments, the fatty acid esters are C14-C20 symmetric branched-chain alkyl fatty acids. For example, the branched-chain alkyl fatty acid is C14, C15, C16, C17, C18, C19, or C20. By symmetric it is meant that each alkyl branch has the same number of carbons. In some embodiments, the branch is at the 3, 4, 5, 6, or 7 position of the fatty acid ester. The synthesis and use of PEG-lipids built on scaffolds S1-S4 is disclosed in WO2023/196445A1 which is incorporated by reference for all that it teaches about PEG-lipids and their use.

Some embodiments of the disclosed ionizable cationic lipids have head groups with small (<250 Da) PEG moieties. These lipids are not what is meant by the term PEG-lipid as used herein. These small PEG moieties are generally too small to impede binding to a similar extent as the larger PEG moieties of the PEG-lipids disclosed above, though they will impact the lipophilicity of ionizable cationic lipid. Moreover, the PEG-lipids are understood to be primarily located in an exterior facing lamella whereas much of the ionizable cationic lipid is in the interior of the LNP.

In certain embodiments, a functionalized PEG-lipid of a LNP or tLNP or this disclosure comprises one or more fatty acid tails, each that is no shorter than C16 nor longer than C20 for straight-chain fatty acids. For branched chain fatty acids, tails no shorter than C14 fatty acids nor longer than C20 are acceptable. In some embodiments, fatty acid tails are C16. In some embodiments, the fatty acid tails are C18. In some embodiments, the functionalized PEG-lipid comprises a dipalmitoyl lipid. In some embodiments, the functionalized PEG-lipid comprises a distearoyl lipid. The fatty acid tails serve as means to anchor the PEG-lipid in the tLNP to reduce or eliminate shedding of the PEG-lipid from the tLNP. This is a useful property for the PEG-lipid whether or not it is functionalized but has greater significance for the functionalized PEG-lipid as it will have a targeting moiety attached to it and the targeting function could be impaired if the PEG-lipid (with the conjugated binding moiety, such as an antibody) were shed from the tLNP.

In some embodiments, an LNP or tLNP comprises about 0.5 mol % to about 3 mol % or 0.5 mol % to 3 mol % PEG-lipid comprising functionalized and non-functionalized PEG-lipid. In certain embodiments, an LNP or tLNP comprises DSG-PEG. In other embodiments, an LNP or tLNP comprises DMG-PEG or DPG-PEG. In certain embodiments, an LNP or tLNP comprises DSPE-PEG. In some embodiments, the functionalized and non-functionalized PEG-lipids are not the same PEG-lipid, for example, the non-functionalized PEG-lipid can be a diacylglycerol and the functionalized PEG-lipid a diacyl phospholipid. tLNP with such mixtures have reduced expression in the liver, possibly due to reduced uptake. In certain embodiments the functionalized PEG-lipid is DSPE-PEG and the non-functionalized PEG-lipid is DSG-PEG. In some embodiments, an LNP or tLNP comprises about 0.4 mol % to about 2.9 mol % or about 0.9 mol % to about 1.4 mol % non-functionalized PEG lipid. In certain embodiments, an LNP or tLNP comprises about 1.4 mol % or 1.4 mol % non-functionalized PEG lipid. In some embodiments, an LNP or tLNP comprises about 0.1 mol % to about 0.3 mol % or 0.1 mol % to 0.3 mol % functionalized lipid. In some instances, the functionalized lipid is DSPE-PEG. In certain instances, an LNP or tLNP comprises about 0.1 mol %, about 0.2 mol %, or about 0.3 mol % DSPE-PEG. In certain instances, an LNP or tLNP comprises 0.1 mol %, 0.2 mol %, or 0.3 mol % DSPE-PEG. In certain instances, the functionalized PEG-lipid is conjugated to a binding moiety. As used herein, the phrase “is conjugated to” and similar constructions are meant to convey a state of being, that is, a structure, and not a process, unless context dictates otherwise.

### Conjugation

Any suitable chemistry can be used to conjugate the binding moiety to the PEG of the PEG-lipid, including maleimide (see Parhiz et al., Journal of Controlled Release 291:106-115, 2018) and click (see Kolb et al., Angewandte Chemie International Edition 40(11):2004-2021, 2001; and Evans, Australian Journal of Chemistry 60(6):384-395, 2007) chemistries. Reagents for such reactions include lipid-PEG-maleimide, lipid-PEG-cysteine, lipid-PEG-alkyne, lipid-PEG-dibenzocyclooctyne (DBCO), and lipid-PEG-azide. Further conjugations reactions make use of lipid-PEG-bromomaleimide, lipid-PEG-alkylnoic amide, lipid-PEG-alkynoic imide, and lipid-PEG-alkyne reactions, as disclosed in PCT/US23/17648 entitled PEG-Lipids and Lipid Nanoparticles, which is incorporated by reference for all that it teaches about conjugation chemistry and alternative PEG-lipids. On the binding moiety side of the reaction one can use an existing cysteine sulfhydryl, or derivatize the protein by adding a sulfur containing carboxylic acid, for example, to the epsilon amino of a lysine to react with maleimide, bromomaleimide, (collectively, “a maleimide”), alkylnoic amide, or alkynoic imide. Alternatively, one can add an alkyne to a sulfhydryl or an epsilon amino of a lysine to participate in a click chemistry reaction.

To modify an epsilon amino of a binding moiety lysine to react with a maleimide functionalized PEG-lipid the binding moiety (e.g., an antibody) can be reacted with N-succinimidyl S-acetylthioacetate (SATA). SATA is then deprotected, for example, using 0.5 M hydroxylamine followed by removal of the unreacted components by G-25 Sephadex Quick Spin Protein columns (Roche Applied Science, Indianapolis, IN). The reactive sulfhydryl group on the binding moiety is then conjugated to maleimide moieties on LNPs of the disclosure using thioether conjugation chemistry. Purification can be performed using Sepharose CL-4B gel filtration columns (Sigma-Aldrich). tLNPs (LNPs conjugated with a targeting antibody) can be stored frozen at −80° C. until needed. Others have conjugated antibody to free functionalized PEG-lipid and then incorporated the conjugated lipid into pre-formed LNP. However, it was found that this procedure is more controllable and produces more consistent results.

There are also several approaches to site-specific conjugation. Particularly but not exclusively suitable for truncated forms of antibody, C-terminal extensions of native or artificial sequences containing a particularly accessible cysteine residue are commonly used. Partial reduction of cystine bonds in an antibody, for example, with tris(2-carboxy)phosphine (TCEP), can also generate thiol groups for conjugation which can be site-specific under defined conditions with an amenable antibody fragment. Alternatively, the C-terminal extension can contain a sortase A substrate sequence, LPXTG (SEQ ID NO: 1) which can then be functionalized in a reaction catalyzed by sortase A and conjugated to the PEG-lipid, including through click chemistry reactions (see, for example, Moliner-Morro et al., Biomolecules 10(12):1661, 2020 which is incorporated by reference herein for all that it teaches about antibody conjugations mediated by the sortase A reaction and/or click chemistry). The use of click chemistry for the conjugation of a targeting moiety, such as various forms of antibody, is disclosed, for example, in WO2024/102,770 which is incorporated by reference in its entirety for all that it teaches about the conjugation of targeting moieties to LNPs that is not inconsistent with this disclosure.

For whole antibody and other forms comprising an Fc region, site-specific conjugation to either (or both) of two specific lysine residues (Lys248 and Lys288) can be accomplished without any change to or extension of the native antibody sequence by use of one of the AJICAP® reagents (see, for example, Matsuda et al., Molecular Pharmaceutics 18:4058-4066, 2021 and Fujii et al., Bioconjugate Chemistry doi.org/10.1021/acs.bioconjchem.3c00040, 2023, which are incorporated by reference herein for all that they teach about conjugation of antibodies with AJICAP reagents). The AJICAP reagents are modified affinity peptides that bind to specific loci on the Fc and react with an adjacent lysine residue. The peptide is then cleaved with base to leave behind a thiol-functionalized lysine residue which can then undergo conjugation through maleimide or haloamide reactions, for example). Functionalization with azide or dibenzocyclooctyne (DBCO) for conjugation by click chemistry is also possible. This and similar technology are further described in US 2020/0190165 (corresponding to WO 2018/199337), US 2021/0139549 (corresponding to WO 2019/240287) and US 2023/0248842 (corresponding to WO 2020/184944) which are incorporated by reference in their entirety for all that they teach about such modified affinity peptides and their use.

Accordingly, in some embodiments the binding moiety is conjugated to the PEG moiety of the PEG-lipid through a thiol modified lysine residue. In some embodiments, the conjugation is through a cysteine residue in a native or added antibody sequence. In some embodiments, a particular cysteine residue is preferentially or exclusively reacted, for example, a cysteine residue in an antibody hinge region. In further instances, a binding moiety with a conjugatable cysteine residue in an antibody hinge region is an Fab′ or similar fragment. In other embodiments, the conjugation is through a sortase A substrate sequence. In still other embodiments, the conjugation is through a specific lysine residue (Lys248 or Lys288) in the Fc region.

### Ionizable Cationic Lipids

Ionizable cationic lipids are useful components for complexing with negatively charged payloads and for promoting delivery of the payload into the cytoplasm of a cell following endocytosis. Accordingly, each of the hereinbelow disclosed genera and species of ionizable cationic lipid can be used in defining the scope of embodiments of the herein disclosed LNP and tLNP compositions and pharmaceutical compositions, and methods of using them. In certain embodiments, ionizable cationic lipid(s) of an LNP having a measured pKa of 6 to 7 can remain essentially neutral in the blood stream and interstitial spaces but ionize after uptake into cells as the endosomes acidify. Upon acidification in the endosomal space, the lipid becomes protonated, and associates more strongly with the phosphate backbone of the nucleic acid, which destabilizes the structure of the LNP and promotes nucleic acid release from the LNP into the cell cytoplasm (also referred to as endosomal escape). Thus, the herein disclosed ionizable cationic lipids constitute means for destabilizing LNP structure (when ionized) or means for promoting nucleic acid release or endosomal escape. In some embodiments the ionizable cationic lipid has a c-pKa from 8 to 11 and cLogD from 9 to 18 or 11-14. In some embodiments, the ionizable cationic lipids have branched structure to give the lipid a conical rather than cylindrical shape. Suitable ionizable cationic lipids are known to those of skill in the art. In some embodiments, the LNP comprises a lipid composition comprising an ionizable cationic lipid having a structure of Formula 1, Formula 2, Formula 3, Formula M5, Formula M1, CICL, CICL-IE, Formula M6 or Formula M2 including species or subgenera thereof, as disclosed in International Application Number PCT/US2023/017647 published as WO 2023/196444, International Application Number PCT/US2024/049649 filed on Oct. 2, 2024, International Application Number PCT/US2024/049627 filed on Oct. 2, 2024, and U.S Provisional Application Nos 63/654,704 filed May 31, 2024, and 63/654,720 filed May 31, 2024, each of which further describe the use of ionizable cationic lipids and LNP incorporating them, and are each incorporated by reference in its entirety.

For simplicity, chemical moieties are defined and referred to throughout primarily as univalent chemical moieties (e.g., alkyl, aryl, and the like). Nevertheless, such terms can also be used to convey corresponding multivalent moieties under the appropriate structural circumstances clear to those skilled in the art. For example, while an “alkyl” moiety generally refers to a monovalent radical (e.g. CH3-CH2-), in certain circumstances a bivalent linking moiety can be “alkyl,” in which case those skilled in the art will understand the alkyl to be a divalent radical (e.g., —CH2-CH2-), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for nitrogen, 2 for oxygen, and 2, 4, or 6 for sulfur, depending on the oxidation state of the sulfur atom).

The term “alkyl” as employed herein refers to saturated straight and branched chain aliphatic groups having from 1 to 12 carbon atoms. As such, “alkyl” encompasses C1, C2, C3, C4, C5, C6, C7, C8, C9, C10, C11 and C12 groups.

The term “alkenyl” as used herein means an unsaturated straight or branched chain aliphatic group with one or more carbon-carbon double bonds, having from 2 to 12 carbon atoms. As such, “alkenyl” encompasses C2, C3, C4, C5, C6, C7, C8, C9, C10, C11 and C12 groups.

In some embodiments, the hydrocarbon chain is unsubstituted. In other embodiments, one or more hydrogens of the alkyl or alkenyl group can be substituted with the same or different substituents.

Aryl refers to an aromatic or heteroaromatic ring lacking one hydrogen leaving a bond that connects to another portion of an organic molecule. Examples of aryl include, without limitation, phenyl, naphthalenyl, pyridine, pyrimidine, pyrazine, pyrrole, furan, thiophene, imidazole, thiazole, oxazole, and the like.

Aryl-alkyl refers to a moiety comprising one or more aryl rings and one or more alkyl moieties. The position of the one or more aryl rings can vary within the alkyl portion of the moiety. For example, the one or more aryl rings can be at an end of the one or more alkyl moieties, be fused into the carbon chain of the one or more alkyl moieties, or substitute one or more hydrogens of one or more alkyl moieties; and the one or more alkyl moieties can substitute one or more hydrogens of the one or more aryl rings. In some embodiments, there is a single ring; while in other embodiments, that are multiple rings.

Branched alkyl is a saturated alkyl moiety wherein the alkyl group is not a straight chain. Alkyl portions such as methyl, ethyl, propyl, butyl, and the like, can be appended to variable positions of the main alkyl chain. In some embodiments, there is a single branch; while in other embodiments, there are multiple branches.

Branched alkenyl refers to an alkenyl group comprising at least one branch off the main chain which can be formed by substituting one or more hydrogens of the main chain with the same or different alkyl groups, e.g., without limitation, methyl, ethyl, propyl, butyl, and the like.

In some embodiments, a branched alkenyl is a single branch structure, while in other embodiments, a branched alkenyl can have multiple branches.

Straight chain alkyl is a non-branched, non-cyclic version of the alkyl moiety described above.

Straight chain alkenyl is a non-branched, non-cyclic version of the alkenyl moiety described above.

In some embodiments as described herein, the ionizable cationic lipids have a structure of Formula 1:

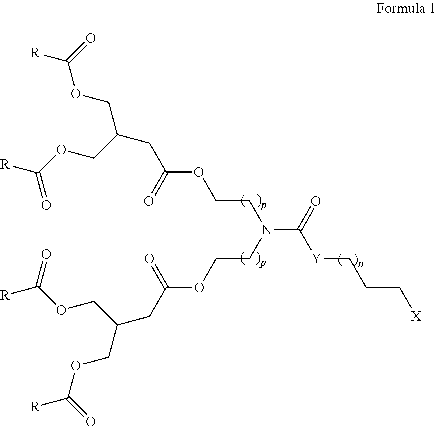

wherein:
Y is O, NH, N—CH3, or CH2,
n is an integer from O to 4,
X is

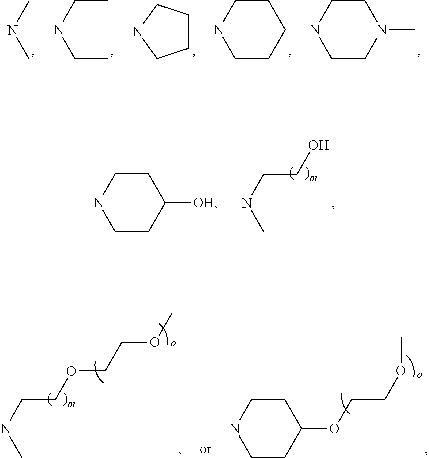

m is an integer from 1 to 3,
o is an integer from 1 to 4,
p is an integer from 1 to 4,
wherein when p=1:
   each R is independently C6 to C16 straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain;
wherein when p=2:
   each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or Cs to C₁₆ aryl-alkyl in which the aryl is phenyl or naphthalenyl and is positioned at either end or within the alkyl chain;
wherein when p=3:
   each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; or Cs to C₁₄ aryl-alkyl in which the aryl is phenyl or naphthalenyl and is positioned at the either end or within the alkyl chain; and
wherein when p=4:
   each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to Cs cycloalkyl positioned at either end or within the alkyl; or C₈ to C₁₂ aryl-alky in which the aryl is phenyl or naphthalenyl and is positioned at the either end or within the alkyl chain.

In certain aspects, the ionizable cationic lipids of this disclosure have a structure of the formula M5:

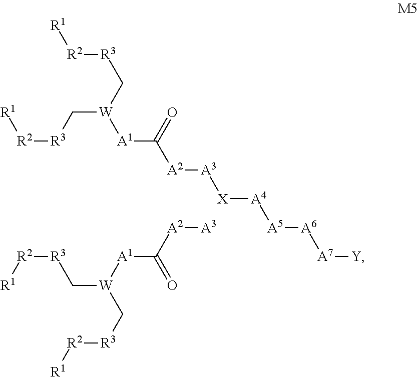

wherein:
each R¹ is independently selected from a C₇-C₁₁ alkyl or a C₇-C₁₁ alkenyl,
A¹ is (CH2)₁₋₂,
A² is O,
A³ is (CH₂)₁₋₅, wherein A³ is not CH₂ if X is N,
X is N, CH, or C—CH₃,
A⁴ is CH₂, C═O, NH, NCH₃, or O,
A⁵ is absent, O, S, NH, or NCH₃ if A⁴ is C═O, or A⁵ is C═O if A⁴ is not C═O,
A⁶ is O, S, NH, NCH₃ or (CH₂)₀₋₂,
A⁷ is (CH₂)₀₋₆, wherein if A⁶ is O, S, NH, NCH₃, A⁷ is (CH₂)₂₋₄,
Y is

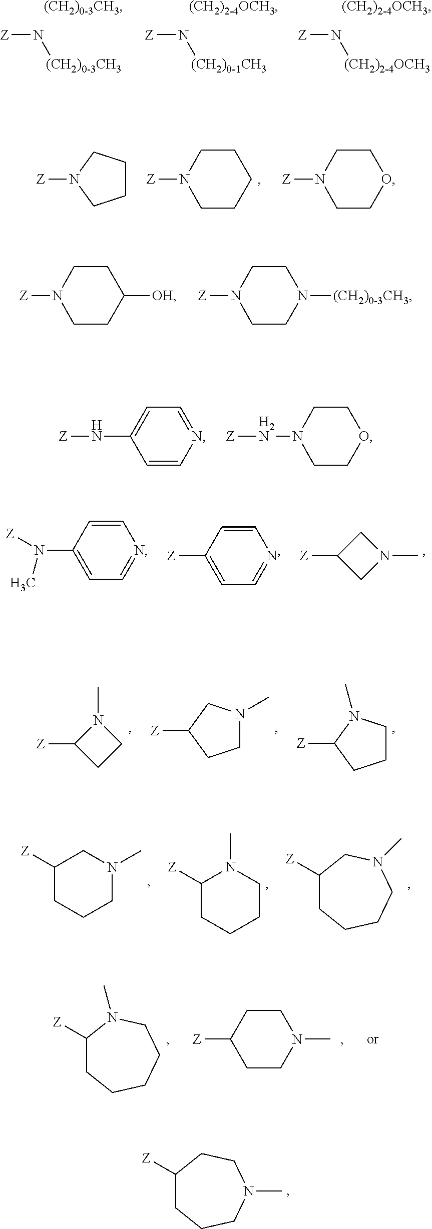

wherein Z is a bond; and
R² is O, R³ is C═O and W is CH or N, or R² is C═O, R³ is O and W is CH;
   wherein A⁶ and A⁷ are not both (CH₂)₀ unless A⁵ is C═O;
   wherein
a), A¹ is CH₂, A³ is (CH₂)₂₋₅, X is N, A⁴ is C═O, A⁵ is O, S, NH, NCH₃, A⁶ is (CH₂)₁₋₂, A⁷ is (CH₂)₁₋₄, or
b) A¹ is CH₂, A³ is (CH₂)₁₋₄, X is CH, A⁴ is CH₂, NH, NCH₃, O, A⁵ is C═O, A⁶ is O, NH, NCH₃, A⁷ is (CH)₂₋₆, or
c) A¹ is (CH₂)2, A³ is (CH₂)₁₋₄, X is C—CH₃, A⁴ is C═O, A⁵ is O, NH, NCH₃, A⁶ is (CH₂)₁₋₂, A⁷ is (CH₂)₁₋₄, or
d) A¹ is CH₂, A³ is (CH₂)₂₋₅, X is N, A⁴ is C═O, A⁵ is absent, A⁶ is (CH₂)₀, A⁷ is (CH₂)₀, and Y is

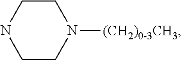

or
e) A¹ is CH₂, A³ is (CH₂)₁₋₅, X is CH, A⁴ is CH₂, NH, NCH₃ or O, A⁵ is C═O, A⁶ is (CH₂)₀, A⁷ is (CH₂)₀, and Y is

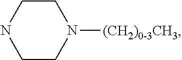

or
f) A¹ is (CH₂)₂, A³ is (CH₂)₁₋₅, X is CCH₃, A⁴ is C═O, A⁵ is absent, A⁶ is (CH₂)₀, A⁷ is (CH₂)₀, and Y is

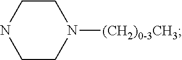

wherein
the number of contiguous atoms present in a span:

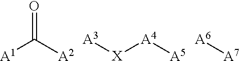

is in the range from 7-17.

As used herein, when a subscript has a value of “0”, the group is absent. For example, when A⁶ is (CH₂)₀, A⁶ is absent.

In certain embodiments of formula M5, R² is O, R³ is C═O and W is CH or N. For example, in certain embodiments of formula M5, R² is O, R³ is C═O and W is CH.

In certain embodiments of formula M5, R² is C═O, R³ is O and W is CH.

In certain embodiments of formula M5, A¹ is CH₂, A³ is (CH₂)₂₋₅, X is N, A⁴ is C═O, A⁵ is O, S, NH, NCH₃, A⁶ is (CH₂)₁₋₂, and A⁷ is (CH₂)₁₋₄. For example, in certain embodiments A¹ is CH₂, A³ is (CH₂)₂₋₅, X is N, A⁴ is C═O, A⁵ is O, A⁶ is (CH₂)₁₋₂, and A⁷ is (CH₂)₁₋₄.

In certain embodiments of formula M5, A¹ is CH₂, A³ is (CH₂)₁₋₄, X is CH, A⁴ is CH₂, NH, NCH₃, O, A⁵ is C═O, A⁶ is O, NH, NCH₃, or CH₂, and A⁷ is (CH)₂₋₆. In certain embodiments as described herein, A¹ is CH₂, A³ is (CH₂)₁₋₄, X is CH, A⁴ is NH, A⁵ is C═O, A⁶ is O, NH, NCH₃, or CH₂, and A⁷ is (CH)₂₋₆. For example, in certain embodiments of formula M5, A¹ is CH₂, A³ is (CH₂)₁₋₄, X is CH, A⁴ is NH, A⁵ is C═O, A⁶ is O, and A⁷ is (CH)₂₋₆. For example, in certain embodiments of formula M5, A¹ is CH₂, A³ is (CH₂)₁₋₄, X is CH, A⁴ is NH, A⁵ is C═O, A⁶ is CH₂, and A⁷ is (CH)₂₋₆. In certain embodiments as described herein, A¹ is CH₂, A³ is (CH₂)₁₋₄, X is CH, A⁴ is CH₂, A⁵ is C═O, A⁶ is O, NH, NCH₃, or CH₂, or A⁷ is (CH)₂₋₆. For example, in certain embodiments of formula M5, A¹ is CH₂, A³ is (CH₂)₁₋₄, X is CH, A⁴ is CH₂, A⁵ is C═O, A⁶ is O, A⁷ is (CH)₂₋₆. In certain embodiments as described herein, A¹ is CH₂, A³ is (CH₂)₁₋₄, X is CH, A⁴ is O, A⁵ is C═O, A⁶ is O, NH, NCH₃, or CH₂, and A⁷ is (CH)₂₋₆. For example, in certain embodiments of formula M5, A¹ is CH₂, A³ is (CH₂)₁₋₄, X is CH, A⁴ is O, A⁵ is C═O, A⁶ is CH₂, and A⁷ is (CH)₂₋₆. In certain embodiments as described herein, A¹ is CH₂, A³ is (CH₂)₁₋₄, X is CH, A⁴ is NCH₃, A⁵ is C═O, A⁶ is O, NH, NCH₃, or CH₂, and A⁷ is (CH)₂₋₆. For example, in certain embodiments of formula M5, A¹ is CH₂, A³ is (CH₂)₁₋₄, X is CH, A⁴ is NCH₃, A⁵ is C═O, A⁶ is CH₂, and A⁷ is (CH)₂₋₆.

In certain embodiments of formula M5, A¹ is (CH₂)₂, A³ is (CH₂)₁₋₄, X is C—CH₃, A⁴ is C═O, A⁵ is O, NH, NCH₃, A⁶ is (CH₂)₁₋₂, or A⁷ is (CH₂)₁₋₄. For example, in certain embodiments, A¹ is (CH₂)₂, A³ is (CH₂)₁₋₄, X is C—CH₃, A⁴ is C═O, A⁵ is O, A⁶ is (CH₂)₁₋₂, or A⁷ is (CH₂)₁₋₄.

In certain embodiments of formula M5, the number of contiguous connective atoms present in a span:

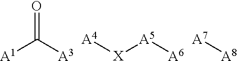

is in the range from 7-17. For example, in certain embodiments, the number of contiguous connective atoms present in a span:

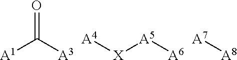

is in the range of 7-11 or 7-10. In certain embodiments, the number of contiguous connective atoms present in a span:

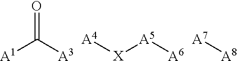

is in the range of 10-17 (e.g., in the range of 10-16, or 10-14, or 10-12). For example, in certain embodiments, the number of contiguous connective atoms present in a span:

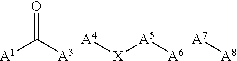

is 10. For example, in certain embodiments, the number of contiguous connective atoms present in a span:

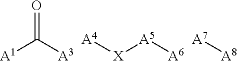

is 7. The present inventors have found that changing the number of contiguous connective atoms present in each span can allow for tuning of the pKa of the cationic lipid.

In some embodiments of formula M5, Y is

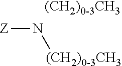

and Z is a bond. In some embodiments of formula M5, Y is

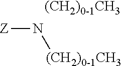

and Z is a bond. For example, in some embodiments of formula M5, Y is

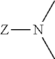

and Z is a bond.

In some embodiments of formula M5, Y is

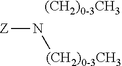

and Z is a bond. In some embodiments of formula M5, Y is

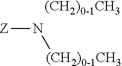

and Z is a bond. For example, in some embodiments of formula M5, Y is

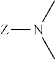

and Z is a bond.

In some embodiments of formula M5, Y is

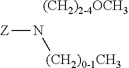

and Z is a bond. For example, on some embodiments of formula M5, Y is

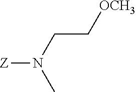

and Z is a bond.

In some embodiments of formula M5, Y is

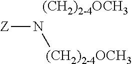

and Z is a bond. For example, in some embodiments of formula M5, Y is

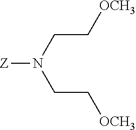

and Z is a bond.

In some embodiments of formula M5, Y is

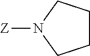

and Z is a bond.

In some embodiments of formula M5, Y is

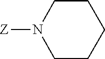

and Z is a bond.

In some embodiments of formula M5, Y is

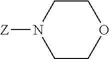

and Z is a bond.

In some embodiments of formula M5, Y is

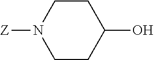

and Z is a bond.

In some embodiment of formula M5, Y is

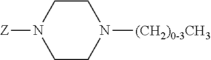

and Z is a bond.

In some embodiments of formula M5, Y is

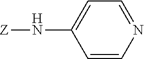

and Z is a bond.

In some embodiments of formula M5, Y is

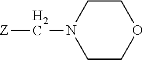

and Z is a bond.

In some embodiments of formula M5, Y is

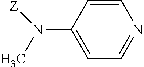

and Z is a bond.

In some embodiments of formula M5, Y is

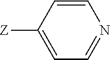

and Z is a bond.

In some embodiments of formula M5, Y is

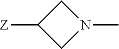

and Z is a bond.

In some embodiments of formula M5, Y is

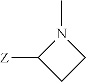

and Z is a bond.

In some embodiments of formula M5, Y is

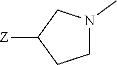

and Z is a bond.

In some embodiments of formula M5, Y is

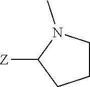

and Z is a bond.

In some embodiments of formula M5, Y is

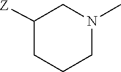

and Z is a bond.

In some embodiments of formula M5, Y is

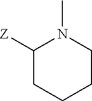

and Z is a bond.

In some embodiments of formula M5, Y is

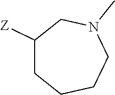

and Z is a bond.

In some embodiments of formula M5, Y is

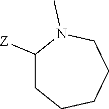

and Z is a bond.

In some embodiments of formula M5, Y is

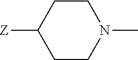

and Z is a bond.

In some embodiments of formula M5, Y is

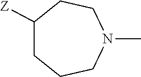

and Z is a bond.

In some embodiments, the ionizable cationic lipid has the structure CICL

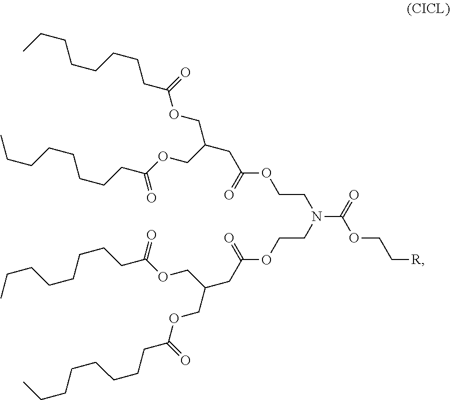

wherein R is

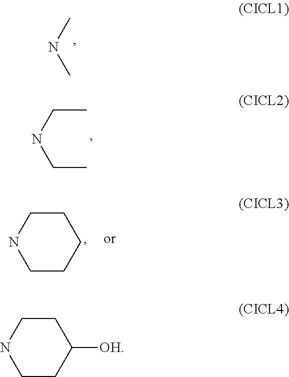

In certain embodiments, the ionizable cationic lipid of CICL is referred to as CICL1 when R is

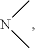

that is

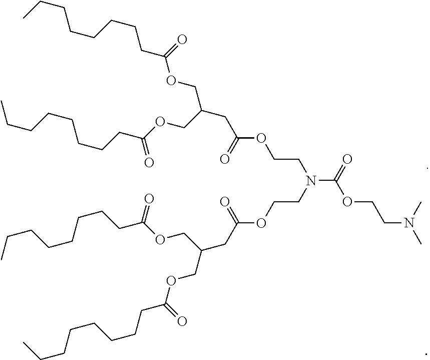 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OCCN(C)C]

In certain aspects, the constrained ionizable cationic lipids of this disclosure have a structure of the formula M6:

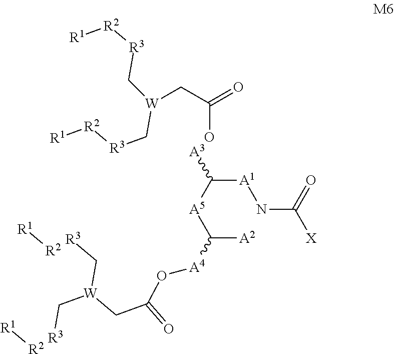

wherein X is

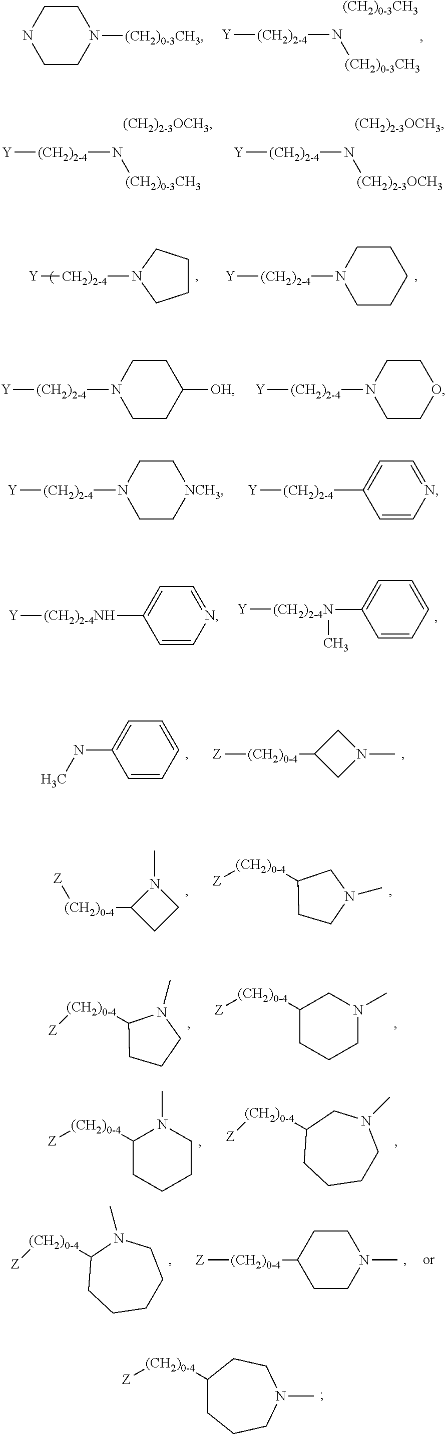

and
Y is O, S, NH, or NCH₃;
Z is O, NH, or NCH₃;
R² is O, R³ is C═O and W is CH or N, or R² is C═O, R³ is O and W is CH; and
each R¹ is independently selected from a C₇-C₁₁ alkyl or a C₇-C₁₁ alkenyl;
each A¹, A², A³, and A⁴ is independently selected from (CH₂)₀ and (CH₂)₁,
A⁵ is selected from (CH₂)₀₋₄, CH═CH, and CH₂—CH═CH—CH₂; and
a wavy bond indicates that any relative or absolute stereo-configuration of the corresponding ring atom, or a mixture of stereo-configurations, can be assumed.

As used herein, when a subscript has a value of “0”, the group is absent. For example, when A¹ is (CH₂)₀, A¹ is absent.

In certain embodiments of formula M6, R² is O, R³ is C═O and W is CH or N. For example, in certain embodiments of formula M6, R² is O, R³ is C═O and W is CH.

In certain embodiments of formula M6, R² is C═O, R³ is O and W is CH.

In various embodiments of M6, A¹ through A⁴ are chosen so that there are only two main chain atoms between the ring nitrogen and each nearest ester oxygen in the nearest tail group.

In certain embodiments of M6, A¹ is (CH₂)₀, A² is (CH₂)₀, A³ is (CH₂)₁, A⁴ is (CH₂)₁, and A⁵ is (CH₂)₁₋₄ or CH₂—CH═CH—CH₂.

In certain embodiments of M6, A¹ is (CH₂)₀, A² is (CH₂)₁, A³ is (CH₂)₁, A⁴ is (CH₂)₀, and A⁵ is (CH₂)₁.

In certain embodiments of M6, A¹ is (CH₂)₁, A² is (CH₂)₁, A³ is (CH₂)₀, A⁴ is (CH₂)₀, and A⁵ is (CH₂)₀.

In certain embodiments of M6, A¹ is (CH₂)₁, A² is (CH₂)₁, A³ is (CH₂)₀, A⁴ is (CH₂)₀, and A⁵ is (CH₂)₁.

In certain embodiments of M6, A¹ is (CH₂)₁, A² is (CH₂)₁, A³ is (CH₂)₀, A⁴ is (CH₂)₀, and A⁵ is (CH₂)₂ or CH═CH.

In some embodiments of formula M6 as described herein, X is

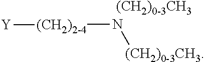

For example, in some embodiments of formula M6, X is

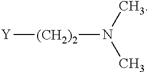

In some embodiments of formula M6 as described herein, X is

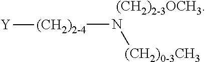

In some embodiments of formula M6 as described herein, X is

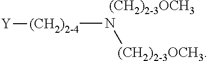

In some embodiments of formula M6 as described herein, X is

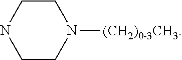

In some embodiments X is

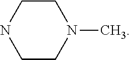

In some embodiments of formula M6 as described herein, X is

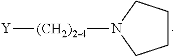

In some embodiments of formula M6 as described herein, X is

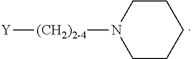

In some embodiments of formula M6 as described herein, X is

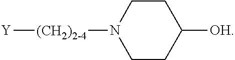

In some embodiments of formula M6 as described herein, X is

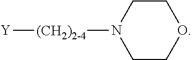

In some embodiments of formula M6 as described herein, X is

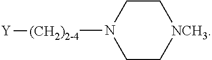

In some embodiments of formula M6 as described herein, X is

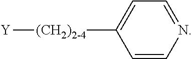

In some embodiments of formula M6 as described herein, X is

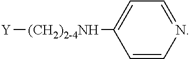

In some embodiments of formula M6 as described herein, X is

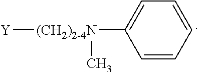

In some embodiments of formula M6 as described herein, X is

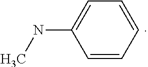

In some embodiments of formula M6, X is

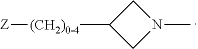

In some embodiments of formula M6, X is

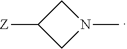

In some embodiments of formula M6, X is

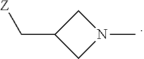

In some embodiments of formula M6, X is

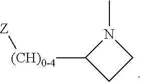

In some embodiments of formula M6, X is

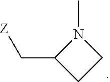

In some embodiments of formula M6, X is

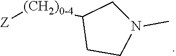

In some embodiments of formula M6, X is

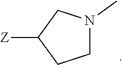

In some embodiments of formula M6, X is

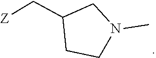

In some embodiments of formula M6, X is

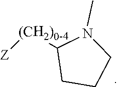

In some embodiments of formula M6, X is

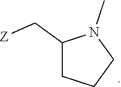

In some embodiments of formula M6, X is

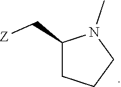

In some embodiments of formula M6, X is

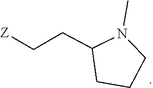

In some embodiments of formula M6, X is

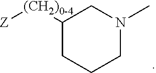

In some embodiments of formula M6, X is

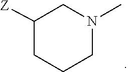

In some embodiments of formula M6, X is

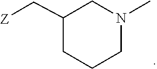

In some embodiments of formula M6, X is

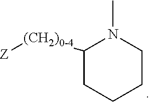

In some embodiments of formula M6, X is

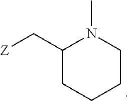

In some embodiments of formula M6, X is

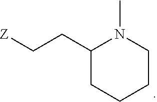

In some embodiments of formula M6, X is

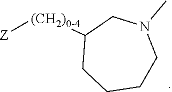

In some embodiments of formula M6, X is

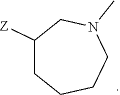

In some embodiments of formula M6, X is

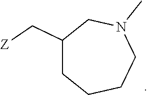

In some embodiments of formula M6, X is

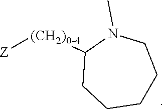

In some embodiments of formula M6, X is

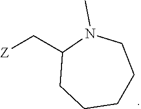

In some embodiments of formula M6, X is

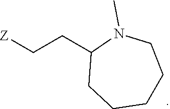

In some embodiments of formula M6, X is

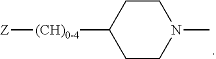

In some embodiments of formula M6, X is

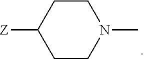

In some embodiments of formula M6, X is

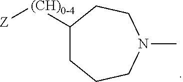

In some embodiments of formula M6, X is

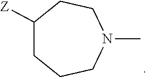

As described above, in some embodiments of formula M6, Y can be selected from O, S, NH, or NCH₃. In some embodiments of formula M6, Y is O. In some other embodiments of formula M6, Y is S.

In some embodiments of formula M6, X is

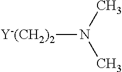

and Y is O. In some embodiments of formula M6, X is

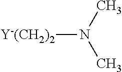

### and Y is S.

As described above, Z can be selected from O, NH, or NCH₃. In some embodiments, Z is O.

In some embodiments of formula M6, X is

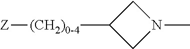

and Z is O. In some embodiments of formula M6, X is

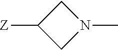

and Z is O. In some embodiments of formula M6, X is

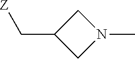

### and Z is O.

In some embodiments of formula M6, X is

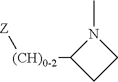

and Z is O. In some embodiments of formula M6, X is

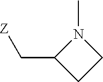

### and Z is O.

In some embodiments of formula M6, X is

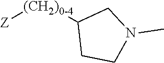

and Z is O. In some embodiments of formula M6, X is

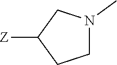

and Z is O. In some embodiments of formula M6, X is

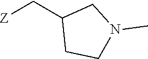

### and Z is O.

In some embodiments of formula M6, X is

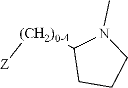

and Z is O. In some embodiments of formula M6, X is

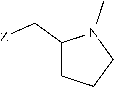

and Z is O. In some embodiments of formula M6, X is

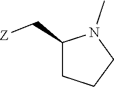

and Z is O. In some embodiments of formula M6, X is

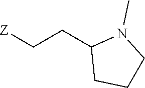

### and Z is O.

In some embodiments of formula M6, X is

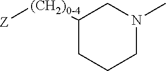

and Z is O. In some embodiments of formula M6, X is

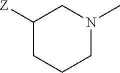

and Z is O. In some embodiments of formula M6, X is

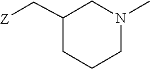

### and Z is O.

In some embodiments of formula M6, X is

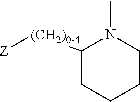

and Z is O. In some embodiments of formula M6, X is

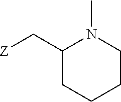

and Z is O. In some embodiments of formula M6, X is

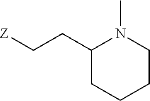

### and Z is O.

In some embodiments of formula M6, X is

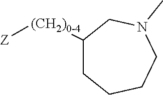

and Z is O. In some embodiments of formula M6, X is

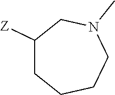

and Z is O. In some embodiments of formula M6, X is

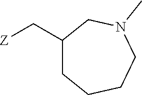

### and Z is O.

In some embodiments of formula M6, X is

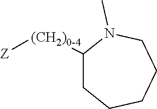

and Z is O. In some embodiments of formula M6, X is

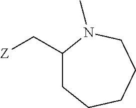

and Y is O. In some embodiments of formula M6, X is

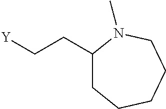

### and Z is O.

In some embodiments of formula M6, X is

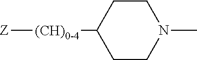

and Z is O. In some embodiments of formula M6, X is

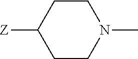

### and Z is O.

In some embodiments of formula M6, X is

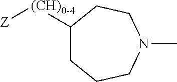

### and Z is O.

In some embodiments of formula M6, X is

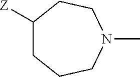

### and Z is O.

In some embodiments, the ionizable cationic lipid has the structure CICL-207:

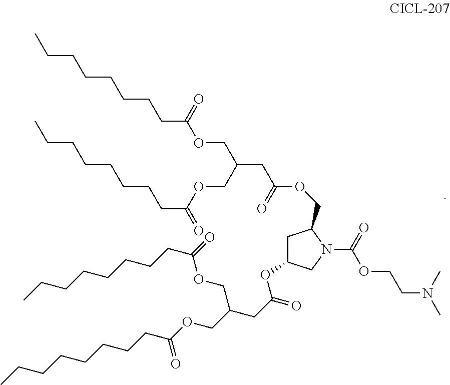 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(C)C]

In some embodiments, the ionizable cationic lipid has the structure CICL-215:

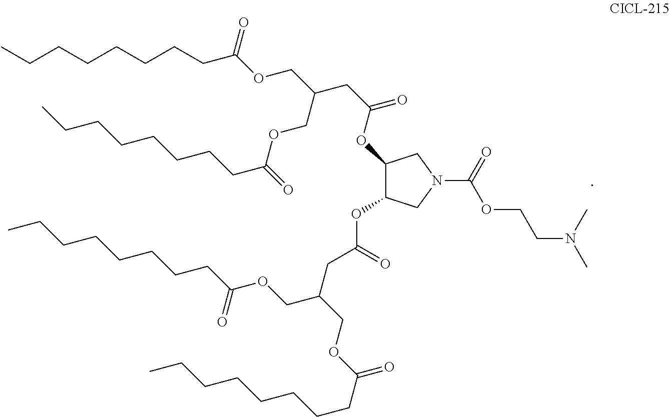 [SMILES: CCCCCCCCC(=O)OCC(COC(=O)CCCCCCCC)CC(=O)O[C@H]1CN(C(=O)OCCN(C)C)C[C@@H]1OC(=O)CC(COC(=O)CCCCCCCC)COC(=O)CCCCCCCC]

In some embodiments, the ionizable cationic lipid has the structure CICL-225:

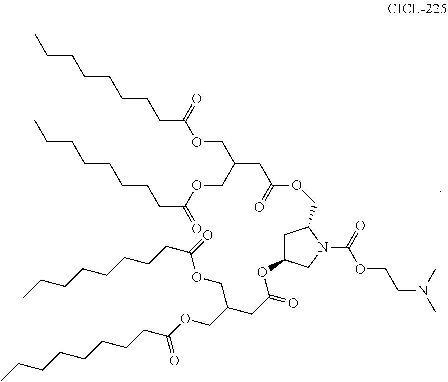 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OCCN(C)C]

As described above, for both formula M5 and M6, each R is independently selected from C₇-C₁₁ alkyl or C₇-C₁₁ alkenyl. In some embodiments of formula M5 and/or M6, each R¹ is independently selected from C₇-C₁₁ alkyl, e.g., C₇-C₁₀ alkyl, or C₇-C₉ alkyl. In certain embodiments of formula M5 and/or M6, each R¹ is independently selected from a linear C₇-C₁₁ alkyl, e.g., a linear C₇-C₁₀ alkyl, or a linear C₇-C₉ alkyl. In some embodiments of formula M5 and/or M6 as described herein, each R¹ is independently selected from (CH₂)6-8CH₃. In some of these and other embodiments, R¹ is (CH₂)7CH₃. In some embodiments of formula M5 and/or M6, each R¹ is independently selected from a linear C₇-C₁₁ alkenyl, e.g., a linear C₇-C₁₀ alkenyl, or a linear C₇-C₉ alkenyl. For example, in some embodiments of formula M5 and/or M6, each R¹ is a linear C₈ alkenyl. In certain other embodiments of formula M5 and/or M6, each R¹ is independently selected from a branched C₇-C₁₁ alkyl, e.g., C₇-C₁₀ alkyl, or C₇-C₉ alkyl. For example, in some embodiments of formula M5 and/or M6, each R¹ is a branched C₈ alkyl. In certain embodiments of formula M5 and/or M6, each R¹ is independently selected from a branched C₇-C₁₁ alkenyl, e.g., C₇-C₁₀ alkenyl, or C₇-C₉ alkenyl. For example, in some embodiments of formula M5 and/or M6, each R¹ is a branched C₈ alkenyl. In some embodiments of formula M5 and/or M6, wherein R¹ is a branched alkyl or alkenyl, the branch point is positioned so that ester carbonyls are not in an a position relative to the branch point, for example they are in a β position relative to the branch point.

In certain embodiments of formula M5 and/or M6 as described herein, each R¹ is the same. In certain embodiments of formula M5 and/or M6, each R¹ nearest a common branch point is the same, but those nearest a first common branch point differ from those nearest a second common branch point. In certain embodiments of formula M5 and/or M6, each R¹ nearest a common branch point is different but the pair of R¹s nearest a first common branch point is the same the pair nearest a second common branch point.

In certain embodiments of formula M6, the ionizable cationic lipid is substantially enantiomerically pure (e.g., at least about 80%, at least about 85%, at least about 90%, at least about 95%, at least about 96%, at least about 97%, at least about 98%, or at least about 99%). In certain embodiments of formula M6, the ionizable cationic lipid is a racemic mixture. In certain embodiments of formula M6, the ionizable cationic lipid is a mixture of two or more stereoisomers. In certain embodiments of formula M6, at least two of the two or more stereoisomers are diastereomers. In certain embodiments of formula M6, at least two of the two or more stereoisomers are enantiomers.

In some embodiments, an LNP or tLNP comprises about 35 mol % to about 65 mol %, about 40 mol % to about 62 mol %, or about 54 mol % to about 60 mol % ionizable cationic lipid. In some embodiments, the lipid composition is at least 40 mol % and/or does not exceed 62 mol % ionizable cationic lipid. In certain embodiments, an LNP of tLNP comprises about 54 mol %, about 58 mol %, or about 62 mol % ionizable cationic lipid. In further embodiments an LNP comprises 35 mol % to 65 mol %, 40 mol % to 62 mol %, or 54 mol % to 60 mol % ionizable cationic lipid. In still further embodiments, an LNP has at least 40 mol % or does not exceed 62 mol % ionizable cationic lipid. In certain embodiments, an LNP comprises 54 mol %, 58 mol %, or 62 mol % ionizable cationic lipid.

With respect to the LNP or the tLNP, in some embodiments the ratio of total lipid to nucleic acid is 10:1 to 50:1 on a weight basis. In some embodiments, that ratio of total lipid to nucleic acid is 10:1, 20:1, 30:1, or 40:1 to 50:1, or 10:1 to 20:1, 30:1, 40:1 or 50:1, or any range bound by a pair of these ratios.

Particular compositions for precursors to tLNPs and tLNPs are disclosed in U.S. patent application Ser. No. 18/731,223 filed May 31, 2024, as well as PCT Application No. PCT/US23/72426 filed Aug. 17, 2023, each of which is incorporated by reference in its entirety. LNP and tLNP compositions can include those of Table 19. In various embodiments, N/P can be from 3 to 9 or any integer-bound sub-range in that range or about any integer in that range. In some embodiments, the tLNP has the lipid composition of Composition Code F5. In some embodiments, the tLNP has the lipid composition of Composition Code F9. In some embodiments, the tLNP has any lipid composition as disclosed in Table 19 (for example Composition Code F5 or F9) except that any lipid of formula M5 or M6 has been substituted for CICL-1, for example, CICL-207, CICL-215, or CICL-225.

mRNA

In certain instances, the mRNA disclosed herein is a “nucleoside-modified mRNA,” which refers to an mRNA comprising at least one modified nucleoside. A “modified nucleoside” refers to a nucleoside with a modification relative to the common nucleosides found in naturally occurring nucleic acids. For example, over one hundred different nucleoside modifications have been identified in RNA (Rozenski, et al., 1999, The RNA Modification Database: 1999 update. Nucl Acids Res 27: 196-197).

In some embodiments, some or all of the uridines of the mRNA have been replaced with one or more types of a pseudouridine, or other modified nucleoside(s). In certain embodiments, “a pseudouridine” refers to N1-methyl pseudouridine (N1Mψ or m¹ψ). In certain embodiments, “a pseudouridine” refers to 5-methoxyuridine (5moU). In another embodiment, “a pseudouridine” refers to m¹acp³Y (1-methyl-3-(3-amino-3-carboxypropyl) pseudouridine. In another embodiment, the term refers to m¹Y (1-methylpseudouridine). In another embodiment, the term refers to Ym (2′-O-methylpseudouridine. In another embodiment, the term refers to m⁵D (5-methyldihydrouridine). In another embodiment, the term refers to m³Y (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the herein disclosed inventions. Further modified nucleosides are disclosed in WO2007024708 which is incorporated by reference in its entirety for all that it teaches about modified nucleosides and their use in RNA.

In certain embodiments, the mRNA sequences can be synthesized by in vitro transcription (IVT). Such in vitro transcription can be accomplished using a phage RNA polymerase such as T7 RNA polymerase and beneficially provides substitution of uridine with a pseudouridine (e.g., without limitation, N1-methylpseudouridine (N1Mψ) or 5-methoxyuridine). Templates used in such IVT method comprise linear DNA molecules comprising the 5′ UTR, ORF and 3′ UTR sequences inserted between a T7 RNA polymerase promoter and a poly(A) tail (e.g., about 90 to about 110 adenine residues) in which the poly(A) tail is on the sense strand at the 3′ end of the linear DNA. Various restriction sites can be used for the insertion. Examples of restriction sites include, without limitation, EcoR1, BamH1, and BsrG1. Advantageous UTRs and UTR pairs are disclosed in International Patent Application No. PCT/US2024/054033 which is incorporated by reference for all that it teaches about the design and production of mRNAs for transfection of and expression in T cells that is not inconsistent with the present disclosure. Typically, template DNA is produced as a bacterial plasmid carrying a selectable marker such as kanamycin resistance. Circular plasmids are linearized at a unique type II restriction enzyme site, located downstream of the poly(A) tail. The linearized plasmid serves as template for IVT. The IVT process can utilize T7 RNA polymerase in reaction conditions known in the art and partial or complete substitution of uridine with a pseudouridine (e.g., without limitation, N1Mψ or 5moU). A Cap1 structure can be added co-transcriptionally using, for example, a cap-AG trinucleotide reagent (m7G(5′)ppp(5′)(2′OMeA)pG). In alternative embodiments, template DNA comprises a T3 or SP6 RNA polymerase promoter instead of the T7 RNA polymerase promoter and the corresponding polymerase is used to synthesize the mRNA.

In certain embodiments, an mRNA has a sequence comprising in 5′ to 3′ order, a 5′ untranslated region (UTR), an open reading frame (ORF) terminated by at least one stop codon, a 3′ UTR, and a poly(A) sequence wherein:
the ORF encodes a CAR, TCR, or TCE; and
the 5′ UTR and 3′ UTR each comprise a sequence, or a variant thereof having >95% sequence identity to the UTR sequence, selected from one of the following pairs:
   a) mouse β-globin (mHBB) 5′ UTR (SEQ ID NO: 228 or SEQ ID NO: 236) and pancreatic triacylglycerol lipase (PNLIP) 3′ UTR (SEQ ID NO: 244 or SEQ ID NO: 253), ribosomal protein S3A (RPS3A) 3′ UTR (SEQ ID NO: 245 or SEQ ID NO: 254), RPS3A-PNLIP tandem 3′ UTR (SEQ ID NO: 246 or SEQ ID NO: 255), PNLIP-RPS3A tandem 3′ UTR (SEQ ID NO: 247 or SEQ ID NO: 256), human hemoglobin subunit α1 (hHBA1) 3′ UTR (SEQ ID NO: 248 or SEQ ID NO: 257), or human hemoglobin subunit al with 3 miRNA122 binding sites (hHBA1-3x miR122 bs) 3′ UTR (SEQ ID NO: 249 or SEQ ID NO: 258);
   b) carboxypeptidase A1 (CPA1) 5′ UTR (SEQ ID NO: 229 or SEQ ID NO: 237) and CPA1 3′ UTR (SEQ ID NO: 250 or SEQ ID NO: 259);
   c) defensin alpha 3 (DEFA3) 5′ UTR (SEQ ID NO: 230 or SEQ ID NO: 238) and DEFA3 3′ UTR (SEQ ID NO: 251 or SEQ ID NO: 260);
   d) human albumin (hAlb) 5′ UTR (SEQ ID NO: 231 or SEQ ID NO: 239) and hHBA1 3′ UTR (SEQ ID NO: 248 or SEQ ID NO: 257);
   e) hemoglobin subunit alpha 1 (HBA) 5′ UTR (SEQ ID NO: 232 or SEQ ID NO: 240) and amino-terminal enhancer of split and mitochondrially encoded 12S rRNA(AES-mtRNR1) 3′ UTR (SEQ ID NO: 252 or SEQ ID NO: 261);
   f) eIF4G aptamer x1 (SEQ ID NO: 233 or SEQ ID NO: 241) and hHBA1 3′ UTR (SEQ ID NO: 248 or SEQ ID NO: 257);
   g) aptamer control (SEQ ID NO: 234 or SEQ ID NO: 242) and hHBA1 3′ UTR (SEQ ID NO: 248 or SEQ ID NO: 257); or
   h) a synthetic 5′ UTR (SEQ ID NO: 235 or SEQ ID NO: 243) and hHBA1 3′ UTR (SEQ ID NO: 248 or SEQ ID NO: 257).

### Antigen Receptors

Three primary types of antigen receptor can be used in immune engineering amplification; CARs, T cell receptors (TCRs), and T cell engagers. CARs comprise one or more intracellular domains that provide an activating or stimulatory signal to T cells and an extracellular domain that can bind to a cell surface antigen. Most commonly, the extracellular domain comprises one or more single-chain Fvs (scFvs). TCRs are the natural antigen receptor of T cells and also have an intracellular signaling domain. TCRs recognize antigens as presented by major histocompatibility complex (MHC) proteins. This offers the advantage that the T cell can be reprogrammed to recognize antigens of intracellular origin in addition to those of cell surface origin but also entails the disadvantages of MHC restriction which limits the proportion of the patient population that can be served with any particular TCR depending on the commonality of the MHC restriction element. T cell engagers (TCEs), like CARs, reprogram T cells to recognize cell surface antigens, thereby redirecting them. However, unlike CARs, TCEs are soluble molecules comprising at least two specificities; one specificity recognizing a T cell activating receptor, typically CD3, and another recognizing a surface antigen of a pathogenic cell to be attacked.

### CARs

There are five generations of CARs that are commonly recognized. “First generation” CARs for use in the invention comprise an antigen binding domain, for example, a single-chain variable fragment (scFv) or VHH, fused to a transmembrane domain, which is fused to a cytoplasmic/intracellular domain of the T cell receptor chain. “First generation” CARs typically have the intracellular signaling (or activation) domain from the CD3ζ-chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation. Use of a CD3ζ intracellular signaling domain in which one or two of the three ITAM motifs has been disrupted can modulate the balance of effector and memory programs (Feucht et al., 2019 Nat Med 25(1):82-88). The intracellular signaling domains of CD3ε or the low affinity receptor for IgG, FcγRIIIA (CD16A) can be used as alternatives to CD3ζ. In some embodiments, the intracellular signaling domain of CD3ε comprises the sequence KNRKAKAKPVTRGAGAGGRQRGQNKERPPPVPNPDYEPIRKGQRDLYSGLNQRRI (SEQ ID NO: 2). In some embodiments, the intracellular signaling domain of FcγRIIIA (CD16A) comprises the sequence of FcγRIIIA: KTNIRSSTRDWKDHKFKWRKDPQDK (SEQ ID NO: 3). In some embodiments, these intracellular signaling domains constitute means for signaling or means for activation.

“Second-generation” CARs for use in the invention comprise an antigen binding domain, for example, a scFv or VHH, fused to a transmembrane domain, which is fused to an intracellular signaling domain capable of activating T cells and a co-stimulatory domain designed to augment T cell potency and persistence (Sadelain et al., 2013, Cancer Discov. 3:388-398). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, CD27, and the like, in the cytoplasmic tail of the CAR to provide additional signals to the cell. “Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of CAR-T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., 2012, Oncoimmunol. 1(9):1577-1583, which is incorporated by reference in its entirety to the extent that it does not conflict with the present disclosure). In some embodiments, these costimulatory domains constitute means for co-stimulation.

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3ζ signaling domain in addition to a constitutive or inducible chemokine component.

The term “Fourth generation” CAR has also been applied to CAR polyprotein constructs that additionally contain anti-cytokine antibodies (scFv) or receptor antagonists with self-cleaving peptides (e.g., T2A, P2A) between the components of the polyprotein. The cytokine receptor antagonist or antibody is secreted by the CAR-T cells and can neutralize cytokines such as IL-10, IL-6, or TNFα to reduce or eliminate adverse side-effects such as CRS (see Li et al., Cell Research (2025) doi.org/10.1038/s41422-024-01068-2, and Xue, L. et al. Cell Discov. 7, 84 (2021) each of which is incorporated for all that they teach about inhibition of CAR-T-related immunologic side effect by co-expression of antibodies or receptor antagonists with the CAR).

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2RP.

Further variations on the basic CAR structure and sources for the various domains are described in Zabel et al., Immunol Lett 2019 212:53-69 which is incorporated by reference for all that it teaches about CAR structure and functional domains thereof to the extent it is consistent with this disclosure.

In some embodiments immunotherapy associated adverse events are further reduced by co-expression of a cytokine receptor antagonist or an anti-cytokine antibody or combinations thereof (for example, to inhibit IL-1β, IL-6, and/or TNFα) in the same cells as the CAR. This can be accomplished by encoding a polyprotein in the tLNP encapsulated mRNA, by encapsulating multiple mRNAs in the tLNP, or by encapsulating individual mRNAs in tLNPs targeted to the same cells.

In some embodiments, a nucleic acid encoding a CAR (or similarly, a TCR, TCE, or any combination therewith) refers to one or more nucleic acid species encoding one or more CARs; for example, a single or multiple species of nucleic acid encoding a single CAR species, or multiple species of nucleic acid encoding multiple CAR species. In some instances, these multiple CAR species have the same specificity while in other instances they have multiple specificities. In some embodiments, a CAR of this disclosure is multispecific, for example, bispecific, comprising multiple antigen binding moieties each specific for separate antigens. In some embodiments, a nucleic acid encoding an antigen receptor can further encode another proteins(s) such as an anti-cytokine antibody or cytokine receptor antagonist, or can be co-encapsulated with such a nucleic acid(s).

### A) Signal Peptide

In certain embodiments, the CAR can comprise a signal peptide at the N-terminus. Non-limiting examples of signal peptides include CD8a signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 1 below.

TABLE 1
Exemplary sequences of signal peptides

| SEQ ID NO: | Sequence | Description |
|---|---|---|
| 4 | MALPVTALLLPLALLLHAARP | CD8α signal peptide |
| 5 | METDTLLLWVLLLWVPGSTG | IgK signal peptide |
| 6 | MLLLVTSLLLCELPHPAFLLIP | GMCSFR-α (CSF2RA) signal peptide |

### B) Extracellular Binding Domain

A CAR comprises an extracellular binding domain, also referred to as a binder or binding moiety. In certain embodiments, the extracellular binding domain can comprise one or more antibodies specific to a single pursued antigen or multiple pursued antigens. The antibody can be an antibody fragment, for example, an scFv, or a single-domain antibody fragment, for example, a VHH. In certain embodiments, the scFv can comprise a heavy chain variable region (VH) and a light chain variable region (VL) of an antibody connected by a linker. The VH and the VL can be connected in either order, i.e., VH-linker-VL or VL-linker-VH. Non-limiting examples of linkers include Whitlow linker, (G4S)n (SEQ ID NO: 233, n can be a positive integer, e.g., 1, 2, 3, 4, 5, 6, etc.) linker, and variants thereof. In certain embodiments, the antigen can be an antigen that is exclusively or preferentially expressed on tumor cells, or an antigen that is characteristic of an autoimmune or inflammatory disease.

Exemplary pursued antigens against which a CAR, TCR, or TCE can have specificity include, but are not limited to, B cell maturation agent (BCMA)†‡, CA9†‡, CD4†‡, CD5†‡, CD19*†‡, CD20 (MS4A1)*†‡, CD22*†‡, FCRL5†‡, GPRC5D t, CD23*†‡, CD30 (TNFRSF8)*†‡, CD33*†‡, CD38*†‡, CD44*†‡, CD70*†‡, CD133‡, CD174, CD274 (PD-L1)*†‡, CD276 (B7-H3)†‡, CEACAM5*†‡, CLL1‡, CSPG4*‡, Kappa*, Lambda*, NCAM1 (CD56)*‡, PD-1 (CD279)†‡, ROR1†‡, CD138 (SDC1)*‡, CD319 (SLAMF7)*†‡, CD248 (TEM1)‡, ULBP1, and ULBP2 (associated with leukemias); CD319 (SLAMF7)*†‡, CD38*†‡, CD138‡, GPRC5†‡, CD267 (TACI){, and BCMA†‡ (associated with myelomas); and Claudin 6 (CLDN6), Claudin 18.2 (CLDN18.2), GD2*†‡, HER2*†‡, EGFR*†‡, EGFRvIII*, CD276 (B7H3)†‡, PSMA*†‡, PSCA‡, CAIX (CA9)†‡, CD171 (L1-CAM)*‡, CEA*‡, CSPG4*‡, DLL3, EPHA2*‡, FAP*†‡, LRRC15†‡, FOLR1*†‡, IL-13Rα*†‡, Mesothelin (MSLN)*†‡, MUC1*†‡, MUC16*†‡, EPCAM*†‡, ERBB2*‡, FOLH1, GPC3*†‡, GPNMB*‡, IL1RAP†‡, IL3RA*‡, IL13RA2 (IL13Rα2)*‡, KDR (VEGFR2)*‡, CD171 (L1CAM)*‡, MET*‡, TROP2*†‡, and ROR1†‡ (associated with solid tumors). Antigens associated with B cell leukemias can also be useful for B cell depletion in non-oncologic applications, however, CD19 (present on pro-B cells, pre-B cells, immature, naïve, germinal center, and memory B cells, and short-lived plasmablasts (sometime referred to as short-lived plasma cells)) and BCMA (present on memory B cells, short-lived plasmablasts, and long-lived plasma cells) are of particularly interest. (wherein * indicates that exemplary antibodies with the indicated specificity from which a binding moiety could be derived can be found in U.S. Pat. No. 11,326,182B2 Table 9 or 10. † indicates that exemplary antibodies with the indicated specificity from which a binding moiety could be derived can be found in Wilkinson & Hale, 2022. Both references cited and incorporated by reference above. ‡ indicates that exemplary antibodies with the indicated specificity from which a binding moiety could be derived can be found in the Therapeutic Antibody Database (TABS) at tabs.craic.com). Other suitable antibodies can be found in Appendix A. Many of these pursued antigens are themselves receptors that could bind to their ligand if expressed on an immune cell. Accordingly, in some embodiments, the extracellular binding domain of the CAR comprises a ligand of a receptor expressed on the target cell. In still further embodiments, the extracellular binding domain of the CAR comprises a ligand binding domain of a receptor for a ligand expressed on the target cell. In any of these embodiments, the extracellular binding domain of the CAR can be codon-optimized for expression in a host cell or have variant sequences to increase functions of the extracellular binding domain. The advantages of the aspects and embodiments disclosed herein are independent of the specificity of the binding moiety. As such, the disclosed aspects and embodiments are generally agnostic to binding specificity. In certain embodiments, a particular binding specificity can be required. A more extensive discussion of antibodies recognizing many of the individual antigens listed above can be found in WIPO Publication WO2024040195A1 and U.S. patent application Ser. No. 18/731,223 which are each incorporated by reference for all that they teach about antibodies and related molecules that can be used to provide binding moieties recognizing pursued antigens.

### C) Hinge Domain

In certain embodiments, the CAR can comprise a hinge domain, also referred to as a spacer. The terms “hinge” and “spacer” can be used interchangeably in this disclosure. Non-limiting examples of hinge domains include CD8a hinge domain, CD28 hinge domain, IgG4 hinge domain, IgG4 hinge-CH₂-CH₃ domain, and variants thereof, the amino acid sequences of which are provided in Table 2 below.

TABLE 2
Exemplary sequences of hinge domains

| SEQ ID NO: | Sequence | Description |
|---|---|---|
| 7 | TTTPAPRPPTPAPTIASQPLSLRPEACRPAA | CD8α hinge domain |
| | GGAVHTRGLDFACD | |
| 8 | IEVMYPPPYLDNEKSNGTIIHVKGKHLCPSP | CD28 hinge domain |
| | LFPGPSKP | |
| 9 | ESKYGPPCPPCP | IgG4 hinge domain |
| 11 | ESKYGPPCPPCPAPEFLGGPSVFLFPPKPKD | IgG4 hinge-CH2-CH3 |
| | TLMISRTPEVTCVVVDVSQEDPEVQFNWY | domain |
| | VDGVEVHNAKTKPREEQFNSTYRVVSVLT | |
| | VLHQDWLNGKEYKCKVSNKGLPSSIEKTIS | |
| | KAKGQPREPQVYTLPPSQEEMTKNQVSLT | |
| | CLVKGFYPSDIAVEWESNGQPENNYKTTPP | |
| | VLDSDGSFFLYSRLTVDKSRWQEGNVFSCS | |
| | VMHEALHNHYTQKSLSLSLGK | |

### D) Transmembrane Domain

In certain embodiments, the CAR can comprise a transmembrane domain. In specific embodiments, the transmembrane domain can comprise a transmembrane region of CD3ζ, CD3ε, CD3γ, CD3δ, CD4, CD5, CD8α, CD8β, CD9, CD16, CD22, CD28, CD32, CD33, CD34, CD3γ, CD40, CD45, CD64, CD8β, CD86, OX40/CD134, 4-1BB/CD137, CD40L/CD154, FAS, FcεRIγ, FGFR2B, TCRα, TCRβ, or VEGFR2, or a functional variant thereof, including the human versions of each of these sequences. Table 3 provides the amino acid sequences of a few exemplary transmembrane domains.

TABLE 3
Exemplary sequences of transmembrane domains

| SEQ ID NO: | Sequence | Description |
|---|---|---|
| 12 | IYIWAPLAGTCGVLLLSLVITLYC | CD8α transmembrane domain |
| 13 | FWVLVVVGGVLACYSLLVTVAFI | CD28 transmembrane domain |
| | IFWV | |

### E) Intracellular Domain

In certain embodiments, the CAR can comprise one or more intracellular signaling domains. The various generations of CARs have included an intracellular domain that provides an activating or stimulatory function, such as from CD3ζ, CD3R, or CD16A. The 2^{nd} and 3^{rd} generation CARs added one or more intracellular domains, respectively, to provide co-stimulatory function, such as from CD28 or 4-1BB among many others. In certain embodiments, the intracellular signaling domain can comprise one or more signaling domains selected from B7-1/CD8β, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain comprises one or more signaling domains selected from a CD3ζ domain, an ITAM, a CD28 domain, 4-11B1 domain, or a functional variant thereof. Table 4 provides amino acid sequences for a few exemplary intracellular signaling domains. 4-111, also known as CD 137, transmits a potent costimulatory signal to T cells, promoting differentiation and enhancing long-term survival of T lymphocytes. CD28 is another co-stimulatory molecule on T cells. CD3 zeta (ζ) associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In certain embodiments, as in the case of tisagenlecleucel as described below, the CD3ζ signaling domain of SEQ ID NO: 16 can have a mutation, e.g., a glutamine (Q) to lysine (K) mutation, at amino acid position 14 (see SEQ ID NO: 17).

TABLE 4
Exemplary sequences of intracellular signaling domains

| SEQ ID NO: | Sequence | Description |
|---|---|---|
| 14 | KRGRKKLLYIFKQPFMRPVQTTQEED | 4-1BB signaling domain |
| | GCSCRFPEEEEGGCEL | |
| 15 | RSKRSRLLHSDYMNMTPRRPGPTRK | CD28 signaling domain |
| | HYQPYAPPRDFAAYRS | |
| 16 | RVKFSRSADAPAYQQGQNQLYNELN | CD3ζ signaling domain |
| | LGRREEYDVLDKRRGRDPEMGGKPR | |
| | RKNPQEGLYNELQKDKMAEAYSEIG | |
| | MKGERRRGKGHDGLYQGLSTATKD | |
| | TYDALHMQALPPR | |
| 17 | RVKFSRSADAPAYKQGQNQLYNELN | CD3ζ signaling domain (with Q |
| | LGRREEYDVLDKRRGRDPEMGGKPR | to K mutation at position 14) |
| | RKNPQEGLYNELQKDKMAEAYSEIG | |
| | MKGERRRGKGHDGLYQGLSTATKD | |
| | TYDALHMQALPPR | |

### F) Exemplary CAR Constructs

In certain embodiments, CARs are used to treat a disease or condition associated with a pursued cell that expresses the antigen pursued by the CAR as described in the uses and methods of treatment disclosed herein. For example, in some embodiments, an anti-CD19 or anti-CD20 or anti-BCMA CAR can be used to pursue and treat B cell malignancies or B cell-mediated autoimmune conditions or diseases. In other embodiments, an anti-FAP CAR can be used to pursue and treat solid tumors or fibrosis (e.g., cardiac fibrosis, cancer-associated fibroblasts). Examples of CARs that can be used in accordance with the embodiments described herein include to those disclosed in U.S. Pat. No. 7,446,190 (anti-CD19), U.S. Pat. No. 10,287,350 (anti-CD19), US2021/0363245 (anti-CD19 and anti-CD20), U.S. Pat. No. 10,543,263 (anti-CD22), U.S. Pat. No. 10,426,797 (anti-CD33), U.S. Pat. No. 10,844,128 (anti-CD123), U.S. Pat. No. 10,428,141 (anti-ROR1), and US2021/0087295 (anti-FAP), each of which is incorporated by reference for all that it teaches about CAR structure and function generically and with respect to the CAR's antigenic specificity and pursued indications to the extent that it is not inconsistent with this disclosure.

In certain embodiments, binding domains from antibodies can be used to construct a CAR to pursue and treat solid tumors or fibrosis. Exemplary binding domains can be obtained from antibodies, such as anti-LRRC15 (WO 2021/102332), anti-FAP (US 2012/0128591; US 2012/0128591; US 2012/0128591; US 2003/0103968, U.S. Pat. No. 6,455,677; US 2009/0304718; US 2009/0304718; US 2012/0258119); anti-ADAM12 (WO 2015/028027; WO 2020/191293); and anti-ITGA11 (WO 2008/075038; US 2011/0256061). Other antibodies that can be used to construct CARs to pursue and treat solid tumors or fibrosis include anti-CTSK, anti-NOX4, anti-SGCD, anti-SYNDIGI, anti-CDH11, anti-PLPP4, anti-SLC24A2, anti-PDGFRB, anti-THY1, anti-ANTXR1, anti-GAS1, anti-CALHM5, anti-COL11A1, anti-COL1A2, anti-FBN1, anti-COL10A1, anti-COL3A1, anti-COL5A2, anti-COL1A1, anti-COL8A2, anti-COL6A3, anti-GLT8D2, anti-SULF1, anti-COL12A1, anti-GXYLT2, anti-NID2, anti-THBS2, anti-COL5A1, anti-FN1, anti-COL6A1, anti-C₃orf80.

An mRNA disclosed herein encoding a CAR includes both the mature CAR and a signal peptide. A mature CAR minimally comprises an antigen binding domain, a transmembrane domain, and an intracellular signaling domain. In some embodiments, a CAR further comprises one or more co-stimulatory domains in the intracellular portion of the CAR. In some embodiments, a CAR further comprises an extracellular hinge or extension domain between the transmembrane domain and the antigen binding domain; this domain can be derived from the same protein as the transmembrane domain. In some embodiments, a CAR can comprise multiple antigen binding domains. In certain embodiments of the mRNA disclosed herein, the CAR is an anti-CD19 CAR, an anti-CD20 CAR, an anti-BCMA CAR, or an anti-FAP CAR.

### F) i) Anti-CD19 CAR

In certain embodiments, two CAR configurations are used for anti-CD19 CAR: CAR1 and CAR2. CAR1 mRNAs encode an amino acid sequence consisting of the following domains in N- to C-terminal order: CD8a signal peptide (SP), anti-CD19 scFv derived from mAb 47G4 (light chain variable domain, VL; linker, L; heavy chain variable domain, VH; 47G4 is disclosed in US2010/0104509), CD8a hinge, CD8a transmembrane domain (TM), CD28 costimulatory domain (co-stim), and CD3ζ signaling domain (stim). The CAR1 amino acid sequence is originally disclosed in U.S. Pat. No. 10,287,350 (WO2015/187528) as SEQ ID NO: 18, from which the CAR1 amino acid sequence and its synthesis are incorporated herein by reference. The amino acid sequence of the mature CAR1 protein (i.e., without a signal peptide) is provided as SEQ ID NO: 23. The incorporation of the CD8a hinge and transmembrane domains in CAR1 helps reduce cytokine release syndrome (cytokine storm) in comparison to similar anti-CD19 CAR molecules that instead incorporate a CD28 hinge and transmembrane domain. For this reason, use of the CD8a hinge and transmembrane region has been considered the superior choice in the CAR-T field. Exemplary mRNA sequences encoding CAR1 include RM_61321 (SEQ ID NO: 199), RM_61324 (SEQ ID NO: 200), RM_61326 (SEQ ID NO: 201), RM_61349 (SEQ ID NO: 202), RM_61350 (SEQ ID NO: 203), RM_61378 (SEQ ID NO: 204), and RM_61379 (SEQ ID NO: 205).

In certain embodiments comprising an anti-CD19 CAR, the anti-CD19 CAR comprises an anti-CD19 binding domain. Some embodiments of an anti-CD19 CAR comprising an anti-CD19 binding domain further comprise a CD28 hinge, transmembrane, and co-stimulatory domains, and a CD3ζ signaling domain. Some embodiments of an anti-CD19 CAR comprising an anti-CD19 binding domain further comprise a hinge and transmembrane domain from CD8α, a CD28 costimulatory domain, and a CD3ζ-chain signaling domain. In certain embodiments, an anti-CD19 binding domain comprises a 47G4 scFv. In certain embodiments, a CAR-T cell comprising an anti-CD19 CAR comprising CD28 hinge, transmembrane, and co-stimulatory domains exhibits more pursued cell killing than a CAR-T cell comprising an anti-CD19 CAR comprising CD8a hinge and transmembrane domains, and a CD28 co-stimulatory domain.

In certain embodiments, the CAR2 mRNAs encode an amino acid sequence (SEQ ID NO: 19) consisting of the following domains in N- to C-terminal order: CD8a signal peptide (SP), anti-CD19 scFv derived from mAb 47G4 (light chain variable domain, VL; linker, L; heavy chain variable domain, VH), CD28 hinge, CD28 transmembrane (TM), CD28 co-stimulatory domain (co-stim), and CD3ζ signaling domain (stim). The amino acid sequence of the immature CAR2 protein (i.e., with a signal peptide) is disclosed in Genbank: QHQ73565.1 and provided as SEQ ID NO: 19. The mature sequence of CAR2 (w/o the signal peptide) is provided as SEQ ID NO: 24. Combining the 47G4 scFv as well as the CD28 hinge and transmembrane domains provides CAR2 an advantage for transient in vivo transfection as opposed to the traditional CAR-T cell comprising an integrated DNA sequence encoding CAR. CAR2 is expressed at a higher level than CAR1 from mRNAs using the same UTRs and codon optimization method and the T cells expressing CAR2 eliminate more CD19⁺ cells. Exemplary mRNA sequences encoding CAR2 include RM_61355 (SEQ ID NO: 206), RM_61356 (SEQ ID NO: 207), RM_61357 (SEQ ID NO: 208), RM_61358 (SEQ ID NO: 209), RM_61455 (SEQ ID NO: 210), RM_61458 (SEQ ID NO: 211), RM_61461 (SEQ ID NO: 212), RM_61482 (SEQ ID NO: 213), RM_61483 (SEQ ID NO: 214), RM_61486 (SEQ ID NO: 215), RM_61487 (SEQ ID NO: 216), RM_61488 (SEQ ID NO: 217), and RM_61489 (SEQ ID NO: 218).

Further examples of anti-CD19 CARs include those incorporating a CD19 binding moiety derived from the mouse antibody FMC63. FMC63 and the derived scFv have been described in Nicholson et al., 1997, Mol. Immun. 34(16-17):1157-1165 and PCT Application Publication Nos. WO 2018/213337 and WO 2015/187528, the entire contents of each of which are incorporated by reference herein for all that they teach about anti-CD19 CARs and their use.

TABLE 5
Exemplary sequences of anti-CD19 scFv and components

| SEQ ID NO: | Amino Acid Sequence | Description |
|---|---|---|
| 28 | DIQMTQTTSSLSASLGDRVTISCRAS | Anti-CD19 FMC63 scFv |
| | QDISKYLNWYQQKPDGTVKLLIYHT | entire sequence, with |
| | SRLHSGVPSRFSGSGSGTDYSLTISN | Whitlow linker |
| | LEQEDIATYFCQQGNTLPYTFGGGT | |
| | KLEITGSTSGSGKPGSGEGSTKGEVK | |
| | LQESGPGLVAPSQSLSVTCTVSGVSL | |
| | PDYGVSWIRQPPRKGLEWLGVIWGS | |
| | ETTYYNSALKSRLTIIKDNSKSQVFL | |
| | KMNSLQTDDTAIYYCAKHYYYGGS | |
| | YAMDYWGQGTSVTVSS | |
| 29 | DIQMTQTTSSLSASLGDRVTISCRAS | Anti-CD19 FMC63 scFv |
| | QDISKYLNWYQQKPDGTVKLLIYHT | light chain variable region |
| | SRLHSGVPSRFSGSGSGTDYSLTISN | |
| | LEQEDIATYFCQQGNTLPYTFGGGT | |
| | KLEIT | |
| 30 | QDISKY | Anti-CD19 FMC63 scFv |
| | | light chain CDR1 |
| N/A | HTS | Anti-CD19 FMC63 scFv |
| | | light chain CDR2 |
| 32 | QQGNTLPYT | Anti-CD19 FMC63 scFv |
| | | light chain CDR3 |
| 33 | GSTSGSGKPGSGEGSTKG | Whitlow linker |
| 34 | EVKLQESGPGLVAPSQSLSVTCTVS | Anti-CD19 FMC63 scFv |
| | GVSLPDYGVSWIRQPPRKGLEWLG | heavy chain variable |
| | VIWGSETTYYNSALKSRLTIIKDNSK | region |
| | SQVFLKMNSLQTDDTAIYYCAKHY | |
| | YYGGSYAMDYWGQGTSVTVSS | |
| 35 | GVSLPDYG | Anti-CD19 FMC63 scFv |
| | | heavy chain CDR1 |
| 36 | IWGSETT | Anti-CD19 FMC63 scFv |
| | | heavy chain CDR2 |
| 37 | AKHYYYGGSYAMDY | Anti-CD19 FMC63 scFv |
| | | heavy chain CDR3 |
| 38 | DIQMTQTTSSLSASLGDRVTISCRAS | Anti-CD19 FMC63 scFv |
| | QDISKYLNWYQQKPDGTVKLLIYHT | entire sequence, with |
| | SRLHSGVPSRFSGSGSGTDYSLTISN | 3xG4S linker |
| | LEQEDIATYFCQQGNTLPYTFGGGT | |
| | KLEITGGGGSGGGGSGGGGSEVKLQ | |
| | ESGPGLVAPSQSLSVTCTVSGVSLPD | |
| | YGVSWIRQPPRKGLEWLGVIWGSET | |
| | TYYNSALKSRLTIIKDNSKSQVFLK | |
| | MNSLQTDDTAIYYCAKHYYYGGSY | |
| | AMDYWGQGTSVTVSS | |
| 39 | GGGGSGGGGSGGGGS | 3xG4S linker |

In some instances, the ant-CD19 CAR is the CAR found in tisagenlecleucel (Vairy eta., 2018, Drug Des Devel Ther. 12: 3885-3898), lisocabtagene maraleucel, or axicabtagene ciloleucel and brexucabtagene autoleucel (Cappell eta., 2023, Nat Rev Clin Oncol 20: 359-371) which use the same CAR. The entire contents of each of foregoing references in this paragraph are incorporated by reference for all that they teach about the design, structure, and activity of anti-CD19 CARs.

TABLE 6
Exemplary sequences of CD19 CARS*

| SEQ ID NO: | Sequence | Description |
|---|---|---|
| 40 | atggccttaccagtgaccgccttgctcctgccgctggccttgctgctc | Tisagenlecleucel |
| | cacgccgccaggccggacatccagatgacacagactacatcctccc | CD19 CAR |
| | tgtctgcctctctgggagacagagtcaccatcagttgcagggcaagt | nucleotide |
| | caggacattagtaaatatttaaattggtatcagcagaaaccagatgga | sequence |
| | actgttaaactcctgatctaccatacatcaagattacactcaggagtcc | |
| | catcaaggttcagtggcagtgggtctggaacagattattctctcaccat | |
| | tagcaacctggagcaagaagatattgccacttacttttgccaacaggg | |
| | taatacgcttccgtacacgttcggaggggggaccaagctggagatc | |
| | acaggtggcggtggctcgggcggtggtgggtcgggtggcggcgg | |
| | atctgaggtgaaactgcaggagtcaggacctggcctggtggcgccc | |
| | tcacagagcctgtccgtcacatgcactgtctcaggggtctcattaccc | |
| | gactatggtgtaagctggattcgccagcctccacgaaagggtctgga | |
| | gtggctgggagtaatatggggtagtgaaaccacatactataattcagc | |
| | tctcaaatccagactgaccatcatcaaggacaactccaagagccaag | |
| | ttttcttaaaaatgaacagtctgcaaactgatgacacagccatttactac | |
| | tgtgccaaacattattactacggtggtagctatgctatggactactggg | |
| | gccaaggaacctcagtcaccgtctcctcaaccacgacgccagcgcc | |
| | gcgaccaccaacaccggcgcccaccategcgtcgcagcccctgtc | |
| | cctgcgcccagaggcgtgccggccagcggcggggggcgcagtg | |
| | cacacgagggggctggacttcgcctgtgatatctacatctgggcgcc | |
| | cttggccgggacttgtggggtccttctcctgtcactggttatcacccttt | |
| | actgcaaacggggcagaaagaaactcctgtatatattcaaacaacca | |
| | tttatgagaccagtacaaactactcaagaggaagatggctgtagctg | |
| | ccgatttccagaagaagaagaaggaggatgtgaactgagagtgaa | |
| | gttcagcaggagcgcagacgcccccgcgtacaagcagggccaga | |
| | accagctctataacgagctcaatctaggacgaagagaggagtacga | |
| | tgttttggacaagagacgtggccgggaccctgagatggggggaaa | |
| | gccgagaaggaagaaccctcaggaaggcctgtacaatgaactgca | |
| | gaaagataagatggcggaggcctacagtgagattgggatgaaagg | |
| | cgagcgccggaggggcaaggggcacgatggcctttaccagggtct | |
| | cagtacagccaccaaggacacctacgacgcccttcacatgcaggcc | |
| | ctgccccctcgc | |
| 41 | MALPVTALLLPLALLLHAARPDIQMTQTTS | Tisagenlecleucel |
| | SLSASLGDRVTISCRASQDISKYLNWYQQKP | CD19 CAR amino |
| | DGTVKLLIYHTSRLHSGVPSRFSGSGSGTDY | acid sequence |
| | SLTISNLEQEDIATYFCQQGNTLPYTFGGGT | |
| | KLEITGGGGSGGGGSGGGGSEVKLQESGPG | |
| | LVAPSQSLSVTCTVSGVSLPDYGVSWIRQPP | |
| | RKGLEWLGVIWGSETTYYNSALKSRLTIIKD | |
| | NSKSQVFLKMNSLQTDDTAIYYCAKHYYY | |
| | GGSYAMDYWGQGTSVTVSSTTTPAPRPPTP | |
| | APTIASQPLSLRPEACRPAAGGAVHTRGLDF | |
| | ACDIYIWAPLAGTCGVLLLSLVITLYCKRGR | |
| | KKLLYIFKQPFMRPVQTTQEEDGCSCRFPEE | |
| | EEGGCELRVKFSRSADAPAYKQGQNQLYN | |
| | ELNLGRREEYDVLDKRRGRDPEMGGKPRR | |
| | KNPQEGLYNELQKDKMAEAYSEIGMKGER | |
| | RRGKGHDGLYQGLSTATKDTYDALHMQAL | |
| | PPR | |
| 42 | atgctgctgctggtgaccagcctgctgctgtgcgagctgccccaccc | Lisocabtagene |
| | cgcctttctgctgatccccgacatccagatgacccagaccacctcca | maraleucel CD19 |
| | gcctgagcgccagcctgggcgaccgggtgaccatcagctgccgg | CAR nucleotide |
| | gccagccaggacatcagcaagtacctgaactggtatcagcagaagc | sequence |
| | ccgacggcaccgtcaagctgctgatctaccacaccagccggctgca | |
| | cagcggcgtgcccagccggtttagcggcagcggctccggcaccga | |
| | ctacagcctgaccatctccaacctggaacaggaagatatcgccacct | |
| | acttttgccagcagggcaacacactgccctacacctttggcggcgga | |
| | acaaagctggaaatcaccggcagcacctccggcagcggcaagcct | |
| | ggcagcggcgagggcagcaccaagggcgaggtgaagctgcagg | |
| | aaagcggccctggcctggtggcccccagccagagcctgagcgtga | |
| | cctgcaccgtgagcggcgtgagcctgcccgactacggcgtgagct | |
| | ggatccggcagccccccaggaagggcctggaatggctgggcgtg | |
| | atctggggcagcgagaccacctactacaacagcgccctgaagagc | |
| | cggctgaccatcatcaaggacaacagcaagagccaggtgttcctga | |
| | agatgaacagcctgcagaccgacgacaccgccatctactactgcgc | |
| | caagcactactactacggcggcagctacgccatggactactggggc | |
| | cagggcaccagcgtgaccgtgagcagcgaatctaagtacggaccg | |
| | ccctgccccccttgccctatgttctgggtgctggtggtggtcggagg | |
| | cgtgctggcctgctacagcctgctggtcaccgtggccttcatcatcttt | |
| | tgggtgaaacggggcagaaagaaactcctgtatatattcaaacaacc | |
| | atttatgagaccagtacaaactactcaagaggaagatggctgtagct | |
| | gccgatttccagaagaagaagaaggaggatgtgaactgcgggtga | |
| | agttcagcagaagcgccgacgcccctgcctaccagcagggccaga | |
| | atcagctgtacaacgagctgaacctgggcagaagggaagagtacg | |
| | acgtcctggataagcggagaggccgggaccctgagatgggcggc | |
| | aagcctcggcggaagaacccccaggaaggcctgtataacgaactg | |
| | cagaaagacaagatggccgaggcctacagcgagatcggcatgaa | |
| | gggcgagcggaggcggggcaagggccacgacggcctgtatcag | |
| | ggcctgtccaccgccaccaaggatacctacgacgccctgcacatgc | |
| | aggccctgcccccaagg | |
| 43 | MLLLVTSLLLCELPHPAFLLIPDIQMTQTTSS | Lisocabtagene |
| | LSASLGDRVTISCRASQDISKYLNWYQQKP | maraleucel CD19 |
| | DGTVKLLIYHTSRLHSGVPSRFSGSGSGTDY | CAR amino acid |
| | SLTISNLEQEDIATYFCQQGNTLPYTFGGGT | sequence |
| | KLEITGSTSGSGKPGSGEGSTKGEVKLQESG | |
| | PGLVAPSQSLSVTCTVSGVSLPDYGVSWIRQ | |
| | PPRKGLEWLGVIWGSETTYYNSALKSRLTII | |
| | KDNSKSQVFLKMNSLQTDDTAIYYCAKHY | |
| | YYGGSYAMDYWGQGTSVTVSSESKYGPPC | |
| | PPCPMFWVLVVVGGVLACYSLLVTVAFIIF | |
| | WVKRGRKKLLYIFKQPFMRPVQTTQEEDG | |
| | CSCRFPEEEEGGCELRVKFSRSADAPAYQQ | |
| | GQNQLYNELNLGRREEYDVLDKRRGRDPE | |
| | MGGKPRRKNPQEGLYNELQKDKMAEAYSE | |
| | IGMKGERRRGKGHDGLYQGLSTATKDTYD | |
| | ALHMQALPPR | |
| 44 | atgcttctcctggtgacaagccttctgctctgtgagttaccacacccag | Axicabtagene |
| | cattcctcctgatcccagacatccagatgacacagactacatcctccc | ciloleucel CD19 |
| | tgtctgcctctctgggagacagagtcaccatcagttgcagggcaagt | CAR nucleotide |
| | caggacattagtaaatatttaaattggtatcagcagaaaccagatgga | sequence |
| | actgttaaactcctgatctaccatacatcaagattacactcaggagtcc | |
| | catcaaggttcagtggcagtgggtctggaacagattattctctcaccat | |
| | tagcaacctggagcaagaagatattgccacttacttttgccaacaggg | |
| | taatacgcttccgtacacgttcggaggggggactaagttggaaataa | |
| | caggctccacctctggatccggcaagcccggatctggcgagggatc | |
| | caccaagggcgaggtgaaactgcaggagtcaggacctggcctggt | |
| | ggcgccctcacagagcctgtccgtcacatgcactgtctcaggggtct | |
| | cattacccgactatggtgtaagctggattcgccagcctccacgaaag | |
| | ggtctggagtggctgggagtaatatggggtagtgaaaccacatacta | |
| | taattcagctctcaaatccagactgaccatcatcaaggacaactccaa | |
| | gagccaagttttcttaaaaatgaacagtctgcaaactgatgacacagc | |
| | catttactactgtgccaaacattattactacggtggtagctatgctatgg | |
| | actactggggtcaaggaacctcagtcaccgtctcctcagcggccgc | |
| | aattgaagttatgtatcctcctccttacctagacaatgagaagagcaat | |
| | ggaaccattatccatgtgaaagggaaacacctttgtccaagtccccta | |
| | tttcccggaccttctaagcccttttgggtgctggtggtggttggggga | |
| | gtcctggcttgctatagcttgctagtaacagtggcctttattattttctgg | |
| | gtgaggagtaagaggagcaggctcctgcacagtgactacatgaaca | |
| | tgactccccgccgccccgggcccacccgcaagcattaccagcccta | |
| | tgccccaccacgcgacttcgcagcctatcgctccagagtgaagttca | |
| | gcaggagcgcagacgcccccgcgtaccagcagggccagaacca | |
| | gctctataacgagctcaatctaggacgaagagaggagtacgatgtttt | |
| | ggacaagagacgtggccgggaccctgagatggggggaaagccg | |
| | agaaggaagaaccctcaggaaggcctgtacaatgaactgcagaaa | |
| | gataagatggcggaggcctacagtgagattgggatgaaaggcgag | |
| | cgccggaggggcaaggggcacgatggcctttaccagggtctcagt | |
| | acagccaccaaggacacctacgacgcccttcacatgcaggccctgc | |
| | cccctcgc | |
| 45 | MLLLVTSLLLCELPHPAFLLIPDIQMTQTTSS | Axicabtagene |
| | LSASLGDRVTISCRASQDISKYLNWYQQKP | ciloleucel CD19 |
| | DGTVKLLIYHTSRLHSGVPSRFSGSGSGTDY | CAR amino acid |
| | SLTISNLEQEDIATYFCQQGNTLPYTFGGGT | sequence |
| | KLEITGSTSGSGKPGSGEGSTKGEVKLQESG | |
| | PGLVAPSQSLSVTCTVSGVSLPDYGVSWIRQ | |
| | PPRKGLEWLGVIWGSETTYYNSALKSRLTII | |
| | KDNSKSQVFLKMNSLQTDDTAIYYCAKHY | |
| | YYGGSYAMDYWGQGTSVTVSSAAAIEVM | |
| | YPPPYLDNEKSNGTIIHVKGKHLCPSPLFPGP | |
| | SKPFWVLVVVGGVLACYSLLVTVAFIIFWV | |
| | RSKRSRLLHSDYMNMTPRRPGPTRKHYQPY | |
| | APPRDFAAYRSRVKFSRSADAPAYQQGQN | |
| | QLYNELNLGRREEYDVLDKRRGRDPEMGG | |
| | KPRRKNPQEGLYNELQKDKMAEAYSEIGM | |
| | KGERRRGKGHDGLYQGLSTATKDTYDALH | |
| | MQALPPR | |

*Nucleotide sequences are presented here (and throughout) as DNA sequences; t should be understood as “u” for corresponding RNA sequences.

TABLE 7
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3xG₄S linker-V_{H}) | | |
| CD8α hinge domain | 790-924 | 264-308 |
| CD8α transmembrane domain | 925-996 | 309-332 |
| 4-1BB signaling domain | 997-1122 | 333-374 |
| CD3ζ signaling domain | 1123-1458 | 375-486 |

TABLE 8
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB signaling domain | 922-1047 | 308-349 |
| CD3ζ signaling domain | 1048-1383 | 350-461 |

TABLE 9
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 signaling domain | 1009-1131 | 337-377 |
| CD3ζ signaling domain | 1132-1467 | 378-489 |

CAR based on 47G4 are disclosed in U.S. Pat. No. 10,287,350 which is incorporated by reference herein for all that it teaches about anti-CD19 CARs and their use. In some embodiments, the extracellular binding domain of the CD19 CAR is derived from an antibody specific to CD19, including, for example, SJ25C1 (Bejcek et al., 1995, Cancer Res. 55:2346-2351), HD37 (Pezutto et al., 1987, J. Immunol. 138(9):2793-2799), 4G7 (Meeker et al., 1984, Hybridoma 3:305-320), B43 (Bejcek et al., 1995 Cancer Res 55(11):2346-2351), BLY3 (Bejcek et al., 1995, Cancer Res 55(11):2346-2351), B4 (Freedman et al., 1987, Blood 70:418-427), B4 HB12b (Kansas & Tedder, 1991, J. Immunol. 147:4094-4102; Yazawa et al., 2005, Proc. Natl. Acad. Sci. USA 102:15178-15183; Herbst et al., J. Pharmacol. Exp. Ther. 335:213-222 (2010)), BU12 (Callard et al., 1992, J Immunology 148(10): 2983-2987), and CLB-CD19 (De Rie, 1989, Cell. Immunol. 118:368-381). In any of these embodiments, the extracellular binding domain of the CD19 CAR can comprise the VH, the VL, and/or one or more CDRs of any of the antibodies.

TABLE 10
Exemplary sequences of 47G4-based anti-CD19 CAR

| SEQ ID NO: | Sequence | Description |
|---|---|---|
| 4 | MALPVTALLLPLALLLHAARP | CD8 signal peptide |
| 46 | EIVLTQSPGTLSLSPGERATLSCRASQSVSS | CD19 antibody |
| | SYLAWYQQKPGQAPRLLIYGASSRATGIPD | (47G4 scFv) |
| | RFSGSGSGTDFTLTISRLEPEDFAVYYCQQ | |
| | YGSSRFTFGPGTKVDIKGSTSGSGKPGSGE | |
| | GSTKGQVQLVQSGAEVKKPGSSVKVSCKD | |
| | SGGTFSSYAISWVRQAPGQGLEWMGGIIPI | |
| | FGTTNYAQQFQGRVTITADESTSTAYMELS | |
| | SLRSEDTAVYYCAREAVAADWLDPWGQG | |
| | TLVTVSS | |
| 8 | IEVMYPPPYLDNEKSNGTIIHVKGKHLCPSP | CD28 hinge |
| | LFPGPSKP | |
| 13 | FWVLVVVGGVLACYSLLVTVAFIIFWV | CD28 |
| | | transmembrane |
| | | domain |
| 15 | RSKRSRLLHSDYMNMTPRRPGPTRKHYQP | CD28 cytoplasmic |
| | YAPPRDFAAYRS | (co-stim) |
| 16 | RVKFSRSADAPAYQQGQNQLYNELNLGRR | CD3ζ (stim) |
| | EEYDVLDKRRGRDPEMGGKPRRKNPQEGL | |
| | YNELQKDKMAEAYSEIGMKGERRRGKGH | |
| | DGLYQGLSTATKDTYDALHMQALPPR | |
| 47 | FVPVFLPAKPTTTPAPRPPTPAPTIASQPLSL | CD8 hinge |
| | RPEACRPAAGGAVHTRGLDFACD | |
| 48 | IYIWAPLAGTCGVLLLSLVITLYCNHRN | CD8 |
| | | transmembrane |
| | | domain |

### f) ii) Anti-CD20 CAR

CD20 is an antigen found on the surface of B cells as early as the pro-B phase and progressively at increasing levels until B cell maturity, as well as on the cells of most B-cell neoplasms. CD20 positive cells are also sometimes found in cases of Hodgkin's disease, myeloma, and thymoma. Examples of anti-CD20 CARs include those incorporating a CD20 binding moiety derived from an antibody specific to CD20, including, for example, MB-106 (Fred Hutchinson Cancer Research Center, see Shadman et al., 2019, Blood 134(Suppl.1):3235), UCART20 (Cellectis, www.cellbiomedgroup.com), or C-CAR066 (Cellular Biomedicine Group, see Liang et al., 2021, J. Clin. Oncol. 39(15) suppl:2508). In some embodiments, the extracellular binding domain of the anti-CD20 CAR is derived from an antibody specific to CD20, including, for example, Leu16, 2.1.2, IF5, 1.5.3, rituximab, obinutuzumab, ibritumomab, ofatumumab, tositumumab, odronextamab, veltuzumab, ublituximab, and ocrelizumab. In some embodiments, the extracellular binding domain of the anti-CD20 CAR comprises an scFv derived from the Leu16 monoclonal antibody, which comprises the heavy chain variable region (VH) and the light chain variable region (VL) of Leu16 connected by a linker (See Wu et al., 2001, Protein Engineering. 14(12):1025-1033), such as CAR22 and CAR25 described herein. In some embodiments, the extracellular binding domain of the anti-CD20 CAR comprises an scFv derived from the monoclonal antibody, 2.1.2 (WO2006130458A2), which comprises the heavy chain variable region (VH) and the light chain variable region (VL) of 2.1.2 connected by a linker, such as CAR7 described herein. The entire contents of each of foregoing references in this paragraph are incorporated by reference for all that they teach about the design, structure, and activity of anti-CD20 CARs.

In certain embodiments, CAR25 is provided herein as a CAR configuration used for anti-CD20 CAR. The CAR25 mRNA encodes an amino acid sequence consisting of the following domains in N- to C-terminal order: mouse Ig-kappa signal peptide (Igk sp), anti-CD20 scFv derived from the Leu16 mAb (light chain variable domain, VL; linker, L; heavy chain variable domain, VH), IgG4 hinge, CD28 transmembrane domain (TM), 4-1BB co-stimulatory domain (co-stim), and CD3ζ signaling domain (stim). The amino acid sequence of the mature CAR25 protein (i.e., without a signal peptide) is provided as SEQ ID NO: 25; the immature CAR25 (with the Igk sp) is provided as SEQ ID NO: 93. An exemplary mRNA sequence encoding CAR25 is RM_61639 (SEQ ID NO: 219).

In certain embodiments comprising an anti-CD20 CAR, the anti-CD20 CAR comprises a Leu16 scFv. In some embodiments, the anti-CD20 CAR comprising a Leu16 scFv further comprises an IgG4 hinge, CD28 transmembrane domain, 4-1BB costimulation, and a CD3ζ signaling domain. Examples of such an anti-CD20 CAR include, without limitation, CAR25 (SEQ ID NO: 25, or with a signal peptide, SEQ ID NO: 20). In some embodiments, the anti-CD20 CAR comprising a Leu16 scFv further comprises an IgG4 hinge, CD28 transmembrane and costimulation domains, 4-1BB costimulation, and a CD3ζ signaling domain. Examples of such an anti-CD20 CAR include CAR22 (SEQ ID NO: 27), or with a signal peptide (SEQ ID NO: 22). Exemplary mRNA sequences encoding CAR22 include RM_61653 (SEQ ID NO: 220), RM_61654 (SEQ ID NO: 221), RM_61655 (SEQ ID NO: 222), and RM_61656 (SEQ ID NO: 223).

TABLE 11
Exemplary sequences of anti-CD20 scFv and components

| SEQ ID NO: | Amino Acid Sequence | Description |
|---|---|---|
| 49 | DIVLTQSPAILSASPGEKVTMTCRAS | Anti-CD20 Leu16 scFv |
| | SSVNYMDWYQKKPGSSPKPWIYAT | entire sequence, with |
| | SNLASGVPARFSGSGSGTSYSLTISR | Whitlow linker |
| | VEAEDAATYYCQQWSFNPPTFGGG | |
| | TKLEIKGSTSGSGKPGSGEGSTKGEV | |
| | QLQQSGAELVKPGASVKMSCKASG | |
| | YTFTSYNMHWVKQTPGQGLEWIGA | |
| | IYPGNGDTSYNQKFKGKATLTADKS | |
| | SSTAYMQLSSLTSEDSADYYCARSN | |
| | YYGSSYWFFDVWGAGTTVTVSS | |
| 50 | DIVLTQSPAILSASPGEKVTMTCRAS | Anti-CD20 Leu16 scFv |
| | SSVNYMDWYQKKPGSSPKPWIYAT | light chain variable region |
| | SNLASGVPARFSGSGSGTSYSLTISR | |
| | VEAEDAATYYCQQWSFNPPTFGGG | |
| | TKLEIK | |
| 51 | RASSSVNYMD | Anti-CD20 Leu16 scFv |
| | | light chain CDR1 |
| 52 | ATSNLAS | Anti-CD20 Leu16 scFv |
| | | light chain CDR2 |
| 53 | QQWSFNPPT | Anti-CD20 Leu16 scFv |
| | | light chain CDR3 |
| 54 | EVQLQQSGAELVKPGASVKMSCKA | Anti-CD20 Leu16 scFv |
| | SGYTFTSYNMHWVKQTPGQGLEWI | heavy chain |
| | GAIYPGNGDTSYNQKFKGKATLTA | |
| | DKSSSTAYMQLSSLTSEDSADYYCA | |
| | RSNYYGSSYWFFDVWGAGTTVTVS | |
| | S | |
| 55 | SYNMH | Anti-CD20 Leu16 scFv |
| | | heavy chain CDR1 |
| 56 | AIYPGNGDTSYNQKFKG | Anti-CD20 Leu16 scFv |
| | | heavy chain CDR2 |
| 96 | SNYYGSSYWFFDV | Anti-CD20 Leu16 scFv |
| | | heavy chain CDR3 |
| 97 | DIVLTQSPAILSASPGEKVTMTCRAS | Anti-CD20 Leu16 scFv |
| | SSVNYMDWYQKKPGSSPKPWIYAT | entire sequence, with |
| | SNLASGVPARFSGSGSGTSYSLTISR | (G₃S)₂-(G₄S) linker |
| | VEAEDAATYYCQQWSFNPPTFGGG | |
| | TKLEIKGSTSGGGSGGGSGGGGSSE | |
| | VQLQQSGAELVKPGASVKMSCKAS | |
| | GYTFTSYNMHWVKQTPGQGLEWIG | |
| | AIYPGNGDTSYNQKFKGKATLTAD | |
| | KSSSTAYMQLSSLTSEDSADYYCAR | |
| | SNYYGSSYWFFDVWGAGTTVTVSS | |
| 98 | GSTSGGGSGGGSGGGGSS | (G3S)₂-(G4S) linker |
| 99 | DIVMTQTPHSSPVTLGQPASISCRSS | Anti-CD20 2.1.2 scFv |
| | QSLVSRDGNTYLSWLQQRPGQPPRL | entire sequence, with |
| | LIYKISNRFSGVPNRFSGSGAGTDFT | (G₄S)₃ linker |
| | LKISRVKAEDVGVYYCMQATQFPLT | |
| | FGQGTRLEIKGGGGSGGGGSGGGGS | |
| | EVQLVQSGAEVKKPGESLKISCKGS | |
| | GYSFTSYWIGWVRQMPGKGLEWM | |
| | GIIYPGDSDTRYSPSFQGQVTISADK | |
| | SISTAYLQWSSLKASDTAMYYCARQ | |
| | GDFWSGYGGMDVWGQGTTVTVSS | |
| 100 | DIVMTQTPHSSPVTLGQPASISCRSS | Anti-CD20 2.1.2 scFv |
| | QSLVSRDGNTYLSWLQQRPGQPPRL | light chain variable region |
| | LKISRVKAEDVGVYYCMQATQFPLT | |
| | FGQGTRLEIK | |
| 101 | RSSQSLVSRDGNTYLS | Anti-CD20 2.1.2 scFv |
| | | light chain CDR1 |
| 102 | KISNRFS | Anti-CD20 2.1.2 scFv |
| | | light chain CDR2 |
| 103 | MQATQFPLT | Anti-CD20 2.1.2 scFv |
| | | light chain CDR3 |
| 39 | GGGGSGGGGSGGGGS | (G4S)3 or 3x G4S linker |
| 104 | EVQLVQSGAEVKKPGESLKISCKGS | Anti-CD20 2.1.2 scFv |
| | GYSFTSYWIGWVRQMPGKGLEWM | heavy chain variable |
| | GIIYPGDSDTRYSPSFQGQVTISADK | region |
| | SISTAYLQWSSLKASDTAMYYCARQ | |
| | GDFWSGYGGMDVWGQGTTVTVSS | |
| 105 | SYWIG | Anti-CD20 2.1.2 scFv |
| | | heavy chain CDR1 |
| 106 | IIYPGDSDTRYSPSFQG | Anti-CD20 2.1.2 scFv |
| | | heavy chain CDR2 |
| 107 | QGDFWSGYGGMDV | Anti-CD20 2.1.2 scFv |
| | | heavy chain CDR3 |

In certain embodiments comprising an anti-CD20 CAR, the anti-CD20 CAR comprises a 2.1.2 scFv. In some embodiments, the anti-CD20 CAR comprising a 2.1.2 scFv further comprises CD28 hinge, transmembrane, and costimulation domains and a CD3 signaling domain. Examples of such an anti-CD20 CAR include, without limitation, CAR7 (SEQ TD NO: 26), or with a signal peptide, SEQ ID NO:21). Exemplary mRNA sequences encoding CAR7 include RM_61657 (SEQ ID NO: 224), RM_61658 (SEQ ID NO: 225), RM_61659 (SEQ ID NO: 226), and RM_61660 (SEQ ID NO: 227).

f) iii) Anti-BCMA CAR

In certain embodiments, the improved mRNA encodes an anti-BCMA chimeric antigen receptor (CAR). BCMA is a tumor necrosis family receptor (TNFR) member expressed on cells of the B cell lineage, with the highest expression on terminally differentiated B cells or mature B lymphocytes. BCMA is involved in mediating survival of plasma cells for maintaining long-term humoral immunity. Expression of BCMA has been recently linked to a number of cancers, such as multiple myeloma, Hodgkin's and non-Hodgkin's lymphoma, various leukemias, and glioblastoma. Examples of anti-BCMA CARs include those incorporating a BCMA binding moiety derived from C₁₁D5.3, a mouse monoclonal antibody as described in Carpenter et al., 2013, Clin. Cancer Res. 19(8):2048-2060. See also PCT Application Publication No. WO 2010/104949. In some embodiments, the extracellular binding domain of the BCMA CAR comprises an scFv derived from another mouse monoclonal antibody, C₁₂A3.2, as described in Carpenter et al., 2013, Clin. Cancer Res. 19(8):2048-2060 and PCT Application Publication No. WO2010104949. In some embodiments, the extracellular binding domain of the BCMA CAR comprises an scFv derived from a mouse monoclonal antibody with high specificity to human BCMA, referred to as BB2121 in Friedman et al., 2018, Hum. Gene Ther. 29(5):585-601. See also, PCT Application Publication No. WO2012163805. In some embodiments, the extracellular binding domain of the BCMA CAR comprises single variable fragments of two heavy chains (VHH) that can bind to two epitopes of BCMA as described in Zhao et al., 2018, J. Hematol. Oncol. 11(1):141, also referred to as LCAR-B38M. See also, PCT Application Publication No. WO 2018/028647. In some embodiments, the extracellular binding domain of the BCMA CAR comprises a fully human heavy-chain variable domain (FHVH) as described in Lam et al., 2020, Nat. Commun. 11(1):283, also referred to as FHVH33. See also, PCT Application Publication No. WO 2019/006072. In some embodiments, the extracellular binding domain of the BCMA CAR comprises an scFv derived from CT103A (or CAR0085) as described in U.S. Pat. No. 11,026,975 B2. Further anti-BCMA CARs are disclosed in U.S. Patent Application Publication Nos. 2020/0246381 and 2020/0339699. The entire contents of each of foregoing references in this paragraph are incorporated by reference for all that they teach about the design, structure, and activity of anti-BCMA CARs.

TABLE 12
Exemplary sequences of anti-BCMA binder and components

| SEQ ID NO: | Amino Acid Sequence | Description |
|---|---|---|
| 57 | DIVLTQSPASLAMSLGKRATISCRAS | Anti-BCMA C11D5.3 |
| | ESVSVIGAHLIHWYQQKPGQPPKLLI | scFv entire sequence, |
| | YLASNLETGVPARFSGSGSGTDFTLT | with Whitlow linker |
| | IDPVEEDDVAIYSCLQSRIFPRTFGG | |
| | GTKLEIKGSTSGSGKPGSGEGSTKG | |
| | QIQLVQSGPELKKPGETVKISCKASG | |
| | YTFTDYSINWVKRAPGKGLKWMG | |
| | WINTETREPAYAYDFRGRFAFSLETS | |
| | ASTAYLQINNLKYEDTATYFCALDY | |
| | SYAMDYWGQGTSVTVSS | |
| 58 | DIVLTQSPASLAMSLGKRATISCRAS | Anti-BCMA C11D5.3 |
| | ESVSVIGAHLIHWYQQKPGQPPKLLI | scFv light chain variable |
| | YLASNLETGVPARFSGSGSGTDFTLT | region |
| | IDPVEEDDVAIYSCLQSRIFPRTFGG | |
| | GTKLEIK | |
| 59 | RASESVSVIGAHLIH | Anti-BCMA C11D5.3 |
| | | scFv light chain CDR1 |
| 60 | LASNLET | Anti-BCMA C11D5.3 |
| | | scFv light chain CDR2 |
| 61 | LQSRIFPRT | Anti-BCMA C11D5.3 |
| | | scFv light chain CDR3 |
| 62 | QIQLVQSGPELKKPGETVKISCKASG | Anti-BCMA C11D5.3 |
| | YTFTDYSINWVKRAPGKGLKWMG | scFv heavy chain |
| | WINTETREPAYAYDFRGRFAFSLETS | variable region |
| | ASTAYLQINNLKYEDTATYFCALDY | |
| | SYAMDYWGQGTSVTVSS | |
| 63 | DYSIN | Anti-BCMA C11D5.3 |
| | | scFv heavy chain CDR1 |
| 64 | WINTETREPAYAYDFRG | Anti-BCMA C11D5.3 |
| | | scFv heavy chain CDR2 |
| 65 | DYSYAMDY | Anti-BCMA C11D5.3 |
| | | scFv heavy chain CDR3 |
| 66 | DIVLTQSPPSLAMSLGKRATISCRAS | Anti-BCMA C12A3.2 |
| | ESVTILGSHLIYWYQQKPGQPPTLLI | scFv entire sequence, |
| | QLASNVQTGVPARFSGSGSRTDFTL | with Whitlow linker |
| | TIDPVEEDDVAVYYCLQSRTIPRTFG | |
| | GGTKLEIKGSTSGSGKPGSGEGSTK | |
| | GQIQLVQSGPELKKPGETVKISCKAS | |
| | GYTFRHYSMNWVKQAPGKGLKWM | |
| | GRINTESGVPIYADDFKGRFAFSVET | |
| | SASTAYLVINNLKDEDTASYFCSND | |
| | YLYSLDFWGQGTALTVSS | |
| 67 | DIVLTQSPPSLAMSLGKRATISCRAS | Anti-BCMA C12A3.2 |
| | ESVTILGSHLIYWYQQKPGQPPTLLI | scFv light chain variable |
| | QLASNVQTGVPARFSGSGSRTDFTL | region |
| | TIDPVEEDDVAVYYCLQSRTIPRTFG | |
| | GGTKLEIK | |
| 68 | RASESVTILGSHLIY | Anti-BCMA C12A3.2 |
| | | scFv light chain CDR1 |
| 69 | LASNVQT | Anti-BCMA C12A3.2 |
| | | scFv light chain CDR2 |
| 70 | LQSRTIPRT | Anti-BCMA C12A3.2 |
| | | scFv light chain CDR3 |
| 71 | QIQLVQSGPELKKPGETVKISCKASG | Anti-BCMA C12A3.2 |
| | YTFRHYSMNWVKQAPGKGLKWMG | scFv heavy chain |
| | RINTESGVPIYADDFKGRFAFSVETS | variable region |
| | ASTAYLVINNLKDEDTASYFCSNDY | |
| | LYSLDFWGQGTALTVSS | |
| 72 | HYSMN | Anti-BCMA C12A3.2 |
| | | scFv heavy chain CDR1 |
| 73 | RINTESGVPIYADDFKG | Anti-BCMA C12A3.2 |
| | | scFv heavy chain CDR2 |
| 74 | DYLYSLDF | Anti-BCMA C12A3.2 |
| | | scFv heavy chain CDR3 |
| 75 | EVQLLESGGGLVQPGGSLRLSCAAS | Anti-BCMA FHVH33 |
| | GFTFSSYAMSWVRQAPGKGLEWVS | entire sequence |
| | SISGSGDYIYYADSVKGRFTISRDISK | |
| | NTLYLQMNSLRAEDTAVYYCAKEG | |
| | TGANSSLADYRGQGTLVTVSS | |
| 76 | GFTFSSYA | Anti-BCMA FHVH33 |
| | | CDR1 |
| 77 | ISGSGDYI | Anti-BCMA FHVH33 |
| | | CDR2 |
| 78 | AKEGTGANSSLADY | Anti-BCMA FHVH33 |
| | | CDR3 |
| 79 | DIQMTQSPSSLSASVGDRVTITCRAS | Anti-BCMA CT103A |
| | QSISSYLNWYQQKPGKAPKLLIYAA | scFv entire sequence, |
| | SSLQSGVPSRFSGSGSGTDFTLTISSL | with Whitlow linker |
| | QPEDFATYYCQQKYDLLTFGGGTK | |
| | VEIKGSTSGSGKPGSGEGSTKGQLQ | |
| | LQESGPGLVKPSETLSLTCTVSGGSI | |
| | SSSSYYWGWIRQPPGKGLEWIGSISY | |
| | SGSTYYNPSLKSRVTISVDTSKNQFS | |
| | LKLSSVTAADTAVYYCARDRGDTIL | |
| | DVWGQGTMVTVSS | |
| 80 | DIQMTQSPSSLSASVGDRVTITCRAS | Anti-BCMA CT103A |
| | QSISSYLNWYQQKPGKAPKLLIYAA | scFv light chain variable |
| | SSLQSGVPSRFSGSGSGTDFTLTISSL | region |
| | QPEDFATYYCQQKYDLLTFGGGTK | |
| | VEIK | |
| 81 | QSISSY | Anti-BCMA CT103A |
| | | scFv light chain CDR1 |
| N/A | AAS | Anti-BCMA CT103A |
| | | scFv light chain CDR2 |
| 83 | QQKYDLLT | Anti-BCMA CT103A |
| | | scFv light chain CDR3 |
| 84 | QLQLQESGPGLVKPSETLSLTCTVSG | Anti-BCMA CT103A |
| | GSISSSSYYWGWIRQPPGKGLEWIGS | scFv heavy chain |
| | ISYSGSTYYNPSLKSRVTISVDTSKN | variable region |
| | QFSLKLSSVTAADTAVYYCARDRG | |
| | DTILDVWGQGTMVTVSS | |
| 85 | GGSISSSSYY | Anti-BCMA CT103A |
| | | scFv heavy chain CDR1 |
| 86 | ISYSGST | Anti-BCMA CT103A |
| | | scFv heavy chain CDR2 |
| 87 | ARDRGDTILDV | Anti-BCMA CT103A |
| | | scFv heavy chain CDR3 |

### f) iv) Anti-FAP CAR

In certain embodiments comprising an anti-FAP CAR, the anti-FAP CAR comprises as scFv based on the antibody 4G5 (see WO2021/061708 and WO2021/061778). In some embodiments comprising an anti-FAP CAR comprising a scFv based on the antibody 4G5 further comprises a hinge and transmembrane from CD8, a 4-11B1 co-stimulatory domain, and a CD3 signaling domain, Examples of an anti-FAP CARs include CARs disclosed in WO2021/061778.

TABLE 13
Exemplary sequences of anti-FAP 4G5-based CARs

| SEQ ID NO: | Amino Acid Sequence | Description |
|---|---|---|
| 88 | MALPVTALLLPLALLLHAARPGS | Signal peptide |
| 89 | QVQLQQPGAELVKPGASVKLSCKA | 4G5 scFv VH N- |
| | SGYTITSYSLHWVKQRPGQGLEWIG | terminal |
| | EINPANGDHNFSEKFEIKATLTVDSS | |
| | SNTAFMQLSRLTSEDSAVYYCTRLD | |
| | DSRFHWYFDVWGAGTTVTVSSGGG | |
| | GSGGGGSGGGGSQIVLTQSPALMSA | |
| | SPGEKVTMTCTASSSVSYMYWYQQ | |
| | KPRSSPKPWIFLTSNLASGVPARFSG | |
| | RGSGTSFSLTISSMEAEDAATYYCQ | |
| | QWSGYPPITFGSGTKLEIK | |
| 90 | SGTTTPAPRPPTPAPTIASQPLSLRPE | CD8α hinge |
| | ACRPAAGGAVHTRGLDFACD | (preceded by SG spacer |
| 12 | IYIWAPLAGTCGVLLLSL VITLYC | CD8α transmembrane |
| | | domain |
| 14 | KRGRKKLLYIFKQPFMRPVQTTQEE | 4-1BB co-stimulatory |
| | DGCCRFPEEEEGGCEL | domain |
| 17 | RVKFSRSADAPAYKQGQNQLYNEL | Cd3ζ signaling domain |
| | NLGRREEYDVLDKRRGRDPEMGGK | |
| | PRRKNPQEGLYNELQKDKMAEAYS | |
| | EIGMKGERRRGKGHDGLYQGLSTA | |
| | TKDTYDALHMQALPPR | |
| 91 | QIVLTQSPALMSASPGEKVTMTCTA | 4G5 scFv VL N-terminal |
| | SSSVSYMYWYQQKPRSSPKPWIFLT | |
| | SNLASGVPARFSGRGSGTSFSLTISS | |
| | MEAEDAATYYCQQWSGYPPITFGS | |
| | GTKLEIKGGGGSGGGGSGGGGSQV | |
| | QLQQPGAELVKPGASVKLSCKASG | |
| | YTITSYSLHWVKQRPGQGLEWIGEI | |
| | NPANGDHNFSEKFEIKATLTVDSSSN | |
| | TAFMQLSRLTSEDSAVYYCTRLDDS | |
| | RFHWYFDVWGAGTTVTVSS | |

### f) v) Anti-GPRC5D CAR

In some embodiments, the mRNA encodes an anti-GPRC5D chimeric antigen receptor (CAR). GPRC5D is a G protein-coupled receptor without known ligands and of unclear function in human tissue. However, this receptor is expressed in myeloma cell lines and in bone marrow plasma cells from patients with multiple myeloma. GPRC5D has been identified as an immunotherapeutic target in multiple myeloma and Hodgkin lymphomas. Examples of anti-GPRC5D CARs include those incorporating a GPRC5D binding moiety such as MCARH109 (Mailankody et al., N Engl J Med. 387(13): 1196-1206 (2022)), BMS-986393, or OriCAR-017 (Rodriguez-Otero et al., Blood Cancer J. 14(1): 24 (2024)). Examples of anti-GPRC5D CARs include those incorporating a GPRC5D binding moiety derived from an antibody specific to GPRC5D, for example, talquetamab (Pillarisetti et al., Blood 135:1232-43 (2020)), or forimtamig. In some embodiments, the extracellular binding domain of the anti-GPRC5D CAR comprises an scFv derived from a 6D9 Mouse antibody with specificity to human GPRC5D (see creative-biolabs.com/car-t/anti-gprc5d-6d9-h-41bb-cd3-car-pcdcarl-26380.htm). In some embodiments, the extracellular binding domain of the GPRC5D CAR comprises an scFv of anti-GPRC5D antibody linked to 4-1BB or CD28 costimulatory domain and CD3ζ signaling domain as described in Mailankody et al., N Engl J Med. 387(13): 1196-1206 (2022); creative-biolabs.com/car-t/anti-gprc5d-6d9-h-41bb-cd3-car-pcdcarl-26380.htm; and Rodriguez-Otero et al., Blood Cancer J. 14(1): 24 (2024). The entire contents of each of foregoing references in this paragraph are incorporated by reference for all that they teach about the design, structure, and activity of anti-GPRC5D CARs and anti-GPRC5D antibodies that can provide an antigen binding domain for a CAR or immune cell engager, and each example constitutes a means for binding GPRC5D. In any of the aforementioned tLNP embodiments, certain embodiments include tLNPs encapsulating a GPRC5D CAR payload encoded by RNA and having a T cell targeting moiety, such as an anti-CD8 antibody

### f) vi) Anti-FCRL5 CAR

In some embodiments, the mRNA encodes an anti-FCRL5 chimeric antigen receptor (CAR). FCRL5 (Fc receptor-like 5), also known as FCRH5, BXMAS1, CD307, CD307E, and IRTA2, is a protein marker expressed on the surface of plasma cells in patients with multiple myeloma. Furthermore, contact with FCRL5 stimulates B-cell proliferation; thus, FCRL5 has been identified as an immunotherapeutic target for this disease. Examples of anti-FCRL5 CARS include those incorporating an FCRL5 binding moiety, such as those described in WO2016090337, WO2017096120, WO2022263855, and WO2024047558. In some embodiments, the extracellular binding domain of the anti-FCRL5 CAR comprises an scFv with specificity to FCRL5, such as ET200-31, ET200-39, ET200-69, ET200-104, ET200-105, ET200-109, or ET200-117. In some embodiments, the extracellular binding domain of the anti-FCRL5 CAR comprises an scFv derived from a mouse antibody with specificity to human FCRL5. Such antibodies include 7D11, F25, F56, and F119, as described in Polson et al., Int. Immunol., 18(9): 1363-1373 (2006); Franco et al., J. Immunol. 190(11): 5739-5746 (2013); Ise et al., Clin. Cancer Res. 11(1): 87-96 (2005); and Ise et al., Clin. Chem. Lab. Med. 44(5): 594-602 (2006), all of which are incorporated by reference herein. In some embodiments, the extracellular binding domain of the anti-FCRL5 CAR comprises a binding moiety derived from the antigen binding domain of an anti-FCRL5 antibody or nanobody, including cevostamab, 2A10H7, 307307, 2A10D6, 13G9, 10A8, 509f6, EPR27365-87, EPR26948-19, or EPR26948-67, or as disclosed in WO2016090337, WO2017096120, WO2022263855, or WO2024047558. In some embodiments, the extracellular binding domain of the anti-FCRL5 CAR comprises a binding moiety derived from an antibody-drug conjugate targeting FCRL5, such as those described in Elkins et al., Mol. Cancer Ther. 11(10): 2222-2232 (2012). In some embodiments, the extracellular binding domain of the anti-FCRL5 CAR is linked to a costimulatory domain, such as a 4-1BB or CD28 costimulatory domain, and a signaling domain, such as a CD3ζ signaling domain. The entire contents of each of the foregoing references in this paragraph are incorporated by reference for all that they teach about the design, structure, properties, and activity of anti-FCRL5 CARs and anti-FCRL5 antibodies that can provide an antigen binding domain for a CAR or immune cell engager. Each example constitutes a means for binding FCRL5. In any of the aforementioned tLNP embodiments, certain embodiments include tLNPs encapsulating a FCRL5 CAR payload encoded by RNA and having a T cell targeting moiety, such as an anti-CD8 antibody.

Each of the CARs with specificity for a particular antigen described herein constitute means for antigen recognition with respect to that antigen and collectively all of the CARs described herein constitute means for antigen recognition. The function may be alternatively stated as antigen recognition by an immune cell or antigen recognition by a T cell and the like.

### TCEs

A variety of antibody-derived multi-specific molecules have been used as TCEs. Heterodimerization of whole antibodies can be promoted with mutations in the heavy chain constant regions as in duobody and knobs-in-holes technology. CrossMab technology exchanges the light chain constant domain with the CH₁domain in one parental antibody to prevent mispairing of light chains. Other TCE constructs are based on scFv and diabody designs such as BiTEs and DARTs. Discussion of these various antibody formats can be found in Tian et al., J Hematol Oncol 14, 75 (2021) and Wilkinson & Hale, MAbs 14(1):2123299, (2022) including its Supplementary Tables, each of which is incorporated by reference for all that they teach about TCE design and architecture that is not inconsistent with the present disclosure.

A TCE serves as a bridge between a signaling receptor on the T cells, most often CD3, and a surface antigen on a pursued cells. When the receptor and antigen specificities of the TCE are engaged the T cell responds in a manner similar to when its TCR engages antigen on another cell. The TCE can bind to both the T cell that produced it and other T cells in the vicinity so that more than just the successfully transfected T cell can react against the pursued cell. Other immune cell engagers perform analogously.

### Binding Moieties

Binding moieties are important for two features of the tLNPs herein disclosed. The binding moiety can serve as the targeting moiety for the tLNP or as an antigen binding domain of the antigen receptor encoded by the encapsulated mRNA. The tLNP of the various disclosed aspects comprise a binding moiety, such as an antibody or antigen binding domain thereof or a cell surface receptor ligand. The fundamental ability of the tLNP to deliver an mRNA into the cytoplasm of a T cell is agnostic with respect to, and does not depend upon, a particular binding specificity. Of course, a binding moiety is a determinant of which T (or other immune) cells into which an mRNA is delivered. There are many known antibodies with specificity for one or another T cell surface marker associated with all or a particular T cell subset(s) that could be used as the target of the binding moiety on a disclosed tLNP and there are several sources that have compiled such information. Similarly, there are many known antibodies with specificity for cancers cells, B cells, and fibrogenic cells that could be used to provide antigen binding domains with specificity for such cells for incorporation into CARs or TCEs. An excellent source of information about antibodies for which an International Non-proprietary Name (INN) has been proposed or recommended is Wilkinson & Hale, MAbs 14(1):2123299, 2022, including its Supplementary Tables, which is incorporated by reference herein for all that it teaches about individual antibodies and the various antibody formats that can be constructed. U.S. Pat. No. 11,326,182 and especially its Table 9 Cancer, Inflammation and Immune System Antibodies, is a source of sequence and other information for a wide range of antibodies including many that do not have an INN and is incorporated herein by reference for all that it teaches about individual antibodies. Sequence information is not always readily available for antibodies mentioned in the art, even when commercially available. This is not necessarily an impediment to their use. Where the antibody or a cell line is commercially available or obtainable from its originator it can be used as the binding moiety of tLNP without any need for sequence information. Even where sequence information is needed, it is well within the capabilities of the skilled artisan to sequence the antibody protein (or have it done by a contract laboratory) so that the antibody's variable region can be incorporated into a scFv, a diabody, a minibody, a TCE, or some other antibody format, or be humanized. In choosing among available antibodies in the art for the development of an agent to be used in humans, a human antibody is preferred to a humanized antibody is preferred to a non-human antibody, other factors being equal. Other factors can include stability and ease of production of the antibody, affinity of the antibody, and cross-reactivity for the cognate antigen in model species to be used in product development.

The tLNP of the various disclosed aspects comprise a binding moiety, such as an antibody or antigen binding domain thereof or a cell surface receptor ligand. As used herein, a “binding moiety” or “targeting moiety” refers to a protein, polypeptide, oligopeptide or peptide, carbohydrate, nucleic acid, or combinations thereof capable of specifically binding to a target or multiple targets. A binding domain includes any naturally occurring, synthetic, semi-synthetic, or recombinantly produced binding partner for a biological molecule or another target of interest. Exemplary binding moieties of this disclosure include an antibody, a Fab′, F(ab′)2, Fab, Fv, rIgG, scFv, hcAb (heavy chain antibody), a single domain antibody, VHH, VNAR, sdAb, nanobody, receptor ectodomain or ligand-binding portions thereof, or ligand (e.g., cytokines, chemokines). An “Fab” (antigen binding fragment) is the part of an antibody that binds to antigens and includes the variable region and CH1 of the heavy chain linked to the light chain via an inter-chain disulfide bond. In other embodiments, a binding moiety comprises a ligand-binding domain of a receptor or a receptor ligand. In some embodiments, a binding moiety can have more than one specificity including, for example, bispecific or multispecific binders. In some embodiments, a binding moiety may be an antibody or an antigen-binding portion thereof, an antigen; a ligand-binding domain of a receptor; or a receptor ligand. In some embodiments, a binding moiety can have more than one specificity including, for example, bispecific or multispecific binders.

In some embodiments, a binding moiety comprises an antibody or an antigen-binding portion thereof. The term “antibody” refers to a protein comprising an immunoglobulin domain having hypervariable regions determining the specificity with which the antibody binds antigen, termed complementarity determining regions (CDRs). The term antibody can thus refer to whole antibodies (also referred to as intact or full-length antibodies) as well as antibody fragments and constructs comprising an antigen binding portion of a whole antibody. While the canonical natural antibody has a pair of heavy and light chains, camelids (from camels, alpacas, llamas, and the like) produce antibodies with both the canonical structure and antibodies comprising only heavy chains. The variable region of the camelid heavy chain-only antibody has a distinct structure with a lengthened CDR3 referred to as VHH or, when produced as a fragment, a nanobody. Antigen binding fragments and constructs of antibodies include F(ab)2, F(ab′), F(ab′)2, F(ab), minibodies, Fv, single-chain Fv (scFv), diabodies, and VH. Such elements can be combined to produce bi- and multi-specific reagents, such as BiTEs (bi-specific T-cell engagers). The term “monoclonal antibody” arose out of hybridoma technology but is now used to refer to any singular molecular species of antibody regardless of how it was originated or produced. Antibodies can be obtained through immunization, selection from a naïve or immunized library (for example, by phage display), alteration of an isolated antibody-encoding sequence, or any combination thereof. Numerous antibodies that can be used as binding moieties are known in the art. An excellent source of information about antibodies for an International Non-proprietary Name (INN) has been proposed or recommended, including sequence information, is Wilkinson & Hale, 2022, MAbs 14(1):2123299, including its Supplementary Tables, which is incorporated by reference herein for all that it teaches about individual antibodies and the various antibody formats that can be constructed. U.S. Pat. No. 11,326,182 and especially its Table 9 entitled “Cancer, Inflammation and Immune System Antibodies,” is a source of sequence and other information for a wide range of antibodies including many that do not have an INN and is incorporated herein by reference for all that it teaches about individual antibodies.

A diabody is a dimer of scFv fragment that consists of the V_{H} and V_{L} regions noncovalently connected by a small peptide linker or covalently linked to each other. A BiTE is a fusion protein having two scFvs of different antibodies, usually an antibody for a tumor-associated antigen and antibody for CD3, on a single peptide chain, thus forming a cytolytic synapse between T cells and pursued antigen-bearing cells. The term “antigen-binding portion” may refer to a portion of an antibody as described that possesses the ability to specifically recognize, associate, unite, or combine with a target or pursued molecule. An antigen-binding portion includes any naturally occurring, synthetic, semi-synthetic, or recombinantly produced binding partner for a specific antigen. Thus, antibodies and antigen-binding portions thereof constitute means for binding to the surface molecule on a cell. In various embodiments, the cell can be an immune cell, a leukocyte, a lymphocyte, a monocyte, a stem cell, a hematopoietic stem cell (HSC) or a mesenchymal stem cell (MSC), according to the specificity of the antibody.

In some embodiments, the antibody or antigen-binding portion thereof may be derived from a mammalian species, for example, mice, rats, or human. Antibody variable regions can be those arising from one species, or they can be chimeric, containing segments of multiple species possibly further altered to optimize characteristics such as binding affinity or low immunogenicity. For human applications, it is desirable that the antibody has a human sequence. In the cases where the antibody or antigen-binding portion thereof is derived from a non-human species, the antibody or antigen-binding portion thereof may be humanized to reduce immunogenicity in a human subject. For example, if a human antibody of the desired specificity is not available, but such an antibody from a non-human species is, the non-human antibody can be humanized, e.g., through CDR grafting, in which the CDRs from the non-human antibody are placed into the respective positions in a framework of a compatible human antibody. Less preferred is an antibody in which only the constant region of the non-human antibody is replaced with human sequence. Such antibodies are commonly referred to as chimeric antibodies in distinction to humanized antibodies.

In some embodiments, the antibody or antigen-binding portion thereof is non-immunogenic. In some embodiments, the antibody may be modified in its Fc region to reduce or eliminate secondary functions, such as FcR engagement, antibody-dependent cellular cytotoxicity (ADCC), antibody-dependent cellular phagocytosis (ADCP), and/or complement-dependent cytotoxicity (CDC).

A binder density on the tLNP can be defined according to the ratio of antibody (binder) to mRNA (w/w) either based on the amount of antibody input in the conjugation reaction or as measured in the tLNP. For an intact antibody (e.g., whole IgG), in some embodiments, preferred ratios are about 0.3 to about 1.0, about 0.3 to about 0.7, about 0.3 to about 0.5, about 0.5 to about 1.0, and about 0.5 to about 0.7 for either the input or final measured binder ratio. In certain embodiments, a tLNP has an antibody ratio of 0.3 to 1.0, 0.3 to 0.7, 0.3 to 0.5, 0.5 to 1.0, and 0.5 to 0.7 for either the input or final measured binder ratio. In some embodiments, if the binder is different in size from an intact antibody (for example a scFv, diabody, or minibody, etc.) the w/w ratio is adjusted for the different size of the binder.

Immune engineering amplification can be relevant to the generation of engineered NK, NKT, and myeloid cells as well as to T cells, resulting in metabolic activation upon CAR expression and target engagement, or activation through secreted or tethered BRMs, rendering the cells more readily transfectable. In certain embodiments, the LNP is a tLNP comprising a binding moiety serving as a targeting moiety that specifically binds to a cell surface protein. In certain embodiments, a LNP or tLNP comprises a binding moiety derived from an anti-CD40*^{‡} antibody, an anti-LRRC15^{†‡} antibody, an anti-CTSK antibody, an anti-ADAM12^{‡} antibody, an anti-CLDN6 antibody, an anti-CLDN 18.2 antibody, an anti-DLL3 antibody, an anti-IL13Rα2 antibody, an anti-ITGA11 antibody, an anti-FAP*^{†‡} antibody, an anti-NOX4 antibody, an anti-SGCD antibody, an anti-SYNDIG1 antibody, an anti-CDH11^{‡} antibody, an anti-PLPP4 antibody, an anti-SLC24A2 antibody, an anti-PDGFRB*^{‡} antibody, an anti-THY1^{‡} antibody, an anti-ANTXR1^{‡} antibody, an anti-GAS1 antibody, an anti-CALHM5 antibody, an anti-SDC1*^{‡} antibody, an anti-HER2*^{†‡} antibody, an anti-TROP2*^{†‡} antibody, an anti-MSLN*^{‡} antibody, an anti-Nectin4^{†‡} antibody, or an anti-MUC16*^{†‡} antibody. In certain embodiments, a LNP or tLNP comprises a binding moiety specific for an immune cell antigen selected from CD1, CD2*^{†‡}, CD3*^{†‡}, CD4*^{†‡}, CD5^{†‡}, CD7^{†‡}, CD8^{†}, CD11b^{‡}, CD14^{†‡}, CD16, CD25^{†‡}, CD26*†‡, CD27*^{†‡}, CD28*^{†‡}, CD30*^{†‡}, CD32*, CD38*^{†‡}, CD39^{‡}, CD40*^{†‡}, CD40L (CD154)*^{†‡}, CD44*^{‡}, CD45^{†‡}, CD56^{†‡}, CD62^{†‡}, CD64*‡, CD68, CD69^{‡}, CD73^{†‡}, CD80*^{‡}, CD83^{‡}, CD86*^{‡}, CD95^{‡}, CD103^{‡}, CD119^{‡}, CD126^{‡}, CD137 (41BB)^{†‡}, CD150^{†‡}, CD153^{‡}, CD161^{‡}, CD166^{‡}, CD183 (CXCR3)^{‡}, CD183 (CXCR5)^{‡}, CD223 (LAG-3)*^{†‡}, CD254^{‡}, CD275^{‡}, CD45RA, CTLA-4*†‡t, DEC205, OX40^{†}, PD-1*^{†‡}, GITR^{†}, TIM-3*^{†‡}, FasL*^{‡}, IL18R1, ICOS (CD278)^{‡}, leu-12, TCR^{†}, TLR1, TLR2^{†‡}, TLR3*^{‡}, TLR4^{†‡}, TLR6, TREM2^{‡}, NKG2D^{‡}, CCR, CCR1 (CD191)^{‡}, CCR2 (CD192)*^{†‡}, CCR4(CD194)*^{†‡}, CCR6(CD196)^{‡}, CCR7^{‡}, low affinity IL-2 receptor^{†‡}, IL-7 receptor^{‡}, IL-12 receptor^{‡}, IL-15 receptor^{‡}, IL-18 receptor^{‡}, and IL-21 receptor^{‡}. (* indicates that exemplary antibodies with the indicated specificity from which a binding moiety could be derived can be found in U.S. Pat. No. 11,326,182B2 Table 9 or 10. † indicates that exemplary antibodies with the indicated specificity from which a binding moiety could be derived can be found in Wilkinson & Hale, 2022. Both references cited and incorporated by reference above. ‡ indicates that exemplary antibodies with the indicated specificity from which a binding moiety could be derived can be found in the Therapeutic Antibody Database (TABS) at tabs.craic.com). Other suitable antibodies can be found in Appendix A or WO2024040195A1 each of which is incorporated herein by reference for all that it teaches about individual antibodies and the antigens they bind.

The following paragraphs provide non-exhaustive examples of known antibodies that bind to cell surface markers on T cells. These antibodies or the antigen binding domains thereof can be used as binding moieties to target the disclosed tLNP. Collectively these antibodies and polypeptides comprising the antigen binding domains thereof constitute means for binding T cell surface markers or means for binding T cells.

In some embodiments, CD2 is a targeted cell surface antigen and a binding moiety comprises the antigen binding domain of an anti-CD2 antibody. CD2 contains three well-characterized epitopes (T11.1, T11.2, and T11.3/CD2R). T11.3/CD2R are membrane proximal and exposure is increased upon T cell activation and CD2 clustering. Accordingly, in some such embodiments, the anti-CD2 antigen binding domain is derived from, RPA-2.10; OKT11, UMCD2, 0.1, and 3T4-8B5 (T11.1 epitope); 9.6 and 1OLD2-4C₁ (T11.2 epitope); 1Mono2A6 (T11.3 epitope), siplizumab (T11.2/T11.3 epitope), HuMCD2, TS2/18, TS1/8, AB75, LT-2, T6.3, MEM-65, OTI4E4, or an antigen-binding portion thereof. Additionally, the ligand of CD2, CD58 (LFA-3) can be used as a CD2 binding moiety as can alefacept, a CD58-Fc fusion. Each of these constitutes a means for binding CD2 (Li et al., 1996, J Mol Biol. 263:209-26; Binder et al., 2020, Front Immunol. 9:11:1090).

In some embodiments, CD3 is a targeted cell surface antigen and a binding moiety comprises the antigen binding domain of an anti-CD3 antibody. Accordingly, in some such embodiments, the antigen binding domain is derived from muromonab-CD3 (OKT3), teplizumab, otelixizumab, visilizumab, cevostamab, teclistamab, elranatamab pavurutamab, vibecotamab, odronextamab, or an antigen-binding portion thereof. Each of these constitutes a means for binding CD3.

In some embodiments, CD4 is a targeted cell surface antigen and a binding moiety comprises the antigen binding domain of an anti-CD4 antibody. Accordingly, in some such embodiments, the antigen binding domain is derived from ibalizumab, inezetamab, semzuvolimab, zanolimumab, tregalizumab, UB-421, priliximab, MTRX1011A, cedelizumab, clenoliximab, keliximab, M-T413, TRX1, hB-F5, MAX.16H5, IT208, or an antigen-binding portion thereof. Each of these constitutes a means for binding CD4.

In some embodiments, CD5 is a targeted cell surface antigen and a binding moiety comprises the antigen binding domain of an anti-CD5 antibody. Accordingly, in some such embodiments, the antigen binding domain is derived from 5D7, UCHT2, L17F12, H65, HE3, OKT1, MAT304, as well as those disclosed in WO1989006968, WO2008121160, U.S. Pat. No. 8,679,500, WO2010022737, WO2019108863, WO2022040608, or WO2022127844, each of which is incorporated by reference for all that they teach about anti-CD5 antibodies and their properties, or an antigen-binding portion thereof. Each of these constitutes a means for binding CD5.

In some embodiments, CD7 is a targeted cell surface antigen and a binding moiety comprises the antigen binding domain of an anti-CD7 antibody. Accordingly, in some such embodiments, the antigen binding domain is derived from TH-69, 3A1E, 3A1F, Huly-m2, WT1, YTH3.2.6, T3-3A1, grisnilimab, as well as those disclosed in U.S. Pat. No. 10,106,609, WO2017213979, WO2018098306, U.S. Ser. No. 11/447,548, WO2022136888, WO2020212710, WO2021160267, WO2022095802, WO2022095803, WO2022151851, or WO2022257835 each of which is incorporated by reference for all that they teach about anti-CD7 antibodies and their properties, or an antigen-binding portion thereof. Each of these constitutes a means for binding CD7.

In some embodiments, CD8 is a targeted cell surface antigen and a binding moiety comprises the antigen binding domain of an anti-CD8 antibody. Accordingly, in some such embodiments, the antigen binding domain is derived from crefmirlimab (IAB22M), 3B5, SP-16, LT8, 17D8, MEM-31, MEM-87, RIV11, UCHT4, YTC182.20, RPA-T8, OKT8, SK1, 51.1, TRX2, MT807-R1, HIT8α, C8/144B, RAVB3, SIDI8BEE, BU88, EPR26538-16, 2ST8.5H7, as well as those disclosed in U.S. Pat. No. 10,414,820, WO2015184203, WO2017134306, WO2019032661, WO2020060924, U.S. Pat. No. 10,730,944, WO2019033043, WO2021046159, WO2021127088, WO2022081516, U.S. Pat. No. 11,535,869, or WO2023004304 each of which is incorporated by reference for all that they teach about anti-CD8 antibodies and their properties, or an antigen-binding portion thereof. Additionally, humanized anti-CD8 antibodies are described in International Patent Application Number PCT/US2024/060426, filed on Dec. 16, 2024, which is incorporated by reference for all that it teaches about these humanized anti-CD8 antibodies and their properties, or an antigen-binding portion thereof. Each of the foregoing anti-CD8 antibodies constitutes a means for binding CD8.

In some embodiments, CD11b is a targeted cell surface antigen and a binding moiety comprises the antigen binding domain of an anti-CD11b antibody. Accordingly, in some such embodiments, the antigen binding domain is derived from ASD141 or MAB107 as well as those disclosed in US20150337039, U.S. Pat. No. 10,738,121, WO2016197974, U.S. Pat. No. 10,919,967, or WO2022147338 each of which is incorporated by reference for all that they teach about anti-CD11b antibodies and their properties, or an antigen-binding portion thereof. Each of these constitutes a means for binding CD11b.

In some embodiments, CD13 is a targeted cell surface antigen and a binding moiety comprises the antigen binding domain of an anti-CD13 antibody. CD13 is also known as aminopeptidase N (APN). Accordingly, in some such embodiments, the antigen binding domain is derived from MT95-4 or Nbl57 (disclosed in WO2021072312 which is incorporated by reference for all that they teach about anti-CD13 antibodies and their properties), as well as those disclosed in WO2023037015 which is incorporated by reference for all that it teaches about anti-CD13 antibodies and their properties, or an antigen-binding portion thereof. Each of these constitutes a means for binding CD13.

In some embodiments, CD14 is a targeted cell surface antigen and a binding moiety comprises the antigen binding domain of an anti-CD14 antibody. Accordingly, in some such embodiments, the antigen binding domain is derived from atibuclimab or r18D11 as well as those disclosed in WO2018191786 or WO2015140591 each of which is incorporated by reference for all that they teach about anti-CD14 antibodies and their properties, or an antigen-binding portion thereof. Each of these constitutes a means for binding CD14.

In some embodiments, CD16a is a targeted cell surface antigen and a binding moiety comprises the antigen binding domain of an anti-CD16a antibody. Accordingly, in some such embodiments, the antigen binding domain is derived from AFM13, sdA1, sdA2, or hu3G8-5.1-N297Q as well as those disclosed in U.S. Ser. No. 11/535,672, WO2018158349, WO2007009065, U.S. Ser. No. 10/385,137, WO2017064221, U.S. Pat. No. 10,758,625, WO2018039626, WO2018152516, WO2021076564, WO2022161314, or WO2023274183 each of which is incorporated by reference for all that they teach about anti-CD16A antibodies and their properties, or an antigen-binding portion thereof. Each of these constitutes a means for binding CD16a.

In some embodiments, CD25 is a targeted cell surface antigen and a binding moiety comprises the antigen binding domain of an anti-CD25 antibody. Accordingly, in some such embodiments, the antigen binding domain is derived from daclizumab, basiliximab, camidanlumab, tesirine, inolimomab, R07296682, HuMax-TAC, CYT-91000, STI-003, RTX-003, or an antigen-binding portion thereof. Each of these constitutes a means for binding CD25.

In some embodiments, CD28 is a targeted cell surface antigen and a binding moiety comprises the antigen binding domain of an anti-CD28 antibody. Accordingly, in some such embodiments, the antigen binding domain is derived from GN1412, acazicolcept, lulizumab, prezalumab, theralizumab, FR104CD, and davoceticept, as well as those disclosed in U.S. Pat. Nos. 8,454,959, 8,785,604, 11,548,947, 11,530,268, 11,453,721, 11,591,401, WO2002030459, WO2002047721, US20170335016, US20200181260, U.S. Ser. No. 11/608,376, WO2020127618, WO2021155071, or WO2022056199 each of which is incorporated by reference for all that they teach about anti-CD28 antibodies and their properties, or an antigen-binding portion thereof. Each of these constitutes a means for binding CD28.

In some embodiments, CD32A is a targeted cell surface antigen and a binding moiety comprises the antigen binding domain of an anti-CD32A antibody. Accordingly, in some such embodiments, the antigen binding domain is derived from VIB9600, humanized IV.3, humanized AT-10, or MDE-8 as well as those disclosed in U.S. Pat. Nos. 9,688,755, 9,284,375, 9,382,321, U.S. Ser. No. 11/306,145, or WO2022067394 each of which is incorporated by reference for all that they teach about anti-CD32A antibodies and their properties, or an antigen-binding portion thereof. Each of these constitutes a means for binding CD32A.

In some embodiments, CD40 is a targeted cell surface antigen and a binding moiety comprises the antigen binding domain of an anti-CD40 antibody. Accordingly, in some such embodiments, the antigen binding domain is derived from cifurtilimab, sotigalimab, iscalimab, dacetuzumab, selicrelumab, bleselumab, lucatumumab, or mitazalimab as well as those disclosed in U.S. Ser. No. 10/633,444, each of which is incorporated by reference for all that they teach about anti-CD40 antibodies and their properties, or an antigen-binding portion thereof. Each of these constitutes a means for binding CD40.

In some embodiments, CD44 is a targeted cell surface antigen and a binding moiety comprises the antigen binding domain of an anti-CD44 antibody. Accordingly, in some such embodiments, the antigen binding domain is derived from R05429083, VB6-008, PF-03475952, or RG7356, as well as those disclosed in WO2008144890, U.S. Pat. No. 8,383,117, WO2008079246, US20100040540, WO2015076425, U.S. Pat. No. 9,220,772, US20140308301, WO2020159754, WO2021160269, WO2021178896, WO2022022749, WO2022022720, or WO2022243838, each of which is incorporated by reference for all that they teach about anti-CD44 antibodies and their properties, or an antigen-binding portion thereof. Each of these constitutes a means for binding CD44.

In some embodiments, CD45 is a targeted cell surface antigen and a binding moiety comprises the antigen binding domain of an anti-CD45 antibody. Accordingly, in some such embodiments, the antigen binding domain is derived from apamistamab, BC8-B10, as well as those disclosed in WO2023183927, WO2023235772, U.S. Pat. No. 7,825,222, WO2017009473, WO2021186056, U.S. Pat. Nos. 9,701,756, 9,701,756, WO2020092654, WO2022040088, WO2022040577, WO2022064191, WO2022063853, or WO2024064771, each of which is incorporated by reference for all that they teach about anti-CD45 antibodies and their properties, or an antigen-binding portion thereof. Each of these constitutes a means for binding CD45.

In some embodiments, CD56 is a targeted cell surface antigen and a binding moiety comprises the antigen binding domain of an anti-CD56 antibody. Accordingly, in some such embodiments, the antigen binding domain is derived from lorvotuzumab, adcitmer, or promiximab, as well as those disclosed in WO2012138537, U.S. Pat. No. 10,548,987, 10,730,941, or US20230144142, each of which is incorporated by reference for all that they teach about anti-CD56 antibodies and their properties, or an antigen-binding portion thereof. Each of these constitutes a means for binding CD56.

In some embodiments, CD64 is a targeted cell surface antigen and a binding moiety comprises the antigen binding domain of an anti-CD64 antibody. Accordingly, in some such embodiments, the antigen binding domain is derived from HuMAb 611 or H22 as well as those disclosed in U.S. Pat. No. 7,378,504, WO2014083379, US20170166638, or WO2022155608 each of which is incorporated by reference for all that they teach about anti-CD64 antibodies and their properties, or an antigen-binding portion thereof. Each of these constitutes a means for binding CD64.

In some embodiments, CD68 is a targeted cell surface antigen and a binding moiety comprises the antigen binding domain of an anti-CD68 antibody. Accordingly, in some such embodiments, the antigen binding domain is derived from Ki-M7, PG-M1, 514H12, ABM53F5, 3F7C6, 3F7D3, Y1/82A, EPR20545, CDLA68-1, LAMP4-824, or an antigen-binding portion thereof. Each of these constitutes a means for binding CD68.

In some embodiments, CD70 is a targeted cell surface antigen and a binding moiety comprises the antigen binding domain of an anti-CD70 antibody. Accordingly, in some such embodiments, the antigen binding domain is derived from cusatuzumab, vorsetuzumab, MDX-1203, MDX-1411, AMG-172, SGN-CD70A, ARX305, PRO1160, as well as those disclosed in U.S. Pat. Nos. 9,765,148, 8,124,738, IS10,266,604, WO2021138264, U.S. Pat. Nos. 9,701,752, 10,108,123, WO2014158821, U.S. Pat. No. 10,689,456, WO2017062271, U.S. Pat. Nos. 11,046,775, 11,377,500, WO2021055437, WO2021245603, WO2022002019, WO2022078344, WO2022105914, WO2022143951, WO2023278520, WO2022226317, WO2022262101, U.S. Pat. No. 11,613,584, or WO2023072307, each of which is incorporated by reference for all that they teach about anti-CD70 antibodies and their properties, or an antigen-binding portion thereof. Each of these constitutes a means for binding CD70.

In some embodiments, CD73 is a targeted cell surface antigen and a binding moiety comprises the antigen binding domain of an anti-CD73 antibody. Accordingly, in some such embodiments, the antigen binding domain is derived from oleclumab, uliledlimab, mupadolimab, AK119, IBI325, BMS-986179, NZV930, JAB-BX102, Sym024, TB19, TB38, HBM1007, 3F7, mAb19, Hu001-MMAE, IPH5301, or INCA00186, as well as those disclosed in U.S. Pat. Nos. 9,938,356, 10,584,169, WO2022083723, WO2022037531, WO2021213466, WO2022083049, U.S. Pat. No. 10,822,426, WO2021259199, U.S. Pat. Nos. 10,100,129, 11,312,783, 11,174,319, 11,634,500, WO2021138467, WO2017118613, U.S. Pat. No. 9,388,249, WO2020216697, U.S. Ser. No. 11/180,554, U.S. Pat. No. 11,530,273, WO2019173692, WO2019170131, U.S. Pat. No. 11,312,785, WO2020098599, WO2020143836, WO2020143710, U.S. Pat. Nos. 11,034,771, 11,299,550, WO2020253568, WO2021017892, WO2021032173, WO2021032173, WO2021097223, WO2021205383, WO2021227307, WO2021241729, WO2022096020, WO2022105881, WO2022179039, WO2022214677, or WO2022242758, each of which is incorporated by reference for all that they teach about anti-CD73 antibodies and their properties, or an antigen-binding portion thereof. Each of these constitutes a means for binding CD73.

In some embodiments, CD123 is a targeted cell surface antigen and a binding moiety comprises the antigen binding domain of an anti-CD123 antibody. CD123 is also known as IL-3Ra. Accordingly, in some such embodiments, the antigen binding domain is derived from flotetuzumab, vibecotamab, or talacotuzumab, as well as those disclosed in U.S. Pat. No. 10,844,128, which is incorporated by reference for all that it teaches about CD123 antibodies and their properties, or an antigen-binding portion thereof. Each of these constitutes a means for binding CD123.

In some embodiments, CD137 is a targeted cell surface antigen and a binding moiety comprises the antigen binding domain of an anti-CD137 antibody. CD137 is also known as 4-1BB. Accordingly, in some such embodiments, the antigen binding domain is derived from YH004, urelumab (BMS-663513), utomilumab (PF-05082566), ADG106, LVGN6051, PRS-343, as well as those disclosed in WO2005035584, WO2012032433, WO2017123650, U.S. Pat. Nos. 11,203,643, 11,242,395, 11,555,077, US20230067770, U.S. Pat. Nos. 11,535,678, 11,440,966, WO2019092451, U.S. Pat. Nos. 10,174,122, 11,242,385, 10,716,851, WO2020011966, WO2020011964, or U.S. Pat. No. 11,447,558, each of which is incorporated by reference for all that they teach about CD137 antibodies and their properties, or an antigen-binding portion thereof. Each of these constitutes a means for binding CD137.

In some embodiments, CD166 is a targeted cell surface antigen and a binding moiety comprises the antigen binding domain of an anti-CD166 antibody. Accordingly, in some such embodiments, the antigen binding domain is derived from praluzatamab, AZN-L50, REA442, or AT002, as well as those disclosed in U.S. Pat. Nos. 10,745,481, 11,220,544, or WO2008117049, each of which is incorporated by reference for all that they teach about CD166 antibodies and their properties, or an antigen-binding portion thereof. Each of these constitutes a means for binding CD166.

In some embodiments, CD200 is a targeted cell surface antigen and a binding moiety comprises the antigen binding domain of an anti-CD200 antibody. Accordingly, in some such embodiments, the antigen binding domain is derived from samalizumab, OX-104, REA1067, B7V3V2, HPAB-0260-YJ, or TTI-CD200, as well as those disclosed in WO2007084321 or WO2019126536, each of which is incorporated by reference for all that they teach about CD200 antibodies and their properties, or an antigen-binding portion thereof. Each of these constitutes a means for binding CD200.

In some embodiments, CD205 (also known as DEC205) is a targeted cell surface antigen and a binding moiety comprises the antigen binding domain of an anti-CD205 antibody.

CD205 is also known as DEC205. Accordingly, in some such embodiments, the antibody comprises 3G9-2D2 (a component of CDX-1401) or LY75_A1 (a component of MEN1309) as well as those disclosed in U.S. Pat. Nos. 8,236,318, 10,081,682, or U.S. Pat. No. 11,365,258, each of which is incorporated by reference for all that they teach about anti-CD205 antibodies and their properties, or an antigen-binding portion thereof. Each of these constitutes a means for binding CD205.

In some embodiments, CTLA-4 is a targeted cell surface antigen and a binding moiety comprises the antigen binding domain of an anti-CTLA-4 antibody. Accordingly, in some such embodiments, the antigen binding domain is derived from botensilimab, ipilimumab, nurulimab, quavonlimab, tremelimumab, zalifrelimab, ADG116, ADG126, ADU-1604, AGEN1181, BCD-145, BMS-986218, BMS-986249, BT-007, CS1002, GIGA-564, HBM4003, IBI310 JK08, JMW-3B3, JS007, KD6001, KN044, ONC-392, REGN4659, TG6050, XTX101, YH001, or an antigen-binding portion thereof. Each of these constitutes a means for binding CTLA-4.

In some embodiments, GITR is a targeted cell surface antigen and a binding moiety comprises the antigen binding domain of an anti-GITR antibody. Accordingly, in some such embodiments, the antigen binding domain is derived from ragifilimab, TRX518, MK-4166, AMG 228, MEDI1873, BMS-986156, REGN6569, ASP1951, MK-1248, FRA154, GWN323, JNJ-64164711, ATOR-1144, or an antigen-binding portion thereof. Each of these constitutes a means for binding GITR.

In some embodiments, a low affinity IL-2 receptor is a targeted cell surface antigen (CD122 and/or CD132) and a binding moiety comprises the antigen binding domain of an anti-IL-2 receptor antibody. Accordingly, in some such embodiments, the antiCD122 antibody comprises ANV419, FB102, MiK-Beta-1 and the anti CD122 antibodies disclosed in WO2011127324, WO2017021540, WO2022212848, WO2022221409, WO2023078113, US20230272090, WO2024073723, or an antigen-binding portion thereof. Accordingly, in some such embodiments, the anti-CD132 antibody comprises REGN7257 and the anti-CD132 antibodies disclosed in WO2020160242, WO2017021540, WO2022212848, WO2023078113, US20230272089, or an antigen-binding portion thereof. Each of these constitutes a means for binding the low affinity IL-2 receptor (CD122 or CD132, as appropriate),

In some embodiments, a high affinity IL-2 receptor is a targeted cell surface antigen (CD25) and a binding moiety comprises the antigen binding domain of an anti-IL-2 receptor antibody. Accordingly, in some such embodiments, the antigen binding domain is derived from daclizumab, basiliximab, camidanlumab, vopitug, inolimomab, HuMAx-TAC, Xenopax, STI-003, RA8, RTX-003, and the anti-CD25 antibodies disclosed in WO2023031403, WO2006108670, WO2019175223, WO2019175215, WO2019175226, WO2004045512, WO2022104009, WO2020102591, or an antigen-binding portion thereof. Each of these constitutes a means for binding the high affinity IL-2 receptor (CD25).

In some embodiments, IL-7 receptor (CD127) is a targeted cell surface antigen and a binding moiety comprises the antigen binding domain of an anti-IL-7 receptor antibody. Accordingly, in some such embodiments, the antigen binding domain is derived from PF-06342674, GSK2618960, OSE-127, lusvertikimab, bempikibart, and the anti-CD127 antibodies disclosed in WO2011104687, WO2011094259, WO2013056984, WO2015189302, WO2017062748, WO2020154293, WO2020254827, WO2021222227, WO2023201316, or an antigen-binding portion thereof. Each of these constitutes a means for binding the CD127.

In some embodiments, IL-12 receptor is a targeted cell surface antigen and a binding moiety comprises the antigen binding domain of an anti-IL-12 receptor antibody. Accordingly, in some such embodiments, the antigen binding domain is derived from CBYY-I0413, REA333, or an antigen-binding portion thereof. Each of these constitutes a means for binding the IL-12 receptor.

In some embodiments, IL-15 receptor a is a targeted cell surface antigen and a binding moiety comprises the antigen binding domain of an anti-IL-15 receptor a antibody.

Accordingly, in some such embodiments, the antigen binding domain is derived from MAB1472-100, MAB5511, JM7A4, 5E3E1, JM7A4, 2639B, or an antigen-binding portion thereof. Each of these constitutes a means for binding the IL-15 receptor a.

In some embodiments, IL-18 receptor a is a targeted cell surface antigen and a binding moiety comprises the antigen binding domain of an anti-IL-18 receptor a antibody.

Accordingly, in some such embodiments, the antigen binding domain is derived from H44, or an antigen-binding portion thereof. Each of these constitutes a means for binding the IL-18 receptor α.

In some embodiments, IL-21 receptor is a targeted cell surface antigen and a binding moiety comprises the antigen binding domain of an anti-IL-21 receptor antibody.

Accordingly, in some such embodiments, the antigen binding domain is derived from 1D1C2, 19F5, 18A5, REA233, or an antigen-binding portion thereof. Each of these constitutes a means for binding the IL-21 receptor a.

In some embodiments, LAG-3 is a targeted cell surface antigen and a binding moiety comprises the antigen binding domain of an anti-LAG-3 antibody. Accordingly, in some such embodiments, the antigen binding domain is derived from relatlimab, tebotelimab, favezelimab, fianlimab, miptenalimab, HLX26, ieramilimab, GSK2831781, INCAGN2385, RO7247669, encelimab, FS118, SHR-1802, Sym022, IBI1110, IBI323, bavunalimab, EMB-02, ABL501, INCA32459, AK129, or an antigen-binding portion thereof. Each of these constitutes a means for binding LAG-3.

In some embodiments, IDO is a targeted cell surface antigen and a binding moiety comprises the antigen binding domain of an anti-IDO antibody. Accordingly, in some such embodiments, the antigen binding domain is derived from and antibody described in WO2022011270 which is incorporated by reference for all that they teach about anti-IDO antibodies and their properties, or an antigen-binding portion thereof. Each of these constitutes a means for binding IDO.

In some embodiments, OX40 is a targeted cell surface antigen and a binding moiety comprises the antigen binding domain of an anti-OX40 antibody. Accordingly, in some such embodiments, the antigen binding domain is derived from MEDI6469, ivuxolimab, rocatinlimab, GSK3174998, BMS-986178, vonlerizumab, INCAGN1949, tavolimab, BGB-A445, INBRX-106, BAT6026, telazorlimab, ATOR-1015, MEDI6383, cudarolimab, FS120, HFB301001, EMB-09, HLX51, Hu222, ABM193, or an antigen-binding portion thereof. Each of these constitutes a means for binding OX40.

In some embodiments, PD-1 is a targeted cell surface antigen and a binding moiety comprises the antigen binding domain of an anti-PD-1 antibody. Accordingly, in some such embodiments, the antigen binding domain is derived from nivolumab, pembrolizumab, camrelizumab, torpalimab, sintilimab, tislelizumab, cemiplimab, spartalizumab, serplulimab, cadonilimab, penpulimab, dostarlimab, zimberelimab, retifanlimab, pucotenlimab, pidilizumab, pidilizumab, balstilimab, ezabenlimab, AK112, geptanolimab, cetrelimab, prolgolimab, tebotelimab, sasanlimab, SG001, vudalimab, MEDI5752, rulonilimab, peresolimab, IBI318, budigalimab, MEDIO680, pimivalimab, QL1706, AMG 404, R07121661, lorigerlimab, nofazinlimab, sindelizumab, or an antigen-binding portion thereof. Each of these constitutes a means for binding PD-1.

In some embodiments, TIM-3 is a targeted cell surface antigen and a binding moiety comprises the antigen binding domain of an anti-TIM-3 antibody. Accordingly, in some such embodiments, the antigen binding domain is derived from TQB2618, sabatolimab, cobolimab, R07121661, INCAGNO2390, AZD7789, surzebiclimab, LY3321367, Sym023, BMS-986258, SHR-1702, LY3415244, LB1410, or an antigen-binding portion thereof. Each of these constitutes a means for binding TIM-3.

In some embodiments, TREM2 is a targeted cell surface antigen and a binding moiety comprises the antigen binding domain of an anti-TREM2 antibody. Accordingly, in some such embodiments, the antigen binding domain is derived from PI37012 as well as those disclosed in U.S. Pat. Nos. 10,508,148, 10,676,525, WO2017058866, U.S. Pat. Nos. 11,186,636, 11,124,567, WO2020055975, U.S. Pat. No. 11,492,402, WO2020121195, WO2023012802, WO2021101823, WO2023047100, WO2022032293, WO2022241082, WO2023039450, or WO2023039612, each of which is incorporated by reference for all that they teach about anti-TREM2 antibodies and their properties, or an antigen-binding portion thereof. Each of these constitutes a means for binding TREM2.

The following paragraphs provide non-exhaustive examples of known antibodies that bind to cell surface markers on immune, tumor, and/or fibrogenic cells. These antibodies or the antigen binding domains thereof can be used as binding moieties in CARs and TCEs. Most of the antigens listed below are expressed by the cancerous cells of the tumor, however a few, such as fibroblast activation protein (FAP), are expressed by stromal cells of the tumor. FAP is also a useful pursued antigen in the treatment of fibrosis. Some of these antigens are found on B cells and thus can also be pursued in treatment of autoimmunity with B cell depletion therapy. Collectively these antibodies and polypeptides comprising the antigen binding domains thereof constitute means for binding cancer (of any one of the indicated cancer types or a combination thereof) cell surface markers, fibrogenic cell surface markers, or B cell surface markers or means for binding cancer cells (of any one of the indicated cancer types or a combination thereof), fibrogenic cells, or B cells, as the case may be. The antigens include:

Activin receptor-like kinase found in colorectal, liver, urogenital cancers and other solid tumors and bound by ascrinvacumab.

Adenocarcinoma antigen found on adenocarcinomas and bound by pintumomab.

α-fetoprotein found on liver cancer and bound by tacatuzumab.

AXL receptor tyrosine kinase found in multiple types of solid tumors: ovarian, cervical, endometrial, thyroid, non-small cell lung cancer, melanoma and sarcoma, and bound by enapotamab.

B cell maturation antigen (BCMA) found in multiple myeloma and bound by belantamab, elranatamab, and teclistamab. This B cell lineage antigen is also a useful pursued antigen in the treatment of B cell-mediated autoimmunity.

CA-125 found on ovarian cancer and bound by igovomab, oregovomab, and sofituzumab.

CanAg (a glycoform of MUC1) found on colorectal and other cancers and bound by cantuzumab.

Carbonic anhydrase 9 found in clear cell renal carcinoma and bound by girentuximab.

Carcinoembryonic antigen (CEA) found on colorectal and gastrointestinal cancers and bound by altumomab and arcitumomab.

Carcinoembryonic antigen-related cell adhesion molecule 5 (CEACAM5) found in colorectal cancer and bound by tusamitamab, labetuzumab, and cibisatamab.

C-C chemokine receptor 4 (CCR4) found on adult T cell leukemia/lymphoma and bound by mogamulizumab.

C-C chemokine receptor 5 (CCR5) found on various solid tumors such as melanoma, pancreatic, breast (including triple negative breast cancer), prostate, colon, lung, liver, and stomach cancers and bound by Leronlimab.

CD4 found on T cells, including those mediating T cell-mediated autoimmunity and bound by tregalizumab, IT1208, UB-421 (humanized), and zanolimumab (human).

CD5 is a pan-T cell marker, the expression of which is often maintained in T cell cancers. It is bound by 5D7, HE3, telimomab and zolimomab.

CD19 found in acute lymphoblastic leukemia (ALL), large B cell lymphoma, diffuse large B cell lymphoma (DLBCL), and B cell non-Hodgkin lymphoma and bound by blinatumomab, coltuximab, denintuzumab, duvortuxizumab, inebilizumab, loncastuximab, tafasitamab, taplitumomab, and XMAB-5574. This B cell lineage antigen is also a useful pursued antigen in the treatment of B cell-mediated autoimmunity.

CD20 found on B cell lymphoid cancers and bound by ibritumomab, obinutuzumab, ocaratuzumab, ocrelizumab, ofatumumab, rituximab, tositumumab, ublituximab, veltuzumab, mosentuzumab, FBTA05, epcoritamab, glofitamab, and odronextamab. This B cell lineage antigen is also a useful pursued antigen in the treatment of B cell-mediated autoimmunity.

CD22 found in non-Hodgkin's lymphoma, hairy cell leukemia, and acute lymphoblastic leukemia and bound by bectumomab, epratuzumab, inotuzumab, moxetumomab, and pinatuzumab. This B cell lineage antigen is also a useful pursued antigen in the treatment of B cell-mediated autoimmunity.

CD23 found in chronic lymphocytic leukemia and bound by lumiliximab and gomiliximab.

CD25 found in B-cell Hodgkin's lymphoma, non-Hodgkin lymphoma, acute lymphoblastic leukemia, and acute myeloid leukemia and bound by basiliximab, camidanlumab, daclizumab, and inolimomab.

CD28 found in chronic lymphocytic leukemia and bound by TGN1412 and lulizumab.

CD30 found in Hodgkin's lymphoma and bound by brentuximab.

CD33 found in acute myeloid leukemia and other myeloproliferative diseases and bound by lintuzumab, vadastuximab, and gemtuzumab.

CD37 found in B cell malignancies including Hodgkin's and non-Hodgkin's lymphoma and bound by lilotomab, naratuximab, otlertuzumab and tetulomab. This B cell lineage antigen is also a useful pursued antigen in the treatment of B cell-mediated autoimmunity.

CD38 found in multiple myeloma and bound by daratumumab and isatuximab.

CD40 found on hematalogic cancers and bound by decetuzumab, bleselumab, iscalimab, lucatumumab, ravagalimab, selicrelumab, teneliximab, and vanalimab, and CD40L bound by toralizumab.

CD44 found in squamous cell carcinoma and bound by bivatuzumab.

CD51 found in metastatic prostate cancer and other solid tumors (including melanoma) and bound by abituzumab and intetumumab.

CD52 (CAMPATH-1) found on lymphatic cancers and bound by ALLO-647, gatralimab, and alemtuzumab.

CD56 found on small-cell lung and ovarian cancers, and Merkel cell carcinoma, and bound by lorvotuzumab.

CD70 found in renal cell carcinoma, non-Hodgkin's lymphoma, acute myeloid leukemia (AML) and myelodysplastic syndrome (MDS) and bound by cusatuzumab and vorsetuzumab.

CD73 (5′-nucleotidase) found on pancreatic, colorectal, and other cancers and bound by oleclumab, dresbuxelimab, and dalutrafusp.

CD74 found on multiple myeloma and other hematological malignancies and bound by milatuzumab.

CD79B found in B cell malignancies (such a non-Hodgkin's lymphoma) and bound by iladatuzumab and polatuzumab. This B cell lineage antigen is also a useful pursued antigen in the treatment of B cell-mediated autoimmunity.

CD80 found in B cell lymphoma and bound by galiximab.

CD123 (IL-3Ra) found in leukemia including myeloid malignancies and bound by flotetuzumab, vibecotamab, and talacotuzumab.

CD159 found in gynecologic malignancies and other cancers and bound by monalizumab.

CD248 (endosialin) found on tumor stroma including in sarcomas and bound by ontuxizumab.

CD276 (B7-H3) found in head and neck cancer, melanoma, squamous cell cancer of the head and neck (SCCHN) and non-small cell lung cancer (NSCLC) and bound by enoblituzumab and omburtamab.

CD319 (SLAMF7) found in various hematologic cancers including multiple myeloma and bound by elotuzumab and azintuxizumab.

Claudin-18 isoform 2 is found on gastric tumors and bound by osemitamab zolbetuximab.

CLL1 is found on acute myeloid leukemia (AML) cells and leukemic stem cells and is bound by 27H4 (human), MCLL0517A (humanized) and CLT030 (an antibody drug conjugate using a humanized anti-CLL1 mAb).

C-type lectin domain family 12 member A (CLEC12A) found on myeloid blasts, atypical progenitor cells and leukemic stem cells and bound by epoditamab.

C-X-C chemokine receptor type 4 (CXCR-4) found on various types of cancer including breast cancer, ovarian cancer, melanoma, and prostate cancer, and bound by ulocuplumab.

C-X-C chemokine receptor type 5 (CXCR5) found on Burkitt lymphoma and T cells and bound by SAR113244 as well as those disclosed in WO2012010582, WO2014177652, US20170342156, WO2019038368, WO2019243159, WO2019108639, WO2022192423, and WO2023010483, each of which is incorporated by reference for all that they teach about antibodies and other CXCR5 binding molecules that does not conflict with the present disclosure.

CX3CR1 (Fractalkine receptor) found on T cells and bound by MAB3652R, 2A9-1, SA011F11, 3D3B8, and R7G1.

Delta-like 3 (DLL3) found on small cell lung cancer and bound by rovalpituzumab.

Delta-like 4 (DLL4) found on pancreatic and non-small cell lung cancers and bound by demcizumab, enoticumab, and navicixizumab.

Epidermal Growth Factor like domain 7 (Egfl7) is found in colorectal cancer, hepatocellular carcinoma, and glioma and bound by parsatuzumab.

Endoglin found on angiosarcoma and bound by carotuximab.

EpCAM found in malignant ascites, colorectal, bladder, prostate, gastric, lung, breast, and ovarian cancers and bound by adecatumumab, catumaxomab, citatuzumab, edrecolomab, oportuzumab, solitomab, and tucotuzumab.

Eph receptor A3 (EPHA3) found on melanoma, breast, prostate, pancreatic, gastric, esophageal, and colon cancer, as well as hematopoietic tumors and bound by ifabotuzumab.

Epidermal growth factor receptor (EGFR) found in squamous cell carcinoma, head and neck cancer, glioma, glioblastoma, nasopharyngeal, colorectal, stomach, and non-small cell lung cancer and is bound by amivantamab, cetuximab, depatuxizumab, futuximab, imgatuzumab, laprituximab, losatuxizumab, matuzumab, modotuximab, necitumumab, nimotuzumab, panitumumab, tomuzotuximab, and zalutumumab.

Fibroblast activation protein (FAP) found on fibroblasts in tumor stroma and bound by sibrotuzumab, 4G5, F19, FAP5, 28H1, VHH-B1, and VHH-B2 OTMX005 and OTMX705. FAP is also a useful pursued antigen in some fibrotic diseases.

Fibroblast growth factor receptor 2 (FGFR2) found in gastric and gastroesophageal junction cancers and adenocarcinomas and bound by bemarituzumab.

Fibronectin extra domain-B found on Hodgkin's lymphoma and bound by radretumab.fc

Folate receptor 1 found in epithelial-derived tumors including ovarian, breast, renal, lung (including non-small cell lung cancers and mesothelioma), colorectal, and brain and bound by farletuzumab and mirvetuximab.

Frizzled receptor (FZD1, 2, 5, 7, and 8 receptors) found on breast and pancreatic cancers, and cancer stem cells, and bound by vantictumab.

G protein-coupled receptor family C group 5-member D (GPRC5D) found in multiple myeloma and bound by talquetamab.

Ganglioside GD2 found on neuroblastoma and bound by dinutuximab and naxitamab.

Ganglioside GD3 found on malignant melanoma and small cell lung cancer and bound by ecromeximab and mitumomab.

Gelatinase B found in gastric and gastroesophageal junction cancers and adenocarcinomas and bound by andecaliximab.

Glutamate carboxypeptidase II found on prostate cancer and bound by capromab.

Glypican 3 found in hepatocellular carcinoma and bound by codrituzumab.

Guanate cyclase 2C (GUCY2C) found on pancreatic and other gastrointestinal cancers and bound by indusatumab.

Hepatocyte growth factor receptor mesenchymal-epithelial transition (MET) found in non-small cell lung cancer and bound by emibetuzumab, ficlatuzumab, onartuzumab, rilotumumab, and telisotuzumab.

Human epidermal growth factor receptor 2 (HER2, ErbB2) found on breast, ovarian, and stomach cancers, adenocarcinoma of the lung, and aggressive forms of uterine cancer, such as uterine serous endometrial carcinoma, and bound by DS-8201, ertumaxomab, gancotamab, margetuximab, pertuzumab, timigutuzumab, trastuzumab, and TRBS07.

Human epidermal growth factor receptor B3 (ErbB3, HER3) found on breast, testicular, squamous and non-squamous non-small cell lung cancers and bound by duligotuzumab, elgemtumab, lumretuzumab, patritumab, serebantumab, zenocutuzumab.

Insulin-like growth factor 1 (IGF-1) found on solid tumors, including adrenocortical and small lung cell carcinomas and bound by cixutumumab, dalotuzumab, figitumumab, ganitumab, robatumumab, teprotumumab, and xentuzumab.

Insulin-like growth factor 2 (IGF-2) found on breast and liver cancers and other solid tumors and bound by dusigitumab and xentuzumab.

Integrin αvβ3 found in melanoma, prostate, ovarian and other cancers and bound by etaracizumab.

Integrin α₅β₁ found in solid tumors and bound by volociximab.

Killer-cell immunoglobulin-like receptor 2D (KIR2D) found on solid (including squamous cell carcinoma of the head and neck) and hematological cancers (including AML) and bound by lirilumab.

Lewis Y antigen found on lung, breast, colon, pancreatic, and other cancers and bound by cBR96 and C242 (nacolomab).

LIV-1 found on metastatic breast cancer as well as in melanoma, and prostate, ovarian, uterine, and cervical cancers and bound by ladiratuzumab.

Leucine-rich repeat containing 15 (LRRC15) found on tumor cells (including triple-negative breast cancer, non-small cell lung cancer, colorectal cancer) and cancer-associated fibroblasts and bound by samrotamab and DUNP19, as well as the antibodies disclosed in WO2005037999, WO2021022304, WO2021067673, WO2021102332, WO2021202642, and WO2022157094, each of which is incorporated by reference for all that they teach about antibodies and TCEs with specificity for LRRC15 that does not conflict with the present disclosure.

Mesothelin found in mesothelioma, lung cancer, ovarian cancer, and pancreatic cancer, and bound by amatuximab and anetumab.

Mucin 1 (MUC1) found in pancreatic, breast, and ovarian cancers and bound by clivatuzumab, gatipotuzumab, and pemtumomab.

Mucin 5AC (Muc5AC) found in colorectal and pancreatic carcinomas and bound by ensituximab.

Nectin 4 found in urothelial cancer and bound by enfortumab.

Notch 1 found in chemoresistant cancers and bound by brontictuzumab.

Notch 2/3 receptor found on pancreatic and lung cancers and bound by tarextumab.

PD-L1 found on urothelial carcinoma, non-small cell lung cancer (NSCLC), triple-negative breast cancer (TNBC), small cell lung cancer (SCLC), hepatocellular carcinoma (HCC), and melanoma and bound by atezolizumab, avelumab.

Phosphate-sodium co-transporter found on breast, thyroid, ovarian and non-small cell lung cancers and bound by lifastuzumab.

Platelet-derived growth factor receptor a (PDGF-Rα) found on solid tumors, particularly soft tissue sarcomas, glioblastoma, and non-small cell lung cancer, and bound by olaratumab and tovetumab. Also a useful pursued antigen in some fibrotic diseases.

Prostate-specific membrane antigen (PSMA) found on prostate cancer and bound by pasotuxizumab.

Prostate stem cell antigen (PSCA) found on prostate cancer and also bladder and pancreatic cancer; bound by AGS-1C4D4, GEM3PSCA, BPX-601, 1G8 as well as the antibody, CAR or TCE disclosed in WO2006112933, WO2013001065, WO2020123766, WO2001040309, WO2009032949, WO2021236645, WO2018223601, and WO2021050656, each of which is incorporated by reference for all that it teaches related to anti-PSCA antigen binding domains and their uses that does not conflict with the present disclosure.

PTK7 (tyrosine protein kinase-like 7) found on ovarian cancer, breast cancer, non-small cell lung and other cancers and bound by cofetuzumab.

Receptor activator of nuclear factor kappa-B ligand (RANKL) found in prostate and breast cancer (and bone metastases thereof) and multiple myeloma and bound by denosumab.

R-spondin 3 (RSPO3) found on solid tumors and bound by rosmantuzumab.

Six Transmembrane Epithelial Antigen of The Prostate 1 (STEAP1) found in prostate cancer and bound by vandortuzumab.

SLIT and NTRK-like protein 6 (SLITRK6) found on neural and brain tumor tissue and bound by sirtratumab.

Syndecan1 (SDC1; CD138) found on multiple myeloma and bound by indatuximab.

TRAIL-R1 found on multiple myeloma, and solid tumors including non-small cell lung cancer, colorectal cancer and liver cancer and bound by mapatumumab.

TRAIL-R2 found on pancreatic cancer, gastric, colorectal cancer, non-small cell lung cancer, cervical and ovarian cancer and bound by conatumumab, lexatumumab, and tigatuzumab.

Transmembrane glycoprotein NMB (GPNMB) found in melanoma and breast cancer and bound by glembatumumab.

Trophoblast glycoprotein (5T4) found on colorectal, ovarian, lung, renal, and gastric cancers and bound by naptumomab.

Tumor antigen CTAA16.88 found on colorectal tumors and bound by votumumab.

Tumor-associated calcium signal transducer 2 (also known as Trop-2) found on carcinomas, including triple negative breast cancer and metastatic urothelial caner, and bound by sacituzumab.

Tumor-associated glycoprotein 72 (TAG-72) found on breast, colon, lung, and pancreatic cancers and bound by anatumomab, minretumomab, and satumomab.

Tumor necrosis factor receptor superfamily member 12A (TWEAKR) found on solid tumors and bound by enavatuzumab.

Tyrosinase-related protein 1 (TYRP1) found in melanoma and bound by flanvotumab.

Tyrosine-protein kinase transmembrane receptor ROR1 found on chronic lymphocytic leukemia (CLL) and other cancers and bound by cirmtuzumab and zilovertamab.

Vimentin found on glioma and bound by pritumumab.

In some embodiments, a binding moiety of a tLNP comprises an antigen binding domain of an anti-CD8 antibody. In some embodiments, an anti-CD8 antibody is a human antibody. In some embodiments, an anti-CD8 antibody is a chimeric or humanized antibody, such as a chimeric or humanized mouse anti-human CD8 antibody. In some instances, an anti-CD8 antibody is a humanized form of a mouse antibody, such as RPA-T8 (also referred to herein as CT8). As expressed on cells in a mammal, CD8 is a dimer, commonly of two a chains or one each of an α and β chain. CT8 recognizes an epitope on the a chain (sometime referred to as CD8a or CD8α). CT8 and its humanized derivatives can bind to both the α2 and αβ dimers. In some embodiments, a humanized antigen binding domain derived from CT8 comprises a heavy chain variable region (VH) comprising an amino acid sequence that has at least 90% identity with the amino acid sequence of SEQ ID NO: 116 or 134 wherein the VH comprises a heavy chain CDR1 (VH-CDR1) comprising the amino acid sequence RYTFTDYX₁LH (SEQ ID NO: 148) wherein X₁ is N, S, Q, or A, a VH-CDR2 comprising the amino acid sequence FIYPYX1GGTG (SEQ ID NO: 149) or FIYPYX₂GGTG (SEQ ID NO: 150) wherein X₂ is N, Q, D, S, or A, and a VH-CDR3 having the amino acid sequence DHRYX₁EGVSFDY (SEQ ID NO: 151); and a light chain variable region (VL) comprising an amino acid sequence that has at least 90% identity with the amino acid sequence of SEQ ID NO: 122 or 140, wherein the VL comprises a CDR1 (VL-CDR1) comprising the amino acid sequence RASESVX₃GFGX₁SFMN wherein X₃ is D, E, S, or A (SEQ ID NO: 152), VL-CDR2 comprising the amino acid sequence LASX₂LES (SEQ ID NO: 153), and a VL-CDR3 having the amino acid sequence QQX₂X₂EX₃PYT (SEQ ID NO: 154). In some embodiments, the antigen binding domain comprises a VL region having the amino acid sequence of one of SEQ ID NOs: 123-125 or 141-143. In some embodiments, the antigen binding domain comprises a VH region having the amino acid sequence of one of SEQ ID NOs: 117-121, 130-133, or 135-139. In some embodiments, the antigen binding domain comprises a VL region having the amino acid sequence of SEQ ID NO: 124 and a VH region having the amino acid sequence of one of SEQ ID NOs: 118 or 130-133. In some embodiments, the antigen binding domain comprises:
(a) a VH comprising the amino acid sequence of SEQ ID NO: 189 and a VL comprising the amino acid sequence of SEQ ID NO: 123;
(b) a VH comprising the amino acid sequence of one of SEQ ID NO: 190-193, and a VL comprising the amino acid sequence of SEQ ID NO: 124; or
(c) a VH comprising the amino acid sequence of one of SEQ ID NO: 190-193, and a VL comprising the amino acid sequence of SEQ ID NO: 125;

In some embodiments, VH-CDR1 has the amino acid sequence RYTFTDYNLH (SEQ ID NO: 109). In some embodiments, VH-CDR2 has the amino acid sequence FIYPYNGGTG (SEQ ID NO: 110). In some embodiments, VH-CDR3 has the amino acid sequence DHRYNEGVSFDY (SEQ ID NO: 111). In some embodiments, VL-CDR1 has the amino acid sequence RASESVDGFGNSFMN (SEQ ID NO: 113). In some embodiments, VL-CDR2 has the amino acid sequence LASNLES (SEQ ID NO: 114). In some embodiments, VL-CDR3 has the amino acid sequence QQNNEDPYT (SEQ ID NO: 115). In some embodiments, the acceptor sequence from which the heavy chain framework regions are derived from IGHV1-46*01/IGHJ6*01, as shown in SEQ ID NO: 116). In some embodiments, the acceptor sequence from which the light chain framework regions are derived from IGKV1-39*01/IGKJ2*01, as shown in SEQ ID NO: 122. In some embodiments, the acceptor sequence from which the heavy chain framework regions are derived from a modified IGHV1-18*01, as shown in SEQ ID NO: 134. In some embodiments, the acceptor sequence from which the light chain framework regions are derived from a modified version of IGKV3D-11*01, as shown in SEQ ID NO: 140. In some embodiments, the heavy chain framework regions are derived from IGHV1-46*01/IGHJ6*01 and the light chain framework regions are derived from IGKV1-39*01/IGKJ2*01. The resultant VH and VL sequences are highly similar regardless of which acceptor sequence they are derived from.

In some embodiments, the heavy chain framework regions are derived from a modified IGHV1-18*01 and the light chain framework regions are derived from a modified version of IGKV3D-11*01. In some embodiments, the heavy chain framework regions are derived from IGHV1-46*01/IGHJ6*01 and the light chain framework regions are derived from a modified version of IGKV3D-11*01. In some embodiments, the heavy chain framework regions are derived from a modified IGHV1-18*01 and the light chain framework regions are derived from IGKV1-39*01/IGKJ2*01. Table 18 shows several humanized variants of anti-CD8 antibody binding moiety and related sequences. Further information about humanized CT8 antibodies and antigen binding fragments thereof is disclosed in International Application No. PCT/US2024/060426 (Attorney docket number 23-1742-WO) entitled Humanized Anti-CD8 Antibodies and Uses Thereof, filed on Dec. 16, 2024, which is incorporated by reference in its entirety for its teachings about such antibodies and antigen binding domains and their uses.

In some embodiments, the targeting moiety is a whole antibody. In some embodiments, the antibody comprises a silenced Fc region. A silenced Fc region fails to bind to Fc gamma receptors and complement protein C1q, thus abolishing immune effector functions. In some instances, the antibody comprises a silenced Fc region having the amino acid sequence of SEQ ID NO: 146 or 147. In some embodiments, the whole humanized anti-CD8 antibody heavy chain with a silenced Fc region comprises the sequence of CBD1033HC (SEQ ID NO: 164). In some embodiments, the whole humanized anti-CD8 antibody light chain comprises the sequence of CBD1033LC (SEQ ID NO: 165). In some embodiments, the whole humanized anti-CD8 antibody comprising a heavy chain with a silenced Fc region comprises a heavy chain comprising the sequence of CBD1033HC (SEQ ID NO: 164) and a light chain comprising the sequence of CBD1033LC (SEQ ID NO: 165). Natural Fc sequence ends with a lysine residue, as shown in SEQ ID NO: 147. In product manufacturing C-terminal clipping of this lysine residue can cause product heterogeneity. To obviate this problem the coding sequence for the Fc can be modified to not encode this amino acid, for example ending at the previous glycine residue in the natural Fc sequence as done in SEQ ID NO: 146. For all embodiments comprising an Fc, whether shown with or without the natural terminal lysine residue, there is an alternative embodiment without or with that residue, respectively.

In some embodiments, the targeting moiety is an anti-CD8 F(ab′) or an anti-CD8 F(ab′) analog. Although any antibody fragment with a structure similar to or derived from that of a classical, proteolytically produced F(ab′) is often referred to as an F(ab′), the term “F(ab′) analog” has been adopted herein to refer to engineered sequences comprising amino acid substitutions and/or that have been truncated and to distinguish them from the paradigmatic natural sequence. Examples of such anti-CD8 F(ab′) and anti-CD8 F(ab′) analog are listed in Table 21.

With respect to these forgoing aspects, in some embodiments, the F(ab′) analog, as appropriate, comprises a relocated interchain disulfide bond, for example, a Cκ S162C substitution paired with an IgG1 or IgG4 CH₁ F174C substitution. In further embodiments, one, the other, or both cysteines involved in forming the native interchain disulfide bond are mutated, for example, Cκ C214S, IgG1 C233S, or IgG4 CH₁ C127S. In some embodiments, the F(ab′) analog has a CH region truncated at T238. In some embodiments, the F(ab′) analog has a CH region truncated at P241. In some embodiments, the F(ab′) analog has a CH region truncated at P241 and has substitutions P240A and P241A.

In some embodiments, the F(ab′) analog of design 0.37 comprises: a Cκ S162C and C214S substitutions (SEQ ID NO: 180); and a IgG1 CH region truncated at P241 and has P240A and P241A substitutions, and F174C and C233S substitutions (SEQ ID NO: 181). Thus, F(ab′) analog of design 0.37 comprises a relocated Cκ S162C-CH F174C interchain disulfide bond and an abolished native Cκ C214S-CH C233S disulfide bond. Examples of F(ab′) analog of design 0.37 includes CBD1033.37 (light chain SEQ ID NO: 182 and heavy chain SEQ ID NO: 183), CBD1381.37 (light chain SEQ ID NO: 182 and heavy chain SEQ ID NO: 188), CBD1382.37 (light chain SEQ ID NO: 182 and heavy chain SEQ ID NO: 189), CBD1444.37 (light chain SEQ ID NO: 190 and heavy chain SEQ ID NO: 183), CBD1622.37 (light chain SEQ ID NO: 190 and heavy chain SEQ ID NO: 188), and CBD1623.37 (light chain SEQ ID NO: 190 and heavy chain SEQ ID NO: 189).

In some embodiments, the F(ab′) analog of design 0.42 comprises: a Cκ S162C and C214S substitutions (SEQ ID NO: 180), and a IgG1 CH region truncated at P241 and F174C and C233S substitutions (SEQ ID NO: 184). Thus, F(ab′) analog of design 0.42 comprises a relocated Cκ S162C-CH F174C interchain disulfide bond and an abolished native Cκ C214S-CH C₂₃₃S disulfide bond. Examples of F(ab′) analog of design 0.42 includes CBD1033.42 (light chain SEQ ID NO: 182 and heavy chain SEQ ID NO: 185), CBD1622.42 (light chain SEQ ID NO: 190 and heavy chain SEQ ID NO: 191), and CBD1623.42 (light chain SEQ ID NO: 190 and heavy chain SEQ ID NO: 192).

In some embodiments, the F(ab′) analog of design 0.44 comprises: a Cκ S162C and C214S substitutions (SEQ ID NO: 180), and a IgG1 CH region truncated at T238 and F174C substitution (SEQ ID NO: 186). Thus, F(ab′) analog of design 0.44 comprises a relocated Cκ S162C-CH F174C interchain disulfide bond and an abolished native disulfide bond due to Cκ C214S substitution. Examples of F(ab′) analog of design 0.44 includes CBD1033.44 (light chain SEQ ID NO: 182 and heavy chain SEQ ID NO: 187), CBD1622.44 (light chain SEQ ID NO: 190 and heavy chain SEQ ID NO: 193), and CBD1623.44 (light chain SEQ ID NO: 190 and heavy chain SEQ ID NO: 194).

### Methods of Treatment

In certain aspects this disclosure provides methods of treating a disease or disorder associated with a pathogenic cell comprising administering to a subject in need thereof in a compact regimen multiple doses of a T cell-targeted tLNP encapsulating an mRNA encoding a T cell antigen receptor, wherein the pathogenic cell in the subject expresses an antigen recognized the T cell antigen receptor, whereby more T cells express the antigen receptor as a result to a subsequent administration than as a result of the initial administration. (The phrase “comprising administering to a subject in need thereof in a compact regimen multiple doses of a T cell-targeted tLNP” may be alternatively stated as “comprising repeatedly administering a T cell-targeted tLNP to a subject in need thereof in a compact regimen”). In some embodiments, greater pharmacologic and/or clinical effect and/or improved safety is achieved after administration of at least 2 doses as compared to the same total dosage administered over a same time interval as a single dose or as multiple doses where each subsequent dose is administered ≥7 days after an immediately preceding dose.

In various embodiments, each subsequent dose is administered 1 to 5 days after the immediately preceding dose such as 1, 2, 3, 4, or 5 days after the immediately preceding dose or the subsequent dose is administered in a time range bound by any pair of those values, for example 2-5, 2-4, or 3-4. In some embodiments, the interval between doses is always the same, for example, every 2^{nd} 3^{rd}, or 4^{th} day. In some embodiments, the tLNP is administer 3 times at 3-day intervals.

In other embodiments, the tLNP is administered 2 times with an interval of 3 days between administrations.

In some embodiments, the dosage for each administration in the compact administration regimen is from about 0.1 mg/kg to 2 mg/kg, for example, about 0.1, 0.2, 0.3, 0.4, 0.5, 0.6, 0.7, 0.8, 0.9, 1.0, 1.2, 1.4, 1.5, 1.75, or 2.0 mg/kg. In other embodiments, the dosage is from about 0.3 mg/kg to 1.0 mg/kg.

As noted above, depletion of the pursued pathogenic cells can lead to a waning of the amplification effect, although the effect can persist locally where the pursued pathogenic cells persist. The degree of depletion will vary with the type of pathogenic cell being pursued. In the context of autoimmunity immune reset can be achieved with a deep depletion of B cells but complete elimination of all B cells is not required (or even desirable). A similar situation of not needing complete elimination of the pursued pathogenic cells exists with respect to the depletion fibrogenic cells, whether in the context of treating fibrotic diseases or tumor stromal cells. In contrast, complete elimination of neoplastic cells is desirable in the treatment of cancer.

Accordingly, the compact administration regimen should be continued, even in the absence of observable systemic amplification, until some independent measure indicates that a sufficient level of depletion has been achieved (or that the treatment is no longer effective for other reasons).

In some embodiments of methods of treating a disease or disorder associated with a pathogenic cell, the T cell is a cytolytic T cell. In various further embodiments, the T cell or cytolytic T cells is a CD8⁺ T cell, a CD4⁺ T cell, or an NKT cell. In some embodiments, the T cell surface antigen targeted by the LNP targeting moiety is CD2, CD3, CD4, CD5, CD7, CD8, CD28, 4-1BB (CD137), CD166, CTLA-4, OX40, PD-1, GITR, LAG-3, TIM-3, CD25, low affinity IL-2 receptor, IL-7 receptor, IL-12 receptor, IL-15 receptor, IL-18 receptor, IL-21 receptor, CXCR5 receptor, or CX3CL1 receptor.

In some embodiments, the T cell antigen receptor is a CAR, a TCR, or a TCE.

In some embodiments, the disease or disorder is cancer, and the pathogenic cell is a neoplastic cell. In some embodiments, the cancer is a hematologic cancer (for example, a leukemia, lymphoma, or myeloma), a carcinoma, or a sarcoma. In further embodiments, the cancer is diffuse large B cell lymphoma, acute myeloid leukemia, Mantle Cell lymphoma, follicular lymphoma, B acute lymphoblastic leukemia, chronic lymphocytic leukemia, myelodysplastic syndrome, sarcoma, carcinoma, breast cancer, colon cancer, ovarian cancer, lung cancer, melanoma, lymphoma, testicular cancer, hematologic cancers, myeloma, and pancreatic cancer. In some embodiments, the cancer is a B cell leukemia or lymphoma. In some embodiments, the antigen expressed by the cancer cell is one of those listed above. In some embodiments, the antigen expressed by the B cell leukemia or lymphoma, is CD19, CD20, or both. In other embodiments, the antigen expressed by the B cell leukemia or lymphoma another of those listed above. In some embodiments, the cancer is acute myelogenous leukemia (AML). In other embodiments, the antigen expressed by the AML cells is CD33 or CLL1. In some embodiments, the cancer is a solid tumor. In some embodiments, the antigen expressed by the solid tumor cell is mesothelin, PSCA, PSMA, MUC1, or Her.

In other embodiments, the disease or disorder is cancer, and the pathogenic cell is a tumor stromal cell. In some embodiments, the tumor stromal cell is a stromal fibroblast, myeloid cell, or endothelial cell. In some instances, the tumor stromal cell is a tumor-associated neovascular endothelial cell. In some embodiments, the antigen expressed by the tumor stromal cell is FAP, LRRC15, or PDGF-Rα.

In some embodiments, the disease or disorder is fibrosis such as, without limitation, cardiac fibrosis, arthritis, idiopathic pulmonary fibrosis, nonalcoholic steatohepatitis, or tumor-associated fibroblasts. In some embodiments, the antigen expressed by the fibrogenic cell is FAP, LRRC15, or PDGF-Rα.

In some embodiments, the disease or disorder is an autoimmune disease. In some embodiments, the autoimmune disease is a T cell-mediated autoimmunity or a B cell-mediated autoimmunity. In some instances, the B cell-mediated autoimmune disease is myositis (such as anti-synthetase myositis), lupus nephritis, membranous nephropathy, systemic lupus erythematosus, anti-neutrophilic cytoplasmic antibody (ANCA) vasculitis, autoimmune hemolytic anemia, neuromyelitis optica spectrum disorder (NMOSD), myasthenia gravis (such as generalized myasthenia gravis including anti-muscle-specific kinase antibody positive myasthenia gravis (myasthenia gravis-MusK), anti-acetylcholine receptor antibody positive myasthenia gravis (myasthenia gravis-AchR), anti-lipoprotein receptor-related protein 4 antibody positive, and seronegative myasthenia gravis), pemphigus vulgaris, rheumatoid arthritis, dermatomyositis, immune mediated necrotizing myopathy (IMNM), polymyositis, systemic sclerosis, diffuse cutaneous systemic sclerosis, limited cutaneous systemic sclerosis, anti-synthetase syndrome (idiopathic inflammatory myopathy), stiff person syndrome, myeloid oligodendrocyte glycoprotein autoantibody associated disease (MOGAD), amyloid light-chain amyloidosis, multiple sclerosis, relapsing-remitting multiple sclerosis, secondary progressive multiple sclerosis, primary progressive multiple sclerosis, non-active secondary progressive multiple sclerosis, Hashimoto thyroiditis, Sjörgen's syndrome, IgA nephropathy, IgG4-related disease, severe combined immunodeficiency, or Fanconi anemia.

In some embodiments, the autoimmune disease is a neuroinflammatory disorder. In various instances, the neuroinflammatory disorder is myasthenia gravis, multiple sclerosis, Hashimoto's encephalopathy, chronic inflammatory demyelinating polyneuropathy, optic neuritis, and neuromyelitis optica.

In some embodiments, the autoimmune disease is a rheumatic disease/vasculitis. In various instances, the rheumatic disease is systemic lupus erythematosus, rheumatoid arthritis, Sjögren's syndrome, systemic scleroderma, ANCA, IgG4-related disease, mixed connective tissue disease, myositis, inflammatory muscle disease, vasculitis, multiple sclerosis, cold agglutinin disease, Goodpasture syndrome, anti-phospholipid syndrome, systemic sclerosis.

In some embodiments, the autoimmune disease is an autoimmune nephropathy. In various instances, the autoimmune nephropathy is membranous nephropathy, lupus nephritis, or IgA nephropathy.

In some embodiments, the disease or disorder is rejection of an allogeneic organ or tissue graft. Pre-existing antibodies and/or B cells, in their role as antigen presenting cells, can facilitate rapid immune rejection through known mechanisms hence depleting a large number of B cells can help prevent allograft rejection. Similarly, B cell depletion can be used in the management of graft-versus-host disease.

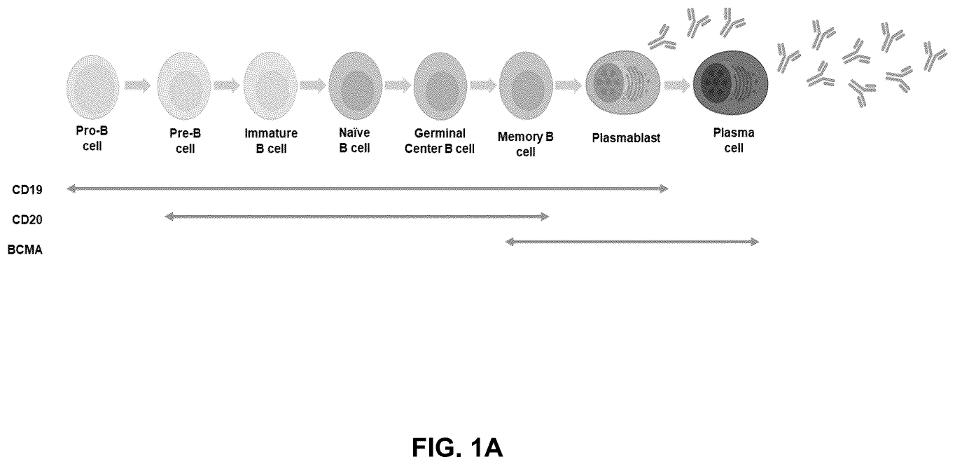

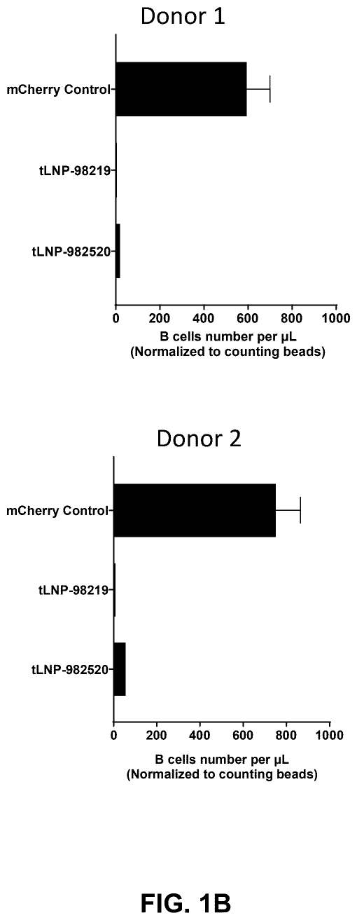

In the treatment of B cell-mediated autoimmunity, transplant rejection, or graft versus host disease (GVHD), CD19 and/or BCMA are appropriate antigens to pursue whereas due to the more restricted expression of CD20 that does not include plasmablasts or plasma cells (see FIG. 1A) CD20 may not be. In addition to plasmablasts and plasma cells memory and germinal center B cells can also play an important role in autoimmunity. Consequently, whether depletion of CD19+ or BCMA+ B cells, or both, will be sufficient or necessary to achieve immune reset and long-term, drug free resolution of the disease will show some variation from patient to patient, condition to condition, and even within a particular condition. Conditions in which pre-existing antibodies from long-lived plasma cells, such as transplant rejection, can require depletion of BCMA+ B cells alone or in combination with depletion of CD19+ B cells. Accumulating evidence suggests (as seen in FIG. 1A) that for some conditions, including myasthenia gravis-MusK, pemphigus vulgaris, IgG4-related disease, and NMOSD, depleting CD19+ B cells will generally lead to immune reset and long-term resolution of disease. For other conditions, including myasthenia gravis-AchR, Sjögren's syndrome, and MOGAD, depletion of plasma cells (e.g., by targeting BCMA+ B cells) can also be required. Other conditions are more variable in that for some patients, depletion of CD19+ B cells will be sufficient while other patients will require depletion of BCMA+ cells in addition to or instead of depletion of CD19+ B cells. Such conditions include systemic lupus erythematosus, membranous nephropathy, thrombocytopenic purpura, myositis, autoimmune hemolytic anemia, and systemic sclerosis. These delineations are provided as guidance that will apply to many patients but are not absolute due to patient-to-patient variation and variability in disease pathology and progression, as alluded to above. Thus, in various embodiments, a patient with autoimmunity, or GVHD, or undergoing, transplant rejection prevention or management, is treated by depleting CD19+ B cells, depleting CD19+ B cells and if long-term immune reset is not achieved, following up with depletion of BCMA+ B cells or both CD19+ and BCMA+ B cells, depletion of both CD19+ and BCMA+ B cells, or depletion of BCMA+ B cells. Preservation of BCMA+ plasma cells occurring when CD19+ B cells are targeted, will also preserve vaccine-induced antibodies. Thus, there is an advantage to not depleting BCMA+ B cells if it is not required to achieve resolution of the disease. In some embodiments, patients who have BCMA+B cells depleted are administered intravenous immunoglobulin (IVIG) and/or are re-vaccinated after B cell repopulation.

Patients with B cell mediated disorders such as autoimmunity, allotransplant rejection, GVHD, and the like can be treated with immunosuppressive therapy using immunosuppressive agents such as such as corticosteroids, methotrexate, mTOR inhibitors, calcineurin inhibitors, mycophenylate mofetil, JAK inhibitors, or monoclonal antibodies that interfere with activity of T cells and/or myeloid cells. The disclosed methods of treating B cell-mediated disorders can be applied to such patients. In some embodiments, immunosuppressive therapy is suspended or discontinued prior to initiation of treatment using an immune engineering amplification method of treatment, for example 1, 2, 3, 4, 5, or 6 weeks prior to initiation of the treatment or within a range bound by any pair of those values. In other embodiments, immunosuppressive therapy continues throughout one or more cycles of treatment and can be optionally continued for a finite period of time thereafter (for example, until remission of disease can be confirmed). In some embodiments, immunosuppressive therapy is not resumed. In other embodiments, the clinical evolution of the subject is evaluated and immunosuppressive therapy is optionally resumed 2-14 weeks after the last dose of tLNP.

The compact administration regimen leads to increased safety of the disease treatment. Because smaller individual dosages are used cytokine release syndrome and related inflammatory adverse events can be eliminated or reduced. Similarly, infusion reactions and organ level toxicities, such as liver toxicity are reduced or eliminated by the lower individual dosages (with lower tLNP Cmax) and the lower cumulative dosages of the LNP also contribute to the avoidance of organ level toxicities. Additionally, in the context of B cell depletion, potential toxicity from an anti-drug antibody (ADA) response or hypersensitivity is blunted by the absence of B cells to initiate or expand such a response whether it would be directed against the antigen receptor or some component of the tLNP. Altogether, the compact regime provides greater tolerability for tLNP-based treatment.

Where B cell depleting agents do not serve as the ultimate therapeutic agent in the compact regimen, they can still be used as the T cell activating agents and provide the additional function of blunting ADA responses. Accordingly, in some aspects, the compact regimen is used in a method of blunting ADA responses during in vivo transfection mediated by tLNP encapsulated nucleic acids (or similarly for other tLNP delivered payloads). Such methods comprise administering 1 or 2 activating doses of an anti-B cell agent followed by 1 or more doses of a second tLNP to serve as the ultimate therapeutic agent according to the general parameters of the compact regimen. The anti-B cell agent, for example, a tLNP-encapsulated anti-B cell CAR, can bind any of several B cell antigens such as CD19, CD20, CD22, CD30, CD79, and BCMA. To blunt induction of de novo and recall antibodies, any of these antigens can be targeted. However, if there is a substantial pre-existing titer of antibody against a component of the therapeutic (or activating) agent, indicating the existence of long-lived plasma cells, then an antigen expressed by long-lived plasma cells, such as BCMA, or alternatively CD38, CD138, FcRL5 or GPRC5D, is the appropriate choice to be targeted.

In a further aspect, a method of blunting ADA responses to a potentially immunogenic pharmaceutical (i.e., a drug) is provided comprising first depleting B cells using a compact regimen as described herein and then during an interval of B cell depletion administering the potentially immunogenic drug. In some embodiments the compact regiment is used in a method of blunting ADA response during in vivo transfection mediated by tLNP encapsulated nucleic acids (or similarly for other tLNP delivered payloads). B cell depletion is carried out according to the compact regimen and the potentially immunogenic therapeutic agent (drug) is administered on any schedule confined to the period in which there are no circulating B cells (or ≤5 or ≤10 circulating B cells per μL of blood), but not necessarily to the compact regimen. In a still further aspect, B cell depletion is carried out by in vivo transfection mediated by tLNP encapsulated nucleic acids encoding an anti-B cell antigen receptor (e.g., a CAR) according to the compact regimen prior to treatment with any potentially immunogenic pharmaceutical, tLNP-based or not). As seen in Examples below, a pair of moderate doses can provide a depletion of circulating B cells lasting about 2 weeks. Longer periods of depletion of circulating B cells can be obtained with higher dosages and/or additional administrations according to the compact regimen. Alternatively, due to the speed with which B cell depletion can be accomplished with the compact regimen, in some embodiments, the B cell depleting tLNP and the potentially immunogenic drug are administered concurrently.

Toxicities and adverse events are sometimes graded according to a 5-point scale. A grade 1 or mild toxicity is asymptomatic or induces only mild symptoms; may be characterized by clinical or diagnostic observations only; and intervention is not indicated. A grade 2 or moderate toxicity may impair activities of daily living (such as preparing meals, shopping, managing money, using the telephone, etc.) but only minimal, local, or non-invasive interventions are indicated. Grade 3 toxicities are medically significant but not immediately life-threatening; hospitalization or prolongation of hospitalization is indicated; activities of daily living related to self-care (such as bathing, dressing and undressing, feeding oneself, using the toilet, taking medications, and not being bedridden) may be impaired. Grade 4 toxicities are life-threatening and urgent intervention is indicated. Grade 5 toxicity produces an adverse event-related death. Thus, in various embodiments, use of the drug in the herein disclosed regimen, dosage, or route of administration, reduces the grade of a toxicity associated with treatment by at least one grade as compared to use of the drug according to another regimen. In other embodiments, by use of the drug according to a specified regimen or dosage, a toxicity is confined to grade 2 or less, grade 1 or less, or produces no observation of the toxicity. In some embodiments, the dosage is adjusted to avoid toxicities greater than grade 2.

Although the compact administration protocols disclosed herein are designed to reduce the risk, occurrence, and severity of inflammatory reactions of various sorts, the possibility of infusion reactions and immunotherapy-associated adverse events such as cytokine release syndrome (CRS), immune effector cell-associated neurotoxicity syndrome (ICANS) and hemophagocytic lymphohistiocytosis/macrophage activation syndrome (HLH/MAS) are still possibilities. The risk, occurrence, and severity of these events can be further reduced by prophylactic anti-inflammatory treatment. Such treatment can comprise treatment with a corticosteroid, an H1 blocker, an H2 blocker, or any combination thereof. Some embodiments of prophylactic anti-inflammatory treatment comprise, or consist of, treatment with a corticosteroid, an H1 blocker, and an H2 blocker. In some embodiments, the anti-inflammatory agents are administered one each day of dosing the tLNP at least 60 minutes prior to the start of the tLNP infusion (or other administration). In alternative embodiments, the anti-inflammatory agents are administered only prior to the first dose of a treatment cycle. In some embodiments, the anti-inflammatory agent(s) is administered parenterally, for example intravenously (IV) or intramuscularly. In other alternative embodiments, the anti-inflammatory agents are administered only prior to the last dose of a treatment cycle. In some embodiments, the anti-inflammatory agent(s) is administered intravenously. Intravenous administration (and in general parenteral administration) assures that the drugs are at therapeutic levels when tLNP administration begins. Oral administration is also possible, but it is more difficult to ensure that therapeutic levels are present in a short period of time.

In some embodiments, the corticosteroid is dexamethasone. In some embodiments, the dexamethasone is administered intravenously at a dosage of from about 5 to about 20 mg, any integer-bound subrange therein, or at any integer value therein, for example 10 mg IV. In alternative embodiments, the corticosteroid is hydrocortisone. In some embodiments, the hydrocortisone is administered intravenously at a dosage of from about 100 to about 400 mg, any integer-bound subrange therein, or at any integer value therein, for example, 200 mg IV. In alternative embodiments, the corticosteroid is methylprednisolone. In some embodiments, the methylprednisolone is administered intravenously at a dosage of from about 25 to about 100 mg, any integer-bound subrange therein, or at any integer value therein, for example, 50 mg IV. These corticosteroids constitute means for reducing inflammation and immune system activity.

In some embodiments, the H1 blocker is diphenhydramine, for example, 50 mg IV. Alternative HI blockers include cetirizine, levocetirizine, loratadine, and fexofenadine. Each of these H1 blockers constitute means for blocking the histamine H1 receptor.

In some embodiments, the H2 blocker is ranitidine, for example, 50 mg IV. Alternative H2 blockers include famotidine and cimetidine. Each of these H2 blockers constitute means for blocking the histamine H2 receptor.

The effectiveness of prophylactic anti-inflammatory treatment with a corticosteroid to reduce the risk, occurrence, and severity immunotherapy associated adverse events is not dependent on the use of the compact administration regimen but can be applied to any in vivo engineered immune cell therapy relying on LNP-mediated in vivo transfection of the immune cell. The same dosages and schedules of administration described above would apply to such aspects as well.

In certain aspects, this disclosure provides methods of treating a B cell-related disorder comprising administering multiple doses of a T cell-targeted tLNP encapsulating an mRNA encoding a T cell antigen receptor in a compact regimen to a subject in need thereof, wherein a population of cells in the subject express an antigen recognized the antigen receptor. (The phrase “comprising administering multiple doses of a T cell-targeted tLNP” may be alternatively stated as “comprising repeatedly administering a T cell-targeted tLNP”). In some embodiments more T cells express the antigen receptor as a result to a subsequent administration than as a result of the initial administration. In some embodiments, greater pharmacologic and/or clinical effect and/or improved safety is achieved after administration of at least 2 doses in the compact regimen as compared to the same total dosage administered over a same time interval as a single dose, or as multiple doses where each subsequent dose is administered ≥7 days after an immediately preceding dose.

Embodiments described with respect to the aspects of methods of treating a disease or disorder apply to these aspects of treating a B cell-related disorder except for those not related to a B cell disorder. In various embodiments, the B cell disorder is an autoimmune disease, a B cell cancer, light-chain amyloidosis, allogeneic transplant rejection, or Waldenstrom's macroglobulinemia. In various embodiments, the autoimmune disease is any form of lupus, any form of myositis, membranous nephropathy, any kidney inflammatory disease, systemic sclerosis, any brain inflammatory disease, myasthenia gravis, autoimmune hemolytic anemia, and any inflammatory peripheral neuropathies.

Naïve B cells and B cells located outside of follicles and germinal centers are generally more susceptible to depletion. Thus, the initial disappearance of B cells from the blood stream, as can be observed following a first infusion according to the herein disclosed treatments, is not a reliable indicator that a deep depletion of B cells extending to organs, including spleen, bone marrow, lymph nodes, etc. The subsequent administrations can result in higher effector to target ratios of more metabolically active cells with higher expression of the exogenous antigen receptor including increased number and trafficking of polyfunctional cells. This facilitates the depletion of memory B cells in lymphoid tissues, thereby achieving the deep depletion needed for immune reset (reversing autoimmunity) or for eradicating cancerous B cells.

Some embodiments of these methods of treatment comprise administration of an effective amount of a tLNP or a composition disclosed herein. Some instances relate to a therapeutically (or prophylactically) effective amount. A therapeutically effective amount is not necessarily a clinically effective amount, that is, while there can be therapeutic benefit as compared to no treatment, a method of treatment may not be equivalent or superior to a standard of care treatment existing at some point in time. Other instances relate to a pharmacologically effective amount, that is an amount or dose that produces an effect that correlates with or is reasonably predictive of therapeutic (or prophylactic) utility. As used herein, the term “therapeutically effective amount” is synonymous with “therapeutically effective dose” and means at least the minimum dose of a compound or composition disclosed herein necessary to achieve the desired therapeutic or prophylactic effect. Similarly, a pharmacologically effective dose means at least the minimum dose of a compound or composition disclosed herein necessary to achieve the desired pharmacologic effect. Some embodiments can refer to an amount sufficient to prevent or disrupt a disease process, or to reduce the extent or duration of pathology. Some embodiments can refer to a dose sufficient to reduce a symptom associated with the disease or condition being treated. An effective dosage or amount of a tLNP or a composition disclosed herein can readily be determined by the person of ordinary skill in the art considering the present disclosure and other relevant criteria (for example, the rate of excretion of the compound or composition used, the pharmacodynamics of the tLNP and its encapsulated mRNA or composition used, the nature of the other compounds to be included in the composition, the particular route of administration, the particular characteristics, history and risk factors of the individual, such as, e.g., age, weight, general health and the like, the response of the individual to the treatment, or any combination thereof) and utilizing his best judgment on the individual's behalf Exemplary dosages are also disclosed herein below.

In some embodiments, the tLNP administration comprises intravenous, intramuscular, subcutaneous, intralesional, intratumoral, intranodal or intralymphatic administration. In some embodiments, administration is by intravenous or subcutaneous infusion or injection. In some embodiments, administration is by intraperitoneal or intralesional infusion or injection. Frequently tLNPs are administered by intravenous infusion and with dosage specified in terms of mass of mRNA (or other payload) administered per mass of subject, typically as mg/kg.

Delivered dosage is determined from three variables: concentration of the medicament, infusion flow rate, and duration of infusion. While dosage can be adjusted by varying infusion duration, set infusion times are commonly employed. In various embodiments, infusion time is set at 60, 70, 80, 90, 100, 110, 120, 130, 140, or 150 minutes, or within a range bound by any pair of those values. In some embodiments, infusion duration is 60, 90, or 120 minutes. Lower concentrations can reduce local irritation and potentially systemic inflammation and can be achieved through use of lower total dosages and/or extended infusion duration. In some embodiments, the concentration of an administered tLNP suspension is in the range of 15 to 500 μg/mL, for example 250 μg/mL. In the clinic, it can be simpler to adjust flow rate than medicament concentration. Infusion pumps with programmable flow rates from 0.1 ml/hr to 999 ml/hr are available (for example, Alaris PC-8015). In various embodiments, flow rate can be from about 0.10 to about 16 ml/kg/hour or any increment of 0.1 within that range or a subrange bound by two such values. In various embodiments, simple flow rates can be in the range of about 0.2 to about 15 mL per minute or any increment of 0.1 within that range or a subrange bound by two such values. Specific flow rates are also disclosed in Example 17.

Treatment activity includes the administration of the medicaments, dosage forms, and pharmaceutical compositions described herein to a patient, especially according to the various methods of treatment disclosed herein, whether by a healthcare professional, the patient his/herself, or any other person. Treatment activities include the orders, instructions, and advice of healthcare professionals such as physicians, physician's assistants, nurse practitioners, and the like, that are then acted upon by any other person including other healthcare professionals or the patient him/herself. This includes, for example, direction to the patient to undergo, or to a clinical laboratory to perform, a diagnostic procedure, such as for cancer diagnosis and staging, or identification of an infectious agent, so that ultimately the patient may receive the benefit appropriate treatment. In some embodiments, the orders, instructions, and advice aspect of treatment activity can also include encouraging, inducing, or mandating that a particular medicament, or combination thereof, be chosen for treatment of a condition—and the medicament is actually used—by approving insurance coverage for the medicament, denying coverage for an alternative medicament, including the medicament on, or excluding an alternative medicament, from a drug formulary, or offering a financial incentive to use the medicament, as might be done by an insurance company or a pharmacy benefits management company, and the like. In some embodiments, treatment activity can also include encouraging, inducing, or mandating that a particular medicament be chosen for treatment of a condition—and the medicament is actually used—by a policy or practice standard as might be established by a hospital, clinic, health maintenance organization, medical practice or physicians group, and the like. All such orders, instructions, and advice are to be seen as conditioning receipt of the benefit of the treatment on compliance with the instruction. In some instances, a financial benefit is also received by the patient for compliance with such orders, instructions, or advice. In some instances, a financial benefit is also received by the healthcare professional for compliance with such orders, instructions, or advice.

Certain embodiments constitute methods of reducing the induction of ADA against a therapeutic agent or other pharmaceutical. Such embodiments can make use of T cell activating agents that are different than the therapeutic agent (conditioning agents or a tLNP encapsulating a nucleic acid encoding an antigen receptor with a different specificity than the therapeutic agent). Such embodiments can also make use of an anti-B cell antigen receptor such as an anti-CD19 or anti-BCMA CAR as the T cell activating agent, whether or not the pursued antigen is a B cell antigen. In some embodiments, only the B cell depleting agents are tLNP-encapsulated nucleic acids encoding an antigen receptor to reprogram T cells and administered according to a compact regimen while the therapeutic agent or other pharmaceutical is administered during the interval of B cell depletion (≤5 or ≤10 circulating B cells per μL of blood). In some instances of these embodiments, the therapeutic agent comprises a tLNP-encapsulated nucleic acid encoding a reprogramming agent which is or is not administered using a compact regimen. In other instances, the pharmaceutical is any potentially immunogenic product administered on any schedule within the interval of B cell depletion. In various embodiments induction of ADA comprises a de novo response, a recall response, a substantial pre-existing titer to a product component, or any combination thereof. Administration of a T cell activating agent that is different than the therapeutic agent and depleting B cells prior to administration of a therapeutic agent each constitute a step for reducing the induction of ADA.

Certain embodiments constitute methods of reducing the risk or occurrence of adverse immune reactions such as CRS, ICANS and/or HLH/MAS. Such embodiments make use of a compact dosing regimen and a transiently-expressed antigen receptor. Such embodiments can further make use of a conditioning agent as the T cell activating agent to further reduce exposure to the therapeutic agent. Other embodiments of reducing the risk or occurrence of adverse immune reactions comprise administration of a low dose of corticosteroid. While the dosage will depend on the corticosteroid used, the dosage will be low in comparison to the typical dosage used in treating inflammatory or autoimmune disorders. In various embodiments, the corticosteroid can be administered prior to a first dose of an in vivo engineering agent in a treatment cycle, the last dose of an in vivo engineering agent in treatment cycle, or every dose of an in vivo engineering agent in a treatment cycle. In some embodiments, the in vivo engineering agent is encapsulated in a tLNP. In other embodiments, the in vivo engineering agent is encapsulated in another type of nanoparticle such as a tissue-tropic LNP, a viral or virus-like particle, or polymer-containing nanoparticle. Use of a conditioning agent as a T cell activating agent in a compact regimen and administration of low dose corticosteroid prior to administration of an in vivo engineering agent each constitute a step for reducing the risk or occurrence of adverse immune reactions.

Certain embodiments comprising administration of low dose corticosteroid prior to administration of an in vivo engineering agent constitute methods of augmenting the safety or increasing the therapeutic index of the agent. When used in the context of a compact regiment as disclosed herein such embodiments constitute methods of augmenting safety or increasing therapeutic index without interfering with immune engineering amplification. Such methods can further comprise a step for B cell depletion. These methods enable intensification of dosing by allowing greater individual and/or cumulative dosages than could otherwise be safely utilized. In the part of the dose-response curve where increased dosage (individual or cumulative) is correlated with increased efficacy, the ability to safely utilize higher dosages (the greater therapeutic index) enables the achievement of higher efficacy.

Certain embodiments constitute methods of increasing the potency of an in vivo engineering agent. Such embodiments make use of a T cell activating agent to potentiate the transfection efficiency of a therapeutic agent by using a compact, produce more reprogrammed cells, and thereby increase the pharmacodynamic effect of the therapeutic agent. The large number of T effector cells thus generated can be useful when there is a large burden of cells expressing the pursued antigen. Use of a conditioning agent or a tLNP encapsulated nucleic acid encoding a reprogramming agent (wherein the activating reprogramming agent has a same or a different specificity than the therapeutic reprogramming agent) in an initial dose of a compact regimen as disclosed herein each constitute a step for increasing the potency of an in vivo engineering agent.

For example, some embodiments of increasing potency comprise 1 or 2 administrations of a tLNP encapsulating an RNA encoding a CAR binding a first antigen and 1 or 2 subsequent administrations of a tLNP encapsulating an RNA encoding a CAR binding a second distinct antigen, all administrations Q72 hrs, wherein the CAR binding the first antigen activates many T cells due to the prevalence of the first antigen (for example CD19) so that a large number of T cells binding the 2^{nd} antigen (for example, FAP, LRRC15, BCMA, or a tumor antigen) are generated to achieve a high response to cells expressing the 2^{nd} antigen (the pursued antigen). If the first antigen is a B cell antigen (such as CD19) then induction of ADA can also be pre-empted.

### Pharmaceutical Compositions and Doses

A pharmaceutical composition is one intended and suitable for the treatment of disease, such as in humans. That is, it provides overall beneficial effect and does not contain amounts of ingredients or contaminants that cause toxic or other undesirable effects unrelated to the provision of the beneficial effect. A pharmaceutical composition will contain one or more active agents and may further contain solvents, buffers, diluents, carriers, and other excipients to aid the administration, solubility, absorption or bioavailability, and or stability, etc. of the active agent(s) or overall composition. A “pharmaceutically acceptable carrier, diluent, or excipient” is a medium generally accepted in the art for the delivery of biologically active agents to mammals, e.g., humans. The tLNPs of the present invention can be formulated as pharmaceutical compositions using a pharmaceutically acceptable carrier, diluent, or excipient and administered by a variety of routes. In particular embodiments, such compositions are for intravenous administration. Such pharmaceutical compositions and processes for preparing them are well known in the art. See, e.g., REMINGTON: THE SCIENCE AND PRACTICE OF PHARMACY (Gennaro et al., eds., 19.sup.th ed., Mack Publishing Co., 1995).

In certain aspects this disclosure provides pharmaceutical compositions comprising the disclosed T cell-targeted tLNP encapsulating an mRNA encoding T cell antigen receptor and pharmaceutically acceptable solvents, buffers, salts, cryopreservatives, and/or other excipients. In some embodiments, the pharmaceutical composition comprises a pharmaceutically acceptable aqueous buffer. In some instances, the buffer comprises tris (tris(hydroxymethyl)aminomethane). In some embodiments, the pharmaceutical composition comprises a pharmaceutically acceptable salt. In some instances, the pharmaceutically acceptable salt is sodium chloride. In some embodiments, the pharmaceutical composition comprises a pharmaceutically acceptable cryopreservative. In some instances, the pharmaceutically acceptable cryopreservatives comprises sucrose and/or glycerol.

In certain aspects this disclosure provides a dose of T cell-targeted tLNP encapsulating a nucleic acid, for example, an mRNA, circular RNA, or self-replicating RNA encoding T cell antigen receptor according to any of the disclosed embodiments, or a pharmaceutical composition comprising said dose of tLNP, suitable for administration in a compact regimen. In some embodiments, the nucleic acid is mRNA. In some embodiments, the dose is provided at a dosage of at least 0.3 mg/kg or in a range of 0.3 to 1.0 mg/kg. In some embodiments, a dose suitable for a 70 kg subject is 21 mg or at least 21 mg. In some embodiments, a dose suitable for a 70 kg subject is in a range of 21 to 70 mg. In some embodiments, a dose of 50 mg is suitable for a subject of about 50 to 167 kg, providing a dosage in the range of about 0.3 to 1.0 mg/kg. In some embodiments, a dose of 40 mg is suitable for a subject of about 40 to 133 kg, providing a dosage in the range of about 0.3 to 1.0 mg/kg. In some embodiments, a dose of 30 mg is suitable for a subject of about 30 to 100 kg, providing a dosage in the range of about 0.3 to 1.0 mg/kg. In some embodiments, a dose of 25 mg is suitable for a subject of about 25 to 83 kg, providing a dosage in the range of about 0.3 to 1.0 mg/kg. In some embodiments, a dose of 20 mg is suitable for a subject of about 20 to 67 kg, providing a dosage in the range of about 0.3 to 1.0 mg/kg. In various embodiments, the cumulative dosage does not exceed 0.5, 1.0, 1.5, 2.0, 2.5, 3.0, 3.5, 4.0, or 4.5 mg RNA/kg/6 days or is in a range bound by any pair of those values. Some embodiments are pharmaceutical compositions formulated for administration in an amount of about 0.03, 0.06, 0.1, 0.2, 0.3, 0.4, 0.5, 0.6, 0.7, 0.8, 0.9, 1.0, 1.2, 1.4, 1.5, 1.75, or 2.0 mg/kg, or in a range of about 0.1 mg/kg to 2 mg/kg, about 0.3 mg/kg to 1.0 mg/kg, or any other range bound by a pair of these values. In all such embodiments, the dose is expressed in terms of the amount of mRNA or other nucleic acid contained in the tLNP composition to be administered. In some embodiments, the dose is contained in a pre-filled syringe.

Unless otherwise defined, all technical terms used herein have the same meaning as commonly understood by a person of ordinary skill in the art to which this disclosure belongs.

For the purposes of promoting an understanding of the principles of the disclosure, reference will now be made to embodiments and specific language will be used to describe the same. It will nevertheless be understood that no limitation of the scope of the disclosure was thereby intended, such alteration and further modifications of the disclosure as illustrated herein, being contemplated as would normally occur to one skilled in the art to which the disclosure relates.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

### Materials and Methods

The following examples make use of CD8-targeted tLNPs encapsulating mRNA encoding either an anti-CD20 CAR (tLNP-982520) or an anti-CD19 CAR (tLNP-98219). The anti-CD8 antibody used as the targeting moiety of the tLNPs was a whole antibody comprising a human IgG1 Fc with Fc-silencing mutations L234A, L235A, and P329A (LALAPA) and a humanized antigen binding domain referred to as CBD1033 each described in International Patent Application No. PCT/US2024/060426, which is incorporated by reference in its entirety. The lipid composition of the tLNP is described in International Application Publication No. WO2024249954, which is incorporated by reference in its entirety, as composition code F9. The anti-CD19 and anti-CD20 CARs are referred to in International Patent Application No. PCT/US2024/054033 as CAR2 and CAR25, respectively, and the mRNAs encoding them as RM_61461 (SEQ ID NO: 212) and RM_61639 (SEQ ID NO: 219). However, the herein disclosed methods are not dependent on the use of these particular reagents. Rather it is the compact administration regimen that is responsible for the immune engineering amplification phenomenon and other tLNPs bearing other lymphocyte-targeting moieties and other lipid compositions encapsulating mRNAs encoding other antigen receptors will experience similar amplification.

CD19 is expressed on a broader range of cells in the B cell lineage from pro-B cells through plasmablasts (FIG. 1A). In contrast, CD20 is expressed on pre-B cells through memory B cells. Accordingly anti-CD19 CARs have generated more interest than anti-CD20 CARs for the treatment of B cell-associated diseases and especially in the treatment of autoimmune diseases.

However, the anti-human CD19 CAR used in these studies is minimally cross-reactive with NHP CD19 (as is common) and an anti-CD20 CAR was used as a surrogate in the NHP experiments described herein providing a reagent with substantial pharmacologic activity in NHP. To support use of an anti-CD20 CAR as a surrogate, PBMC from two healthy human donors comprising both T and B cells were transfected by exposure to tLNP-982520 or tLNP-98219 for 1 hour at 3 dose levels in vitro—0.2, 0.6, and 2 μg—followed by measurement of B cell concentrations by flow cytometry at 72 hours. Treatment with either tLNP led to comparable depletion of B cell (FIG. 1B) demonstrating the usefulness of the tLNP encapsulating an anti-CD20 CAR as a surrogate for the tLNP encapsulating an anti-CD19 CAR.; data shown from the 0.6 μg dose.

### Example 1

### Increased Pharmacological Effect Afforded by Repeat Dosing with Smaller Cumulative Dose

To assess the ability of in vivo generated CAR-T cells recognizing a B cell surface marker to deplete tissue B cells, cynomolgus macaques were infused with CD8-targeted tLNPs encapsulating mRNA encoding a pharmacologically active anti-CD20 CAR either 3 times 0.3 mg/kg per dose, 3 days apart (Q72 hrs), or once with 2 mg/kg. (Dosages report the amount of mRNA in the tLNP administered throughout.) Animals were necropsied at 3 days post final infusion, spleens were harvested, sectioned, and stained for B cells using an anti-CD20 antibody

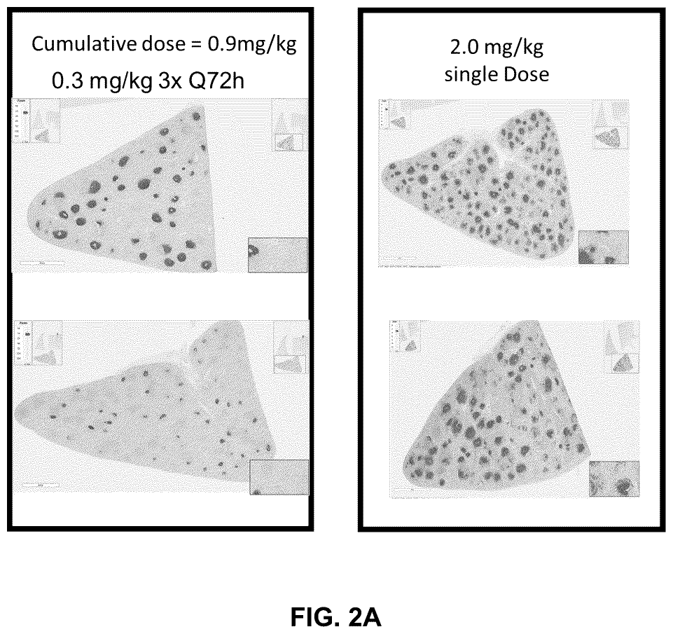

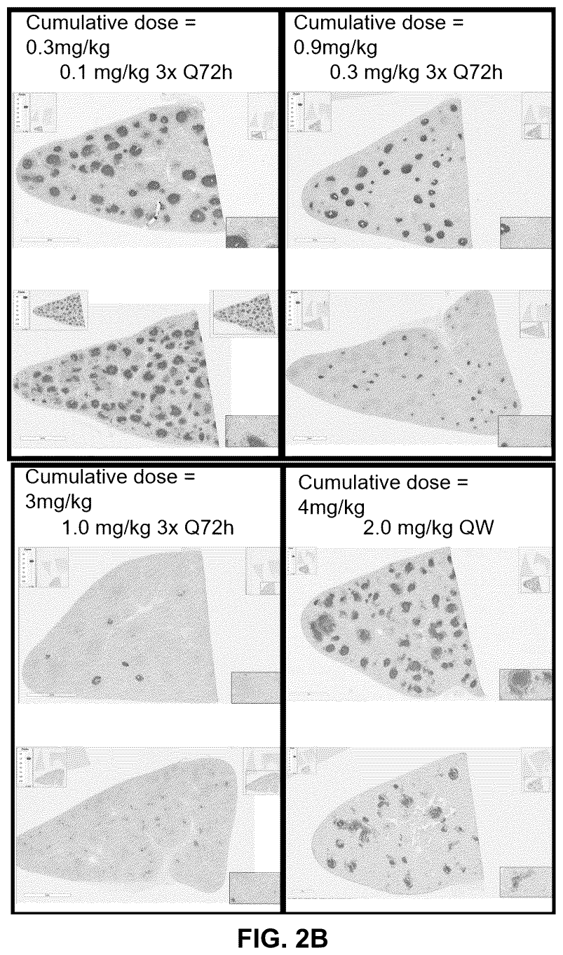

The results (FIG. 2A) showed profound pharmacological effects reflected in deep depletion of B cells in the follicle structures, for the compact dose regimen, 3×0.3 mg/kg Q72 hrs representing a cumulative dose of 0.9 mg/kg; as compared to a higher cumulative dose of 2 mg/kg, delivered in a single infusion, that did not achieve the same magnitude of response. The results are representative for 2 animals/group and staining for CD20.

To further explore effects of timing and dosage, cynomolgus macaques were infused with CD8-targeted tLNPs encapsulating mRNA encoding a pharmacologically active anti-CD20 CAR either 3 times with 0.1 mg/kg, 0.3 mg/kg or 1.0 mg/kg per dose, 3 days apart, or twice with 2 mg/kg 1 week apart (QW). Animals were necropsied at 3 days post final infusion, spleens were harvested, sectioned, and stained for B cells using an anti-CD20 antibody.

The results (FIG. 2B) showed profound pharmacological effect reflected in deep depletion of B cells in the follicle structures, by using a compact dose regimen 3×0.3 mg/kg Q72 hrs representing a cumulative dose of 0.9 mg/kg or 3×1.0 mg/kg Q72 hrs representing a cumulative dose of 3.0 mg/kg, as compared to a higher cumulative dose of 4 mg/kg, delivered as 2×Q7 days or QW, that has not achieved the same magnitude of response. The results are representative for 2 animals/group and staining for CD20. These data showed that smaller doses repeatedly administered in a compact schedule can achieve greater depletion of B cells—shrinking and disappearance of B cell follicles—than a single administration of a larger dose exceeding the cumulative dose of the repeated doses as indicated.

### Example 2

### Post-Treatment Tissue Cell Proportions

The proportion of B cells as a percentage of CD45⁺ leukocytes in various tissues was determined following several dosing protocols. Cynomolgus macaques were infused with CD8-targeted tLNPs encapsulating mRNA encoding a pharmacologically active anti-CD20 CAR either 3 times with 0.3 mg/kg, or 1.0 mg/kg per dose 3 days apart; or twice, with 1 mg/kg or 2 mg/kg 1 week apart, or once, with 2 mg/kg. Animals were necropsied at 9 days post 1st infusion, cells were isolated from bone marrow, liver, lymph node, and spleen and stained with multiple immune cells markers to define the % of B cells within the total CD45⁺ immune cell population.

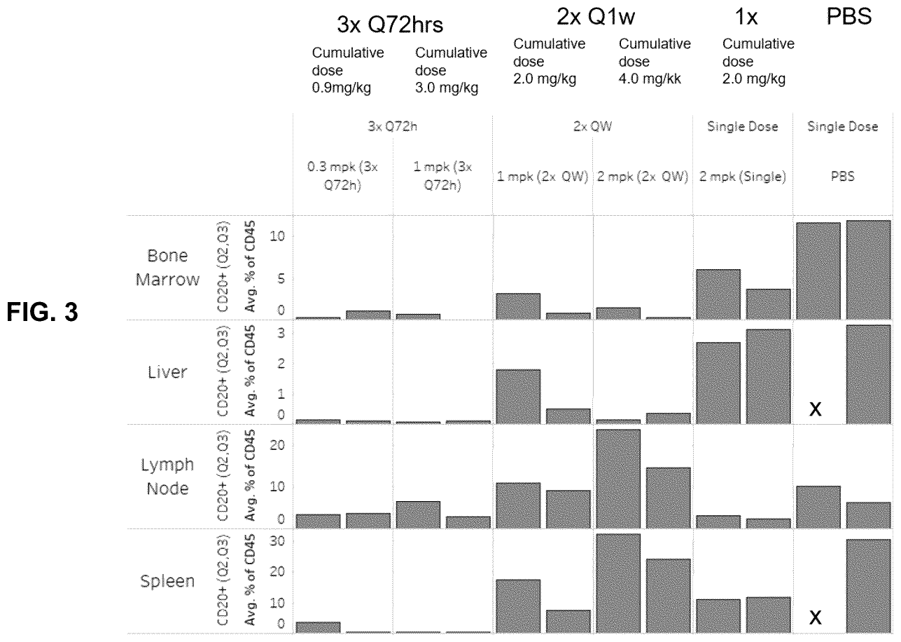

The results showed profound pharmacological effects reflected in deep depletion of B cells in bone marrow, lymph nodes, liver, and spleen by using a compact dose regimen 3×0.3 mg/kg Q72 hrs representing a cumulative dose of 0.9 mg/kg; or 3×1.0 mg/kg Q72 hrs representing a cumulative dose of 3.0 mg/kg, as compared to a higher cumulative dose of 2 mg/kg or 4 mg/kg respectively, delivered as 2×Q7 days, or 2 mg/kg delivered as single infusion, that has not achieved the same magnitude of response. Data from individual animals are shown in FIG. 3.

The results showed that dividing a dose into multiple lower doses administered within a relatively short time period resulted in increased pharmacological effect of the tLNP formulation as compared to doses administered farther apart in time even if higher cumulative dosages were administered. These data showed generally greater B cell depletion with a compact regimen (3×Q72) than a larger single administration or a 2×QW schedule and larger doses.

### Example 3

### Compact Administration Schedule had Greater Effect on AUC of CAR Expression than Cumulative Dose

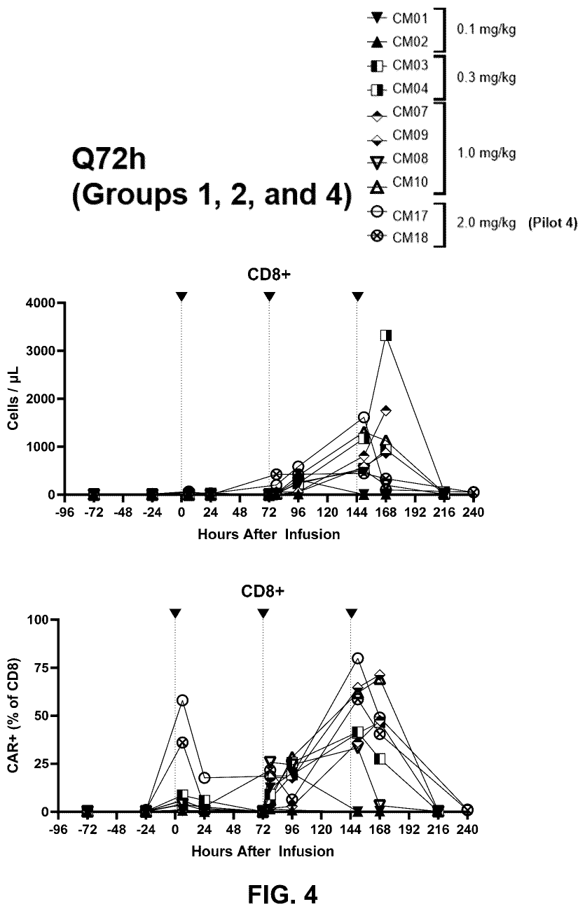

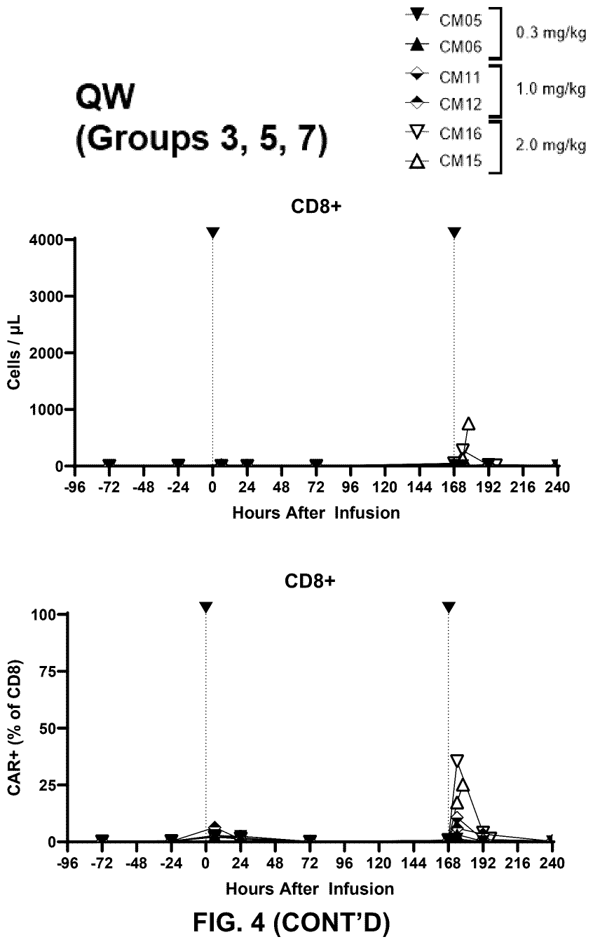

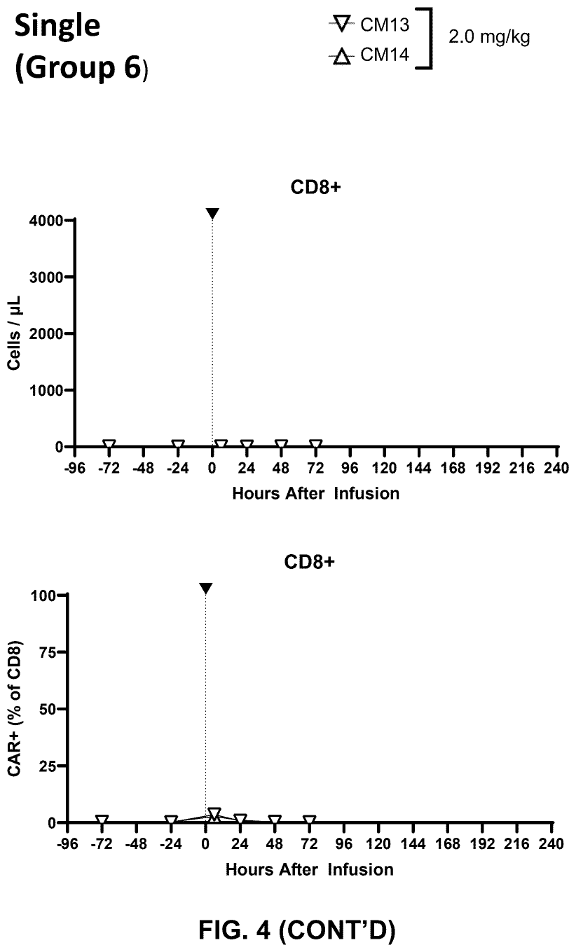

The increased B cell depletion achieved with the compact administration schedule could arise from generation of a greater number of more active CAR-expressing cells. Cynomolgus macaques were infused with CD8-targeted tLNPs encapsulating mRNA encoding a pharmacologically active anti-CD20 CAR either 3 times, 3 days apart; or twice, 1 week apart, or once, with various doses of CD8-targeted tLNPs. CAR expression was evaluated by flow cytometry utilizing a CAR scFv-specific reagent and T cell markers for co-staining. To measure the % engineering rate, the cells were gated on CD8+ T cells in blood. FIG. 4 shows longitudinal levels of CAR+ T cells/μl or % CAR+ cells within CD8+ T cell population.

The results showed a high CAR engineering rate (up to about 70% CD8+ T cells), mirrored by a high number of CAR+ cells/μl obtained by using this compact dose regimen (3×Q72 hrs). The gradually higher number of engineered cells obtained upon 3rd dose is explainable by the increasing number of CAR T cells relative to pursued cells in vivo, owing to increased transfectability of T cells and diminished CAR internalization absence of cognate antigen.

Importantly, dose regimens with cumulative total dose of 0.9 mg/kg or 3 mg/kg, delivered as 3×0.3 mg/kg Q72 hrs or 3×1.0 mg/kg Q72 hrs, achieved higher engineering as compared to a cumulative dose of 2 mg/kg delivered once, or 4 mg/kg respectively, delivered 2× at 1 week interval. This shows that dividing a dose into multiple lower doses administered repeatedly in a compact schedule, results in increased tLNP engineering rates. This increased pharmacologic effect is also associated with reduced on-target related cytokine production as seen in Example 4.

The results of this experiment coincide well with the asserted underlying mechanisms of the compact regimen. That is, most T cells are in a resting state but upon being reprogrammed with a CAR, engage cognate antigen and become activating, accompanied by internalization of the CAR making detection interfering with detection of the CAR by flow cytometry (see FIG. 4). At this phase, B cell killing takes place predominantly in the periphery (tissues with interconnected vasculature). With the second and third doses, more T cells are already activated enabling higher engineering efficiency and increasing the total number of transfected cells with proliferation following antigen engagement after the first transfection being a contributing factor. The resultant increase in E:T (effector:target) ratio, and the already diminished number of B cells, makes CAR expression more readily detectable by flow cytometry, particularly following the third dose (see FIG. 4). This also leads to increased trafficking of polyfunctional cells and the killing of B cells in lymphoid tissues as needed to achieve immune reset and disrupt autoimmune pathology. These data showed the greater transfection efficiency achieved with the compact administration regimen (Q72); the arrowheads indicating when the tLNPs were administered.

### Example 4

### Cytokine Levels Accompanying tLNP Infusion and CAR Expression were Tunable

One of the potential adverse effects of CAR-T cell therapy is cytokine release syndrome (CRS, aka “cytokine storm”). It was possible that the reduced dosages used in the compact administration regimen would lead to a reduction in cytokine production and thereby provide a way to achieve the desired pharmacological (and clinical) effect without increasing the risk of CRS.

Cynomolgus macaques were infused with 0.1 mg/kg, 0.3 mg/kg, 1.0 mg/kg or 2.0 mg/kg of the CD8-targeted tLNPs encapsulating mRNA encoding a pharmacologically active anti-CD20 CAR or 2.0 mg/kg CD8-targeted tLNPs encapsulating mRNA encoding a negative control CAR (anti-human CD19 non-cross reactive to non-human primate (NIP)). Cytokines were measured longitudinally post 1^{st} infusion and shown as average for each group.

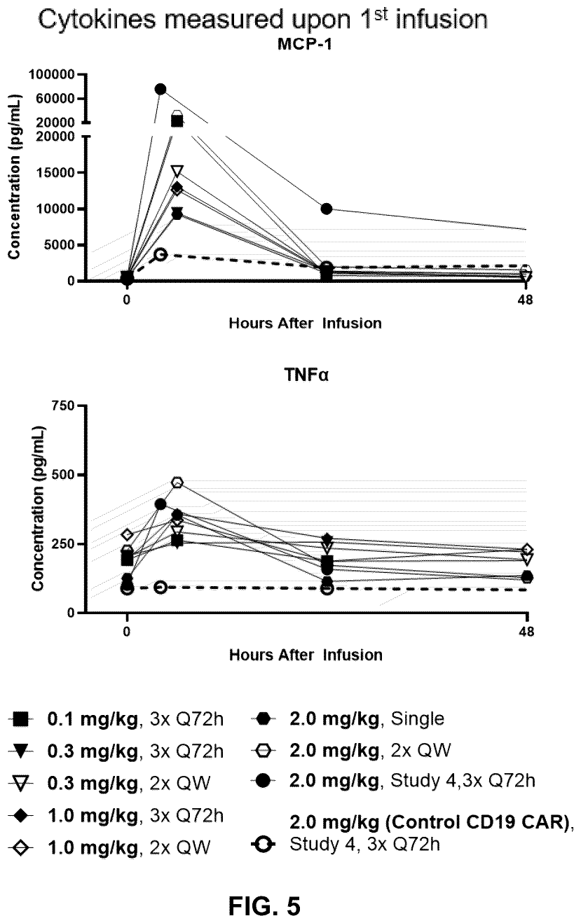

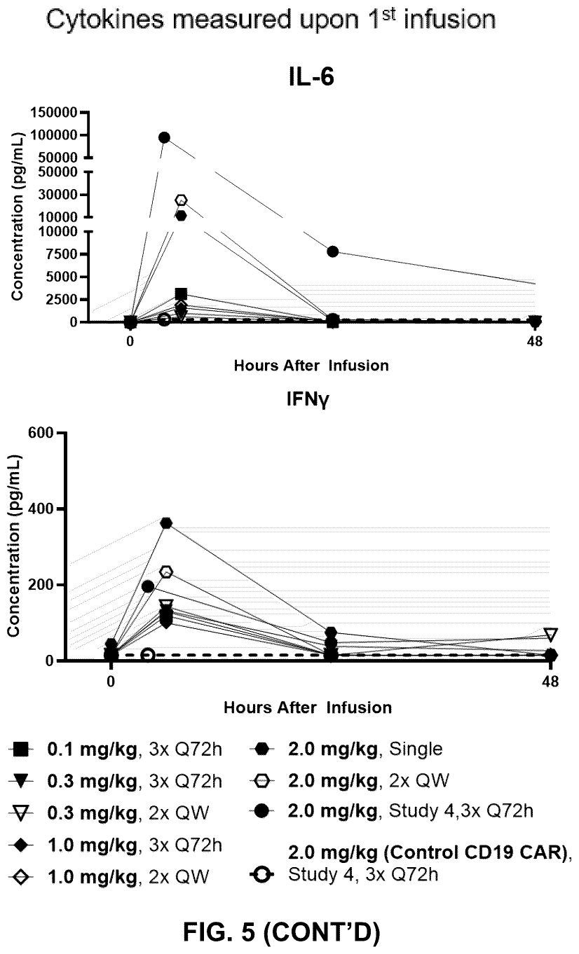

The results showed transient elevation of cytokine levels, relative to the negative control, correlated with on-target pharmacological activity. There was also a dose effect, with considerably lower peak levels of pro-inflammatory cytokines such as IL-6, at dose levels of 1.0 and 0.3 mg/kg, that nonetheless afforded deep pharmacological activity when used repeatedly as 3×Q72 hrs format (FIG. 5).

The results showed that by using lower doses of tLNP high levels of pro-inflammatory cytokines were avoided, supporting the safety of such regimens in contrast to delivering a high dose inoculum that may result in very high levels of cytokines manifested through cytokine release syndrome.

### Example 5

### Transfection of Resting T Cells with CD8-Directed tLNP

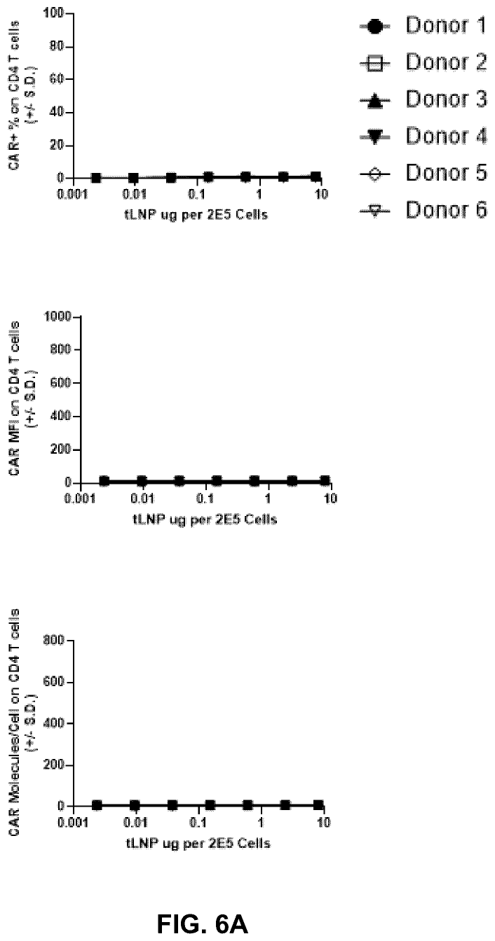

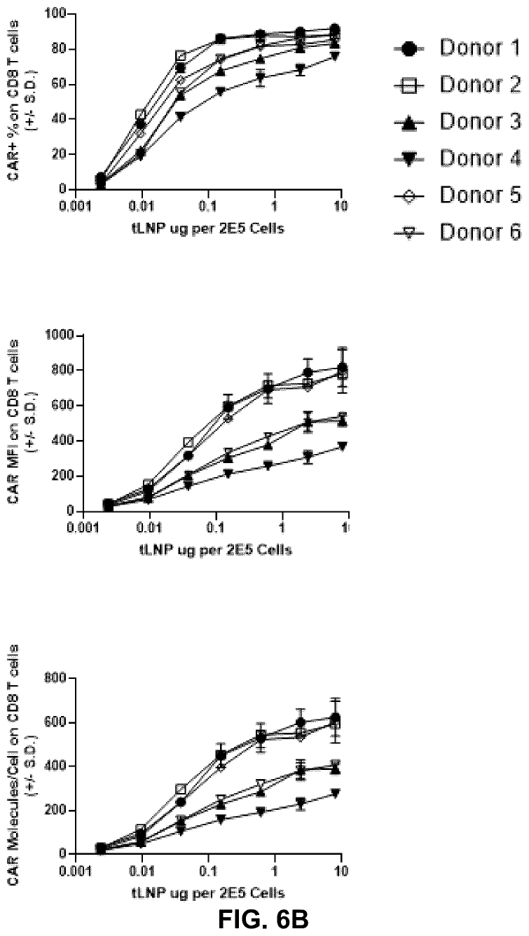

The ability of CD8-directed tLNP encapsulating mRNA encoding an anti-CD19 CAR to transfect resting (unactivated) human T cells and for the mRNA to be expressed was assessed. Human T cells were isolated from the blood of 6 donors and plated at 2×10⁵ cells per well. CD8-direct tLNP were added to the wells in amounts that provided various amount of mRNA ranging from 0.003 to 9.0 μg of the mRNA. 24 hours later CAR expression on CD4+ and CD8⁺ T cells was assessed by flow cytometry. Virtually no expression was observed on the CD4+ T cells, but the CAR was expressed by approximately 75-95% of CD8⁺ T cells (FIGS. 6A-6B, top panels). Expression level was also assessed as both mean fluorescence intensity (MFI) and molecules per cell as determined by molecular equivalent of soluble fluorochrome (MESF) (FIGS. 6A-6B, middle and bottom panels, respectively). These data demonstrated that the CAR was robustly expressed in resting (un-activated) T cells at 24 hr after transfection in a dose proportional manner.

### Example 6

### Transfected Cells Became Activated when Exposed to Cognate Antigen

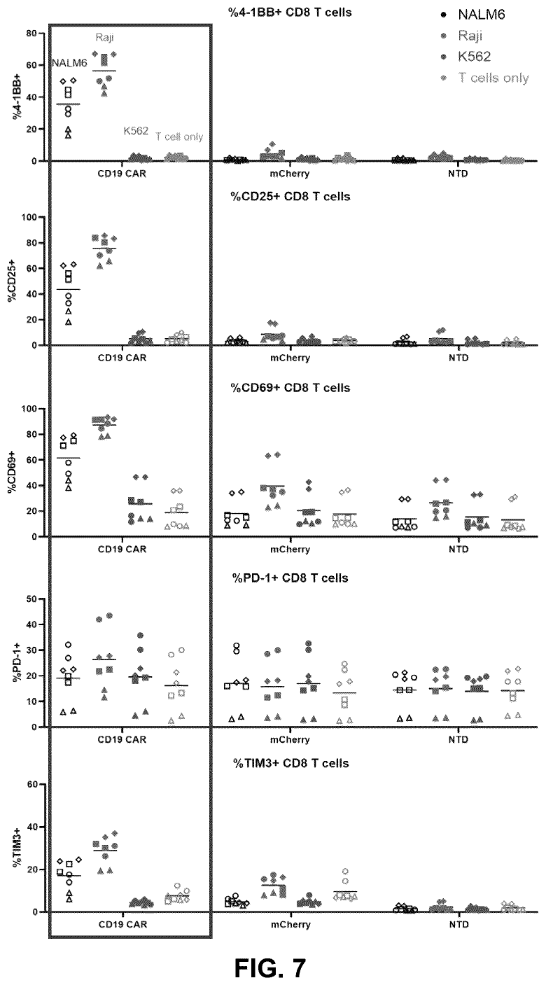

To demonstrate that the transfected, CAR-expressing T cells became activated when encountering cognate antigen, these tLNP-engineered CAR T cells (from the previous Example) were co-cultured 24 hours after transfection with CD19⁺ pursued cells (NALM6, Raji), CD19⁻ pursued cells (K562), or no additional cells for a further 24 hours. As shown in FIG. 7, this resulted in T cell activation for the CAR-expressing cells co-cultured with the CD19⁺ pursued cells, as observed by expression of activation markers measured by flow cytometry at 24 hrs post co-culture. Altogether, these results show that CAR expression in resting T cells enables their rapid activation in presence of cognate antigen.

### Example 7

### Activated T Cells Are More Efficiently Transfected than Unactivated T Cells

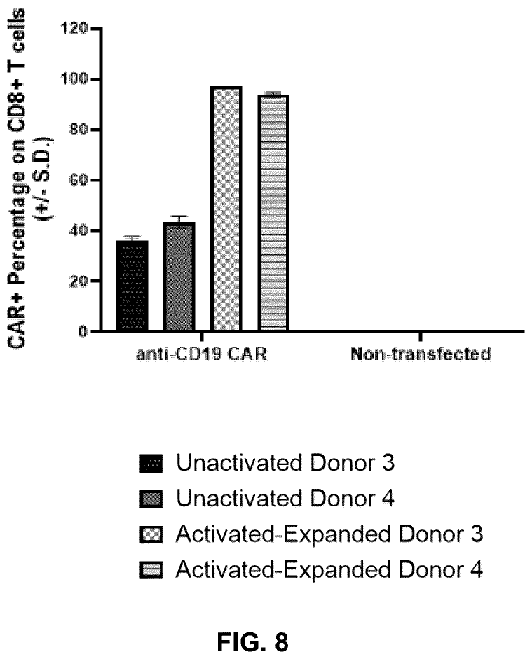

To demonstrate that activated T cells were more readily transfected, previously activated T cells (by polyclonal stimulation) and unactivated T cells from two human donors were transfected in vitro with CD8-targeted tLNPs encapsulating mRNA encoding an anti-CD19 CAR using 0.6 μg RNA per 2×10⁵ cells. CAR expression from each construct in duplicates is shown as CAR⁺ percentage of total CD8⁺ T cells at 24 hours post transfection. The CAR expression was measured by using anti-Whitlow (PE) antibody detecting the linker peptide within the scFv portion of the CAR. While the unactivated T cells had a transfection efficiency, as measured by CAR expression, of around 40%, the activated T cells had a transfection efficiency >90% (FIG. 8).

Altogether, these data indicated that the process of in vivo Immune Engineering Amplification, as demonstrated in Examples above, occurs after the first dose, when rested CAR T cells activate upon engagement with their cognate antigen. T cell activation led to their proliferation and a larger pool of activated and effector T cells to be transfected upon subsequent dosing. Hence, repeat infusions resulted in increased expansion of CAR effector cells relative to pursued cells resulting in an amplified pharmacological effect.

### Example 8

### B Cell Recovery Following Depletion was Predominantly Naïve B Cells

Following treatment to deplete B cells, repopulating B cells were phenotyped by flow cytometry. Immune reset, that is, the recovery to normal levels of B cells without the recurrence of autoimmunity, is associated with the initial recovery being predominated by naïve B cells.

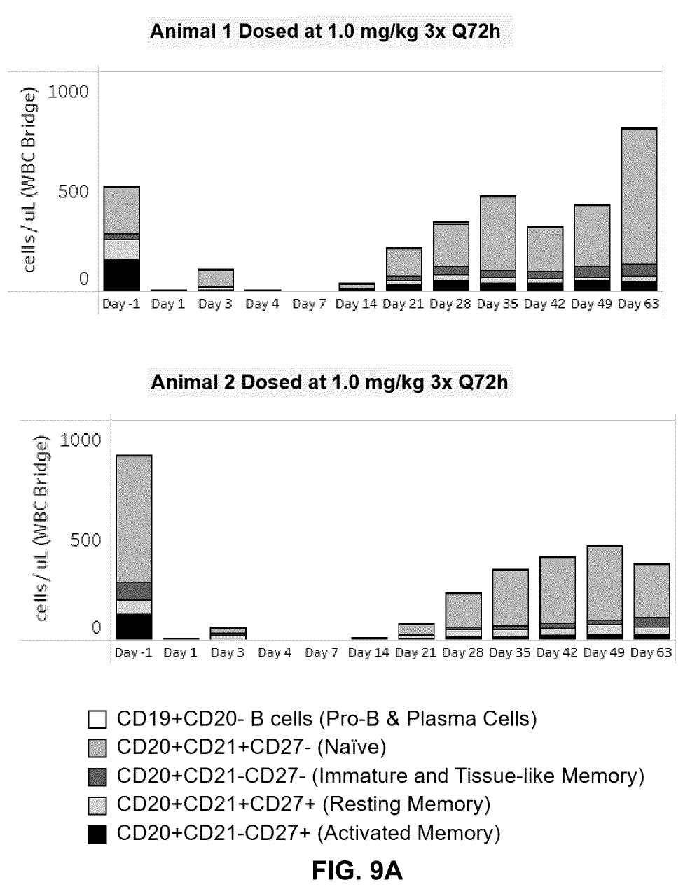

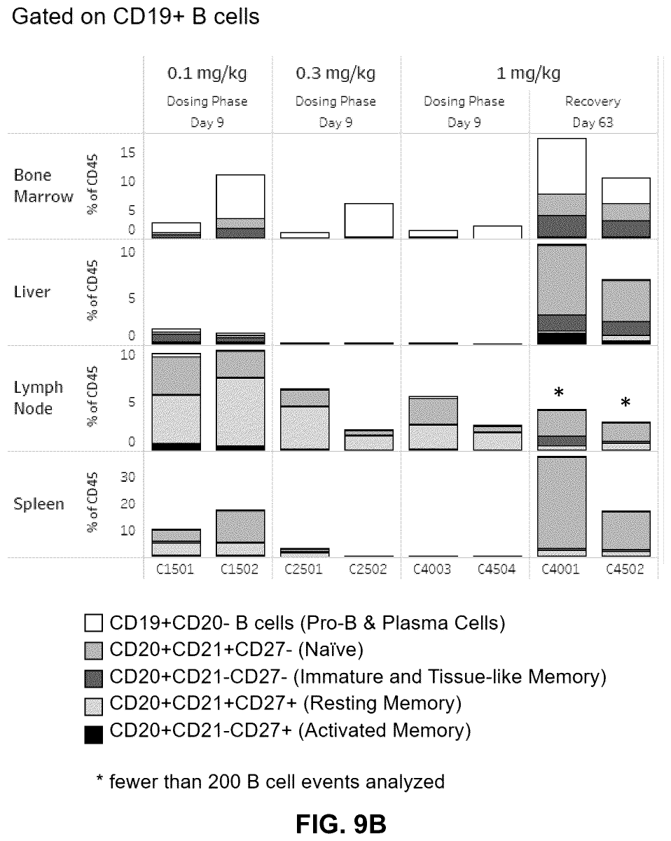

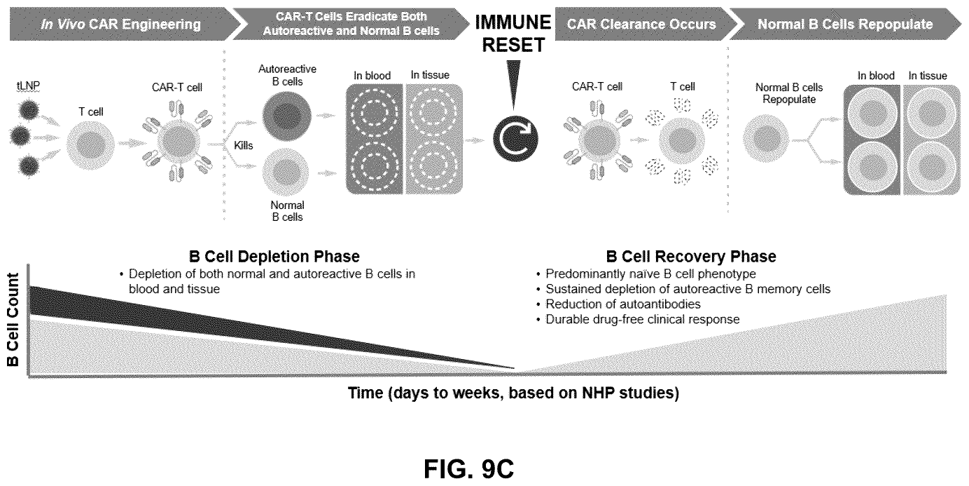

Cynomolgus macaques were dosed with CD8-targeted tLNPs encapsulating mRNA encoding a pharmacologically active anti-CD20 CAR at 1 mg/kg using a compact dose regimen, 3×Q72 hrs, on days 0, 3 and 6. Blood samples were obtained on days −1, 1, 3, 4, 7, 14, 21, 28, 35, 42 and 49, and liver, spleen, bone marrow, and lymph node samples were obtained at necropsy on day 63. Liver, spleen, bone marrow, and lymph node samples were obtained at necropsy from other animals dosed at 0.1 or 0.3 mg/kg at day 9. These treatments led to deep B cell depletion in blood and organs, although the depletion at 0.1 mg/kg was less profound (FIGS. 9A and 9B). B cell recovery, predominantly with naïve B cells, showing a sustained bias during the observation interval, manifested through reduced proportion of memory B cells (FIGS. 9A and 9B). The large proportion of pro-B cells in bone marrow throughout the experiment was expected as these cells are CD20⁻ and thus are not depleted by the treatment. The number of B cells in lymph node continued to be low at 2 months/day 63, with a possible deepening of B cell depletion especially involving the memory compartment (FIG. 9B), despite the fact that the CAR in mRNA format may not persist beyond a few days. The majority of the B cells were also naïve or immature. These data indicated that the depth of the B depletion using this regimen was biologically significant, leading to major and persisting changes in this population of cells, consistent with the immune reset mechanism.

Immune reset is illustrated in FIG. 9C which shows depletion of both naïve and memory B cells, which comprise the autoreactive B cells, following generation of CAR-T cells that pursue B cells using the compact administration, immune engineering application regimen, post-treatment recovery of naïve B cells preceding recovery of the memory B cell population—without autoreactive cells, and the waning of autoreactive antibodies and T cell titers.

### Example 9

### Integration of Immune Engineering Amplification for Immune Reset with Other Treatments of Autoimmunity

Immune Engineering Amplification can be used to achieve an immune reset in autoimmune diseases in which B cells play a contributing role (B cell-mediated autoimmunity). This is accomplished by using a compact administration regimen of T cell-targeted LNPs to deliver mRNA encoding an anti-B cell marker antigen receptor such as a TCR or CAR. The tLNPs are administered 2, 3, 4, or more times at intervals of 2 to 5 days. This regimen can be used alone or combined with other autoimmunity treatments to magnify a global immune reset.

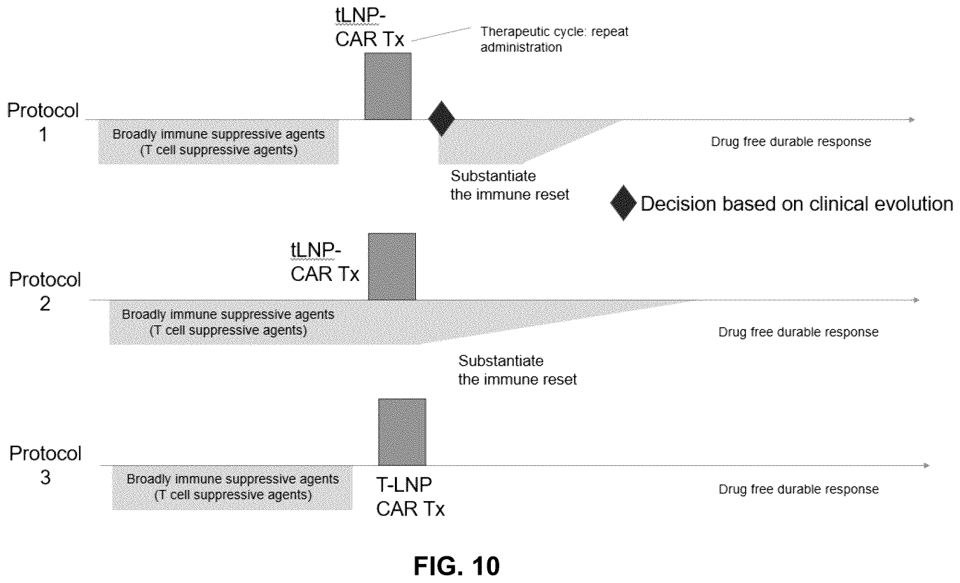

Integration of immune engineering amplification with broadly immunosuppressive or immunomodulating agents, particularly T cell-suppressive agent, such as corticosteroids, methotrexate, mTOR inhibitors, calcineurin inhibitors, mycophenylate mofetil, JAK inhibitors, monoclonal antibodies that interfere with activity of T cells and/or myeloid cells or other agents used as standard of care, is illustrated in FIG. 10. As shown for Protocol 1, a patient receiving a broadly immunosuppressive agent has this treatment suspended and, after an interval (1 to 6 weeks) for wash-out, the patient undergoes the immune engineering amplification regimen. At an interval (2 to 14 weeks) after conclusion of dosing of the tLNP, treatment with the immunosuppressive agent is resumed, or not, based on clinical evolution of the disease. If treatment with the immunosuppressive agent is resumed, treatment is subsequently tapered off once immune reset has been substantiated. Protocol 1 is particularly appropriate in disease indications where there is a substantial component attributable to T cells or macrophages.

As compared to Protocol 1, in Protocol 2 treatment with the immunosuppressive agent is not suspended during the immune engineering amplification regimen but is tapered off once immune reset has been substantiated. Protocol 2 is particularly appropriate when there is potential complementary activity between the immune modulating agents used as standard of care with the engineering of T cells to express an anti-B cell antigen receptor to ameliorate or prevent toxicities that may arise in the course of tLNP treatment, without interfering with the potency of engineered T cells.

In protocol 3, treatment with the immunosuppressive agent is simply ceased in advance of the immune engineering amplification regimen. In all three protocols the patient experiences a drug-free durable elimination of the autoimmune condition.

### Example 10

### Transfectability and Functionality of T Cells from Subjects with an Autoimmune Disease

To assess the applicability of tLNP-delivered mRNA-encoded T cell reprogramming agents to treatment of autoimmune diseases, peripheral blood cells from autoimmune disease patients were evaluated and benchmarked against healthy subjects with respect to phenotype, tLNP-mediated CAR engineering efficiency, and functional activity. Peripheral blood cells were obtained from healthy subjects and patients with systemic lupus erythematosus (SLE), anti-synthetase syndrome (AS), dermatomyositis (DM), multiple sclerosis (MS), rheumatoid arthritis (RA), scleroderma, Sjögren's syndrome, and immune-mediated necrotizing myopathy (IMNM) with various prior treatments, including in some cases immune suppressive or B cell depleting agents as described in Table 14. Blank cells in the Table indicate that the data were not available. Samples were processed (as described below) as they became available and not simultaneously.

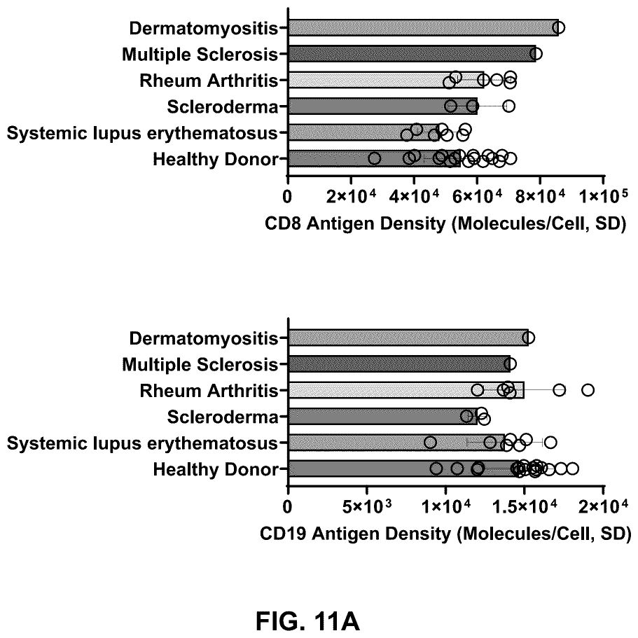

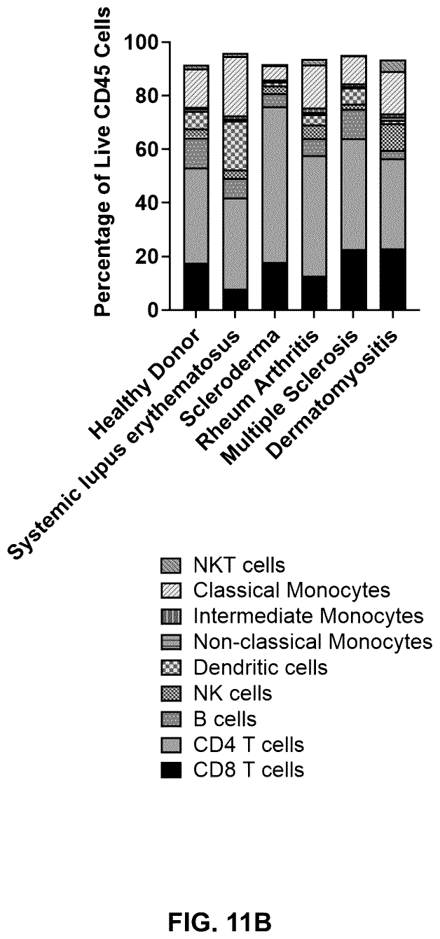

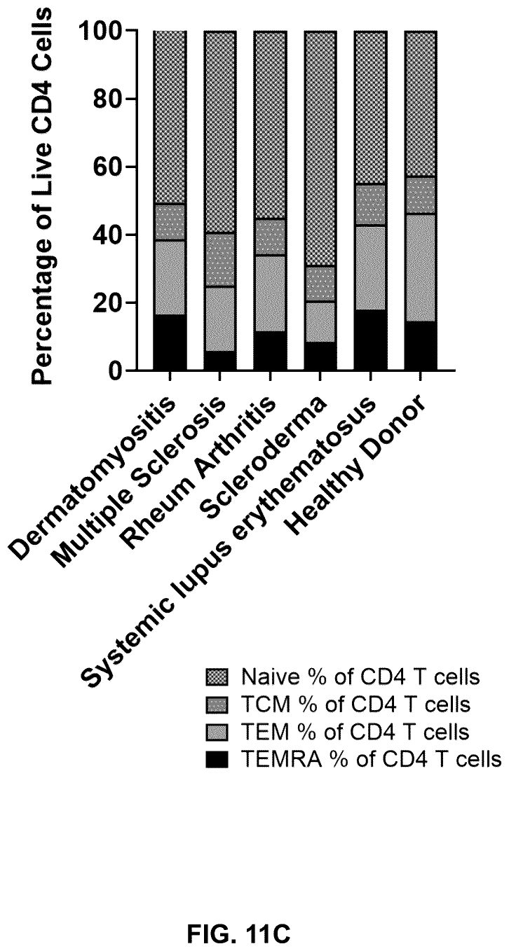

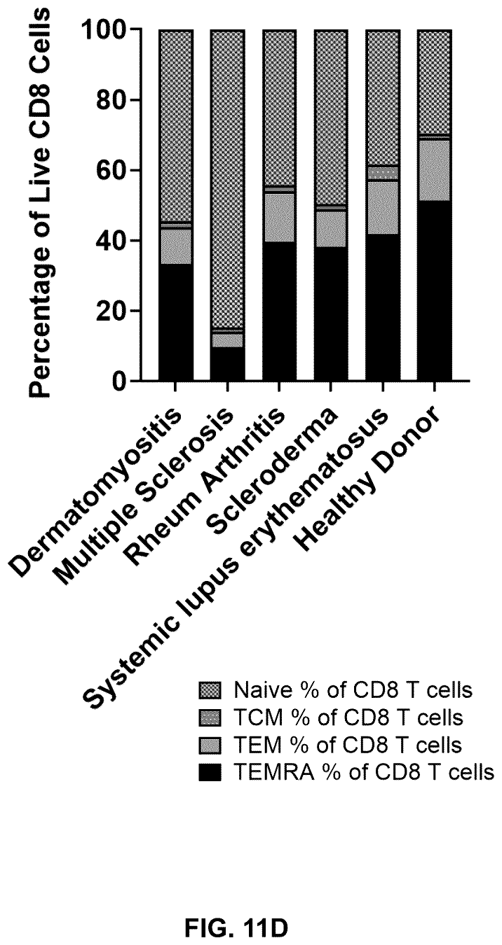

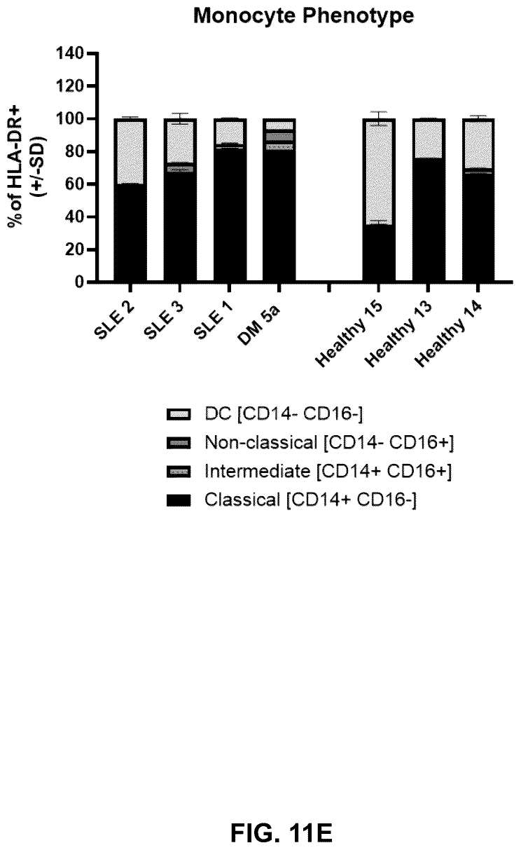

Human PBMC from autoimmune disease patients and healthy donors were thawed and immediately stained with antibodies for immunophenotyping. CD8 antigen density was measured by anti-CD8-PE antibody staining accompanied with BD Quantibrite™ Beads to quantify CD8 expression level on CD8⁺ cells. CD19 antigen density was measured by anti-CD19⁻PE antibody staining accompanied with BD Quantibrite™ Beads to quantify CD19 expression level on CD19+ cells. The compiled data indicated CD8+ cells derived from autoimmune patients have levels of CD8 antigen at least comparable to healthy donors, and CD19 cells derived from autoimmune patients have comparable CD 19 antigen level to healthy donors (FIG. 11A).

Accordingly, target antigen density on CD8+ cells from autoimmune patients was sufficient to be transfectable with CD38-targeted tLNP. Similarly, pursued antigen CD19 density on autoimmune patient PBMCs was sufficient for binding by anti-CD19 CARs. Samples from patients on B cell depleting therapies were not included in this analysis due to lack of sufficient cell number.

Phenotypically, the composition of major immune cell populations was comparable across subjects with and without an autoimmune disease (FIG. 11B) other than the absence of B cells in donors previously treated with rituximab. Similarly, the proportions of subsets of CD4+ T cells, CD8+ T cells, and monocytes were comparable between diseased and healthy donors (FIGS. 11C, 11D, and 11E, respectively).

TABLE 14
Patient Characteristics

| | | | | | | Concurrent Medication |
|---|---|---|---|---|---|---|
| | Donor | | | Sex | | (during blood/PBMC |
| Disease | ID | Antibody | Age | (M/F) | Race | collection) |
| Anti- | 7 | anti-Jo1 | 49 | F | White | Prednisone 50 mg/d, |
| synthetase | | | | | | Rituximab, |
| syndrome | | | | | | Mycophenolate 1500 |
| | | | | | | mg/12 hrs, IVIG |
| | | | | | | 2 g/kg/monthly |
| | 8 | anti-PL7 | 38 | F | White | IVIG 1 g/Kg/monthly, |
| | | | | | | Tacrolimus 2 mg/12 hrs, |
| | | | | | | Prednisone 20 mg/d |
| | 9 | anti-Jo1 | 43 | F | Black | Rituximab/4 infusions a |
| | | | | | | year, Prednisone |
| | | | | | | 3 mg/every 48 hrs, |
| | | | | | | Methotrexate 15 |
| | | | | | | mg/weekly |
| Dermato- | 4 | anti-NXP2 | 32 | F | Black | Prednisone, Tofacitinib |
| myositis | | | | | | 5 mg 2 times a day |
| | 5 | anti-TIF1 | 55 | F | White | Mycophenolate |
| | 6 | anti-Mi2 | 77 | M | White | Tofacitinib 5 mg 2 times |
| | | | | | | a day, Prednisone |
| | | | | | | 20 mg/d |
| Immune- | 10 | anti- | 51 | F | Black | Methotrexate 15 |
| mediated | | HMGCR | | | | mg/weekly, Prednisone |
| necrotizing | | | | | | 15 mg/d |
| myopathy | 11 | anti- | 63 | M | White | Rituximab 1 g, |
| (IMNM) | | HMGCR | | | | Prednisone 40 mg/d, |
| | | | | | | Methotrexate 17 |
| | | | | | | mg/weekly, IVIG |
| | | | | | | 2 g/kg/monthly |
| | 12 | anti- | 66 | F | Black | Prednisone 30 mg/d, |
| | | HMGCR | | | | Mycophenolate 500 |
| | | | | | | mg/d, Methotrexate 15 |
| | | | | | | mg/weekly, IVIG |
| | | | | | | 2 g/kg/monthly |
| Multiple | 19 | | 33 | M | Hispanic/ | |
| Sclerosis | | | | | Latino | |
| Rheumatoid | 20 | | 42 | F | White | Arava, Folic Acid, |
| Arthritis | | | | | | Methotrexate, Metopolol, |
| | | | | | | Humera, Enbrel |
| | 21 | | 62 | F | White | Celebrex, Humira |
| | | | | | | Injection, Methotrexate, |
| | | | | | | Amlodipine, Atenolol, |
| | | | | | | Folic Acid, Mehotrexate, |
| | | | | | | Metoprolol |
| | 22 | | 56-57 | F | White | Amlodipine, Atenelol, |
| | | | | | | Celebrex, Humira |
| | | | | | | injection, Methotrexate, |
| | | | | | | Claritin, Gabapentin, |
| | | | | | | Hydroxychloroquine |
| | | | | | | (Plaquenil), Meloxicam, |
| | | | | | | Tramadol |
| | 23 | | 41 | F | White | |
| | 24 | | 50 | F | White | |
| | 25 | | 69 | F | White | |
| Scleroderma | 26 | | 56 | F | White | none |
| | 27 | | 77 | F | White | Prednisone, daily tablet |
| | 28 | | 85 | F | White | Cellcept, Plaquenil |
| | 29 | | 61 | M | White | Prednisone |
| | 30 | | 71 | F | White | none |
| | 31 | | 71 | F | White | Mycopheolate/Norvasc |
| | 32 | | 43 | F | White | Cellcept |
| | 33 | | 73 | F | White | none |
| Systemic | 2 | | 55 | F | Black | Levothyroxine 112 mcg |
| lupus | | | | | | po QD, Norvasc 5 mg po |
| erythematosus | | | | | | QD, Spironolactone/BID, |
| (SLE) | | | | | | Vitamin D/QD, |
| | | | | | | Zetia/10 mg/QD, |
| | | | | | | gabapentin/200 mg/PRN, |
| | | | | | | Omeprazole 40 mg QD |
| | 3 | | 47 | F | Black | Hydroxyzine/25 mg/BID |
| | 1 | | 52 | F | White | Tramadol/50 mg/PRN, |
| | | | | | | Lisinopril/5 mg/QD, |
| | | | | | | Effexor/300 mg/QD, |
| | | | | | | Ibuprofen/600 mg/PRN, |
| | | | | | | Depo Provera/every |
| | | | | | | 3/mo, Zomig/25 mg/PRN, |
| | | | | | | Gabapentin/300 mg/TID, |
| | | | | | | Flexeril PRN, Metformin |
| | | | | | | 500 mg BID, Metoprolol |
| | | | | | | 200 mg QD, Diltiazem |
| | | | | | | 180 mg QD, Atorvastatin |
| | | | | | | 40 mg QD, Cevimeiline |
| | | | | | | sjorgrems |
| | 34 | | 26 | F | White | Aspirin, Wellbutrin, |
| | | | | | | Methotrexate, Lexapro, |
| | | | | | | Hydroxychloroquine, |
| | | | | | | Folic Acid, Pepcid, etc. |
| | 35 | | 60 | F | Non- | Diclofenac gel, |
| | | | | | Hispanic/ | Wellbutrin, Dulox |
| | | | | | Non- | |
| | | | | | Latino | |
| | 36 | | 61 | F | Non- | Duloxetine (Cymbalta), |
| | | | | | Hispanic/ | pramipexole, bupropion, |
| | | | | | Non- | Hydrochlorothiazide, |
| | | | | | Latino | singular, albuterol, |
| | | | | | | olmesartan, omeprazole, |
| | | | | | | Myrbetriq, trospium |
| | 37 | | 69 | F | White | Cymbalta, |
| | | | | | | Hydroxychloroquine, |
| | | | | | | Levothyroxine, |
| | | | | | | Lisinopril, Mobic, |
| | | | | | | Prilosec |
| | 38 | | 57 | M | White | Toprol-XL, Albuterol |
| Sjogren's | 39 | | 77 | F | White | Acyclovir 400 mg, |
| Syndrome | | | | | | Aspirin 81 mg, |
| | | | | | | Alprazolam 1 mg, |
| | | | | | | Atenolol 50 mg, Baclofen |
| | | | | | | 10 mg, Vitamin B12 |
| | | | | | | 1000 mcg, |
| | | | | | | Dexamethasone |
| | | | | | | 0.5 mg/5 ml, Vitamin D2 |
| | | | | | | 50,000 IU, Fluticasone |
| | | | | | | 50 mcg, Folic Acid 1mg, |
| | | | | | | Furosemide 40 mg, |
| | | | | | | Hydrocodone |
| | | | | | | Acetaminophen 10 mg- |
| | | | | | | 325 mg, |
| | | | | | | Hydroxychloroquine |
| | | | | | | 200 mg, Methotrexate |
| | | | | | | Sodium 2.5 mg, |
| | | | | | | Mirtazapine 15 mg, |
| | | | | | | Omeprazole 20 mg, |
| | | | | | | Potassium Chloride ER |
| | | | | | | 10 meq |
| Healthy | 15 | | 54 | F | Black | None |
| Donor | 13 | | 61 | F | White | Gabapentin PRN, |
| | | | | | | Topamax PRN |
| | 14 | | 38 | F | White | Vitamin/QD |
| | | | | | | Lexapro 20 mg QD |
| | | | | | | Lamictal 150 mg QD |
| | 16 | | 64 | F | Black | |
| | 17 | | 31 | F | White | |
| | 18 | | 57 | M | Native | |
| | | | | | American | |
| | 40 | | 28 | F | White | |
| | 41 | | 42 | F | White | |
| | 42 | | 57 | F | White | |
| | 43 | | 82 | F | White | |
| | 44 | | 55 | F | White | None |
| | 45 | | 61 | F | White | Omeprazole |
| | 46 | | 59 | M | White | None |
| | 47 | | 52 | F | White | |
| | 48 | | 54 | M | White | |
| | 49 | | 51 | F | White | |
| | 50 | | 31 | M | Hispanic/ | |
| | | | | | Latino | |
| | 51 | | 44 | F | White | |
| | 52 | | 71 | F | White | |
| | 53 | | 34 | M | Black | |
| | 54 | | 34 | M | Black | |
| | 55 | | 34 | M | White | |
| | 56 | | 36 | F | White | |

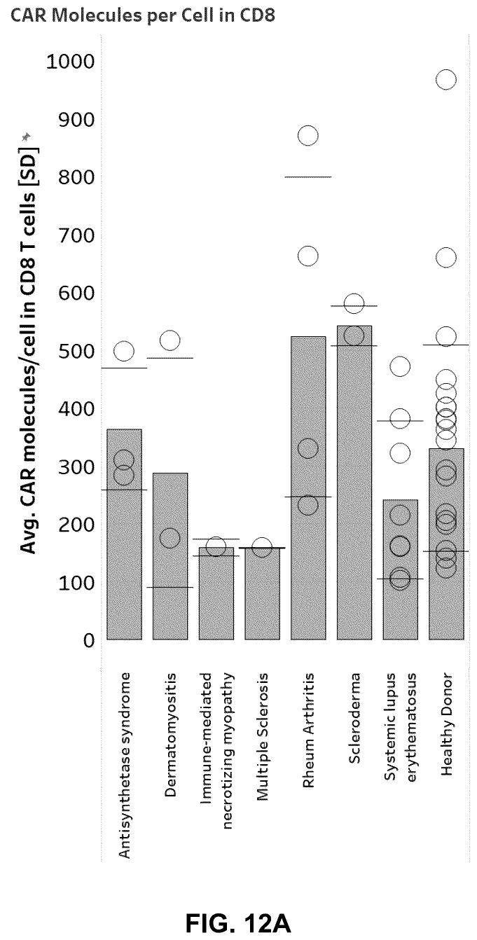

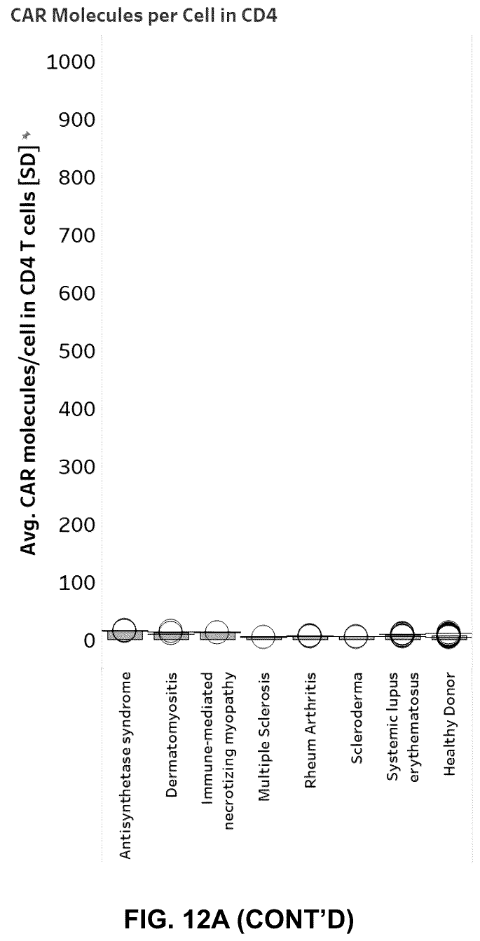

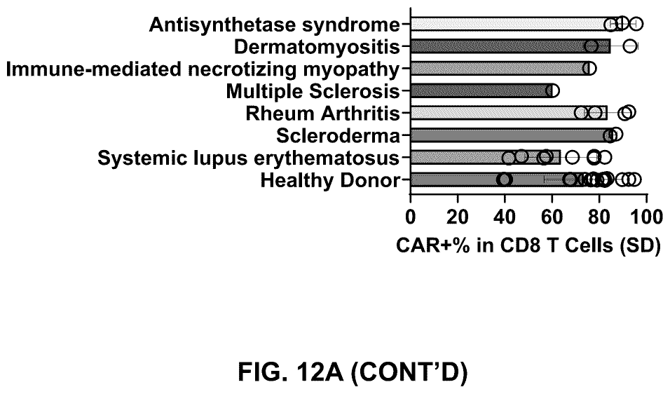

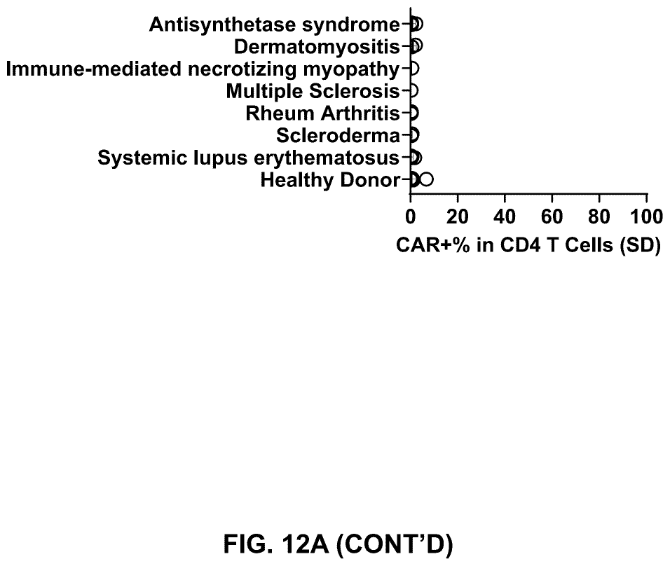

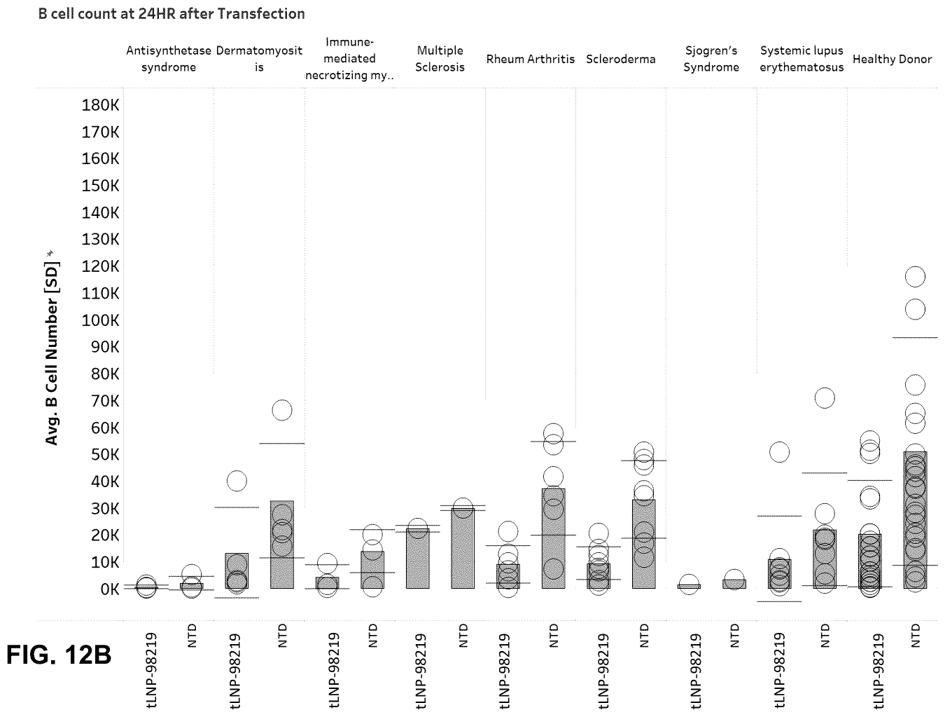

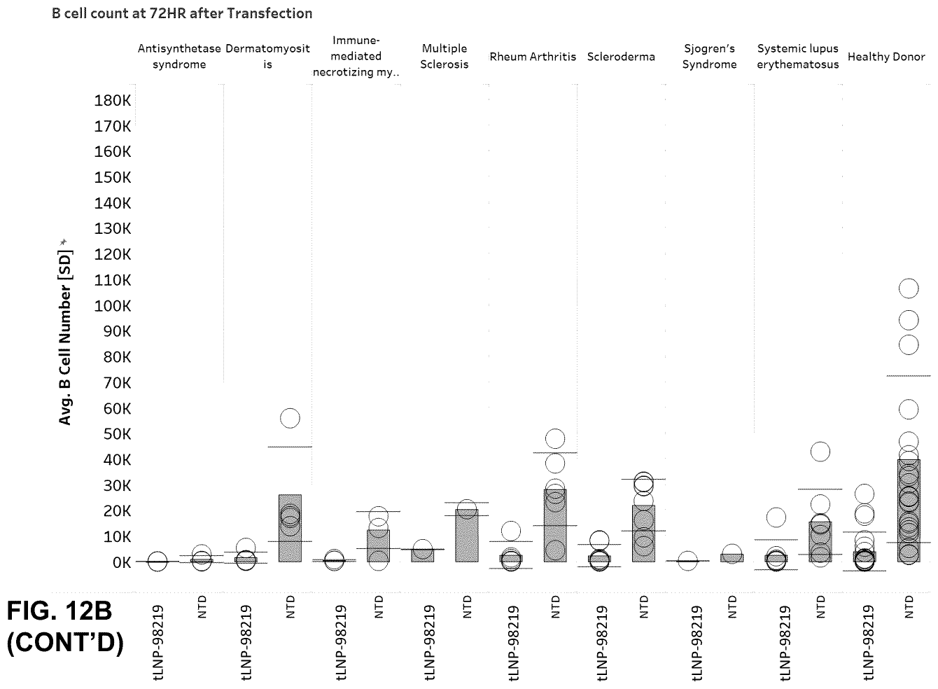

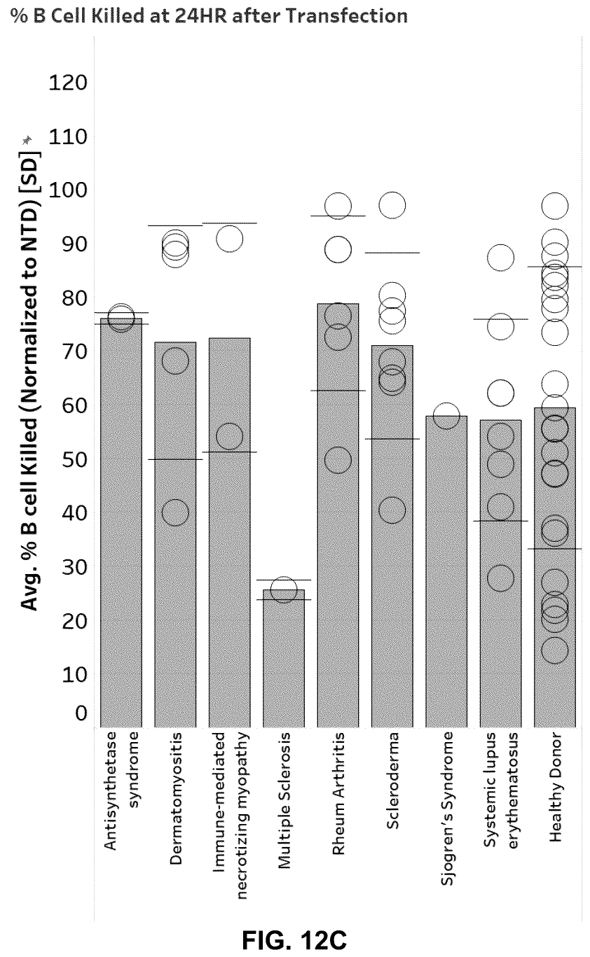

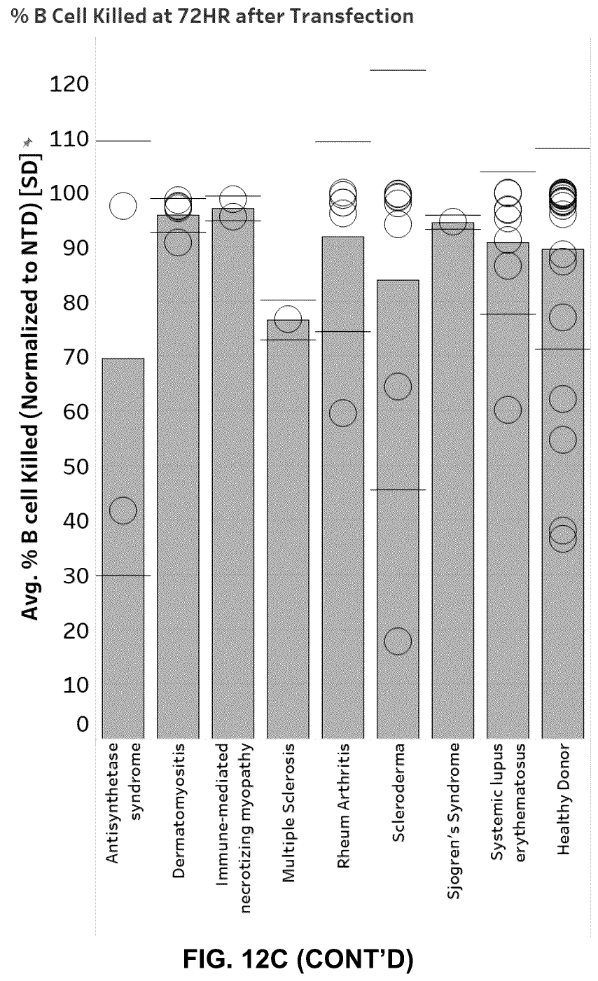

T cells (0.2 million) isolated from PBMC from autoimmune disease patients and healthy donors were transfected in vitro, in duplicate samples if there was a sufficient number of cells, for 1 hour with CD8-targeted tLNP encapsulating an mRNA encoding an anti-CD19 CAR containing 0.6 μg mRNA, without prior T cell activation or expansion. CAR expression was measured at 24 hours by binding of recombinant CD19 protein conjugated with PE to CAR expressing T cells using flow cytometry. In vitro engineering of unmanipulated lymphocytes with the CD8-targeted tLNP encapsulating an mRNA encoding an anti-CD19 CAR resulted in high levels (40-90%) of transfection and expression in the autoimmune patients' T cells of at least a similar level as in healthy T cells irrespective of prior treatments (FIG. 12A). There was no significant transfection of or expression in CD4+ T cells.

These reprogrammed T cells were cytotoxic against the autologous primary B cells present in the PBMC. The B cell population was measured by flow cytometry with anti-CD20 antibody staining and B cell number was counted by adding counting beads during flow cytometry staining and acquisition. Total numbers of B cells per well were reduced as compared to untransfected controls at 24 hours post transfection and to a greater extent—in some samples, eliminated—at 72 hours post transfection (FIG. 12B). The proportion of B cells killed was calculated as Percentage of B cells Killed=100% x (B cell number in non-transfected control—B cells number in tLNP treated group)/(B cell number in non-transfected control). At 72 hours post transfection the proportion of B cells killed approached 100% in many samples (FIG. 12C). In samples from Anti-synthetase syndrome (AS 7), AS 9, and IMNM 11 donors, there were no or few B cells due to prior treatment of those donors with rituximab. Samples with less than 100 B cells per 4×10⁵ total PBMC at baseline were excluded from B cell killing analyses. These data indicated B cells from autoimmune subjects can be killed by CAR-T cells to a similar degree as B cells from healthy donors.

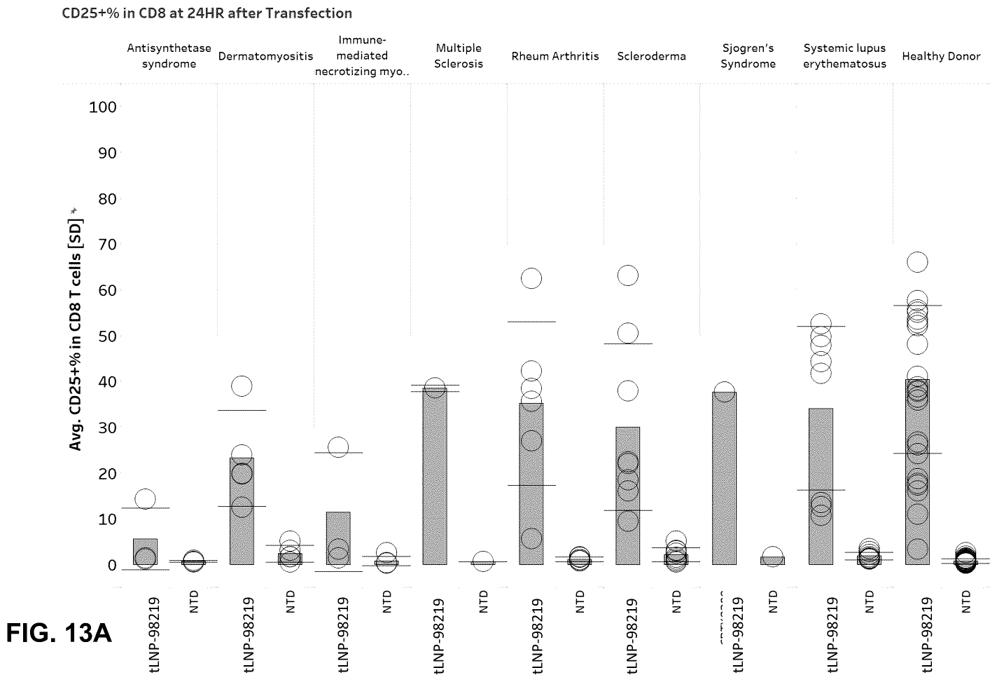

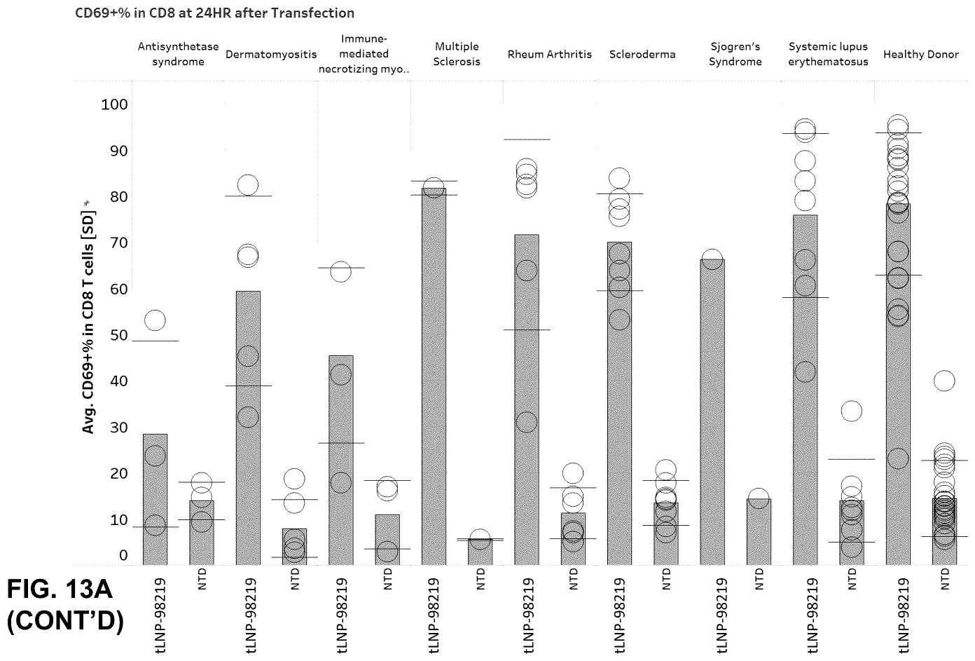

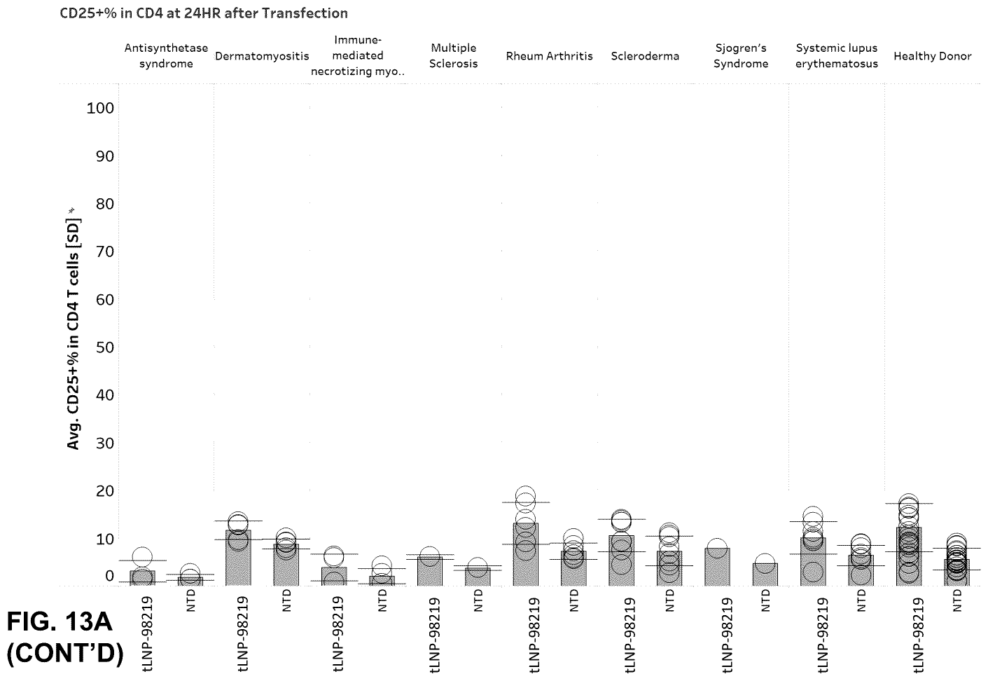

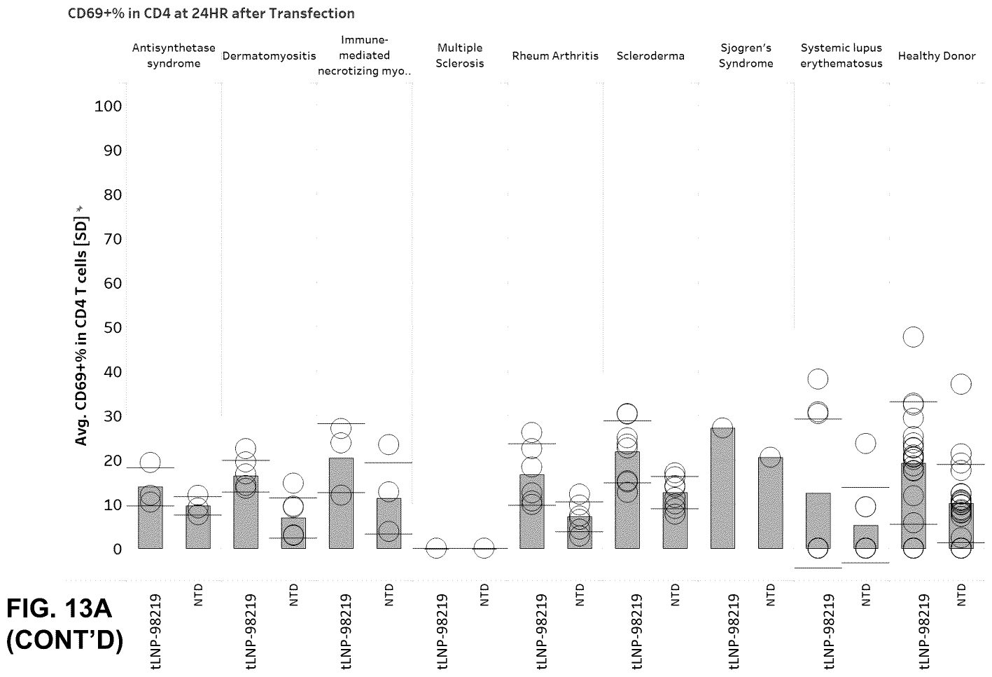

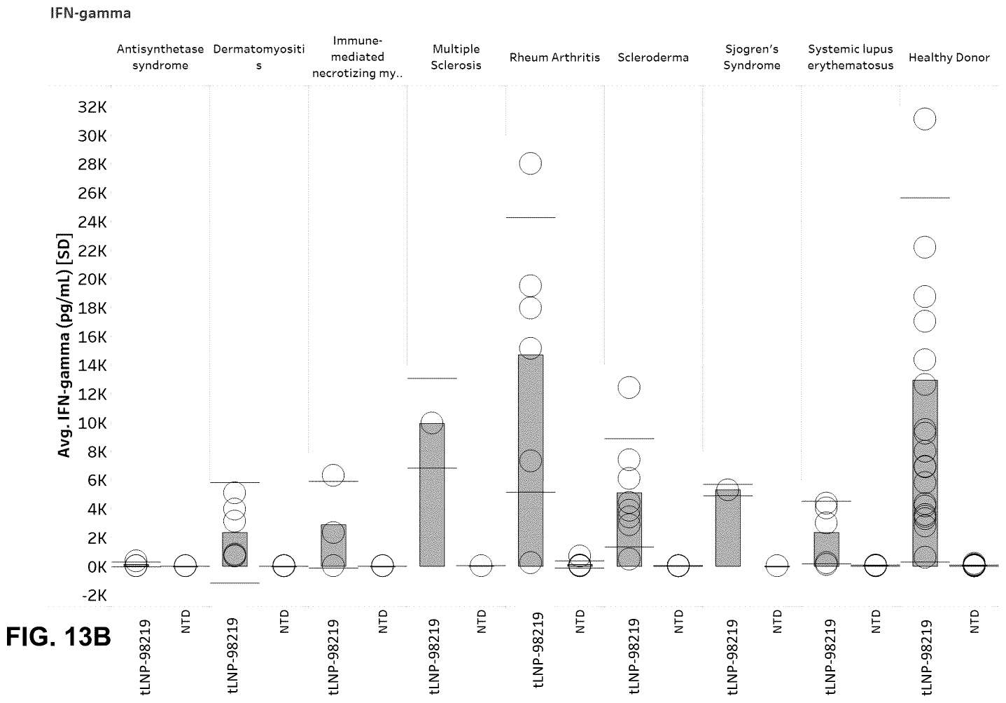

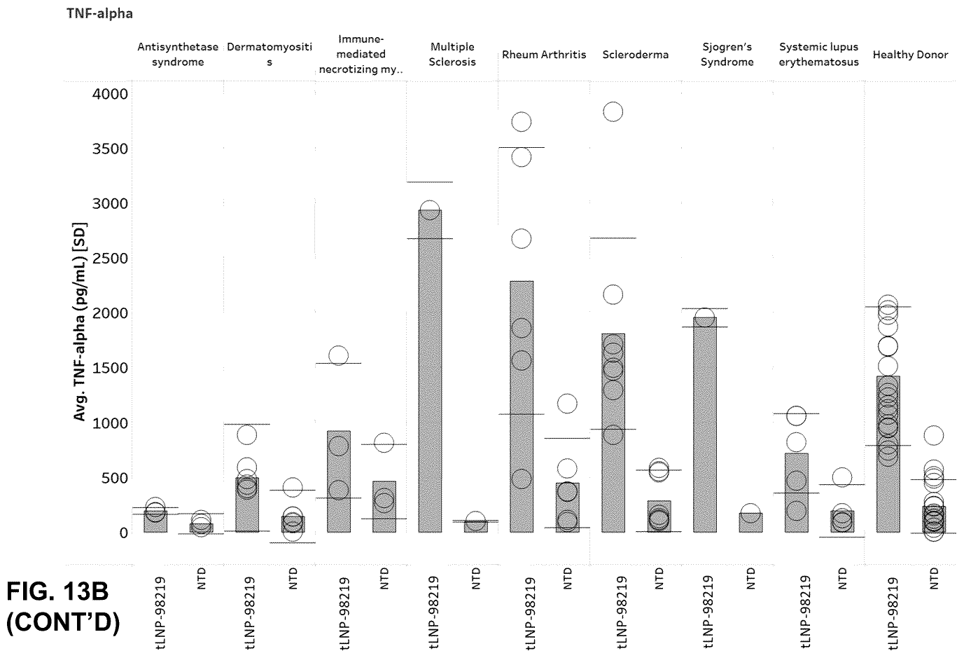

The CAR-T cells exposed to the autologous primary B cells present in the PBMC were stimulated by antigen engagement and expressed activation markers, assayed by antibody staining for CD69 and CD25 together with other T cell lineage markers (such as CD4 and CD8) at 24 hours post transfection (FIG. 13A). These data indicated all tLNP transfected disease and healthy CD8+ T cells expressed increased levels of multiple activation markers, such as CD69 and CD25, compared to untransfected controls. The low levels of increased activation marker expression observed in the CD4+ population was consistent with a bystander effect.

Cytokine secretion by reprogrammed T cells upon exposure to the autologous primary B cells present in the PBMC was also measured by collecting supernatant from the PBMC cultures at 48 hours post transfection and assaying using the NHP XL Cytokine Luminex Performance Premixed Kit (Catalog #: FCSTM21). These data indicated CAR-T cells generated from T cells obtained from subjects with an autoimmune disease can make cytokines upon B cell engagement of cognate CD19 antigen (FIG. 13B).

In the presence of syngeneic B cells expressing cognate CD19 antigen, these tLNP-engineered CAR-T cells activated rapidly, produced cytokines, engaged and rapidly eliminated the B cells over a 72 hour interval. The phenotypic characteristics and functionality of the engineered T cells from autoimmune disease patients and healthy subjects were comparable.

The tLNP effectively delivered the mRNA and reprogrammed CD8⁺ T cells from autoimmune disease patients to highly express the anti-CD19 CAR, irrespective of prior treatments including immune suppressive agents, and mediated rapid eradication of primary B cells in vitro.

These data promise that the T cells of autoimmune patients can be effectively transfected and reprogrammed through in vivo engineering with T cell-targeted tLNPs to deplete B cells and thereby treat B cell involved autoimmune diseases.

### Example 11

### Potentiation of Transfectability, Expression, and Function of T cells by Prior Exposure to Cognate Antigen while Transiently Expressing a CAR

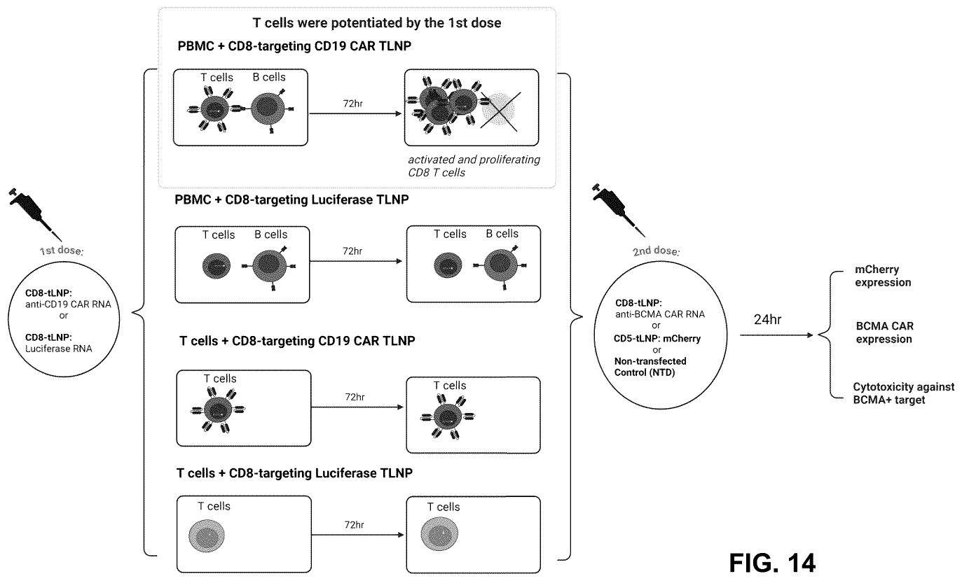

To demonstrate the potentiation effect underlying immune engineering amplification, human PBMCs and pan T cells were first transfected with tLNPs delivering an mRNA-encoded anti-CD19 CAR or various control reagents with or without exposure to cognate antigen, and 72 hours later transfected with tLNPs delivering an mRNA-encoding anti-BCMA CAR or various control reagents. CAR expression and functionality following the second transfection, measured as tumor cell killing, were assessed allowing the role and importance of the different components to be elucidated. The overall experimental scheme is illustrated in FIG. 14.

PBMC and pan T cells each from two human donors were thawed and transfected with CD8-targeted tLNPs encapsulating mRNA encoding either an anti-CD19 CAR (RM_61461) or luciferase. Transfection efficiency was measured by flow cytometry 24 hours after transfection to confirm successful transfection. Transfection efficiency of CD8+ cells with the anti-CD19 CAR was ˜75% for the PBMCs and ˜87% for the pan T cells (data not shown). The cells were re-transfected 72 hours after the first transfection, this time with CD8-targeted tLNPs encapsulating mRNA encoding an anti-BCMA CAR or CD5-targeted tLNPs encapsulating mRNA encoding mCherry. tLNPs were mixed well and incubated with cells for 1 hour inside a 37° C. CO2 incubator and then washed 3 times with culture medium. Cells were kept inside 37° C. CO2 incubator until ready for CAR expression analysis or functional assessment. CAR and mCherry expression were analyzed by flow cytometry 24 hours after the 2^{nd} transfection (96-hours after the first transfection). 24 hours after the second transfection, transfected cells were co-cultured with tumor cell lines (BCMA+ CD19− RPMI8226 and K562 constitutively expressing luciferase and GFP) at E:T ratios of 6:1, 2:1, and 0.6:1 to assess cytotoxicity against BCMA-expressing cells. Cytotoxic activity was monitored by loss of GFP area under the IncuCyte live-cell analysis system (Sartorius) for a total of ˜96 hours. In flow cytometry dead cells were excluded by Fixable Aqua live/dead dye, CD4-BV650 and CD8-BV421 were used to gate CD4-expressing and CD8-expressing T cells. CAR expression was analyzed by staining cells with a PE-conjugated human BCMA recombinant protein (ACRO Biosciences). mCherry expression was analyzed in the PE/Texas Red channel.

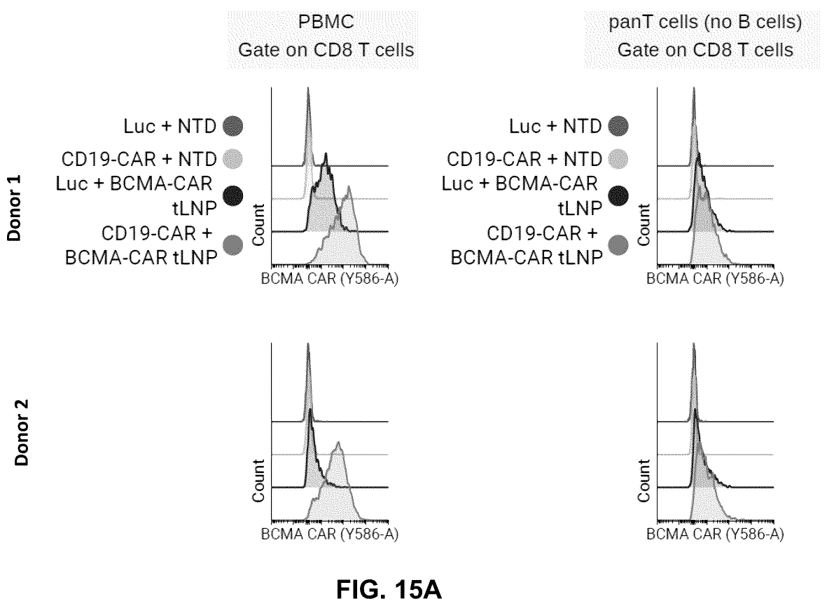

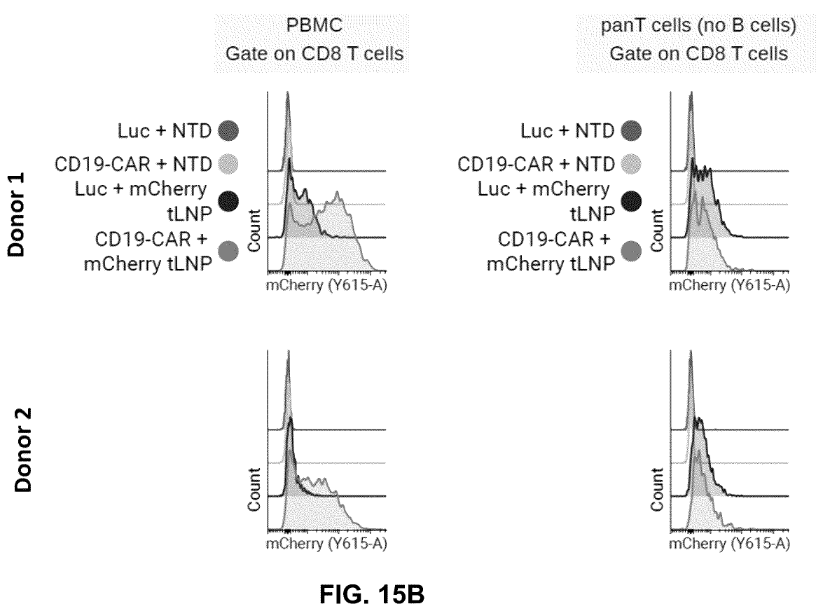

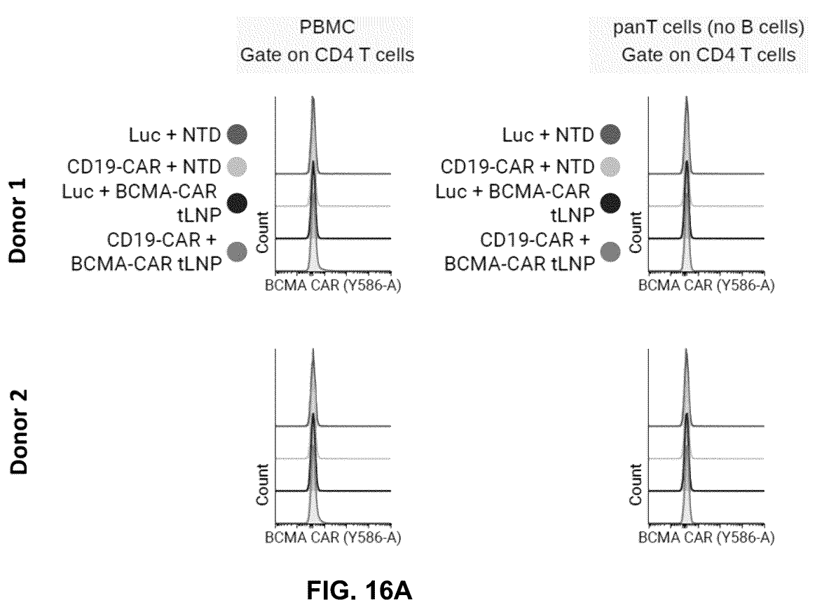

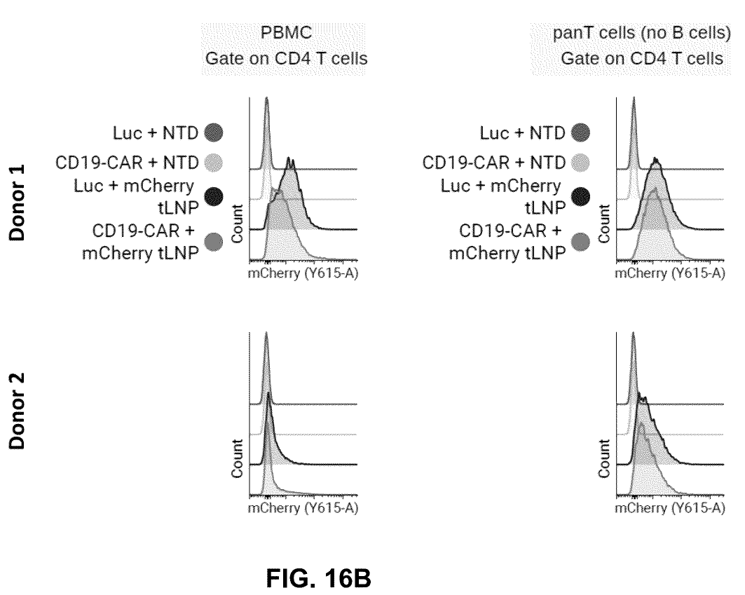

As seen in FIG. 15A, for transfection of PBMC anti-BCMA CAR expression (following the second transfection) was higher for cells that had been transfected with the anti-CD19 CAR in the first transfection than it was for cells that had been transfected with luciferase in the first transfection (left histograms) while for pan T cells (lacking B cells and thus lacking cognate antigen for the anti-CD19 CAR) there was no elevation of expression level of the anti-BCMA CAR in the cells that had received the anti-CD19 CAR in the first transfection. A similar pattern of expression levels was seen when the second transfection conferred mCherry expression with only a first transfection in the presence of the CAR's cognate antigen leading to an elevated level of expression (FIG. 15B). Thus, T cells expressing a CAR in an environment providing cognate antigen are able to express higher levels of protein (anti-BCMA CAR or mCherry) upon re-transfection as compared to T cells that had not experienced recent antigen receptor engagement. There was no expression of the BCMA CAR in CD4+ cells as the tLNPs were targeted to CD8 (FIG. 16A). For mCherry payloads, the tLNPs were targeted an anti-CD5 antibody and thus expression was seen in CD4+ cells, though there was no elevation of mCherry expression level associated with the prior exposure to the tLNP with the anti-CD19 CAR mRNA in the first transfection (FIG. 16B).

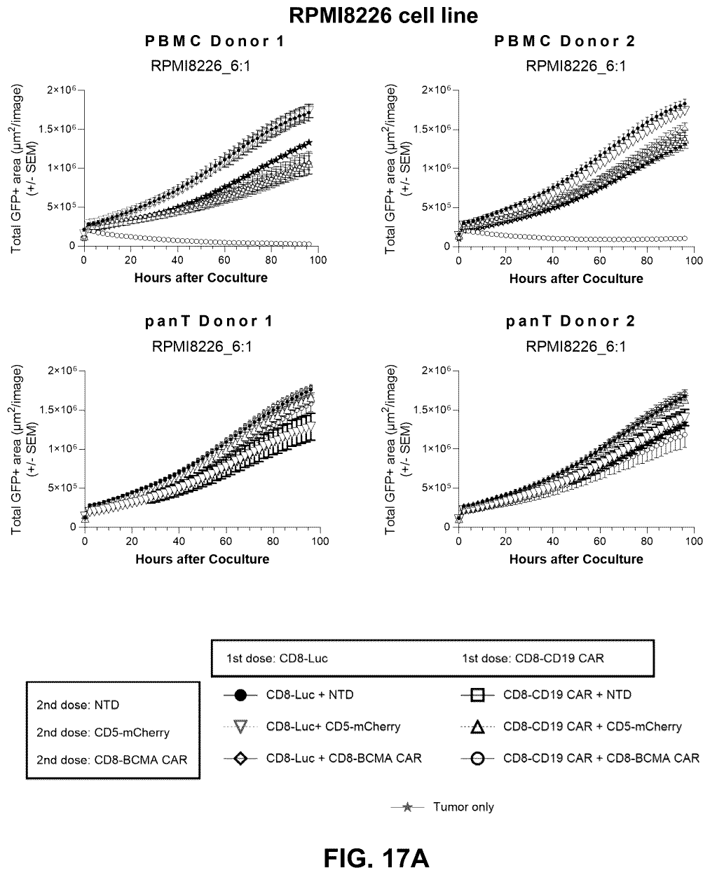

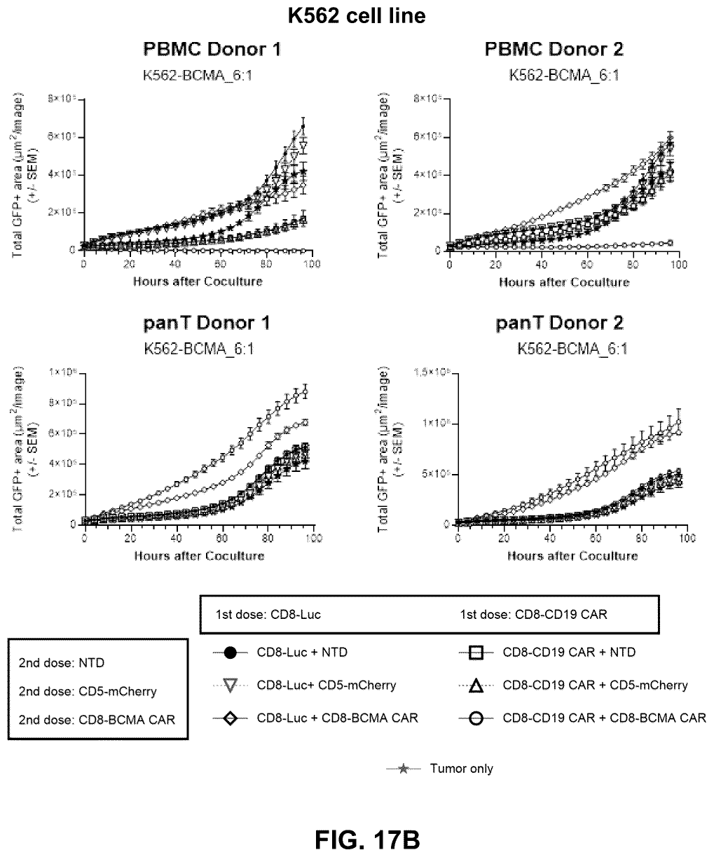

As seen in FIGS. 17A and 17B, cells that received primary transfection with the CD8-targeted tLNPs encapsulating mRNA encoding the anti-CD19 CAR and were exposed to target CD19+ cells (that is, the PBMC cultures) exhibited robust cytotoxicity to both BCMA-expressing tumor cell lines. This cytotoxicity is associated with expression of the anti-BCMA CAR but as these tumor cells are CD19−, there can be no contribution to killing from any remaining anti-CD19 CAR. FIGS. 17A and 17B show results using an effector to target ratio (E:T) of 6:1. At an E:T of 2:1 there was still substantial killing but only minimal inhibition of tumor growth at an E:T of 0.6:1 (data not shown).

Minimal if any inhibition of tumor growth was exhibited by cells from any of the other transfection combinations. Enhanced tumor growth was observed in some of the other transfection combinations, likely reflecting responsiveness of the tumor cell line to cytokines being secreted by the tumor cells. To the extent that tumor growth was reduced (beyond experimental variation) in cultures without the anti-BCMA CAR it was believed to be the result of antigen-independent killing by highly activated T cells.

These data clearly demonstrated that the exposure of CAR-T cells to cells expressing cognate antigen activated them in a manner that led to greater expression of a subsequently transfected agent and greater functional activity (cytotoxicity). That is, T cells transiently expressing a CAR were potentiated by exposure to cells expressing the antigen recognized by the CAR so that subsequent transfection led to greater expression and functional activity or the re-transfected T cells. This experiment also indicates that an anti-B cell CAR (e.g., anti-CD19, anti-CD20) can be used as the initial potentiating/activating dose to amplify a second immune engineering agent administered in subsequent doses, such as a CAR directed to any other disease-related antigen. The attendant depletion of B cells resulting from the initial anti-B cell CAR can be expected to reduce the risk, occurrence, or severity of any anti-drug antibody response specific for the second reprogramming agent.

### Example 12

### Rapid and Preferential In Vivo Engineering of CAR-T Cell in Blood and Tissue

This study assessed the pharmacokinetics of in vivo CAR expression in various tissues and cell types. Wild-type female C57BL/6 mice were dosed with either 100 μL phosphate buffered saline (PBS) or 1.5 mg/kg tLNP-9m8219 by tail vein intravenous (IV) injection. tLNP-9m8219 differed from tLNP-98219 in that the binding moiety of the tLNP recognized mouse CD8 instead of human and NHP CD8. Five (5) mice each were then sacrificed at 6 hours, 24 hours, 48 hours, and 72 hours. PBS-treated mice were also sacrificed at 6 hours. Blood, spleen, lymph nodes, and bone marrow were collected, processed to single cell suspensions, and analyzed for immune cell populations and CAR expression by flow cytometry. For the purposes of measuring expression on the target cell population (CD8+ T cells), CD4− T cells were used due to decreased anti-CD8 antibody staining after dosing. This could have been due to downregulation of the CD8 receptor or masking of the receptor by the anti-mouse CD8 antibody on the tLNP. Since the CD8 staining is inconsistent between timepoints, and the CD4 and CD8 double negative (DN) population was small in the PBS treated animals, CAR expression was measured on total CD4− T cells as the most consistent way to represent expression on CD8+ T cells. CAR expression level was measured as median fluorescence intensity of CAR+ cells.

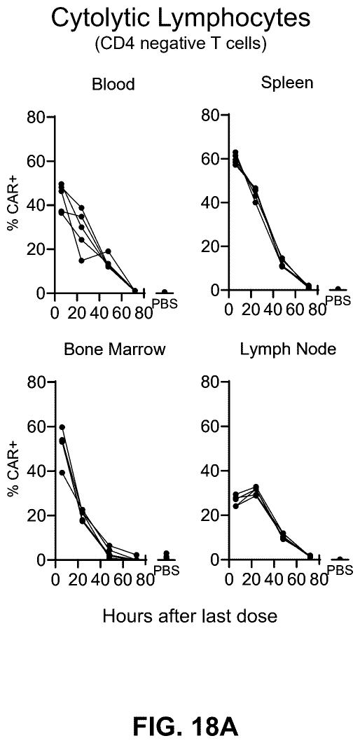

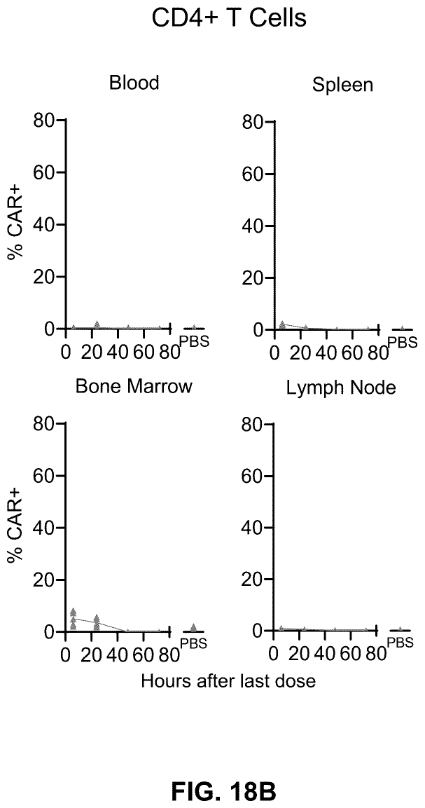

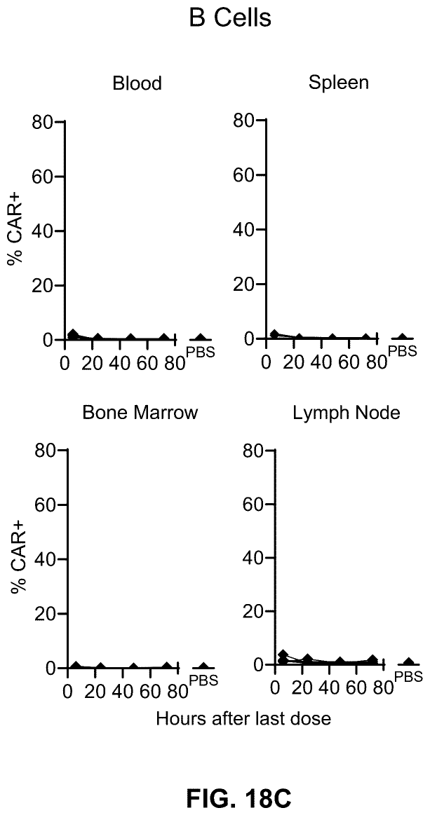

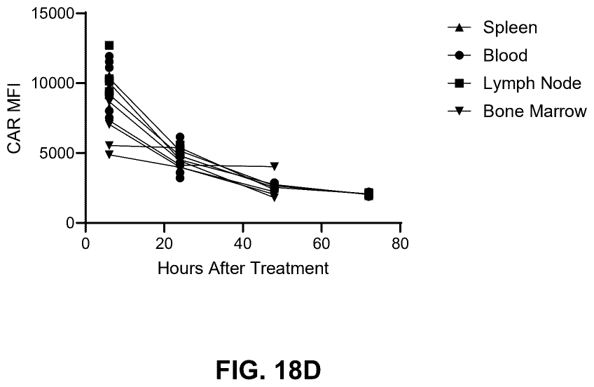

The percentage of cells expressing CAR in the spleen was greatest at the initial 6-hour timepoint and decreased to near baseline by 72 hours. Expression percentage was highest in the target CD4− T cell population (FIG. 18A) as expected, with expression in about 60% of CD4-T cells at 6 hours. Expression was also seen, but at a lower level, in NK cells and monocytes. Minimal expression above background was seen in CD4+ T cells (FIG. 18B) and B cells (FIG. 18C).

CAR expression was also measured in blood. Expression percentage again was greatest at the initial 6-hour timepoint after dosing, with between 35% and 50% of CD4− T cells expressing CAR (FIG. 18A). Low levels of expression (4-14%) were also seen in NK cells at 6 hours after dosing. No expression was seen in monocytes, CD4+ T cells (FIG. 18B) and B cells (FIG. 18C).

CAR expression was further measured in the bone marrow from femur. Similar to blood and spleen, expression percentage was greatest at the initial 6-hour timepoint after dosing and was highest in CD4− T cells (39-60%). Expression was also observed in NK cells (16-36%), monocytes (1-10%), and CD4+ cells (2-8%) (FIG. 18B). No expression was observed in B cells (FIG. 18C).

CAR expression was measured in lymph node tissue, where the peak of expression was at 24 hours after dosing as compared to 6 hours after dosing for spleen, blood, and bone marrow and the maximum expression level was lower. CD4− cells (FIG. 18A) and monocytes had similar percentages of CAR+ cells (˜30%). NK cells also expressed CAR (17-26%), and no expression was observed on in CD4+ T cells (FIG. 18B) and B cells (FIG. 18C). The delayed peak and lower expression level of CAR in lymph node is understood to reflect the absence of fenestrated vasculature reducing accessibility of this tissue to the IV administered tLNPs and suggests that these CAR+ cells are at least largely made up of T cells that trafficked into the lymph node rather than having become transfected there.

CAR expression level was greatest at the initial 6-hour timepoint and decreased thereafter in all four tissues (FIG. 18D).

Expression of the CAR in monocytes is likely due to the high phagocytic activity of these cells. Although theoretically the CD3ζ domain of the CAR could activate monocytes, they do not appear to play a substantial role in killing cells recognized by the CAR, at least in vitro (see Example 17, below).

Taken together, these data demonstrated that intravenous administration of CD8-targeted tLNPs leads to robust transfection and rapid, transient expression in the targeted lymphocytes in blood and peripheral organs of the immune system. Such transfection was highly preferential within the lymphocytic population with minimal expression in lymphocytes lacking CD8 expression, that is, B cells and CD4+ T cells.

### Example 13

### In Vivo Assessment of B Cell Depletion and CAR Expression of mRNA Construct Encoding Anti-CD19 CAR2 in PBMC Engrafted NSG (NSG-PBMC) Mouse Model

To evaluate the transfection efficiency and the ability of T cells transfected with an mRNA construct encoding an anti-CD19 CAR2 to kill B cells in vivo, CD8-targeted F9 tLNP encapsulating mCherry mRNA or RM_61461 mRNA (herein after tLNP98-mCherry and tLNP-98219, respectively) were injected intravenously into NSG immunodeficient mice engrafted with human PBMCs (tLNP-98mCherry treated or tLNP-98219 treated mice). This allowed characterization of the kinetics of T cell engineering and B cell depletion following treatment.

Immunodeficient NSG mice were injected with 10 million human PBMCs per animal. After 20 days of engraftment mice were evaluated for frequency of human CD45+ cells in circulation and staged in groups with similar averages. Day 21 post PBMC transfer, mice were dosed with a single intravenous (IV) injection via the tail vein of 30 μg/animal tLNP-98mCherry mRNA or tLNP-98219. Mice were sacrificed at different time points after dosing and their spleens harvested to assess transfection and B cell depletion.

Average human B cell frequencies (as a percent of human immune cells [CD45+]) in the spleens of mCherry treated mice at 1-, 3- and 6-hours post treatment were between 40-50%.

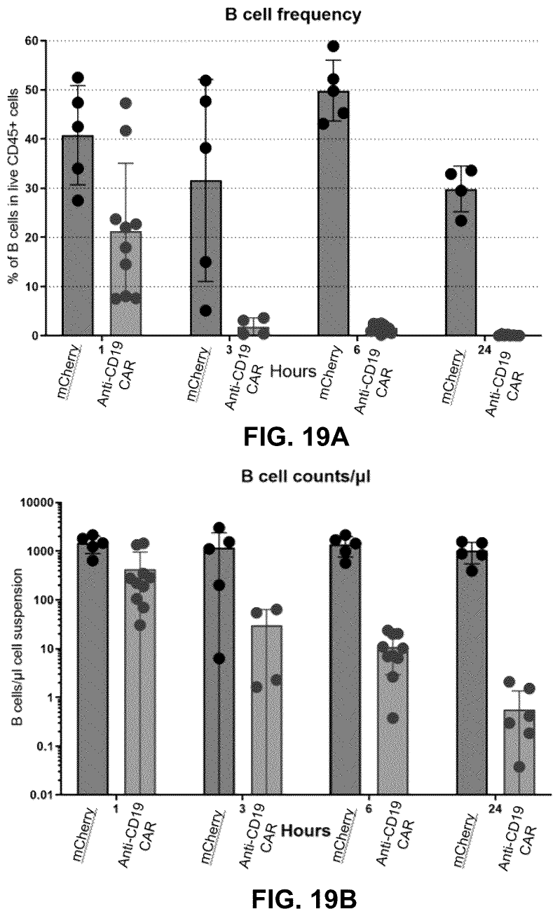

However, at 24-hours post treatment, the frequency of human B cells decreased to 30% (FIG. 19A). The total number of human B cells per microliter of sample (FIG. 19B) remained at an average of 1000 human B cells across all time points. In contrast, tLNP-98219 treated mice demonstrated rapidly decreasing human B cell frequencies over time, with a mean value of 22% human B cells observed at 1-hour post treatment (FIG. 19A), followed by almost complete human B cell depletion at 3-, 6-, and 24-hours post treatment. Similar findings were observed in the analysis of the total number of B cells per microliter of sample (FIG. 19B) where approximately 500 human B cells/μL were observed at 1 hour post treatment, followed by 10 B cells/μL at 3- and 6-hours post treatment. The lowest total cell counts were observed at 24 hours post treatment, with ≥2 human B cells/μL in all mice. The decrease in B cell numbers observed in tLNP-98219 treated mice indicated that expression of anti-CD19 CAR in CD8+ cells was responsible for B cell depletion.

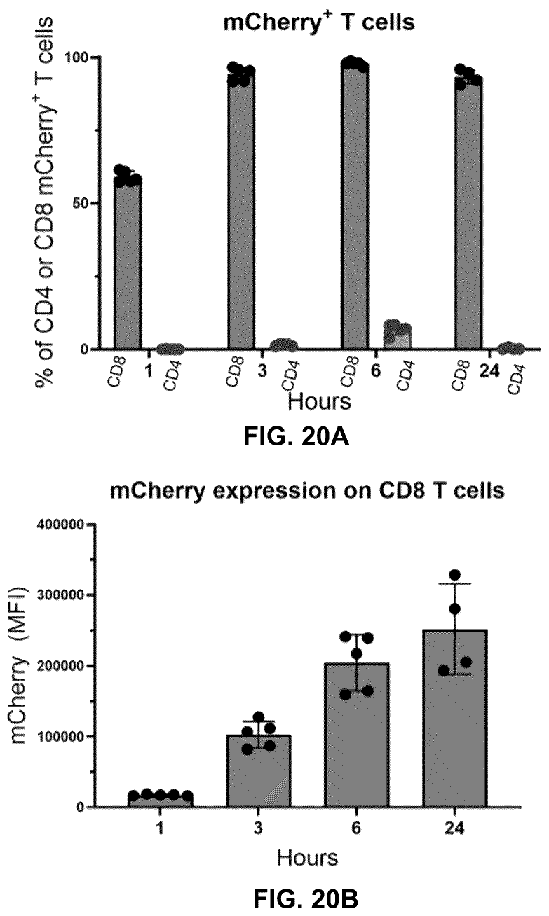

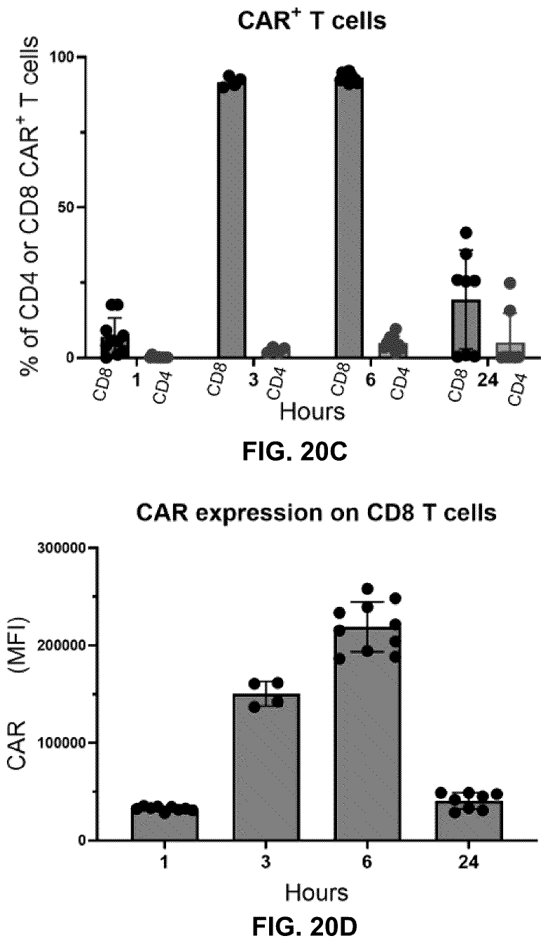

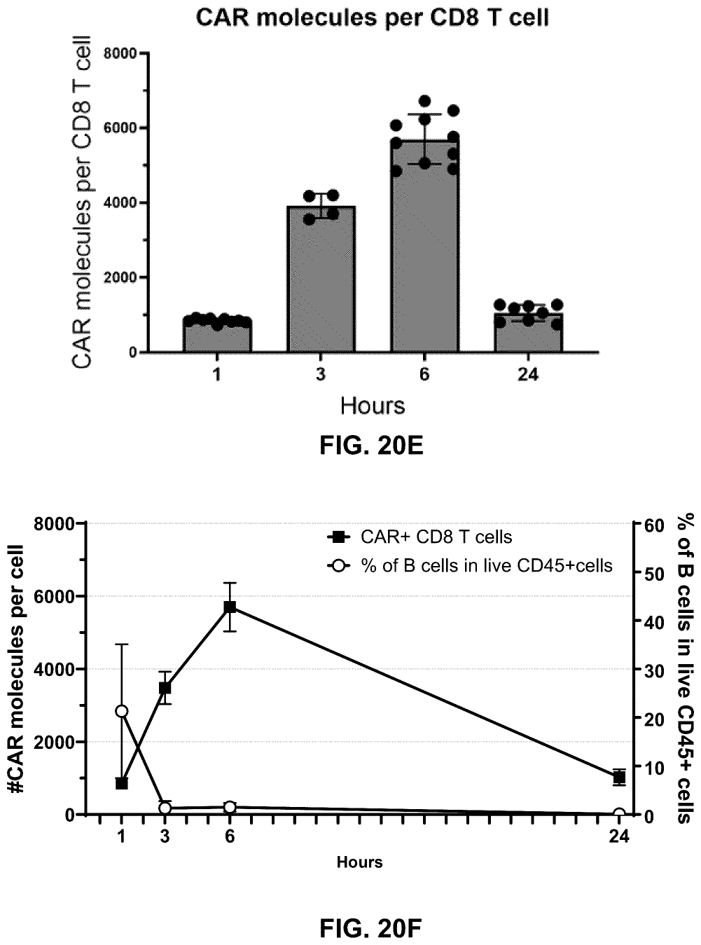

Expression of mCherry and CAR in splenic CD8+ T cells was evaluated. mCherry was expressed by 60% of the CD8+ T cells in mice treated with mCherry tLNP as early as 1 hour post treatment, and 97% of CD8+ T cells at 3 hours post treatment. The frequency of CD8+ T cells expressing mCherry remained at similarly high levels for 6- and 24-hours post treatment (FIG. 20A). Although this plateau was observed in the frequency of mCherry+ CD8+ T cells, the magnitude of mCherry expression, as measured by mean fluorescence intensity (MFI) (FIG. 20B), increased across time points.

In contrast, the frequency of human CD8+ T cells expressing the anti-CD19 CAR in mice treated with tLNP-98219 was approximately 6% at 1-hour post treatment, but then increased to 85% and 95% at 3- and 6-hours post treatment, respectively (FIG. 20C). Anti-CD19 CAR2 expression then dropped at 24-hours post treatment with a range from 0-40% of the CD8+T cells expressing the CAR (FIG. 20C). Correlating with these observations, expression of the anti-CD19 CAR as assessed by MFI demonstrated an increase of expression from 1- to 6-hours post treatment (peaking at 6 hours), with a decrease of expression at 24-hours post treatment (FIG. 20D). Quantification of CAR molecules per CD8+ T cell indicated that at the peak of expression in this study (6-hours post treatment), the number of anti-CD19 CAR molecules per CD8+ T cell were within the range of about 5000 and 7000 molecules per cell (FIG. 20E). B cell depletion correlated with the rise in expression level of anti-CD19 CAR (FIG. 20F).

Minimal expression of mCherry and CAR2 was observed in CD4+ cells, indicating specific CD8 cell targeting efficiency of the tLNP. mCherry expression in CD4 T cells averaged 0.02% at 1 hour post treatment, and increased to 1.4% average at 3 hours, peaking at 6.88% 6 hours post treatment. Finally, at 24 hours post treatment mCherry expression returned to low levels with an average of 0.09% of CD4 T (FIG. 20A).

CAR2 expression in spleen CD4 T cells was hardly detected 1 hour post treatment with only one mouse with 1% of CD4 T cells expressing the CAR. This was followed with a slight peaking at 6 hours post treatment where CAR was detected in a range between 1.8 and 9.6% of the CD4 CAR T cells. At 24 hours post treatment, 5 out of 10 mice presented lower than 0.2% of CAR+ CD4 T cells with two mice out of ten with 24.8 and 15.7% of CAR+ CD4 T cells. The averages CAR+ CD 4 T cells per time point were 0.196% at 1 hour post treatment, 2.52% at 3 hours, 4.96% at 6 hours and 5.89% 24 hours post treatment (FIG. 20C).

Expression of mCherry and CAR2 in CD4 T cells could not be evaluated in MFI terms nor molecules per cell due to the low number of CD4 T cells that were positive on each group (Less than 100 CAR2 or mCherry positive cells in the majority of treated animals).

The data obtained in this study indicated that tLNP-98219 can induce B cell depletion in the spleen of NSG-PBMC mice as early as 3 hours post treatment, with nearly all human B cells depleted from the spleen by 24 hours after treatment. Both tLNP-98219 and tLNP-mCherry showed preferential transfection of CD8+ T cells compared to CD4+ T cells. CAR expression in human CD8+ T cells peaked at 6 hours post treatment, with more than 90% of all CD8+ T cells in the spleen expressing the human anti-CD19 CAR with an average of 5000 to 7000 CAR molecules per cell. The data also showed that human anti-CD19 CAR expression is markedly reduced 24 hours post treatment, possibly due to interactions between the CAR and its cognate antigen.

### Example 14

### Determination of Minimum Effective Dosage of tLNP Encapsulating Anti-CD19 CAR mRNA In Vivo

To evaluate the minimum effective dosage of tLNP-98219, experiments similar to those described in Example 13 were carried out on NCG immunodeficient mice engrafted with human PBMCs, except that various dosages of tLNP-98219 were tested and the effects were measured at 6 hours post-treatment. 1.5 μg, 3 μg, 7.5 μg, 15 μg, and 45 μg dosages corresponded to about 0.075 mg/kg, about 0.15 mg/kg, about 0.375 mg/kg, about 0.75 mg/kg, and about 2.25 mg/kg, respectively, for mice of approximately 20 g. The dosages referred to the amount of mRNA being provided. The tLNPs were formulated with F9 lipid content.

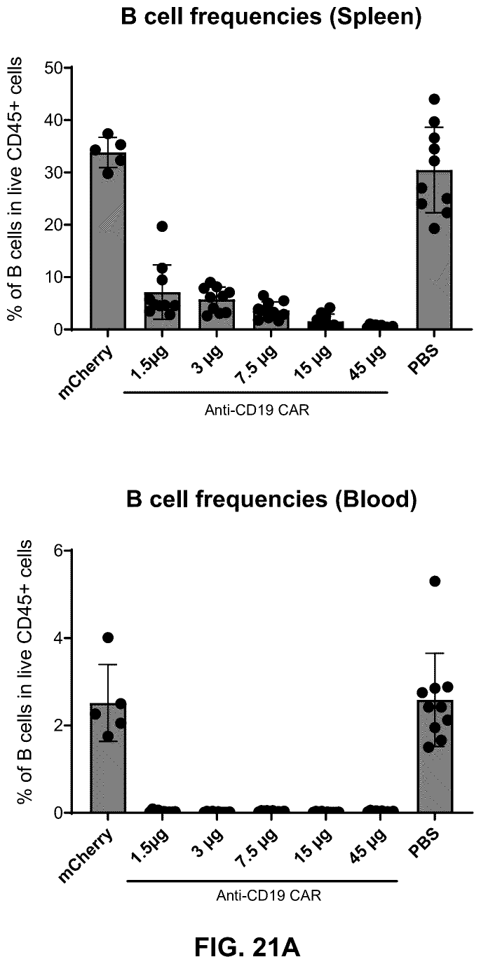

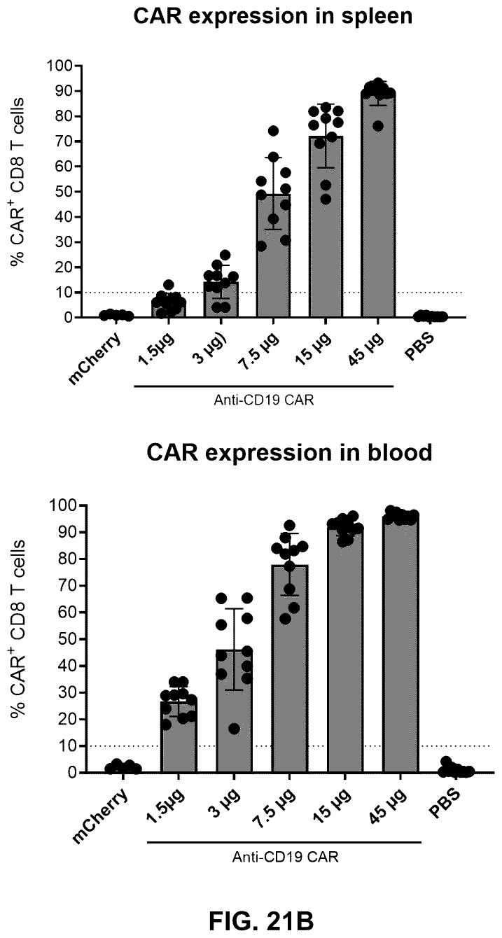

B cell frequencies at 6 hours post-treatment were equivalent in the PBS-treated and the mCherry-treated animals (FIG. 21A), indicating there was no impact of the tLNP delivery vehicle. Treatment with tLNP-98219 at any of the doses induced a complete depletion of all B cells in the blood. Analysis of the spleen, where there is higher B cell engraftment than that observed in the blood, demonstrated a dose response to tLNP-98219, with an almost complete B cell depletion in the tissue at doses of 15 μg and 45 μg per animal. These doses are the equivalent of 0.75 mg/kg and 2.25 mg/kg, respectively. In animals dosed with 7.5 μg and 3 μg, some B cells were still present in the spleen, but the B cell frequency remained below 10% of human CD45+ cells, compared to the mCherry and PBS controls with an average of 30-35% B cells. Finally, the lowest dose of 1.5 μg per animal (0.075 mg/kg) also showed a significant reduction, and only 3 out of 10 mice had B cells above 10% in the human CD45+ compartment.

In parallel, CAR expression was evaluated in CD8 T cells (FIG. 21B). In the tLNP-98219-treated groups, the frequency of CAR+ CD8 T cells was higher in the blood than in the spleen for all doses. However, in both blood and spleen, the frequency of CAR+ CD8 T cells demonstrated a dose dependency with higher engineering rates correlating with increased doses.

The engineering rates in the blood ranged from 30% at the 1.5 μg dose, to nearly 100% at the 45 μg dose, of CD8 T cells expressing CAR in blood.

The data obtained in this study indicate that a CD8-targeted tLNP encapsulating mRNA construct encoding anti-CD19 CAR2, tLNP-98219 can induce B cell aplasia in humanized NCG-PBMC mice 6 hours post-treatment with doses as low as 1.5 μg per mouse (approximately 0.075 mg/kg). Frequency of CAR expression and depletion of splenic B cells was dose dependent in the tested dosage range.

### Example 15

### Determination of Optimal Dose Regimen of tLNP Encapsulating Anti-CD19 CAR mRNA In Vivo

To evaluate the optimal dose regimen of tLNP-98219, experiments similar to those described in Example 14 were carried out, except with NSG immunodeficient mice engrafted with CD34+ human cells (purchased from Jackson Laboratory) and with a different dose and various dose regimens. Mice were treated intravenously (IV) with tLNP-98219, tLNP-982520 (CD8-targeted F9 tLNPs encapsulating mRNA encoding an anti-CD20 CAR), or tLNP-mCherry (control tLNP) either daily, every two days, or every three days, for a total of three treatments and 30 μg per animal per treatment. Starting at 24 hours post final treatment, and then weekly thereafter, peripheral bleeds were performed to evaluate the frequency of circulating human B cells.

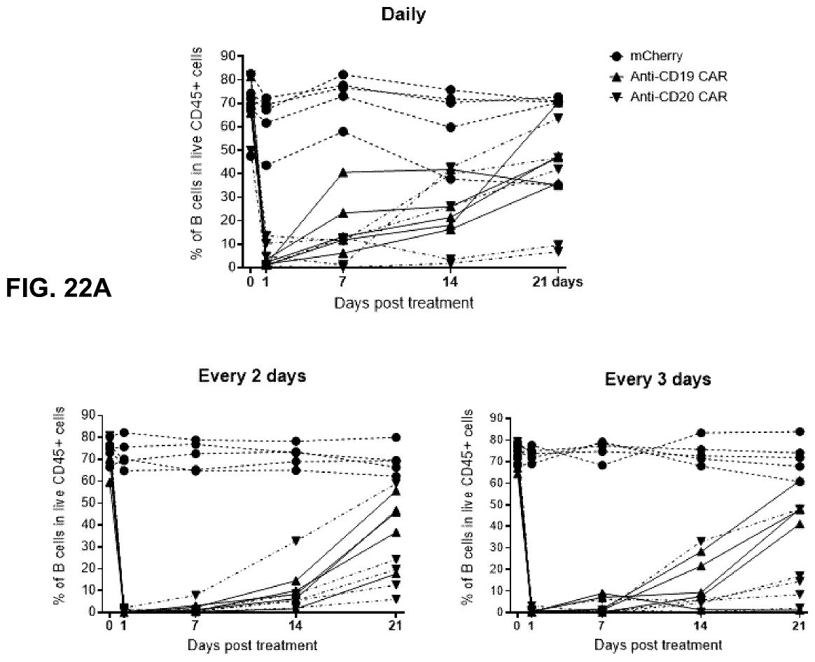

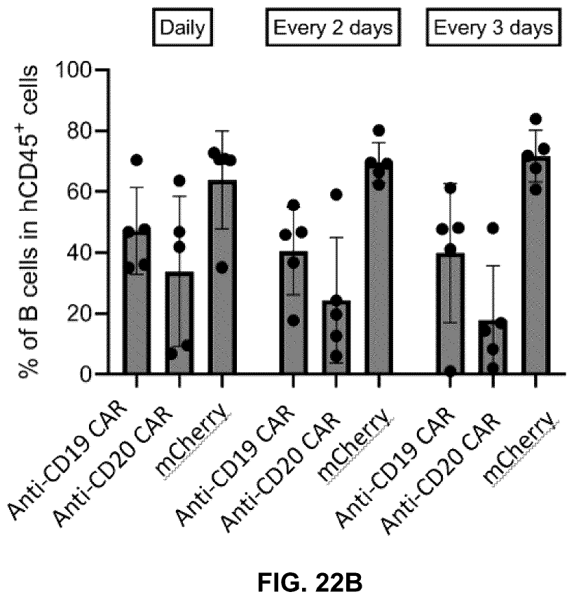

B cell frequencies were monitored longitudinally by flow cytometry in the blood of treated mice. All dose regimens with tLNP-98219 or tLNP-982520 induced a near complete B cell depletion 24 hours post third dose; there was no B cell depletion with any dose regimen with tLNP-mCherry (as expected). Treatment with tLNP-98219 or tLNP-982520 every two or three days resulted in a complete elimination of B cells from the peripheral circulation (FIG. 22A). Only the daily dosing of tLNP-98219 resulted in a complete elimination of B cells following daily dosing, whereas B cells were detectable in some of the tLNP-982520 treated mice.

Evaluation of the recovery of the circulating B cell frequencies demonstrated a marked increase at Day 7 post-treatment following daily dosing with either tLNP-98219 or tLNP-982520. In contrast, recovery of B cells following dosing every 2 or 3 days with the same test articles was limited at Day 7 post-treatment and only evident at Day 14 post-treatment (FIG. 22A). At Day 21 post-treatment, B cell frequencies remained lower than tLNP-mCherry-treated animals, particularly in those animals dosed every 2 or 3 days, with a trend towards lower B cell frequencies in tLNP-982520 (anti-CD20 CAR) treated mice (FIG. 22B).

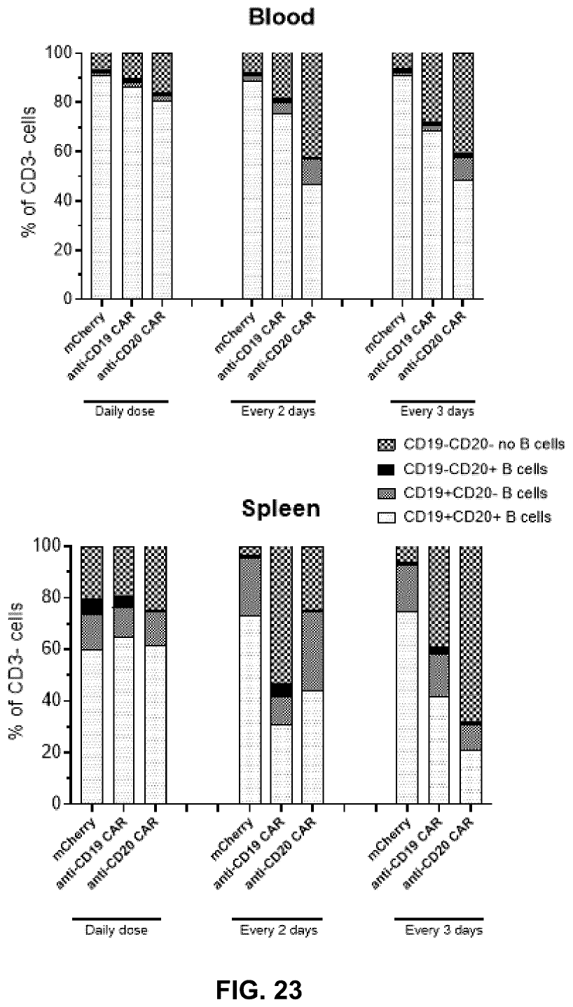

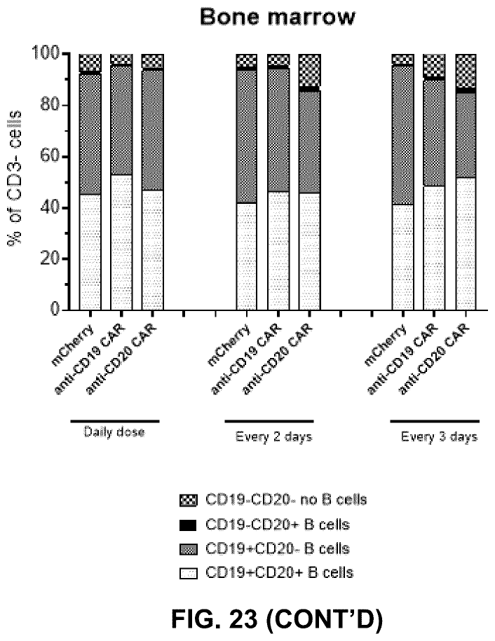

To exclude the possibility that targeting CD19 or CD20 led to differences in the re-populating B cell immunophenotype, the cell composition of the CD3− population in blood and tissues were evaluated at the terminal time point (Day 21 post treatment). Gating the human CD45+CD3− cells, CD19 and CD20 were used to define four subsets, namely, CD19+CD20− B cells, CD19+CD20+B cells, CD19−CD20+ B cells, and non-B cells (CD19−CD20−) (FIG. 23). Analysis of the blood demonstrated that the repopulating B cells were predominantly a CD19+CD20+ immunophenotype. In the spleen, there was a larger population of CD19+CD20− B cells, but no consistent differences were observed between treatments. Almost equivalent proportions of CD19+CD20+ and CD19+CD20− B cells were observed in the bone marrow across all treatments. Therefore, differences in B cell repopulation were not due to differences in the B cell immunophenotype of the repopulating cells.

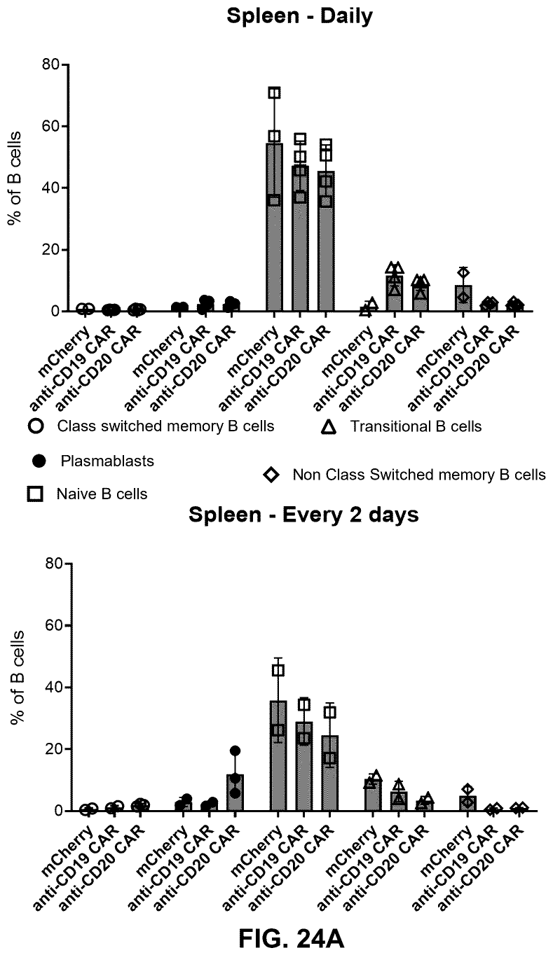

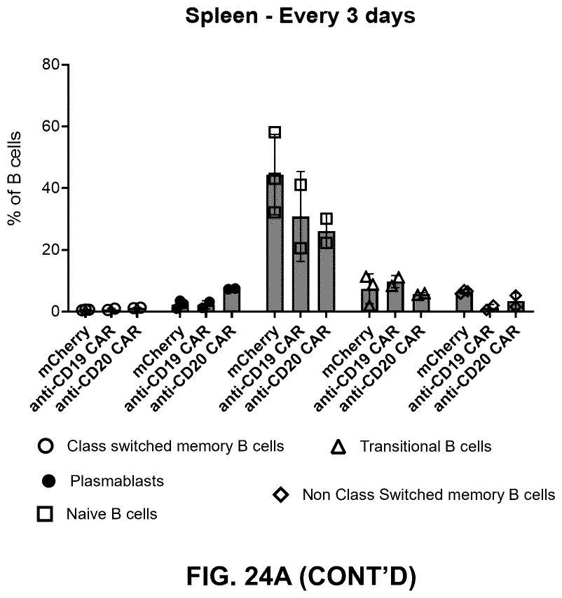

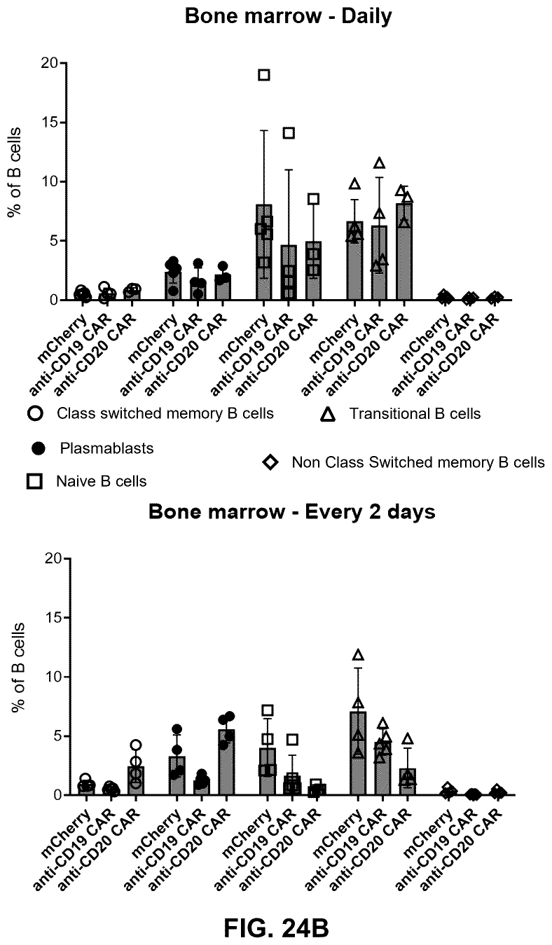

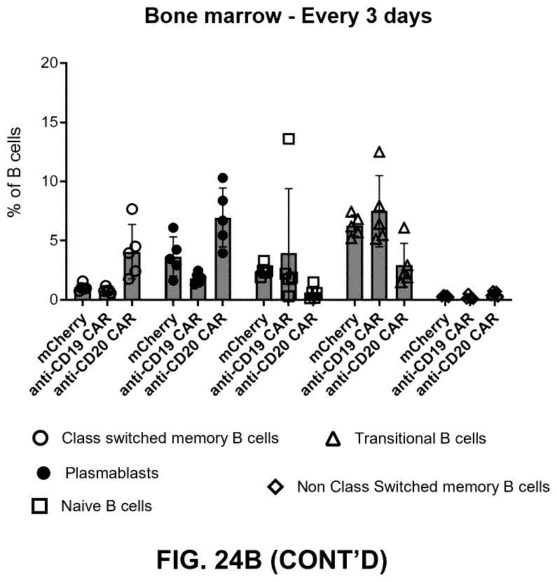

B cells subsets, including plasmablasts, naïve, transitional, and non-class-switched memory, were evaluated at Day 21 post-treatment in blood, spleen, and bone marrow using the flow cytometry. These subsets were defined as follows: B cells in the human CD45+ subpopulation were separated into three subsets after CD19+ cells were gated. The three subsets were determined by CD27 and IgD expression: CD27+IgD−, CD20+IgD+ (non-class switched memory B cells), and CD27−IgD+; from the CD27+IgD− population, plasmablasts (CD20−CD38+) and class-switched memory B cells (CD20+CD38+); and from the CD27−IgD+ population, B cells were further divided into naïve B cells (CD24−CD38−) and transitional B cells (CD24+CD38+) based on the expression of CD24 and CD38. Analysis of blood B cell subsets was excluded due to the low number of B cells (CD19+) present in the blood at the time of the analysis, where almost all mice treated with tLNP-98219 and tLNP-982520 presented less than 500 B cells (CD19+) per sample, the cutoff for analysis. In the spleen, naïve B cells were the major B cell subset, and no major differences were observed between treatments (FIG. 24A). In the bone marrow, the biggest B cell population had a CD19+CD27-IgD-immunophenotype, which is of unclear function in this model, and therefore not classified within any of the evaluated subsets (data not shown). Within the evaluated subsets in the bone marrow, a reduction in plasmablasts was observed in mice treated with tLNP-98219 every 2 or 3 days, but not with tLNP-982520. Conversely, a reduction in transitional B cells was observed in the bone marrow of mice treated with tLNP-982520 every 2 or 3 days, but not with tLNP-98219 (FIG. 24B).

The data obtained in this study indicated that B cell depletion was more durable in this mouse model following tLNP treatment cycles of every 2 or 3 days with tLNP-98219 that targets CD19 on B cells or tLNP-982520 that targets CD20 on B cells compared to daily treatment with the same tLNPs. This correlated with observed higher CAR expression in CD8+ T cells in mice dosed with tLNP-98219 or tLNP-982520 every 2 or 3 days (data now shown). While B cell subsets in this model were largely of a naïve immunophenotype, differences were observed in B cell subsets in the bone marrow following treatment with tLNP-98219 or tLNP-982520 every 2 or 3 days. The reduction of plasmablasts in tLNP-98219-treated mice, or transitional B cells in tLNP-982520-treated mice, likely reflected a difference in CD19 and CD20 expression in the B cell subsets.

### Example 16

### Evaluation of Dosage and Dosing Frequency Parameters in Non-Human Primate

To further evaluate dosage and dosing frequency, cynomolgus macaques were administered tLNP-982520 via IV infusion, providing an anti-CD20 CAR that is pharmacologically active in cynomolgus macaques, at varying dosages, number of doses, and frequency between administrations of doses as shown in Table 15.

TABLE 15

| Test | | | | | | |
|---|---|---|---|---|---|---|
| Article | Group | n* | Dosage | | Frequency | Duration |
| PBS | 1 | 1 | 0 | | 3× Q72h | Dosing phase |
| | | | | | | 14 days |
| tLNP- | 2 | 3 | 0.75 | mg/kg | 3× Q72h | Dosing Phase |
| 982520 | 3 | 3 | 1.0 | mg/kg | 3× Q72h | 14 days + 4 |
| | 4 | 3 | 1.0 | mg/kg | 2× Q72h | week Recovery |
| | 5 | 3 | 1.5 | mg/kg | | |
| | 6 | 3 | 1.0/2.0 | mg/kg | | |

*1 animal per group was sacrificed to conclude the dosing phase 168 hours after the final dose; all remaining animals entered a 4-week recovery phase.

Some animals received 2 doses, where the 2nd dose was administered 72 hours after the preceding dose at 1.0 mg/kg or 1.5 mg/kg per administration or a 1^{st} dose of 1.0 mg/kg and a 2^{nd} dose of 2.0 mg/kg. Some animals received 3 doses, each dose was administered 72 hours after the preceding dose at 0.75 mg/kg and 1.0 mg/kg per administration. A control animal was infused with PBS. Data from this and previous studies were compiled and compared, including where some monkeys received a single dose of 1.0 mg/kg or 2.0 mg/kg, or 3 doses of 0.5 mg/kg.

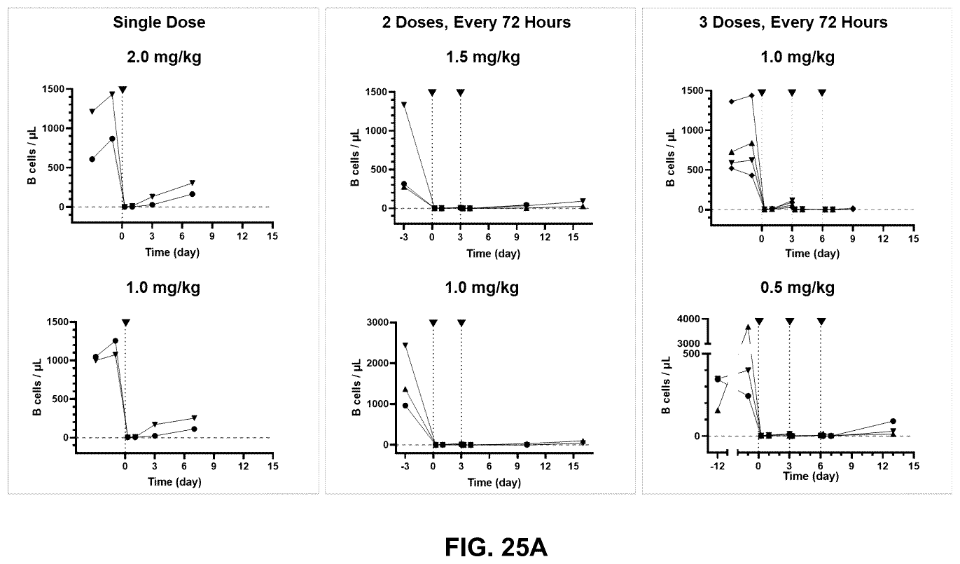

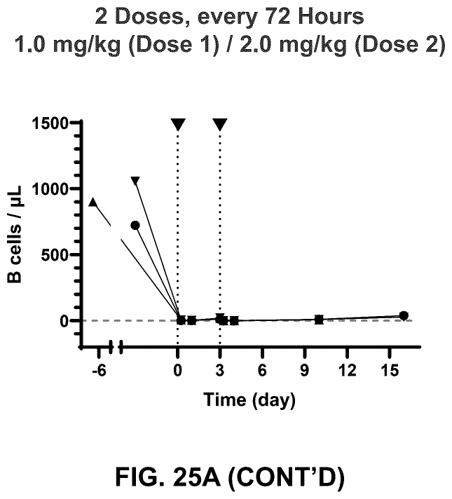

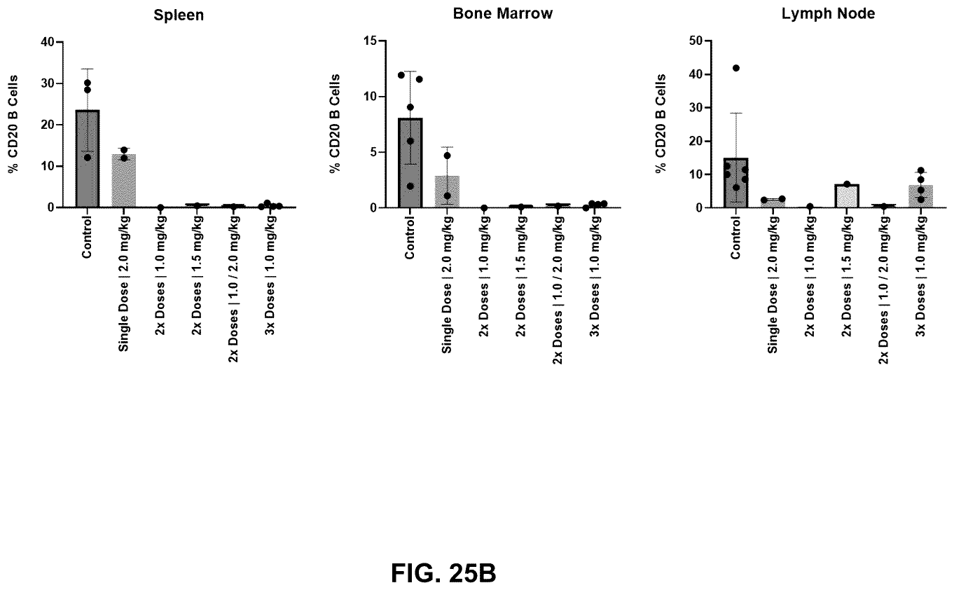

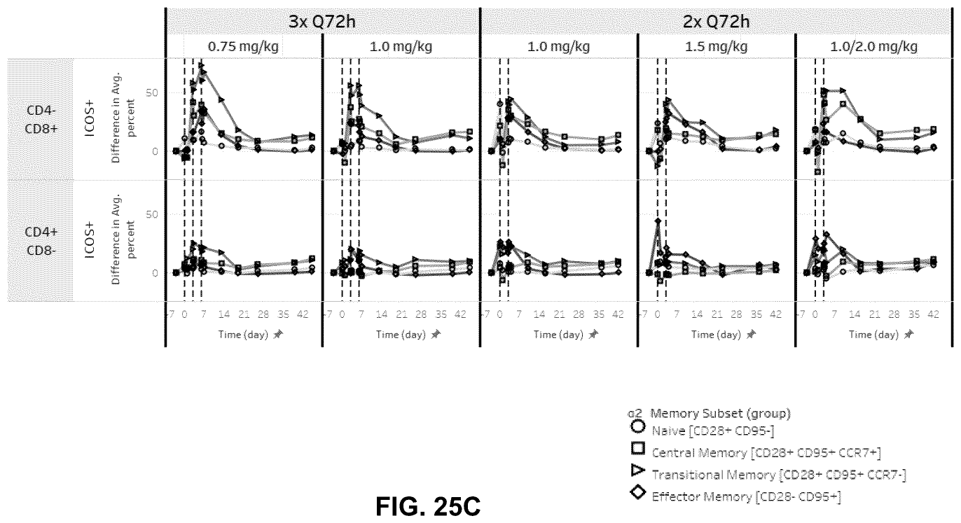

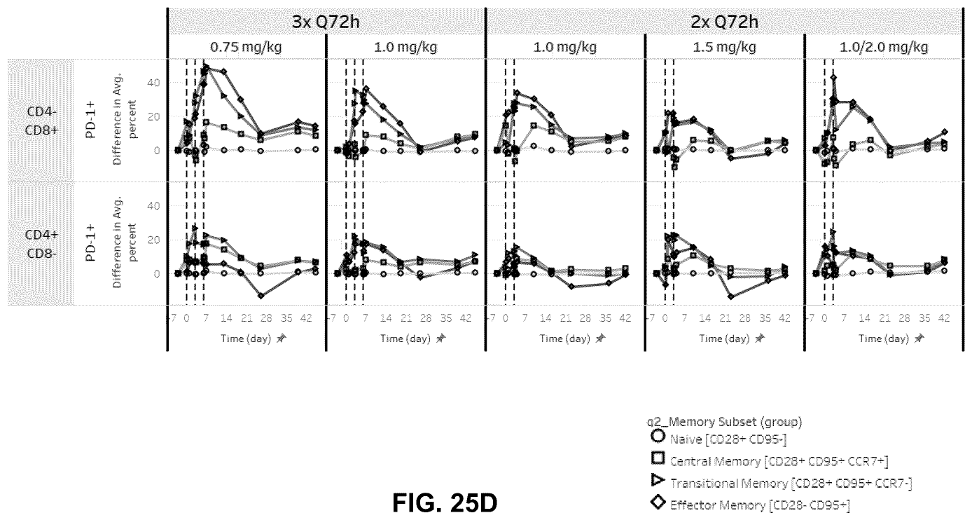

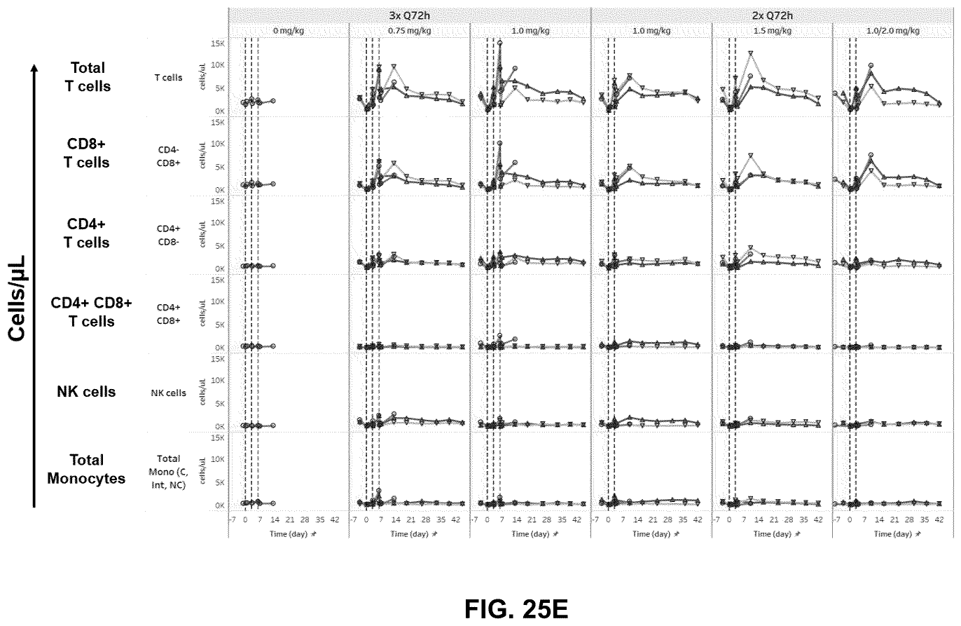

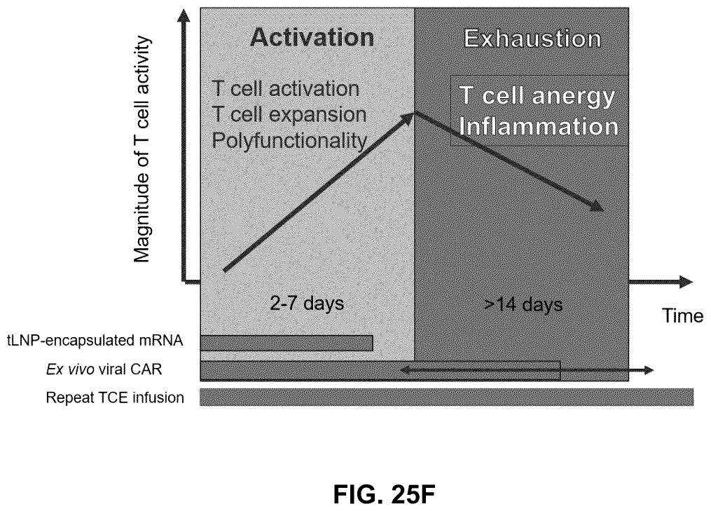

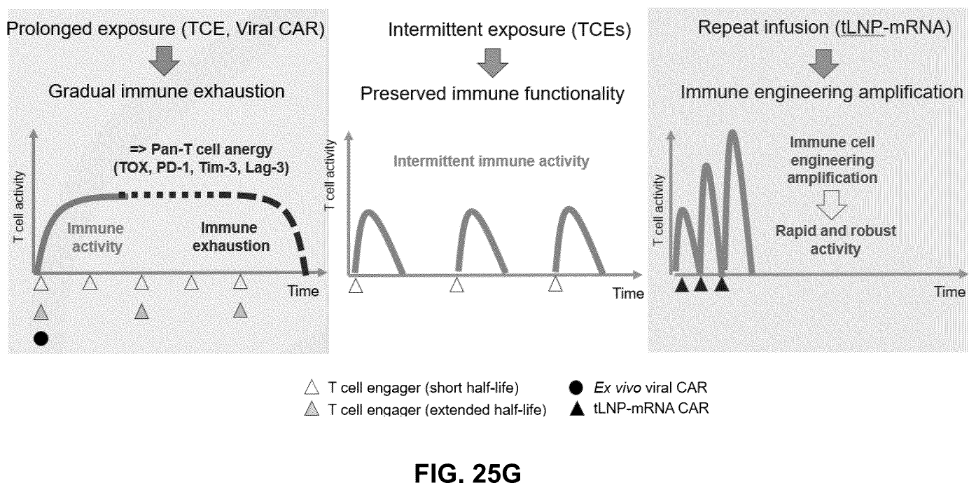

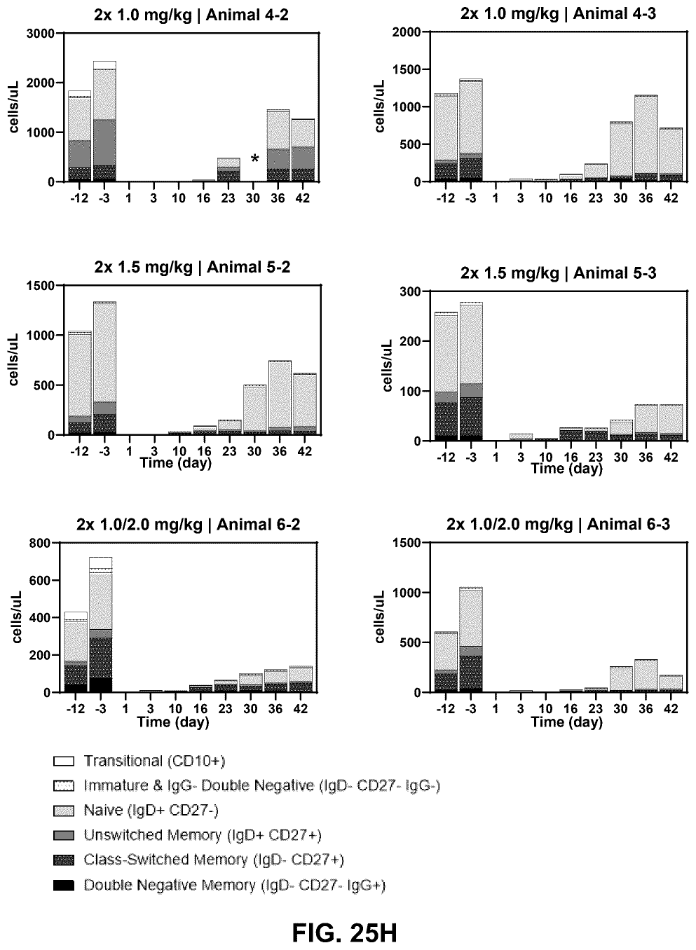

B cells (CD45+CD3-CD159a-CD20+) were enumerated by flow cytometry as the percentage of CD45+ cells that were CD20+; B cell counts/μl in blood were calculated based on number of white blood cell (WBC)/μl x % of B cells (FIG. 25A). The percentage of B cells amongst CD45+ leukocytes from bone marrow, spleen and lymph nodes was measured at the time point of sacrifice (corresponding to the last timepoint measured in blood) (FIG. 25B).

Administration in 2 or 3 doses at 72-hour intervals, each at a dosage of between 0.5 mg/kg and 1.5 mg/kg, depleted B cells in blood and maintained the depletion up to at least 9 days post administration (FIG. 25A). The tissues collected after the last B cell blood count also showed low level of B cells with 2 doses and 3 doses at 1.0 mg/kg compared to a single dose at 2.0 mg/kg (FIG. 25B). Altogether, the results show that a compact regimen comprising 2 doses or 3 doses of administrations, each at a dosage of between 0.5 mg/kg per infusion to 1.5 mg/kg per infusion induced more profound B cell depletion in blood and tissues as compared to a single administration using a large dose (2.0 mg/kg per infusion).

This compact dosing regimen using CD8-targeted tLNPs encapsulating an anti-B cell CAR induced temporary activation and expansion of CD8⁺ T cells without evidence of anergy, exhaustion, or lymphopenia. 2× or 3× administration (Q72 hrs) of CD8-targeted tLNP loaded with anti-B cell (CD20) CAR in mRNA format resulted in substantial but transient activation (ICOS, PD-1; FIGS. 25C and 25D, respectively) and expansion of CD8⁺ T cells, followed by retraction towards pre-treatment state without protracted expression of PD-1 or lymphopenia (FIG. 25E).

Overall, this contributed to the immune cell engineering amplification mechanism afforded by the compact dose regimen as it drove the total number of effector cells higher, with beneficial impact on the pharmacological effect. In addition, this regimen did not lead to short-term or long-term diminution of T cell numbers or anergy/exhaustion typically associated with prolonged expression of PD-1, the prototypical marker of exhaustion when continuously expressed (otherwise, its significance is of activation marker) (FIG. 25F).

This contrasted with other approaches that comprise continuous or supra-physiological activation of T cells such as in context of immune cell engagers delivered by continuous or repeat infusions, or with extended half-life, or ex vivo engineered viral CAR T cells with tonic signaling or protracted persistence (FIG. 25G).

It was not possible to infer without experimentation whether this substantial T cell engineering and activation, mirrored by a profound pharmacological effect, can be achieved without detrimental consequences on the immune system such as T cell anergy, exhaustion or lymphopenia, described with other platforms (FIGS. 25F-25G). This feature of the compact regimen comprising 2 or 3 administrations spaced at 72 hr intervals afforded very high T cell engineering and desired pharmacological effect without detrimental consequences; hence, this protocol is expected to obviate safety problems such as increased rates of infection associated with treatment with immune cell engagers.

The recovery phenotype of B cells in the peripheral blood following a 2-dose regimen of tLNP-982520 was predominantly naïve, demonstrating the tLNP in vivo engineered CAR-T cells provided deep B cell depletion consistent with “immune reset”. The timing and phenotype of peripheral B cell recovery was monitored for each 2-dose regimen in Table 15 (n=2/regimen). Initiation of B cell recovery was observed between Study Days 16-36 (12-32 days after the 2^{nd} dose). The phenotype of the returning B cells was determined by the expression of four markers: IgD, IgG, CD27 and CD10. Naïve phenotypes included transitional (CD10), immature (IgD⁻ CD27⁻ IgG), and naïve (IgD⁺CD27⁻); while mature phenotypes included unswitched (IgD⁺ CD27⁺), class-switched (IgD⁻ CD27⁺), and double negative (IgD⁻ CD27⁻ IgG⁺) memory cells. B cell recovery was predominantly of a naïve phenotype in 5 of the 6 animals (FIG. 25H), particularly in relationship to the pre-dose phenotype profile for each animal, consistent with previous studies. These data demonstrated the potential for a 2-dose tLNP in vivo CAR-T regimen to support deep B cell depletion, which was reflected by their predominantly naïve phenotype on par with what has been observed in autoimmune patients treated with ex vivo CD19 CAR-T cell therapy (Muller et al., 2024 NEJM 390(8):687-700).

All two dose regimens of tLNP-982520 delivered 72 hours apart were well tolerated at the dose levels tested. Mild and transient elevations of liver function test (LFT) enzymes, ALT and AST (alanine transaminase and aspartate transaminase) were observed in a few animals (FIG. 251) and were comparable to other NHP studies. Transient on-target cytokine elevations were observed after each dose, with the highest levels observed after the first dose (FIG. 25J).

Across this and other studies, 75 cynomolgus macaques have received at least 1 dose of tLNP-98219 or tLNP-982520 and 49 have received ≥2 doses 72 hours apart at dose levels of 0.1 to 2.0 mg/kg. Single and two-dose regimens were well tolerated at all dose levels tested. Depletion of B cells was observed in the peripheral blood of all animals. At dosages ≥1.0 mg/kg administered twice, B cell depletion was observed in blood and tissues followed by recovery beginning around day 14 (after initial administration) with the repopulating cells being predominantly naïve B cells. Mild and transient elevations of LFT enzymes were observed upon administration of both tLNPs, and transient on-target cytokine increases were observed with tLNP-982520 whose mRNA encoded a pharmacologically active CAR. Three-dose regimens also demonstrated a favorable safety and tolerability profile in NHP when the 3^{rd} dose was ≤1.0 mg/kg. At the end of the dosing phase (7 days post last dose), there was no clear difference between 2- and 3-dose regimens in the depth of B cell depletion. Peripheral blood B cell rate of recovery during recovery phase was slower in animals on a 3-dose regimen than in those on a 2-dose regimen. Recovery was dose- and age-dependent as well, with higher dosages and increased age being associated with slower recovery of B cell numbers. Recovery of a predominantly naïve phenotype of B cells in peripheral blood was observed in a large majority of the animals, the variability likely reflecting the outbred nature of the population. At sacrifice at the end of the recovery period all 2- and 3-dose animals had predominantly naïve B cells in their spleen and bone marrow. Memory B cell depletion in lymph node was generally incomplete and a subset of memory B cells was present during recovery. Proliferation of CD8⁺ T cells was consistently noted in response to this treatment.

### Example 17

### Elucidation of the Roles of Immune Cell Types in B Cell Killing, T Cell Activation, and Cytokine Production

In vitro transfection of whole and individual cell type-depleted PBMC populations was conducted to assess the roles of the various types of immune cells in the killing of CAR targeted cells and cytokine production. Human PBMCs from two donors were thawed and subjected to cell depletion of one cell population at a time (CD4+ T cells, CD8+ T cells, CD19+B cells, CD14+ and/or CD16+ monocytes, and CD56+NK cells) by using Miltenyi LD Columns with cells labeled with CD4, CD8, CD19, or CD56 MicroBeads. For monocytes cell depletion, CD16 MicroBeads were multiplexed with CD14 MicroBeads to deplete cells expressing either CD16 or CD14 cells (pan-monocyte cell population which include classical, non-classical and intermediate monocytes). Following depletion, WT PBMC (undepleted PBMC), CD4-depleted PBMC, CD8-depleted PBMC, CD19-depleted (that is, B cell-depleted) PBMC, NK-depleted, and monocyte-depleted PBMCs were subjected to in vitro transfection with tLNP-98219 at three doses: 0.01 g, 0.0375 g and 0.6 g of mRNA encoding an anti-CD19 CAR. A non-transfected control (NTD) control was included. Twenty (20) and 66 hours after in vitro tLNP-mediated transfection of human PBMC, the cells were stained with antibodies to quantify B cells and measure T cell activation by flow cytometry. At the end of surface antibody staining, cell pellets were resuspended in 95 μL of cell staining buffer (BioLegend, catalog number 420201) and 5 μL CountBright™ Absolute Counting Beads (Invitrogen, catalog number C36950) in a total of 100 μL. B cells population were gated by using live CD3-CD20+ marker expression. The number of B cells per well was calculated as the Number of live B cells acquired by flow cytometry x Number of counting beads added per well/Number of counting beads acquired by flow cytometry from the same sample. Forty-eight (48) hours after in vitro tLNP-mediated transfection of human PBMC, culture media was assayed for cytokine secretion using a multiplex analysis with the R&D Systems NHP XL Cytokine Premixed Kit (R&D Systems, catalog number FCSTM21-09), a kit that is compatible with human cytokine assessment. A panel of eight cytokines—IFNγ, TNFα, GM-CSF, IL-6, IL-8, IP-10, MCP-1, and IFNβ—was measured using the Luminex xMAP INTELLIFLEX instrument.

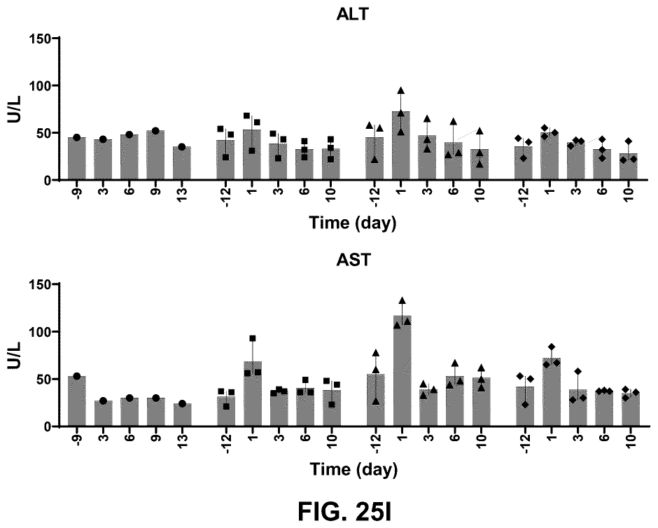

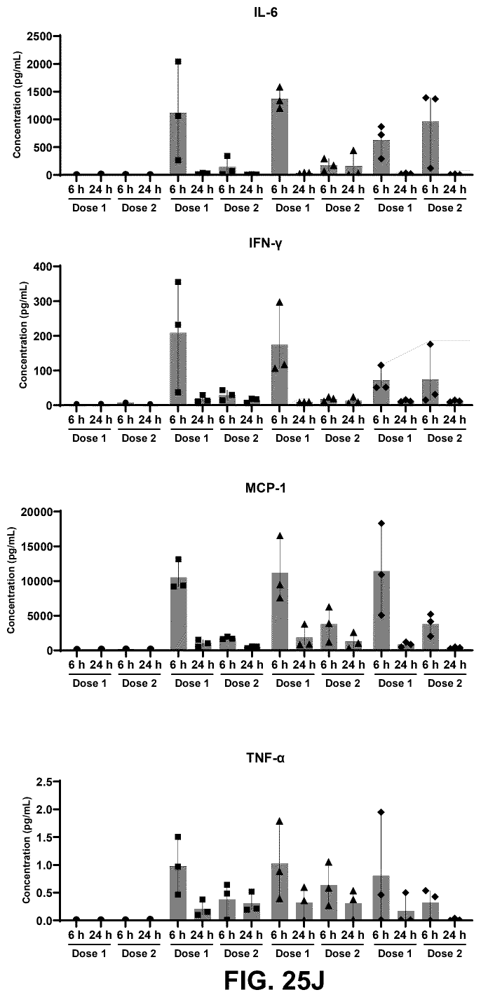

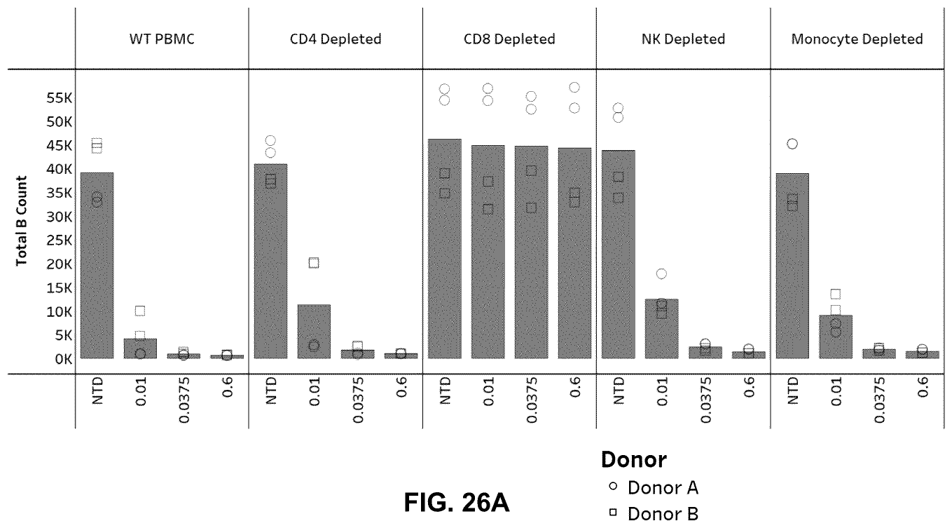

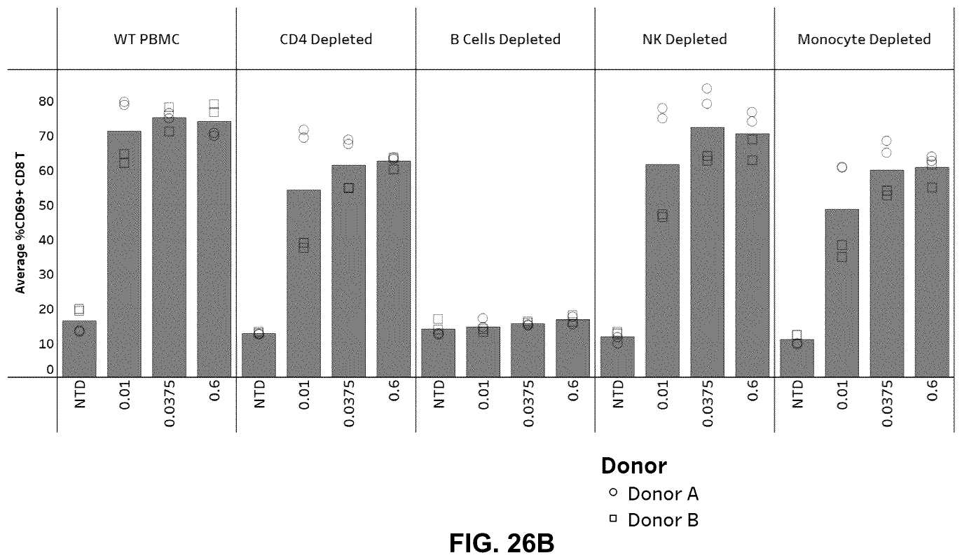

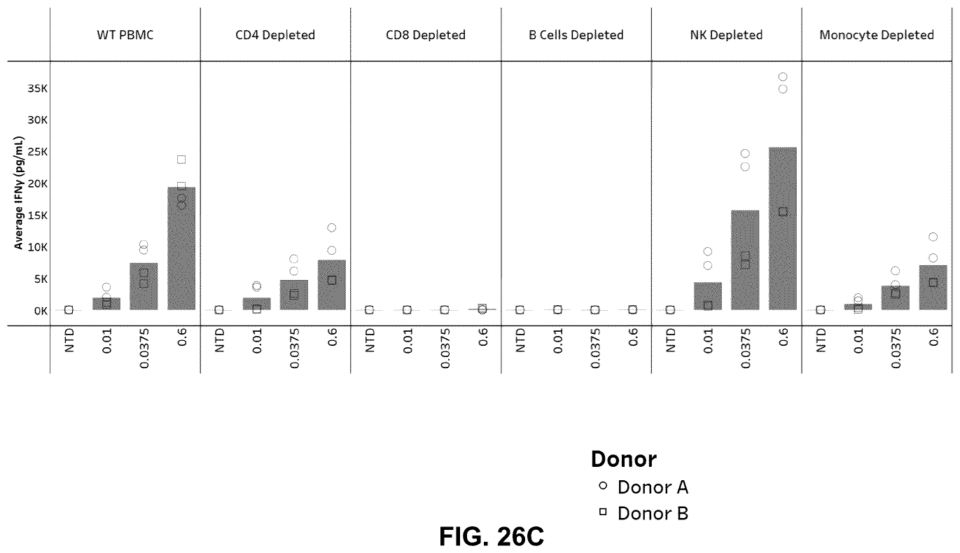

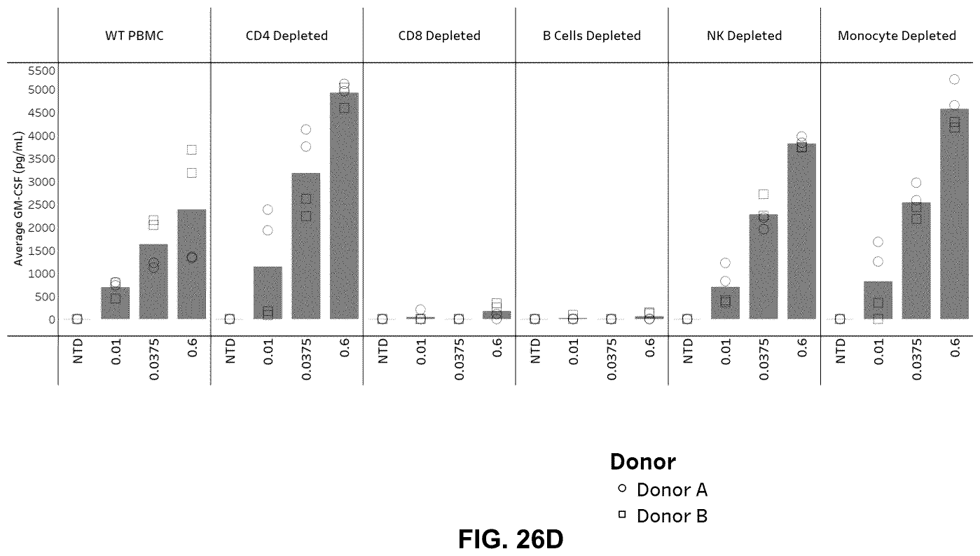

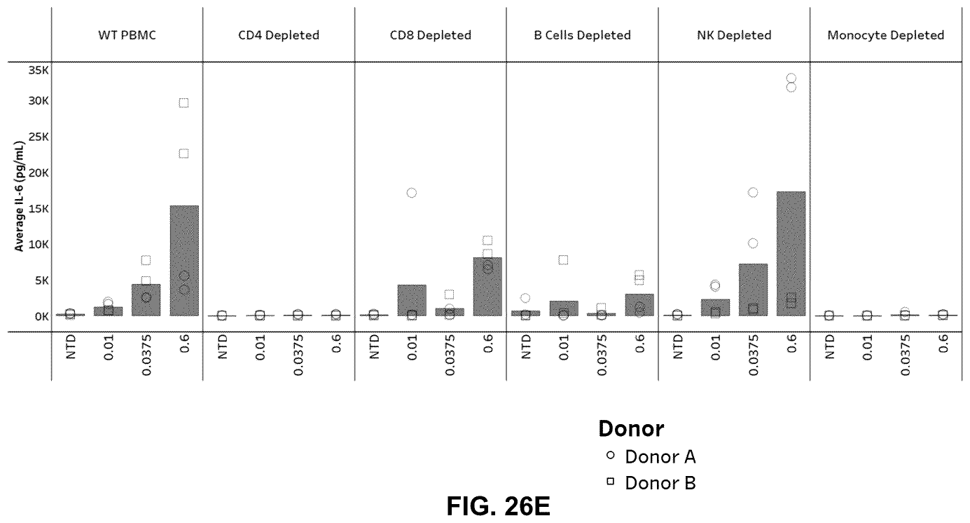

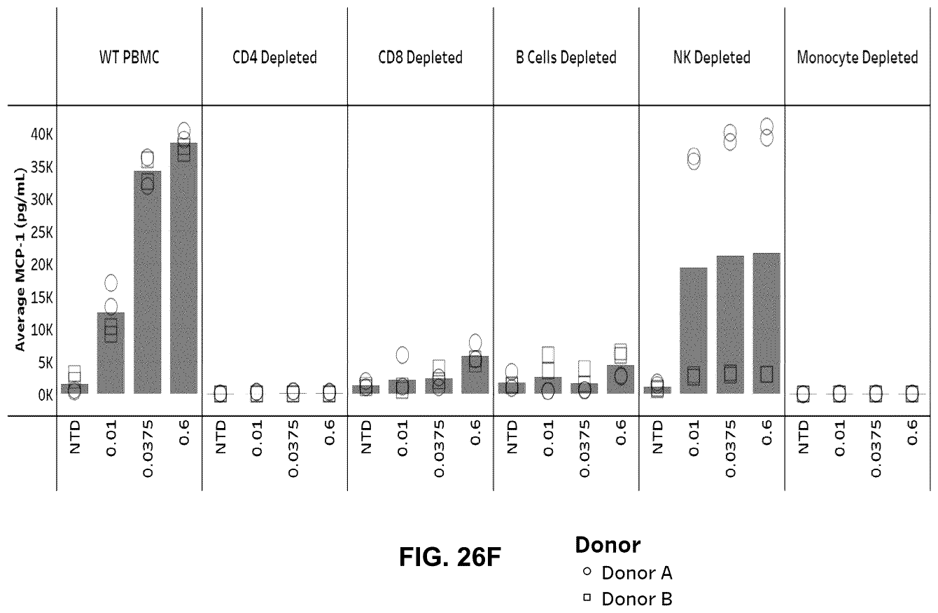

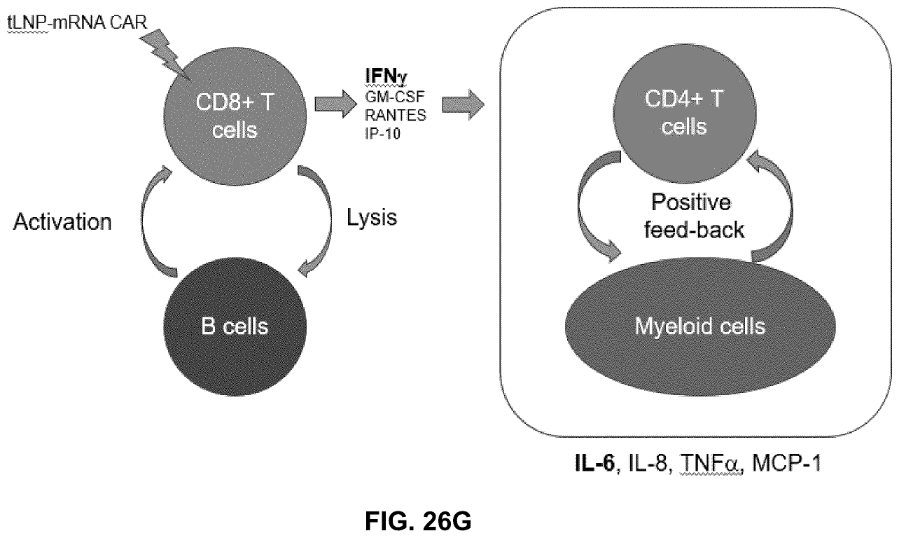

The CD8-depleted cultures exhibited essentially no B cell killing, while CD4-, NK-, and monocyte-depleted PBMC cultures exhibited similar levels of B cell killing as the undepleted PBMC cultures (FIG. 26A; 66-hours post-transfection). However, at the lowest dosage, the undepleted PBMC cultures exhibited markedly incomplete B cell killing and this was seen to a somewhat greater extent in the CD4-, NK-, and monocyte-depleted cultures suggesting that these cell types make some minor contribution to B cell killing that becomes apparent only under dosage-limited conditions. Overall, the data indicate that CD8+ T cells are overwhelmingly the primary cell type responsible for B cell killing.

Transfection of undepleted PBMC populations led to robust expression of the activation marker CD69 by the transfected CD8+ T cells at all three dosages. The B cell-depleted cultures did not induce activation of the transfected CD8+ T cells (FIG. 26B; 20 hours post transfection) showing a dependence of activation on the presence of cells expressing the cognate antigen (that is, CD19) of the transfected CAR, with activation apparently depending on engagement of the antigen receptor, the CAR. The NK-depleted cultures showed comparable levels of activation while the CD4− and monocyte-depleted cultures showed a small diminution (FIG. 26B). Similar results were obtained when activation was assessed by expression of activation markers CD25, 4-1BB, PD-1, and TIM-3 (data not shown).

Two general patterns of cytokine secretion were observed. In one pattern, cytokine secretion was primarily dependent on the presence and presumptive engagement of CD8+ T cells and B cells as depletion of either CD8+ cell or B cells eliminated secretion. In this pattern, depletion of CD4+ cell or monocytes led to reduced cytokine secretion while depletion of NK cells led to augmented secretion. This first pattern was observed with IFNγ and GM-CSF (FIG. 26C and FIG. 26D, respectively; 48 hours post infection). In the second pattern, cytokine secretion showed dependence on all cell types except NK cells, suggesting a cooperative effect. In this pattern, CD4− and monocyte-depleted cultures showed no secretion while CD8− and B cell-depleted cultures showed greatly reduced levels of cytokine secretion. This second pattern was exhibited by IL-6 and MCP-1 (FIG. 26E and FIG. 26F, respectively; 48 hours post infection). No simple pattern was noted for secretion of TNFα, IL-8, IP-10, and IFNβ.

These data suggest the following model. Upon transfection with mRNA encoding an antigen receptor (in this Example, an anti-CD19 CAR) by a CD8-targeted tLNP, CD8+ T cells express the antigen receptor which engages cells expressing the cognate antigen (in this Example, B cells) leading to activation of the CD8+ T cell and lysis of the cell expressing the cognate antigen. The activated CD8+ T cell secretes cytokines such as IFNγ and GM-CSF, as well as others, which act upon CD4+ T cells to produce further cytokines in a positive feedback loop with monocytes, producing elevated levels of cytokines such as IL-6 and MCP-1 (FIG. 26G).

### Example 18

### Low Dose Corticosteroid Reduced Pro-Inflammatory Cytokine Release without Impacting the Pharmacological Activity of tLNP Encapsulating CAR mRNA

Low dose corticosteroid has been used in conjunction with cancer immunotherapies such as immune checkpoint inhibitor (Goodman et al., Clin Cancer Res. 29(14): 2580-2587, 2023) to reduce immune-related adverse events; however, due to its immunosuppressive property, corticosteroid can interfere with the therapeutic efficiency of the immunotherapy. Furthermore, it is unclear if corticosteroid can be used with the disclosed CAR therapy using tLNPs that employs distinct mechanisms of action from other immunotherapies.

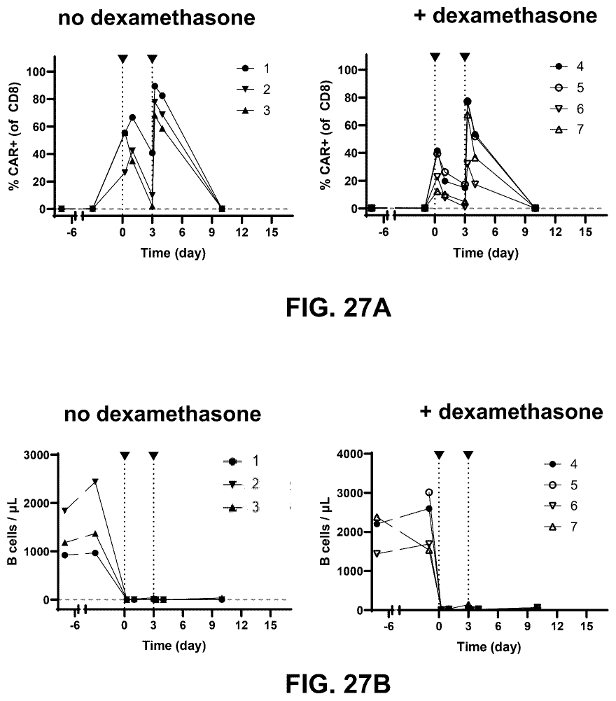

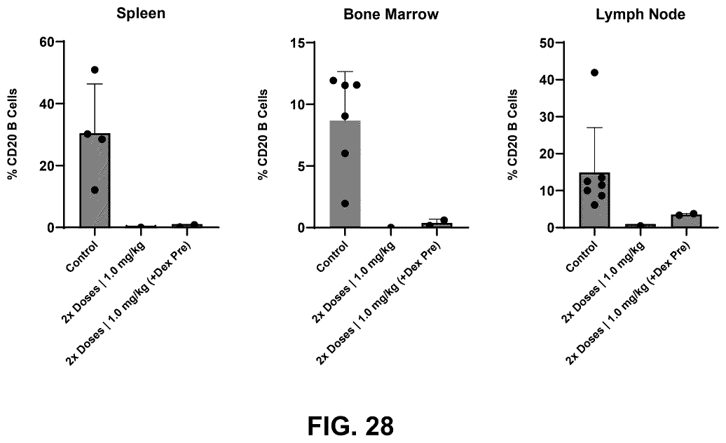

To test whether corticosteroid diminished the pharmacological activity of the disclosed compact regimen comprising multiple doses of a CD8-targeted tLNP, cynomolgus macaques were administered 5 mg/kg diphenhydramine and 1 mg/kg dexamethasone intramuscularly approximately 1 hour prior to administration of the tLNPs. The tLNPs were administered by intravenous infusion over 60 minutes (tLNP-982520; anti-CD20 CAR; 1 animal) or 90 minutes of (tLNP-982520 and tLNP-98219 (anti-CD19 CAR), 3 animals each, as well as PBS as a control, 1 animal) in a 2-dose regimen (2xQ72 hr). The anti-CD20 CAR was pharmacologically active in cynomolgus macaques while the anti-CD19 CAR was not. The percentage of CAR-expressing CD8+ T cells and the number of B cells depleted in blood were measured as described above and compared to the results obtained with the animals in Example 16 (above) which did not include a dexamethasone pre-treatment. Regardless of dexamethasone treatment, the trend of CAR engineering rate was maintained in which the subsequent dose significantly amplified the engineering rate from the first dose (from 40% to 60% after the first dose to about 80% after the second dose) (FIG. 27A). Similarly, B cell depletion in blood using tLNP encapsulating CAR mRNA was unaffected by dexamethasone (FIG. 27B). The slower infusion showed a trend to somewhat lower percentages of CAR+ cells, possibly reflecting a lower maximal concentration of tLNP; however, this reduction did not translate to reduced B cell depletion (FIG. 27B). Furthermore, the number of B cells in spleen and bone marrow after the treatment were also not affected by dexamethasone (FIG. 28). The number of B cells in lymph node from dexamethasone pre-treated animals was marginally higher compared to without dexamethasone but was still significantly lower than control treatment (without tLNP). The data indicated that corticosteroid had minimal impact on the immune engineering amplification and pharmacological activity achieved by the compact regimen.

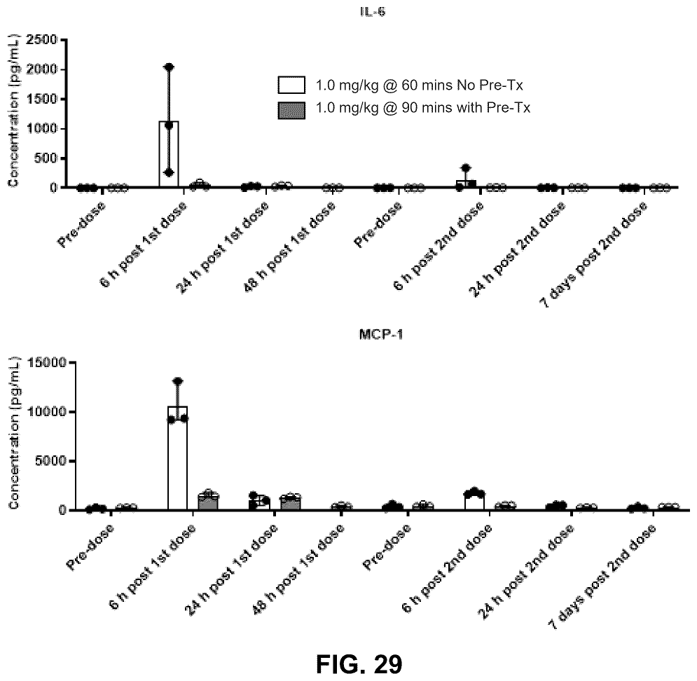

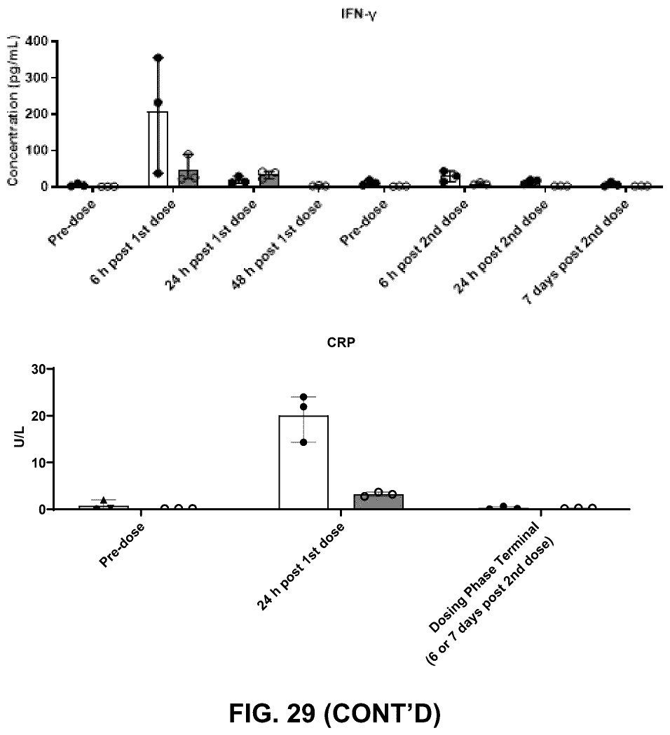

To test the use of corticosteroid in suppressing pro-inflammatory cytokine release, which is triggered by CAR therapy using tLNPs and can contribute to CRS, transient on-target cytokine elevations were measured over time course after each dose of tLNP with and without dexamethasone pre-treatment. Without dexamethasone, the highest level of cytokine was observed after 6 hours post first dose and second dose for IL-6, MCP-1, and IFN-7; and after 24 hours post first dose for C-reactive protein (CRP) (FIG. 29). These levels were significantly reduced by at least 4-folds when the animals were pre-treated with dexamethasone (FIG. 29). The data indicated that pre-treatment with low dose corticosteroid resulted in lower levels of pro-inflammatory cytokines. This represented an increased therapeutic index of the treatment by pre-empting elevation of pro-inflammatory cytokines and acute phase response while not interfering with the pharmacological activity. This further suggests that the combination of dexamethasone pretreatment with tLNP-mediated CAR-T therapy can support more intensive dose regimens whether or not using a compact administration schedule.

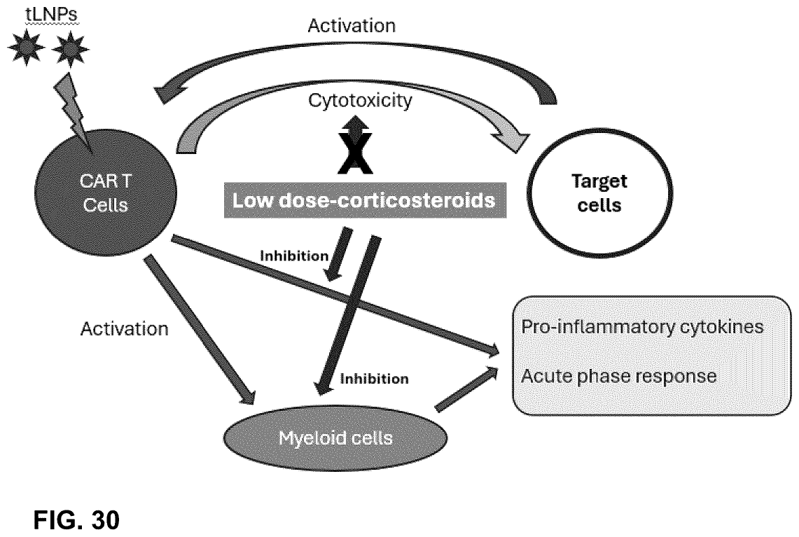

Altogether these data indicated that low dose corticosteroid treatment can inhibit acute phase responses and inflammatory cytokine production without blocking the cytotoxic activity of in vivo CAR-T cells (FIG. 30).

### Example 19

### Effect of B Cell Depletion on Induction of Anti-Drug Antibodies

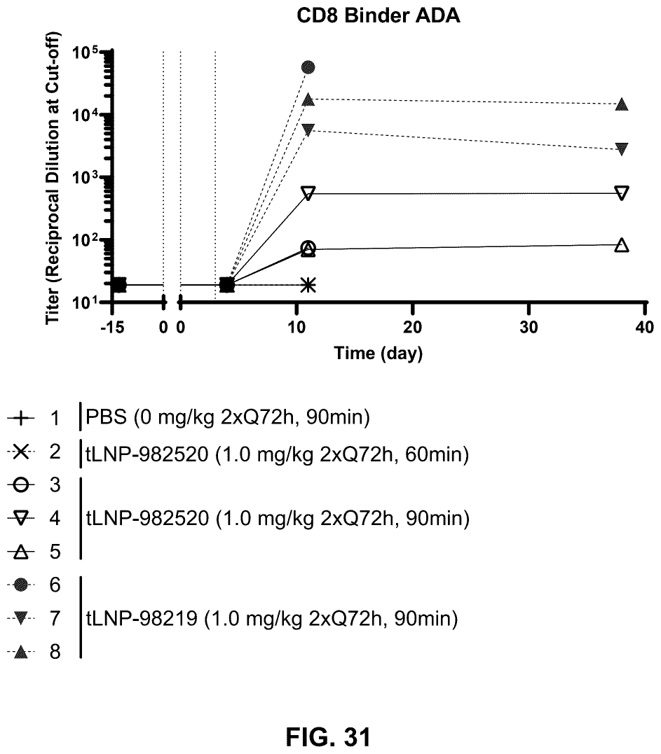

The presence of anti-drug antibodies in serum samples from the animals in the immediately preceding Example was assayed to learn whether the B cell depletion achieved with the compact administration schedule would pre-empt induction of high tittered anti-product antibodies. The results obtained with the pharmacologically active anti-CD20 CAR (which depleted B cells in the cynomolgus macaques) was compared to those with the anti-CD19 CAR which was not pharmacologically active. The products (CD8-targeted tLNP encapsulating mRNA encoding CAR) were essentially identical except for the CAR encoded (tLNP-982520 for anti-CD20 CAR or tLNP-98219 for the anti-CD19 CAR). The comparison showed lower total antibody titers against components of the product: the anti-CD8 targeting antibody (FIG. 31), PEG, and the encoded CAR (data not shown) when the treatment led to B cell depletion (i.e., when the treatment produced an anti-CD20 CAR).

This showed that a compressed dose regimen resulting in rapid and massive T cell engineering leading to B cell depletion (timeframe hours—days instead of weeks) was able to kinetically outcompete the onset of B cell response manifested through high ADA levels. This approach would result in diminished titers of potentially detrimental ADAs (neutralizing the product or eliciting immunopathology upon follow up infusions) thereby allowing repeated treatment cycles or additional treatments with tLNPs as needed. In addition, one can envisage utilization of this approach and composition to critically enable treatment with potentially immunogenic products (tLNP based or not) by prior B cell depletion according to the compact regimen.

This contrasts to other approaches which are unable to achieve this performance as they cannot afford such a rapid and substantial pharmacological effect—for example viral based CAR T cells relying on slower activation and expansion of T cells as opposed to pre-existing effector T cells.

It was not possible to infer without experimentation whether this ADA protective effect would occur, as it could not be predicted whether kinetic competition of B cell depletion due to rapid T cell engineering afforded by this very composition (tLNP-CAR in mRNA format, with a CAR having a B cell depleting effect) in conjunction with the immune cell engineering amplification afforded by this compact treatment cycle would win out over induction of antibody immunity that occurs within 5-7 days after antigen exposure. The results demonstrated that B cell depletion disrupted induction of antibody.

### Example 20

### Phase 1 Open Label Clinical Trial of Anti-CD19 CAR mRNA in CD8-Targeted LNPs

This study is a Phase 1, single-center, open-label, single and multiple ascending dose study to evaluate the safety, tolerability, pharmacokinetics, and pharmacodynamics of tLNP-98219 by intravenous (IV) administration in patients with a B cell-mediated autoimmune disease. Subjects of the study include healthy volunteers and autoimmune patients. Eligible patients include those with myositis, anti-synthetase syndrome, lupus nephritis, membranous nephropathy, myasthenia gravis, systemic sclerosis, stiff person syndrome, Sjögren's syndrome, multiple sclerosis, rheumatoid arthritis, idiopathic thrombocytopenic purpura, systemic lupus erythematosus, and myelin oligodendrocyte glycoprotein (MOG) autoantibody disease.

The planned enrollment is up to 40 subjects to receive intravenous infusions of CD8-targeted LNP encapsulating an mRNA encoding an anti-CD19 CAR (hereinafter in this Example, the tLNP) in up to 8 cohorts as follows. Dose escalation may continue unless stopping rules are met or until a cohort is identified in which complete depletion of circulating CD19+B cells (defined as <5 cells/μL) for ≥14 days is observed in all subjects.

Dosing may be suspended or terminated at the Sponsors' request at any time and for any reason, including evidence of adequate pharmacodynamic effect observed at a given dose level and regimen. The Scientific Review Committee (SRC) may recommend exploring lower or intermediate dose levels in the single ascending dose (SAD) or multiple ascending dose (MAD) portions, provided the dose level does not exceed the highest planned dose.

TABLE 16

| | | Cumulative | | | |
|---|---|---|---|---|---|
| Cohort | Dose regimen | dose | | n* | Dose escalation criteria |
| A | 0.07 mg/kg × 1 | 0.07 | mg/kg | 4 | Safety, stopping rules, |
| (SAD) | | | | | PK, PD (including |
| | | | | | circulating CD19⁺ |
| | | | | | B cell count) |
| B | 0.3 mg/kg × 1 | 0.3 | mg/kg | 4 | Safety, stopping rules, |
| (SAD) | | | | | PK, PD (including |
| | | | | | circulating CD19⁺ |
| | | | | | B cell count) |
| C | 0.5 mg/kg × 1 | 0.5 | mg/kg | 4 | Safety, stopping rules, |
| (SAD) | | | | | PK, PD (including |
| | | | | | circulating CD19⁺ |
| | | | | | B cell count) |
| D | 1.0 mg/kg × 1 | 1.0 | mg/kg | 4 | Safety, stopping rules, |
| (SAD) | | | | | PK, PD (including |
| | | | | | circulating CD19⁺ |
| | | | | | B cell count) |
| E | 0.07 mg/kg × 2 | 0.14 | mg/kg | 6 | Safety, stopping rules, |
| (MAD) | Q72h | | | | PK, PD (including |
| | | | | | circulating CD19⁺ |
| | | | | | B cell count) |
| F | 0.3 mg/kg × 2 | 0.6 | mg/kg | 6 | Safety, stopping rules, |
| (MAD) | Q72h | | | | PK, PD (including |
| | | | | | circulating CD19⁺ |
| | | | | | B cell count) |
| G | 0.5 mg/kg × 2 | 1.0 | mg/kg | 6 | Safety, stopping rules, |
| (MAD) | Q72h | | | | PK, PD (including |
| | | | | | circulating CD19⁺ |
| | | | | | B cell count) |
| H | 1.0 mg/kg × 2 | 2.0 | mg/kg | 6 | Pre-defined maximal |
| (MAD) | Q72h | | | | dose regimen |

*One sentinel subject will be dosed first in each cohort with the remainder of dosing in the cohort to follow only after initial assessment of safety and tolerability (1 week after final dose of study drug).
Q72h = every 72 hours.

The safety of subjects is carefully monitored on an ongoing basis with special attention to signs and symptoms of cytokine release syndrome (CRS) and immune effector cell-associated neurotoxicity syndrome (ICANS).

Subjects receive pre-medication prior to each dose to reduce the risk of infusion-related reactions. Each of the following pre-medications are given on the day of dosing at least 60 minutes prior to the start of the infusion:
IV corticosteroid (e.g., dexamethasone 10 mg, or equivalent)
IV H1 blocker (e.g., diphenhydramine 50 mg, or equivalent)
IV H2 blocker (e.g., ranitidine 50 mg, or equivalent)

Subjects are admitted prior to first dose and monitored in the Phase 1 unit until at least 72 hours after completion of their final dose of study drug and until reconstituting B cells are detected in the circulation (defined as 2 increasing and consecutive daily measurements >10/μL).

If grade 1 CRS or ICANS lasting ≥24 hours, or grade ≥2 CRS or ICANS at any time are observed, IV tocilizumab (8 mg/kg×1) and/or dexamethasone (10 mg Q6h until symptoms resolve), respectively, should be administered in consultation with the principal investigator (PI) and medical monitor.

### Stopping Rules:

Occurrence of a serious adverse event (SAE) deemed related to the tLNP (other than expected on-target lymphopenia, cytokine release syndrome (CRS), or immune effector cell-associated neurotoxicity syndrome (ICANS)) results in interruption of any further dosing in the study pending a safety review by a Safety Review Committee.

Occurrence of two or more identical adverse events (AEs) at Grade ≥3 (CTCAE v5.0) in the same cohort (other than expected on-target lymphopenia, CRS, or ICANS) deemed related to the tLNP, results in interruption of any further dosing pending a safety review by the SRC.

Occurrence of any Grade ≥3 AE of CRS or ICANS (by ASBMT Consensus Grading) results in interruption of any further dosing in the study pending a safety review by the SRC.

Occurrence of two Grade 2 AE of CRS or ICANS (by ASBMT Consensus Grading) results in interruption of any further dosing in the study pending a safety review by the SRC.

The Main Portion of the study includes a Screening Period of up to 4 weeks and an 8-week Treatment Period. Subjects are admitted prior to first dose and monitored in the Phase 1 unit until at least 72 hours after completion of their final dose of study drug and until reconstituting B cells are detected in the circulation (defined as 2 increasing and consecutive daily measurements >10/μL). After discharge, subjects return to the clinic weekly to evaluate safety, tolerability, pharmacokinetics, and pharmacodynamics up to and including the end of study visit.

### Study Endpoints

Safety and Tolerability: Safety assessments include reporting of AEs and SAEs, clinical laboratory tests, cytokines, anti-drug antibodies (ADA), ECGs, vital signs, physical examination, and vaccine titers.

Pharmacokinetics: Chimeric antigen receptor (CAR) engineering rate (expressed as % of T cells and T cell subsets).

Pharmacodynamics: Circulating CD19+ B cell count, phenotype of reconstituting B cells.

### Administration

Subjects are administered a fixed concentration of 0.25 mg/mL. This provides flexibility to provide the planned dosages without needing to adopt concerningly high volumes or flow rates in larger subjects even in patients with volume overload (canonically those with heart, kidney, or liver failure). Dosages and flow rates for a nominal 70 kg subject for 90- and 60-minute infusions to achieve the indicated dosages are shown below.

TABLE 17

| | | | Infusion rate |
|---|---|---|---|
| Dosage | Subject Body | Infusion | (mL/min for |
| (mg/ml) | Mass (kg) | Vol (mL) | 90 or 60 min) |
| 0.07 | 70 | 19.6 | 0.22/0.33 |
| 0.3 | 70 | 84 | 0.93/1.4 |
| 0.5 | 70 | 140 | 1.56/2.33 |
| 1.0 | 70 | 280 | 3.11/4.67 |

These results are then used to establish the safety, tolerability, pharmacokinetics, and pharmacodynamics of tLNP-98219 after intravenous (IV) administration in patients with a B cell-mediated autoimmune disease as enumerated above.

TABLE 18
Humanized anti-CD8A whole antibody and related sequences. HC:
heavy chain. LC: light chain. X is any amino acid. X1 is N,
S, Q, or A. X2 is N, Q, D, S, or A. X3 is D, E, S, or A.

| | | SEQ |
|---|---|---|
| Name | Note | ID NO |
| CBD1017vh | VH of mouse anti-CD8A antibody clone CT8 | 108 |
| VH-CDR1 CBD1017 | VH-CDR1 of mouse anti-CD8A antibody clone CT8 | 109 |
| VH-CDR2 CBD1017 | VH-CDR2 of mouse anti-CD8A antibody clone CT8 | 110 |
| VH-CDR3 CBD1017 | VH-CDR3 of mouse anti-CD8A antibody clone CT8 | 111 |
| CBD1017vl | VL of mouse anti-CD8A antibody clone CT8 | 112 |
| VL-CDR1 CBD1017 | VL-CDR1 of mouse anti-CD8A antibody clone CT8 | 113 |
| VL-CDR2 CBD1017 | VL-CDR2 of mouse anti-CD8A antibody clone CT8 | 114 |
| VL-CDR3 CBD1017 | VL-CDR3 of mouse anti-CD8A antibody clone CT8 | 115 |
| 1-46*01 | IgHV1-46*01/IGHJ6*01 germline VH | 116 |
| h1017-H1 | Humanized CT8 VH variant based on VH1-46 | 117 |
| h1017-H2 | Humanized CT8 VH variant based on VH1-46 | 118 |
| h1017-H3 | Humanized CT8 VH variant based on VH1-46 | 119 |
| h1017-H4 | Humanized CT8 VH variant based on VH1-46 | 120 |
| h1017-H5 | Humanized CT8 VH variant based on VH1-46 | 121 |
| 1-39*01 | IgKV1-39*01/IGKJ2*01 germline VL | 122 |
| h1017-L1 | Humanized CT8 VL variant based on VK1-39 | 123 |
| h1017-L2 | Humanized CT8 VL variant based on VK1-39 | 124 |
| h1017-L3 | Humanized CT8 VL variant based on VK1-39 | 125 |
| VH hOKT8 | VH of humanized mouse anti-CD8A antibody clone OKT8 | 126 |
| VL hOKT8 | VL of humanized mouse anti-CD8A antibody clone OKT8 | 127 |
| VH OKT8 | VH of mouse anti-CD8A antibody clone OKT8 | 128 |
| VL OKT8 | VL of mouse anti-CD8A antibody clone OKT8 | 129 |
| VH CBD1380 | N55S of h1017-H2 | 130 |
| VH CBD1381 | N55Q of h1017-H2 | 131 |
| VH CBD1382 | N55A of h1017-H2 | 132 |
| VH CB1383 | N55D of h1017-H2 | 133 |
| Mod 1-18 | Modified IgHV1-18*01 germline VH | 134 |
| h1017-H6 | Humanized CT8 VH variant based on VH1-18 | 135 |
| h1017-H7 | Humanized CT8 VH variant based on VH1-18 | 136 |
| h1017-H8 | Humanized CT8 VH variant based on VH1-18 | 137 |
| h1017-H9 | Humanized CT8 VH variant based on VH1-18 | 138 |
| h1017-H10 | Humanized CT8 VH variant based on VH1-18 | 139 |
| Mod 3D-11 | Modified IgKV3D-11*01 germline VL | 140 |
| h1017-L4 | Humanized CT8 VL variant based on VK3D-11 | 141 |
| h1017-L5 | Humanized CT8 VL variant based on VK3D-11 | 142 |
| h1017-L6 | Humanized CT8 VL variant based on VK3D-11 | 143 |
| IGKC_human | Kappa chain constant region used in the whole | 144 |
| | antibody constructs (UniProt P01834) | |
| IGG1_human | Wild type IgG1 constant regions (UniProt P0DOX5) | 145 |
| IgG1_human | Fc-silenced IgG1 constant regions used in the whole antibody | 146 |
| LALAPA EM | constructs (with E358 and M360 allotypic reversions) | |
| IgG1_human | Fc-silenced IgG1 constant regions used in the whole antibody | 147 |
| LALAPA DL | constructs (with D358 and L360 allotypic residues) | |
| VH-CDR1 N33var | VH N33 degenerate | 148 |
| VH-CDR2 N55var1 | VH N55 degenerate (set w/o D) | 149 |
| VH-CDR2 N55var1 | VH N55 degenerate (set w/D) | 150 |
| VH-CDR3 N103var | VH N103 degenerate | 151 |
| VL-CDR1 D30 and | VL D30 and N34 degenerate | 152 |
| N34var | | |
| VH-CDR2 N57var | VL N57 degenerate | 153 |
| VH-CDR3 N95, | VL N95, N96, and D98 degenerate | 154 |
| N96, and D98var | | |
| h1017-1-46 (1) | Humanized CT8 VH based on VH1-46 with CDR variants (1) | 155 |
| h1017-1-46 (2) | Humanized CT8 VH based on VH1-46 with CDR variants (2) | 156 |
| h1017-1-39 | Humanized CT8 VL based on VK1-39 with CDR variants | 157 |
| h1017-1-18 (1) | Humanized CT8 VH based on VH1-18 with CDR variants | 158 |
| h1017-1-18 (2) | Humanized CT8 VH based on VH1-18 with CDR variants | 159 |
| h1017-3D-11 | Humanized CT8 VL based on VK3D-11 with CDR variants | 160 |
| VH-CDR2v2 | Modified VH-CDR2 of mouse anti-CD8A clone CT8 | 161 |
| CBD1017 N55S | | |
| VH-CDR2v3 | Modified VH-CDR2 of mouse anti-CD8A clone CT8 | 162 |
| CBD1017 N55Q | | |
| VH-CDR2v4 | Modified VH-CDR2 of mouse anti-CD8A antibody clone CT8 | 163 |
| CBD1017 N55A | | |
| CBD1033 HC | h1017-H2 Fc-silenced IgG1 | 164 |
| CBD1033 LC | h1017-L2 Kappa chain | 165 |
| CBD1035 LC | | |
| CBD1037 LC | | |
| CBD1039 LC | | |
| CBD1380 LC | | |
| CBD1381 LC | | |
| CBD1382 LC | | |
| VL CBD1443 | D30E of h1017-L2 | 166 |
| VL CBD1446 | | |
| VL CBD1444 | D30S of h1017-L2 | 167 |
| VL CBD1447 | | |
| VL CBD1575 | | |
| VL CBD1576 | | |
| VL CBD1622 | | |
| VL CBD1623 | | |
| VL CBD1445 | D30A of h1017-L2 | 168 |
| VL CBD1448 | | |
| VH CBD1449 | N55S of h1017-H4 | 169 |
| VH CBD1450 | N55Q of h1017-H4 | 170 |
| VH CBD1575 | | |
| VH CBD1451 | N55A of h1017-H4 | 171 |
| VH CBD1576 | | |
| CBD1035 HC | h1017-H3 Fc-silenced IgG1 | 172 |
| CBD1039 HC | h1017-H5 Fc-silenced IgG1 | 173 |
| CBD1040 HC | | |
| CBD1040 LC | h1017-L3 Kappa chain | 174 |
| CBD1380 HC | N55S of h1017-H2 Fc-silenced IgG1 | 175 |
| CBD1381 HC | N55Q of h1017-H2 Fc-silenced IgG1 | 176 |
| CBD1382 HC | N55A of h1017-H2 Fc-silenced IgG1 | 177 |
| CBD1037 HC | h1017-H4 Fc-silenced IgG1 | 178 |

TABLE 19
LNP Compositions

| Composition | | |
|---|---|---|
| Code | Lipid Composition [Ratios] | N/P |
| BF1 | ALC-0315:DSPC:CHOL:DMG-PEG(2k):DSPE-PEG(2k)-MAL | 6 |
| | [50:10:38.5:1.4:0.1] | |
| F1 | CICL1:DSPC:CHOL:DMG-PEG(2k):DSPE-PEG(2k)-MAL | 6 |
| | [50:10:38.5:1.4:0.1] | |
| F2 | CICL1:DSPC:CHOL:DMG-PEG(2k):DSPE-PEG(2k)-MAL | 3 |
| | [50:10:38.5:1.3:0.2] | |
| F3 | CICL1:DSPC:CHOL:DMG-PEG(2k):DSPE-PEG(2k)-MAL | 9 |
| | [50:10:38.5:1.425:0.075] | |
| F4 | CICL1:DSPC:CHOL:DMG-PEG(2k):DSPE-PEG(2k)-MAL | 6 |
| | [42:10:46.5:1.4:0.1] | |
| F5 | CICL1:DSPC:CHOL:DMG-PEG(2k):DSPE-PEG(2k)-MAL | 6 |
| | [58:10:30.5:1.4:0.1] | |
| F6 | CICL1:DSPC:CHOL:DSG-PEG(2k):DSPE-PEG(2k)-MAL | 6 |
| | [35:10:53.5:1.4:0.1] | |
| F7 | CICL1:DSPC:CHOL:DSG-PEG(2k):DSPE-PEG(2k)-MAL | 6 |
| | [42:10:46.5:1.4:0.1] | |
| F8 | CICL1:DSPC:CHOL:DSG-PEG(2k):DSPE-PEG(2k)-MAL | 6 |
| | [50:10:38.5:1.4:0.1] | |
| F9 | CICL1:DSPC:CHOL:DSG-PEG(2k):DSPE-PEG(2k)-MAL | 6 |
| | [58:10:30.5:1.4:0.1] | |
| F10 | CICL1:DSPC:CHOL:DSG-PEG(2k):DSPE-PEG(2k)-MAL | 6 |
| | [62:10:26.5:1.4:0.1] | |
| F11 | CICL1:DSPC:CHOL:DMG-PEG(2k):DSPE-PEG(2k)-MAL | 6 |
| | [58:7:33.5:1.4:0.1] | |
| F12 | CICL1:DSPC:CHOL:DPG-PEG(2k):DSPE-PEG(2k)-MAL | 6 |
| | [58:7:33.5:1.4:0.1] | |
| F13 | CICL1:DSPC:CHOL:DSG-PEG(2k):DSPE-PEG(2k)-MAL | 6 |
| | [58:7:33.5:1.4:0.1] | |
| F14 | CICL1:DSPC:CHOL:DMG-PEG(2k):DSPE-PEG(2k)-MAL | 6 |
| | [58:7:34:0.9:0.1] | |
| F15 | CICL1:DSPC:CHOL:DSG-PEG(2k):DSPE-PEG(2k)-MAL | 6 |
| | [58:10:30:1.9:0.1] | |
| F16 | CICL1:DSPC:CHOL:DSG-PEG(2k):DSPE-PEG(2k)-MAL | 6 |
| | [50:10:39.5:0.4:0.1] | |
| F17 | CICL1:DSPC:CHOL:DSG-PEG(2k):DSPE-PEG(2k)-MAL | 6 |
| | [50:10:39:0.9:0.1] | |
| F18 | CICL1:DSPC:CHOL:DSG-PEG(2k):DSPE-PEG(2k)-MAL | 6 |
| | [50:10:38.5:1.4:0.1] | |
| F19 | CICL1:DSPC:CHOL:DSG-PEG(2k):DSPE-PEG(2k)-MAL | 6 |
| | [50:10:38:1.9:0.1] | |
| F20 | CICL1:DSPC:CHOL:DSG-PEG(2k):DSPE-PEG(2k)-MAL | 6 |
| | [50:10:37.5:2.4:0.1] | |
| F21 | CICL1:DSPC:CHOL:DSG-PEG(2k):DSPE-PEG(2k)-MAL | 6 |
| | [50:10:37:2.9:0.1] | |
| F22 | CICL1:DSPC:CHOL:DSG-PEG(2k):DSPE-PEG(2k)-MAL | 6 |
| | [58:10:31:0.9:0.1] | |
| F23 | CICL1:DSPC:CHOL:DSG-PEG(2k):DSPE-PEG(2k)-MAL | 6 |
| | [58:10:30:1.9:0.1] | |
| F24 | CICL1:DSPC:CHOL:DSG-PEG(2k):DSPE-PEG(2k)-MAL | 6 |
| | [58:10:29.5:2.4:0.1] | |
| F25 | CICL1:DSPC:CHOL:DSPE-PEG(0.75k):DSPE-PEG(2k)-MAL | 6 |
| | [58:10:30.5:1.4:0.1] | |
| F26 | CICL1:DSPC:CHOL:DSPE-PEG(1k):DSPE-PEG(2k)-MAL | 6 |
| | [58:10:30.5:1.4:0.1] | |
| F27 | CICL1:DSPC:CHOL:DMPE-PEG(1k):DSPE-PEG(2k)-MAL | 6 |
| | [58:10:30.5:1.4:0.1] | |
| F29 | CICL1:DSPC:CHOL:DSG-PEG(5k):DSPE-PEG(5k)-MAL | 6 |
| | [58:10:31.4:0.5:0.1] | |
| F30 | CICL1:DSPC:CHOL:DMG-PEG(2k):DSG-PEG(2k)-MAL | 6 |
| | [58:10:30.5:1.4:0.1] | |
| F31 | CICL1:DSPC:CHOL:DSG-PEG(2k):DSG-PEG(2k)-MAL | 6 |
| | [58:10:30.5:1.4:0.1] | |
| F32 | CICL1:DSPC:CHOL:DSPE-PEG(2k):DSPE-PEG(2k)-MAL | 6 |
| | [58:10:30.5:1.4:0.1] | |
| F33 | CICL1:DSPC:CHOL:DSG-PEG(2k):DSPE-PEG(5k)-MAL | 6 |
| | [58:10:30.5:1.4:0.1] | |
| F34 | CICL1:DSPC:CHOL:DSG-PEG(2k):DSPE-PEG(2k)-MAL | 6 |
| | [58:13:27.5:1.4:0.1] | |
| F35 | CICL1:DMPC:CHOL:DSG-PEG(2k):DSPE-PEG(2k)-MAL | 6 |
| | [58:10:30.5:1.4:0.1] | |
| F36 | CICL1:DPPC:CHOL:DSG-PEG(2k):DSPE-PEG(2k)-MAL | 6 |
| | [58:10:30.5:1.4:0.1] | |
| F37 | CICL1:DAPC:CHOL:DSG-PEG(2k):DSPE-PEG(2k)-MAL | 6 |
| | [58:10:30.5:1.4:0.1] | |
| F38 | CICL1:18:1 PA:CHOL:DSG-PEG(2k):DSPE-PEG(2k)-MAL | 6 |
| | [58:10:30.5:1.4:0.1] | |
| F40 | CICL1:DSPC:CHOL:20(S)-Hydroxycholesterol:DSG-PEG(2k):DSPE- | 6 |
| | PEG(2k)-MAL [58:10:22.9:7.6:1.4:0.1] | |
| F41 | CICL1:DSPC:CHOL:β-Sitosterol:DSG-PEG(2k):DSPE-PEG(2k)-MAL | 6 |
| | [58:10:22.9:7.6:1.4:0.1] | |
| F42 | CICL1:DSPC:CHOL:β-Sitosterol:DSG-PEG(2k):DSPE-PEG(2k)-MAL | 6 |
| | [58:10:15.25:15.25:1.4:0.1] | |

TABLE 20
Humanized anti-CD8α F(ab′) variants and classic
and engineered F(ab′) constant regions.

| Name | Notes | LC | HC |
|---|---|---|---|
| .6 IgG1 F(ab′) CH | Wildtype human IgG1 F(ab′) | | SEQ ID NO: 262 |
| CBD1033.6 | h1017-L2 | SEQ ID NO: 263 | SEQ ID NO: 264 |
| | h1017-H2 | | |
| | IgG1 F(ab′) | | |
| | Kappa | | |
| S162C and C214S | Kappa chain | SEQ ID NO: 180 | |
| Kappa chain | S162C | | |
| | C214S | | |
| .37 Truncated P241, | IgG1 F(ab′) | | SEQ ID NO: 181 |
| P240A and P241A, | Truncated P241 | | |
| F174C, C233S IgG1 | P240A and P241A | | |
| F(ab′) CH | F174C, C233S | | |
| CBD1033.37 | h1017-L2 | SEQ ID NO: 182 | SEQ ID NO: 183 |
| | h1017-H2 | | |
| | Truncated P241, P240A and | | |
| | P241A IgG1 F(ab′) | | |
| | CK-S162C and CH1-F174C | | |
| | engineered disulfide bond | | |
| | CK-C214S and CH1-C233S | | |
| | to abolish native disulfide bond | | |
| .42 Truncated P240, | IgG1 F(ab′) | | SEQ ID NO: 184 |
| F174C, C233S IgG1 | Truncated P240 | | |
| F(ab′) CH | F174C, C233S | | |
| CBD1033.42 | h1017-L2 | SEQ ID NO: 182 | SEQ ID NO: 185 |
| | h1017-H2 | | |
| | Truncated P240 IgG1 F(ab′) | | |
| | CK-C214S and CH1-C233S | | |
| | to abolish native disulfide bond | | |
| | CK-S162C and CH1-F174C | | |
| | engineered disulfide bond. | | |
| .44 Truncated T238, | IgG1 F(ab′) | | SEQ ID NO: 186 |
| F174C IgG1 | Truncated at T238 | | |
| F(ab′) CH | F174C | | |
| CBD1033.44 | h1017-L2 | SEQ ID NO: 182 | SEQ ID NO: 187 |
| | h1017-H2 | | |
| | Truncated T238 IgG1 F(ab′) | | |
| | CK-C214S to abolish native | | |
| | disulfide bond | | |
| | CK-S162C and CH1-F174C | | |
| | engineered disulfide bond | | |
| CBD1381.37 | h1017-L2 | SEQ ID NO: 182 | SEQ ID NO: 188 |
| | N55Q of h1017-H2 | | |
| | Truncated P241, P240A and | | |
| | P241A IgG1 F(ab′) | | |
| | CK-C214S and CH1-C233S | | |
| | to abolish native disulfide bond | | |
| | CK-S162C and CH1-F174C | | |
| | engineered disulfide bond | | |
| CBD1382.37 | h1017-L2 | SEQ ID NO: 182 | SEQ ID NO: 189 |
| | N55A of h1017-H2 | | |
| | Truncated P241, P240A and | | |
| | P241A IgG1 F(ab′) | | |
| | CK-C214S and CH1-C233S | | |
| | to abolish native disulfide bond | | |
| | CK-S162C and CH1-F174C | | |
| | engineered disulfide bond | | |
| CBD1444.37 | D30S of h1017-L2 | SEQ ID NO: 190 | SEQ ID NO: 183 |
| | h1017-H2 | | |
| | Truncated P241, P240A and | | |
| | P241A IgG1 F(ab′) | | |
| | CK-C214S and CH1-C233S | | |
| | to abolish native disulfide bond | | |
| | CK-S162C and CH1-F174C | | |
| | engineered disulfide bond | | |
| CBD1622.37 | D30S of h1017-L2 | SEQ ID NO: 190 | SEQ ID NO: 188 |
| | N55Q of h1017-H2 | | |
| | Truncated P241, P240A and | | |
| | P241A IgG1 F(ab′) | | |
| | CK-C214S and CH1-C233S | | |
| | to abolish native disulfide bond | | |
| | CK-S162C and CH1-F174C | | |
| | engineered disulfide bond. | | |
| CBD1623.37 | D30S of h1017-L2 | SEQ ID NO: 190 | SEQ ID NO: 189 |
| | N55A of h1017-H2 | | |
| | Truncated P241, P240A and | | |
| | P241A IgG1 F(ab′) | | |
| | CK-C214S and CH1-C233S | | |
| | to abolish native disulfide bond | | |
| | CK-S162C and CH1-F174C | | |
| | engineered disulfide bond | | |
| CBD1622.42 | D30S of h1017-L2 | SEQ ID NO: 190 | SEQ ID NO: 191 |
| | N55Q of h1017-H2 | | |
| | Truncated P240 IgG1 F(ab′) | | |
| | CK-C214S and CH1-C233S | | |
| | to abolish native disulfide bond | | |
| | CK-S162C and CH1-F174C | | |
| | engineered disulfide bond | | |
| CBD1623.42 | D30S of h1017-L2 | SEQ ID NO: 190 | SEQ ID NO: 192 |
| | N55A of h1017-H2 | | |
| | Truncated P240 IgG1 F(ab′) | | |
| | CK-C214S and CH1-C233S | | |
| | to abolish native disulfide bond | | |
| | CK-S162C and CH1-F174C | | |
| | engineered disulfide bond | | |
| CBD1622.44 | D30S of h1017-L2 | SEQ ID NO: 190 | SEQ ID NO: 193 |
| | N55Q of h1017-H2 | | |
| | Truncated T238 IgG1 F(ab′) | | |
| | CK-C214S to abolish native | | |
| | disulfide bond | | |
| | CK-S162C and CH1-F174C | | |
| | engineered disulfide bond. | | |
| CBD1623.44 | D30S of h1017-L2 | SEQ ID NO: 190 | SEQ ID NO: 194 |
| | N55A of h1017-H2 | | |
| | Truncated T238 IgG1 F(ab′) | | |
| | CK-C214S to abolish native | | |
| | disulfide bond | | |
| | CK-S162C and CH1-F174C | | |
| | engineered disulfide bond. | | |

TABLE 21
Summary of full-length and F(ab′) humanized anti-CD8α variants constructs.

| Name | VH | HC constant region | VL | LC constant region |
|---|---|---|---|---|
| CBD1032 | h1017-H1 | Fc-silenced IgG1 | h1017-L1 | Kappa chain |
| | (SEQ ID NO: 117) | (SEQ ID NO: 146) | (SEQ ID NO: 123) | (SEQ ID NO: 144) |
| CBD1033 | h1017-H2 | Fc-silenced IgG1 | h1017-L2 | Kappa chain |
| | (SEQ ID NO: 118) | (SEQ ID NO: 146) | (SEQ ID NO: 124) | (SEQ ID NO: 144) |
| CBD1034 | h1017-H2 | Fc-silenced IgG1 | h1017-L3 | Kappa chain |
| | (SEQ ID NO: 118) | (SEQ ID NO: 146) | (SEQ ID NO: 125) | (SEQ ID NO: 144) |
| CBD1035 | h1017-H3 | Fc-silenced IgG1 | h1017-L2 | Kappa chain |
| | (SEQ ID NO: 119) | (SEQ ID NO: 146) | (SEQ ID NO: 124) | (SEQ ID NO: 144) |
| CBD1036 | h1017-H3 | Fc-silenced IgG1 | h1017-L3 | Kappa chain |
| | (SEQ ID NO: 119) | (SEQ ID NO: 146) | (SEQ ID NO: 125) | (SEQ ID NO: 144) |
| CBD1037 | h1017-H4 | Fc-silenced IgG1 | h1017-L2 | Kappa chain |
| | (SEQ ID NO: 120) | (SEQ ID NO: 146) | (SEQ ID NO: 124) | (SEQ ID NO: 144) |
| CBD1038 | h1017-H4 | Fc-silenced IgG1 | h1017-L3 | Kappa chain |
| | (SEQ ID NO: 120) | (SEQ ID NO: 146) | (SEQ ID NO: 125) | (SEQ ID NO: 144) |
| CBD1039 | h1017-H5 | Fc-silenced IgG1 | h1017-L2 | Kappa chain |
| | (SEQ ID NO: 121) | (SEQ ID NO: 146) | (SEQ ID NO: 124) | (SEQ ID NO: 144) |
| CBD1040 | h1017-H5 | Fc-silenced IgG1 | h1017-L3 | Kappa chain |
| | (SEQ ID NO: 121) | (SEQ ID NO: 146) | (SEQ ID NO: 125) | (SEQ ID NO: 144) |
| CBD1041 | h1017-H6 | Fc-silenced IgG1 | h1017-L4 | Kappa chain |
| | (SEQ ID NO: 135) | (SEQ ID NO: 146) | (SEQ ID NO: 141) | (SEQ ID NO: 144) |
| CBD1042 | h1017-H7 | Fc-silenced IgG1 | h1017-L4 | Kappa chain |
| | (SEQ ID NO: 136) | (SEQ ID NO: 146) | (SEQ ID NO: 141) | (SEQ ID NO: 144) |
| CBD1043 | h1017-H7 | Fc-silenced IgG1 | h1017-L5 | Kappa chain |
| | (SEQ ID NO: 136) | (SEQ ID NO: 146) | (SEQ ID NO: 142) | (SEQ ID NO: 144) |
| CBD1044 | h1017-H7 | Fc-silenced IgG1 | h1017-L6 | Kappa chain |
| | (SEQ ID NO: 136) | (SEQ ID NO: 146) | (SEQ ID NO: 143) | (SEQ ID NO: 144) |
| CBD1045 | h1017-H8 | Fc-silenced IgG1 | h1017-L5 | Kappa chain |
| | (SEQ ID NO: 137) | (SEQ ID NO: 146) | (SEQ ID NO: 142) | (SEQ ID NO: 144) |
| CBD1046 | h1017-H8 | Fc-silenced IgG1 | h1017-L6 | Kappa chain |
| | (SEQ ID NO: 137) | (SEQ ID NO: 146) | (SEQ ID NO: 143) | (SEQ ID NO: 144) |
| CBD1047 | h1017-H9 | Fc-silenced IgG1 | h1017-L5 | Kappa chain |
| | (SEQ ID NO: 138) | (SEQ ID NO: 146) | (SEQ ID NO: 142) | (SEQ ID NO: 144) |
| CBD1048 | h1017-H9 | Fc-silenced IgG1 | h1017-L6 | Kappa chain |
| | (SEQ ID NO: 138) | (SEQ ID NO: 146) | (SEQ ID NO: 143) | (SEQ ID NO: 144) |
| CBD1049 | h1017-H10 | Fc-silenced IgG1 | h1017-L5 | Kappa chain |
| | (SEQ ID NO: 139) | (SEQ ID NO: 146) | (SEQ ID NO: 142) | (SEQ ID NO: 144) |
| CBD1050 | h1017-H10 | Fc-silenced IgG1 | h1017-L6 | Kappa chain |
| | (SEQ ID NO: 139) | (SEQ ID NO: 146) | (SEQ ID NO: 143) | (SEQ ID NO: 144) |
| CBD1128 | h1017-H1 | Fc-silenced IgG1 | h1017-L2 | Kappa chain |
| | (SEQ ID NO: 117) | (SEQ ID NO: 146) | (SEQ ID NO: 124) | (SEQ ID NO: 144) |
| CBD1129 | h1017-H2 | Fc-silenced IgG1 | h1017-L1 | Kappa chain |
| | (SEQ ID NO: 118) | (SEQ ID NO: 146) | (SEQ ID NO: 123) | (SEQ ID NO: 144) |
| CBD1380 | N55S of h1017-H2 | Fc-silenced IgG1 | h1017-L2 | Kappa chain |
| | (SEQ ID NO: 130) | (SEQ ID NO: 146) | (SEQ ID NO: 124) | (SEQ ID NO: 144) |
| CBD1381 | N55Q of h1017-H2 | Fc-silenced IgG1 | h1017-L2 | Kappa chain |
| | (SEQ ID NO: 131) | (SEQ ID NO: 146) | (SEQ ID NO: 124) | (SEQ ID NO: 144) |
| CBD1382 | N55A of h1017-H2 | Fc-silenced IgG1 | h1017-L2 | Kappa chain |
| | (SEQ ID NO: 132) | (SEQ ID NO: 146) | (SEQ ID NO: 124) | (SEQ ID NO: 144) |
| CBD1383 | N55D of h1017-H2 | Fc-silenced IgG1 | h1017-L2 | Kappa chain |
| | (SEQ ID NO: 133) | (SEQ ID NO: 146) | (SEQ ID NO: 124) | (SEQ ID NO: 144) |
| CBD1443 | h1017-H2 | Fc-silenced IgG1 | D30E of h1017-L2 | Kappa chain |
| | (SEQ ID NO: 118) | (SEQ ID NO: 146) | (SEQ ID NO: 166) | (SEQ ID NO: 144) |
| CBD1444 | h1017-H2 | Fc-silenced IgG1 | D30S of h1017-L2 | Kappa chain |
| | (SEQ ID NO: 118) | (SEQ ID NO: 146) | (SEQ ID NO: 167) | (SEQ ID NO: 144) |
| CBD1445 | h1017-H2 | Fc-silenced IgG1 | D30A of h1017-L2 | Kappa chain |
| | (SEQ ID NO: 118) | (SEQ ID NO: 146) | (SEQ ID NO: 168) | (SEQ ID NO: 144) |
| CBD1446 | h1017-H4 | Fc-silenced IgG1 | D30E of h1017-L2 | Kappa chain |
| | (SEQ ID NO: 120) | (SEQ ID NO: 146) | (SEQ ID NO: 166) | (SEQ ID NO: 144) |
| CBD1447 | h1017-H4 | Fc-silenced IgG1 | D30S of h1017-L2 | Kappa chain |
| | (SEQ ID NO: 120) | (SEQ ID NO: 146) | (SEQ ID NO: 167) | (SEQ ID NO: 144) |
| CBD1448 | h1017-H4 | Fc-silenced IgG1 | D30A of h1017-L2 | Kappa chain |
| | (SEQ ID NO: 120) | (SEQ ID NO: 146) | (SEQ ID NO: 168) | (SEQ ID NO: 144) |
| CBD1449 | N55S of h1017-H4 | Fc-silenced IgG1 | h1017-L2 | Kappa chain |
| | (SEQ ID NO: 169) | (SEQ ID NO: 146) | (SEQ ID NO: 124) | (SEQ ID NO: 144) |
| CBD1450 | N55Q of h1017-H4 | Fc-silenced IgG1 | h1017-L2 | Kappa chain |
| | (SEQ ID NO: 170) | (SEQ ID NO: 146) | (SEQ ID NO: 124) | (SEQ ID NO: 144) |
| CBD1451 | N55A of h1017-H4 | Fc-silenced IgG1 | h1017-L2 | Kappa chain |
| | (SEQ ID NO: 171) | (SEQ ID NO: 146) | (SEQ ID NO: 124) | (SEQ ID NO: 144) |
| CBD1575 | N55Q of h1017-H4 | Fc-silenced IgG1 | D30S of h1017-L2 | Kappa chain |
| | (SEQ ID NO: 170) | (SEQ ID NO: 146) | (SEQ ID NO: 167) | (SEQ ID NO: 144) |
| CBD1576 | N55A of h1017-H2 | Fc-silenced IgG1 | D30S of h1017-L2 | Kappa chain |
| | (SEQ ID NO: 171) | (SEQ ID NO: 146) | (SEQ ID NO: 167) | (SEQ ID NO: 144) |
| CBD1622 | N55Q of h1017-H2 | Fc-silenced IgG1 | D30S of h1017-L2 | Kappa chain |
| | (SEQ ID NO: 131) | (SEQ ID NO: 146) | (SEQ ID NO: 167) | (SEQ ID NO: 144) |
| CBD1623 | N55A of h1017-H2 | Fc-silenced IgG1 | D30S of h1017-L2 | Kappa chain |
| | (SEQ ID NO: 132) | (SEQ ID NO: 146) | (SEQ ID NO: 167) | (SEQ ID NO: 144) |
| CBD1033.6 | h1017-H2 | IgG1 F(ab′) | h1017-L2 | Kappa chain |
| | (SEQ ID NO: 118) | (SEQ ID NO: 262) | (SEQ ID NO: 124) | (SEQ ID NO: 144) |
| CBD1033.37 | h1017-H2 | Truncated P241, | h1017-L2 | S162C and C214S |
| | (SEQ ID NO: 118) | P240A and P241A, | (SEQ ID NO: 124) | Kappa chain |
| | | F174C, C233S | | (SEQ ID NO: 180) |
| | | IgG1 F(ab′) | | |
| | | (SEQ ID NO: 181) | | |
| CBD1033.42 | h1017-H2 | Truncated P240, | h1017-L2 | S162C and C214S |
| | (SEQ ID NO: 118) | F174C, C233S | (SEQ ID NO: 124) | Kappa chain |
| | | IgG1 F(ab′) | | (SEQ ID NO: 180) |
| | | (SEQ ID NO: 184) | | |
| CBD1033.44 | h1017-H2 | Truncated T238, | h1017-L2 | S162C and C214S |
| | (SEQ ID NO: 118) | F174C IgG1 F(ab′) | (SEQ ID NO: 124) | Kappa chain |
| | | (SEQ ID NO: 186) | | (SEQ ID NO: 180) |
| CBD1381.37 | N55Q of h1017-H2 | Truncated P241, | h1017-L2 | S162C and C214S |
| | (SEQ ID NO: 131) | P240A and P241A, | (SEQ ID NO: 124) | Kappa chain |
| | | F174C, C233S | | (SEQ ID NO: 180) |
| | | IgG1 F(ab′) | | |
| | | (SEQ ID NO: 181) | | |
| CBD1382.37 | N55A of h1017-H2 | Truncated P241, | h1017-L2 | S162C and C214S |
| | (SEQ ID NO: 132) | P240A and P241A, | (SEQ ID NO: 124) | Kappa chain |
| | | F174C, C233S | | (SEQ ID NO: 180) |
| | | IgG1 F(ab′) | | |
| | | (SEQ ID NO: 181) | | |
| CBD1444.37 | h1017-H2 | Truncated P241, | D30S of h1017-L2 | S162C and C214S |
| | (SEQ ID NO: 118) | P240A and P241A, | (SEQ ID NO: 167) | Kappa chain |
| | | F174C, C233S | | (SEQ ID NO: 180) |
| | | IgG1 F(ab′) | | |
| | | (SEQ ID NO: 181) | | |
| CBD1622.37 | N55Q of h1017-H2 | Truncated P241, | D30S of h1017-L2 | S162C and C214S |
| | (SEQ ID NO: 131) | P240A and P241A, | (SEQ ID NO: 167) | Kappa chain |
| | | F174C, C233S | | (SEQ ID NO: 180) |
| | | IgG1 F(ab′) | | |
| | | (SEQ ID NO: 181) | | |
| CBD1623.37 | N55A of h1017-H2 | Truncated P241, | D30S of h1017-L2 | S162C and C214S |
| | (SEQ ID NO: 132) | P240A and P241A, | (SEQ ID NO: 167) | Kappa chain |
| | | F174C, C233S | | (SEQ ID NO: 180) |
| | | IgG1 F(ab′) | | |
| | | (SEQ ID NO: 181) | | |
| CBD1622.42 | N55Q of h1017-H2 | Truncated P240, | D30S of h1017-L2 | S162C and C214S |
| | (SEQ ID NO: 131) | F174C, C233S | (SEQ ID NO: 167) | Kappa chain |
| | | IgG1 F(ab′) | | (SEQ ID NO: 180) |
| | | (SEQ ID NO: 184) | | |
| CBD1623.42 | N55A of h1017-H2 | Truncated P240, | D30S of h1017-L2 | S162C and C214S |
| | (SEQ ID NO: 132) | F174C, C233S | (SEQ ID NO: 167) | Kappa chain |
| | | IgG1 F(ab′) | | (SEQ ID NO: 180) |
| | | (SEQ ID NO: 184) | | |
| CBD1622.44 | N55Q of h1017-H2 | Truncated T238, | D30S of h1017-L2 | S162C and C214S |
| | (SEQ ID NO: 131) | F174C IgG1 F(ab′) | (SEQ ID NO: 167) | Kappa chain |
| | | (SEQ ID NO: 186) | | (SEQ ID NO: 180) |
| CBD1623.44 | N55A of h1017-H2 | Truncated T238, | D30S of h1017-L2 | S162C and C214S |
| | (SEQ ID NO: 132) | F174C IgG1 F(ab′) | (SEQ ID NO: 167) | Kappa chain |
| | | (SEQ ID NO: 186) | | (SEQ ID NO: 180) |

### EMBODIMENTS

Embodiment 1. A method of increasing in vivo transfection efficiency of T cells, comprising administering to a mammalian subject in a compact regimen multiple doses of a T cell-targeted lipid nanoparticle (tLNP) encapsulating an RNA encoding a T cell activating agent, wherein the tLNP delivers the RNA encoding the T cell activating agent to the targeted T cells in the subject and the targeted T cells express the T cell activating agent, wherein the compact dose regimen comprises administering a second dose after an initial dose within 1 to 5 days, whereby more T cells express the T cell activating agent as a result of a subsequent administration than as a result of the initial administration, and wherein any subsequent dose is administered within 1 to 5 days of the immediately preceding dose.

Embodiment 2. The method of embodiment 1, wherein the second dose is administered 2 days, 3 days, or 4 days after the initial dose.

Embodiment 3. The method of embodiment 1 or 2, wherein the compact dose regimen comprises 2 or 3 doses.

Embodiment 4. The method of embodiment 3, wherein the 2 to 3 doses are administered at 72-hour intervals (2xQ72h or 3xQ72h).

Embodiment 5. The method of any one of embodiments 1-4, wherein the tLNP dosage for each administration ranges from about 0.1 to about 1.5 mg RNA/kg.

Embodiment 6. The method of any one of embodiments 1-5, wherein (a) the initial tLNP dose is the same as each subsequent dose, (b) the initial tLNP dose is lower than each subsequent dose, or (c) the initial tLNP dose is higher than each subsequent dose.

Embodiment 7. The method of any one of embodiments 1-5, wherein (a) any subsequent dose is higher than the initial dose, (b) any subsequent dose is lower that the initial dose, (c) the last dose is higher than all preceding doses.

Embodiment 8. The method of any one of embodiments 1-7, wherein the tLNP encapsulated RNA is mRNA, circular RNA, or self-replicating RNA.

Embodiment 9. The method of any one of embodiments 1-8, wherein the tLNP encapsulated RNA is mRNA.

Embodiment 10. The method of any one of embodiments 1-9, wherein the encoded T cell activating agent is a chimeric antigen receptor (CAR), a T cell receptor (TCR), a T cell engager (TCE), a conditioning agent, or any combination thereof.

Embodiment 11. The method of embodiment 10, wherein the encoded T cell activating agent is a CAR, wherein the CAR comprises a binding moiety specific for an antigen that is CD19, BCMA, FAP, CAIX (CA9), CD, CD5, CD20 (MS4A1), CD22, CD23, CD30 (TNFRSF8), CD33, CD38, CD44, CD56 (NCAM1), CD70, CD133, CD138 (SDC1), CD171 (L1-CAM), CD174, CD248 (TEM1), CD267 (TACI), CD274 (PD-L1), CD276 (B7-H3), CD279 (PD-1), CD319 (SLAMF7), CEA, CEACAM5, Claudin 6 (CLDN6), Claudin 18.2 (CLDN18.2), CLL1, CSPG4, DLL3, EGFR, EGFRvIII, EPCAM, EPHA2, ERBB2, FCRL5, FOLH1, FOLR1, GD2, GPC3, GPNMB, GPRC5D, HER2, IL1RAP, IL3RA, IL-13Rα, IL13RA2 (IL13Rα2), KDR (VEGFR2), LRRC15, MET, Mesothelin (MSLN), MUC1, MUC16, PSCA, PSMA, ROR1, TROP2, ULBP1, or ULBP2.

Embodiment 12. The method of embodiment 10 or 11, wherein the CAR is specific for CD19 and has the amino acid sequence of CAR1 or CAR2 after cleavage of the signal sequence.

Embodiment 13. The method of embodiment 12, wherein the mRNA encoding CAR2 has the nucleotide sequence RM_61355 (SEQ ID NO: 206), RM_61357 (SEQ ID NO: 208), RM_61461 (SEQ ID NO: 212), RM_61482 (SEQ ID NO: 213), RM_61483 (SEQ ID NO: 214), RM_61486 (SEQ ID NO: 215), RM_61487 (SEQ ID NO: 216), RM_61488 (SEQ ID NO: 217), RM_61489 (SEQ ID NO: 218), RM_61458 (SEQ ID NO: 211), RM_61455 (SEQ ID NO: 210), RM_61356 (SEQ ID NO: 207), or RM_61358 (SEQ ID NO: 209).

Embodiment 14. The method of any one of embodiments 1-13, wherein the tLNP comprises an ionizable cationic lipid of Formula 1:

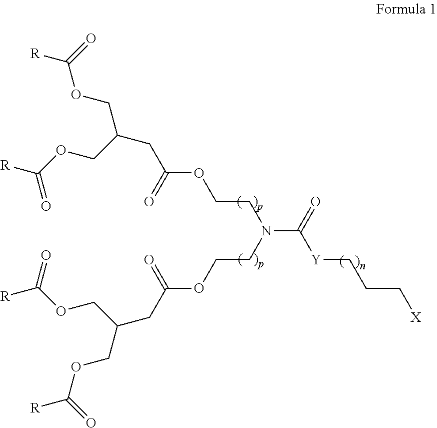

wherein
   Y is O, NH, N—CH₃, or CH₂,
   n is an integer from 0 to 4,
   X is

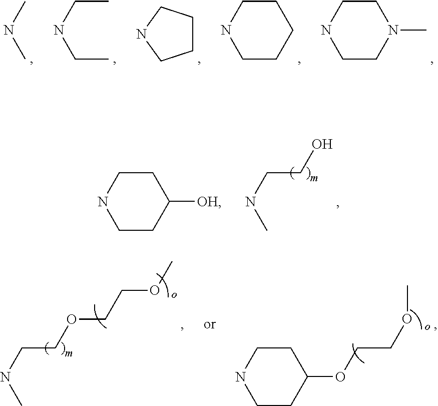

m is an integer from 1 to 3,
is an integer from 1 to 4, and
p is an integer from 1 to 4,
wherein when p=1:
   each R is independently C₆ to C₁₆ straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain;
wherein when p=2:
   each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or Cs to C₁₆ aryl-alkyl in which the aryl is phenyl or naphthalenyl and is positioned at either end or within the alkyl chain;
wherein when p=3:
   each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain; and
wherein when p=4:
each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or Cs to C₁₂ aryl-alky in which the aryl is phenyl or naphthalenyl and is positioned at the either end or within the alkyl chain.

Embodiment 15. The method of any one of embodiments 1-13, wherein the tLNP comprises an ionizable cationic lipid of Formula M5:

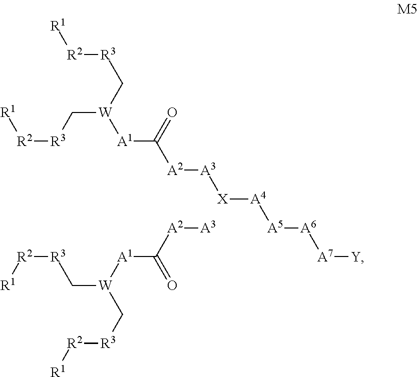

wherein
   each R¹ is independently selected from a C₇-C₁₁ alkyl or a C₇-C₁₁ alkenyl,
   A¹ is (CH₂)₁₋₂,
   A² is O,
   A³ is (CH₂)₁₋₅, wherein A³ is not CH₂ if X is N,
   X is N, CH, or C—CH₃,
   A⁴ is CH₂, C═O, NH, NCH₃, or O,
   A⁵ is absent, O, S, NH, or NCH₃ if A⁴ is C═O, or A⁵ is C═O if A⁴ is not C═O,
   A⁶ is O, S, NH, NCH₃ or (CH₂)₀₋₂,
   A⁷ is (CH₂)₀₋₆, wherein if A⁶ is O, S, NH, NCH₃, A⁷ is (CH₂)₂₋₄,
   Y is

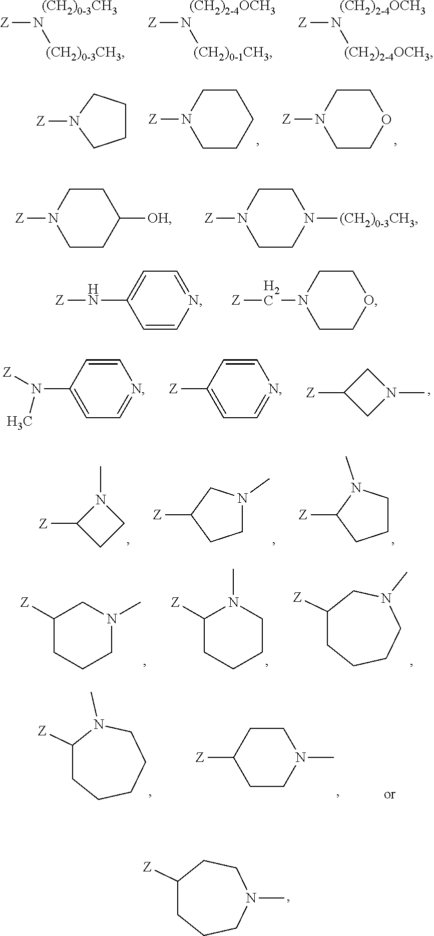

wherein Z is a bond; and
R² is O, R³ is C═O and W is CH or N, or R² is C═O, R³ is O and W is CH;
wherein A⁶ and A⁷ are not both (CH₂)₀ unless A⁵ is C═0;
wherein
   a) A¹ is CH₂, A³ is (CH₂)₂₋₅, X is N, A⁴ is C═O, A⁵ is O, S, NH, NCH₃, A⁶ is (CH₂)₁₋₂, A⁷ is (CH₂)₁₋₄, or
   b) A¹ is CH₂, A³ is (CH₂)₁₋₄, X is CH, A⁴ is CH₂, NH, NCH₃, O, A⁵ is C═O, A⁶ is O, NH, NCH₃, A⁷ is (CH)₂₋₆, or
   c) A¹ is (CH₂)₂, A³ is (CH₂)₁₋₄, X is C—CH₃, A⁴ is C═O, A⁵ is O, NH, NCH₃, A⁶ is (CH₂)₁₋₂, A⁷ is (CH₂)₁₋₄, or
   d) A¹ is CH₂, A³ is (CH₂)₂₋₅, X is N, A⁴ is C═O, A⁵ is absent, A⁶ is (CH₂)₀, A⁷ is (CH₂)₀, and Y is

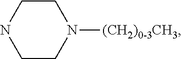

or
e) A¹ is CH₂, A³ is (CH₂)1-5, X is CH, A⁴ is CH₂, NH, NCH₃ or O, A⁵ is C═O, A⁶ is (CH₂)₀, A⁷ is (CH₂)₀, and Y is

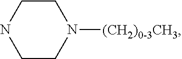

or
f) A¹ is (CH₂)₂, A³ is (CH₂)₁₋₅, X is CCH₃, A⁴ is C═O, A⁵ is absent, A⁶ is (CH₂)₀, A⁷ is (CH₂)₀, and Y is

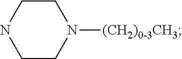

wherein
   the number of contiguous atoms present in a span:

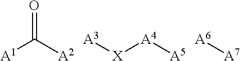

is in the range from 7-17.

Embodiment 16. The method of any one of embodiments 1-13, wherein the tLNP comprises an ionizable cationic lipid of Formula M6:

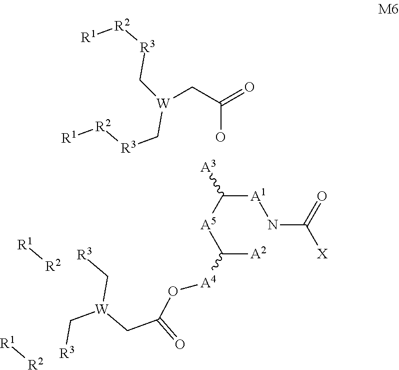

wherein X is

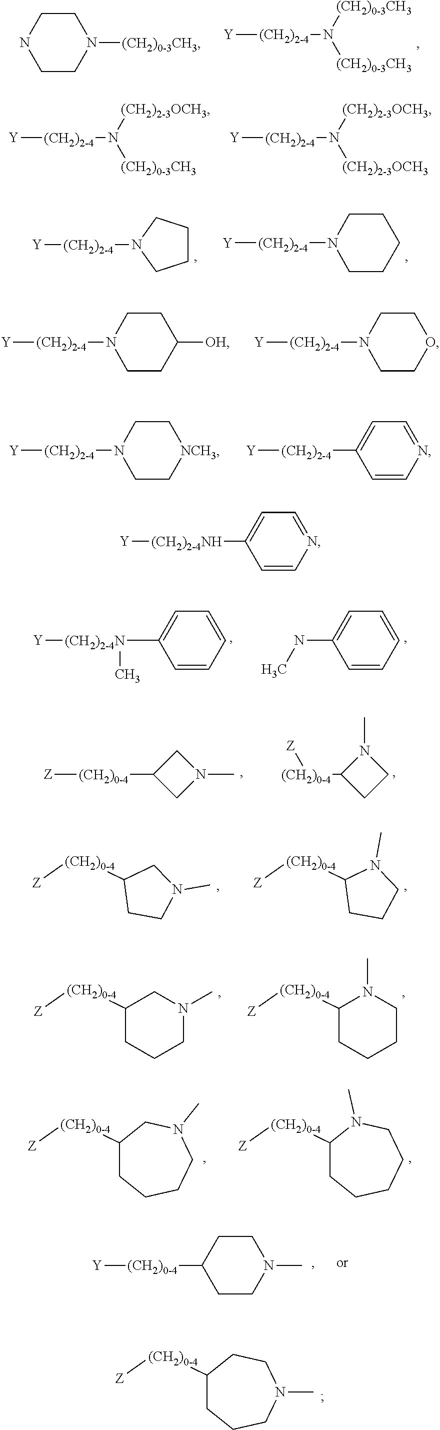

and
Y is O, S, NH, or NCH₃;
Z is O, NH, or NCH₃;
R² is O, R³ is C═O and W is CH or N, or R² is C═O, R³ is O and W is CH; and
each R¹ is independently selected from a C₇-C₁₁ alkyl or a C₇-C₁₁ alkenyl;
each A¹, A², A³, and A⁴ is independently selected from (CH₂)₀ and (CH₂)₁, A⁵ is selected from (CH₂)₀₋₄, CH═CH, and CH₂—CH═CH—CH₂; and
a wavy bond indicates that any relative or absolute stereo-configuration of the corresponding ring atom, or a mixture of stereo-configurations, can be assumed.

Embodiment 17. The method of embodiment 14, wherein the ionizable cationic lipid comprises

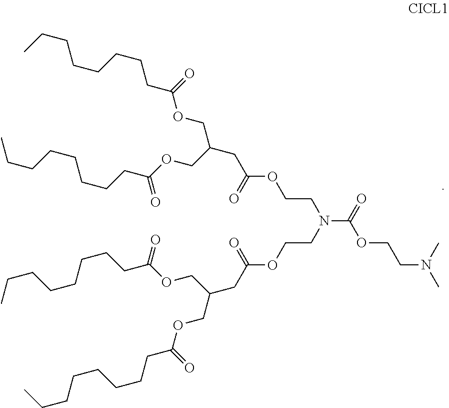 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OCCN(C)C]

Embodiment 18. The method of embodiment 16, wherein the ionizable cationic lipid comprises:

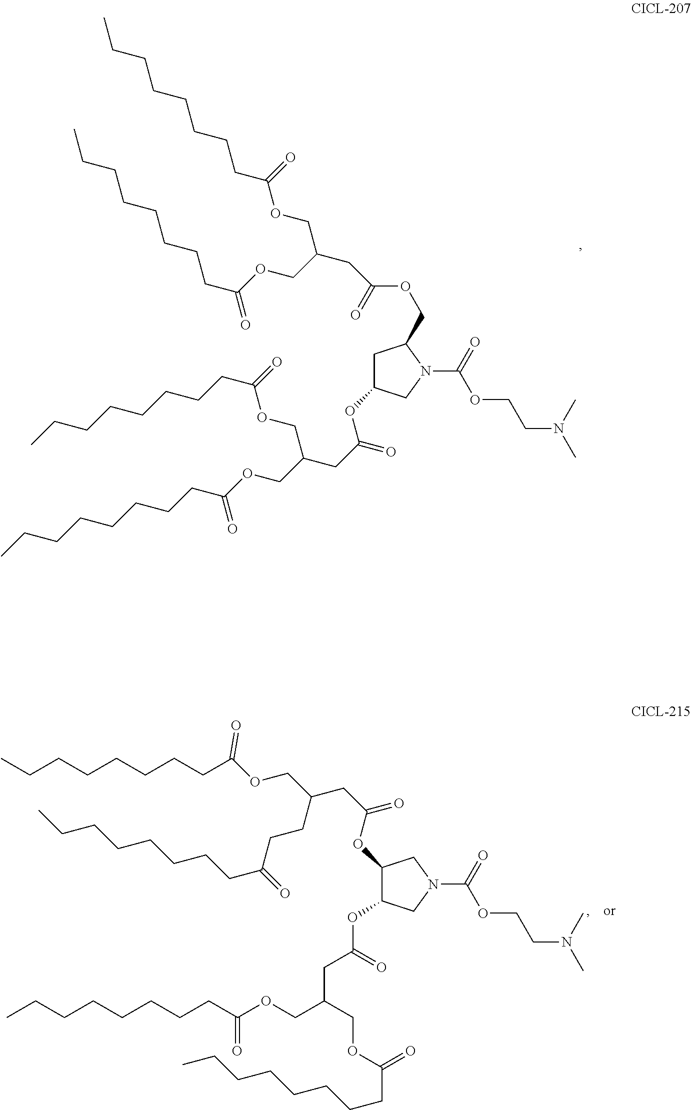

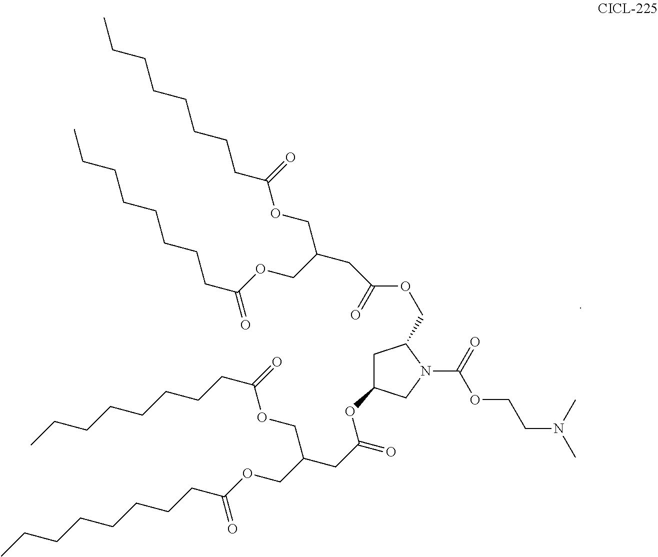 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OCCN(C)C]

Embodiment 19. The method of any one of embodiments 1-16, wherein the tLNP comprises about 35 to about 65 mol % ionizable cationic lipid, about 0.5 to about 3 mol % PEG-lipid comprising functionalized PEG-lipid and non-functionalized PEG-lipid, about 7 to about 13 mol % phospholipid, and about 27 to about 50 mol % sterol.

Embodiment 20. The method of embodiment 19, wherein the tLNP comprises about 58% ionizable cationic lipid, about 30.5 mol % cholesterol, about 10 mol % distearoylphosphatidylcholine (DSPC), about 1.4 mol % distearoylglycerol-polyethylene glycol, and about 0.1 mol % distearoylphosphatidylethanolamine-polyethylene glycol (DSPE-PEG).

Embodiment 21. The method of embodiment 20, wherein the DSPE-PEG is conjugated to a targeting moiety comprising an antibody or antigen binding portion thereof.

Embodiment 22. The method of embodiment 21, wherein the antibody or antigen binding portion thereof comprises a F(ab′) analog.

Embodiment 23. The method of embodiment 21 or 22, wherein the antibody or antigen binding portion thereof is specific for CD8, CD7, CD5, or CD2.

Embodiment 24. The method of any one of embodiments 1-23, wherein the targeted T cell is a CD8⁺ T cell.

Embodiment 25. The method of any one of embodiments 1-24, wherein a low dose corticosteroid is administered about 1 hour before the first or last dose of tLNP.

Embodiment 26. The method of any one of embodiments 1-24, wherein a low dose corticosteroid is administered about 1 hour before each dose of tLNP.

Embodiment 27. The method of embodiment 25 or 26, wherein the low dose corticosteroid is dexamethasone, hydrocortisone, or methylprednisolone.

Embodiment 28. The method of embodiment 25 or 26, wherein the low dose corticosteroid is dexamethasone.

Embodiment 29. The method of embodiment 28, wherein the dosage of dexamethasone is from 5 to 20 mg administered intravenously.

Embodiment 30. The method of any one of embodiments 1-29, wherein the T cell activating agent of each of the multiple doses is a CAR, TCR, or TCE.

Embodiment 31. The method of any one of embodiments 1-29, wherein the T cell activating agent of each of the multiple doses is a CAR, TCR, TCE, or a combination thereof.

Embodiment 32. The method of embodiment 30 or embodiment 31, wherein the specificity of the CAR, TCR, or TCE of the first or first and second of the multiple doses is different from the specificity of the CAR, TCR, or TCE of subsequent doses.

Embodiment 33. The method of embodiment 32, wherein the CAR, TCR, or TCE of the first or first and second of the multiple doses bind an antigen having non-restricted expression.

Embodiment 34. The method of embodiment 33, wherein the antigen having non-restricted expression is CD19, CD20, CD22, CD38, CD123, CD138, DEC205, BCMA, FcRL5, or GPRC5D.

Embodiment 35. The method of embodiment 32, wherein the CAR, TCR, or TCE of the subsequent doses binds an antigen of restricted expression.

Embodiment 36. The method of embodiment 35, wherein the antigen of restricted expression is EGFRvIII, Her2/Neu, PSCA, PSMA, mesothelin, FAP, trop2, DLL3, GPC3, Claudin 18.2, GD2 and as TCR targets HPV E6,E7, NYESO1, PRAME, Melan A, Tyrosinase, PSMA, SSX2, or EBV latent antigen.

Embodiment 37. The method of embodiment 30, wherein the specificity of the CAR, TCR, or TCE of each of the multiple doses is the same.

Embodiment 39. The method of any one of embodiments 1-29, wherein the T cell activating agent of the first or first and second of the multiple doses is a conditioning agent and the T cell activating agent of the subsequent doses is a CAR, TCR, or TCE.

Embodiment 40. The method of any one of embodiments 1-39, wherein administering comprises intravenous infusion.

Embodiment 41. A method of treating a disease or disorder associated with a pathogenic cell comprising administering to a subject in need thereof in a compact regimen multiple doses of a T cell-targeted tLNP encapsulating an RNA encoding a T cell activating agent, wherein the tLNP delivers the RNA encoding the T cell activating agent to the targeted T cells in the subject and the targeted T cells express the T cell activating agent, wherein the compact dose regimen comprises administering each dose within 1 to 5 days of the immediately preceding dose, wherein the T cell activating agent of the initial dose, or initial and second dose, is a conditioning agent, a CAR, a TCR, or a TCE and wherein the T cell activating agent of each dose subsequent to the initial or initial and second dose is a CAR, a TCR, or a TCE that is specific for an antigen expressed by the pathogenic cell.

Embodiment 42. The method of embodiment 41, wherein the CAR, the TCR, or the TCE of the initial dose, or the initial and second dose, is different from the CAR, the TCR, or the TCE of the subsequent dose(s).

Embodiment 43. The method of embodiment 41, wherein the CAR, the TCR, or the TCE of the initial dose, or the initial and second dose is same as the CAR, the TCR, or the TCE of the subsequent dose.

Embodiment 44. The method of embodiment 41, wherein the disease or disorder is cancer.

Embodiment 45. The method of embodiment 41, wherein the pathogenic cell is a neoplastic cell.

Embodiment 46. The method of embodiment 41, wherein the pathogenic cell is a tumor stromal cell.

Embodiment 47. The method of embodiment 41, wherein the disease or disorder is a fibrotic disorder.

Embodiment 48. The method of embodiment 46 or 47, wherein the antigen recognized by the CAR, TCR, or TCE is Fibroblast Activation Protein (FAP) or leucine-rich repeat containing 15 (LRRC15).

Embodiment 49. The method of embodiment 41, wherein the disease or condition is a B cell-related disorder and the antigen recognized by the CAR, TCR, or TCE is a B cell lineage antigen.

Embodiment 50. The method of embodiment 49, wherein the disease or disorder or B cell-related disorder is a B cell leukemia or lymphoma.

Embodiment 51. The method of embodiment 49, wherein the B cell-related disorder is light-chain amyloidosis or Waldenstrom's macroglobulinemia.

Embodiment 52. The method of embodiment 49, wherein the B cell-related disorder is allogeneic transplant rejection.

Embodiment 53. The method of embodiment 49, wherein the disease or disorder or B cell-related disorder is a B cell-mediated autoimmune disease.

Embodiment 54. The method of embodiment 53, wherein the autoimmune disease is myositis, anti-synthetase myositis, anti-synthetase syndrome, lupus nephritis, membranous nephropathy, systemic lupus erythematosus, autoimmune hemolytic anemia, neuromyelitis optica spectrum disorders, myasthenia gravis, pemphigus vulgaris, systemic sclerosis, idiopathic inflammatory myopathy, multiple sclerosis, Sjögren's syndrome, IgA nephropathy, severe combined immunodeficiency, or Fanconi anemia.

Embodiment 55. The method of any one of embodiments 53 or 54, wherein depletion of organ B cells achieved by the compact administration regimen is sufficient that once the regimen is completed repopulating B cells are predominantly naïve B cells.

Embodiment 56. The method of any one of embodiments 49-55, wherein the antigen recognized by the CAR, TCR, or TCE is CD19, CD20, BCMA, or any combination thereof.

Embodiment 57 The method of any one of embodiments 49-56, whereby more T cells express the CAR, TCR, or TCE as a result of a subsequent administration than if the initial administration had not been administered.

Embodiment 58. The method of any one of embodiments 49-57, wherein greater pharmacologic and/or clinical effect and/or improved safety is achieved after administration of at least 1 initiating dose and one dose subsequent to the initiating dose in the compact regimen as compared to the same total dosage administered over a same time interval as a single dose, or as multiple doses where each subsequent dose is administered ≥7 days after an immediately preceding dose.

Embodiment 59. The method of embodiment 58, wherein the improved safety comprises a reduced risk or occurrence of cytokine release syndrome, anti-drug antibody hypersensitivity, or adverse events of grade 3 or greater.

Embodiment 60. The method of embodiment 58, wherein the improved safety comprises absence of adverse events of grade 3 or greater.

Embodiment 61. The method of any one of embodiments 1-60, wherein the compact regimen comprises administering each subsequent dose of the multiple doses within 2 to 5 days of the immediately preceding previous dose.

Embodiment 62. The method of embodiment 61, wherein each subsequent dose of the multiple doses is within 2 to 3 days of the immediately preceding previous dose.

Embodiment 63. The method of embodiment 62, wherein a dose is administered every 3rd day.

Embodiment 64. The method of any one of embodiment 61-63, wherein a total of 2-6 doses, 2-4 doses, 3 doses, or 2 doses are administered in a cycle of treatment.

Embodiment 65. The method of any one of embodiments 41-64, wherein the dosage is about 0.1 to about 2.0 mg RNA/kg per dose.

Embodiment 66. The method of embodiment 65, wherein the dosage is about 0.3 to about 1.0 mg/kg per dose.

Embodiment 67. The method of any one of embodiments 61-66, wherein the cumulative dosage is ≤3 mg RNA/kg/6 days.

Embodiment 68. The method of any one of embodiments 41-67, wherein the T cell-targeted tLNP comprises a targeting moiety that binds to CD8, CD2, CD5, CD7, or CD4.

Embodiment 69. The method of any one of embodiment 41-68, wherein the T cell activating agent of each dose subsequent to the one or two initial doses is a CAR.

Embodiment 70. The method of any one of embodiments 41-69, wherein the T cell is a cytolytic T cell.

Embodiment 71. A pharmaceutical composition comprising a T cell-targeted tLNP encapsulating an mRNA encoding a T cell-activating agent suitable for administration at a dosage of at least 0.3 mg RNA/kg or in a range of about 0.3 to about 1.0 mg/kg in a compact regimen.

Embodiment 72. The pharmaceutical composition of embodiment 71, comprising a 50 mg dose suitable for a subject of 50 to 167 kg, a 40 mg dose suitable for a subject of 40 to 133 kg, a 30 mg dose suitable for a subject of about 30 to 100 kg, a 25 mg dose suitable for a subject of about 25 to 83 kg or a 20 mg dose suitable for a subject of about 20 to 67 kg.

Embodiment 73. The pharmaceutical composition of embodiment 71 or 72, contained in a prefilled syringe.

Embodiment 74. The pharmaceutical composition of any one of embodiments 71-73, further comprising tris, NaCl, sucrose, and/or glycerol.

Embodiment 75. The method of any one of embodiments 41-70, further comprising administering a low dose of corticosteroid to the subject prior to the first dose, the last dose, or each dose of the multiple doses.

Embodiment 76. The method of embodiment 75, wherein the corticosteroid is administered about an hour prior to the first dose or last dose of the multiple doses.

Embodiment 77. The method of embodiment 75, wherein the corticosteroid is administered about an hour prior to prior to each dose of the multiple doses.

Embodiment 78. The method of any one of embodiment 75-77, wherein the corticosteroid is dexamethasone, hydrocortisone, or methylprednisolone.

Embodiment 79. The method of embodiment 78, wherein the dexamethasone is administered to the subject at a dosage of from about 5 mg to about 20 mg.

Embodiment 80. The method of embodiment 78, wherein the hydrocortisone is administered to the subject at a dosage of from about 100 mg to about 400 mg.

Embodiment 81. The method of embodiment 78, wherein the methylprednisolone is administered to the subject at a dosage of from about 25 mg to about 100 mg.

Embodiment 82. A method of increasing in vivo transfection efficiency of T cells for introducing a therapeutic agent into the T cells comprising administering to a mammalian subject in a compact regimen, at least one dose of a T cell activating agent and subsequently administering within 1, 2, 3, 4, or 5 days at least one dose of a therapeutic agent wherein the therapeutic agent comprises a T cell-targeted lipid nanoparticle (tLNP) encapsulating an RNA encoding a CAR, TCR, or TCE, wherein a population of cells in the subject expresses an antigen recognized by the CAR, TCR, or TCE, whereby more T cells express the CAR, TCR, or TCE as a result of the initial administration of the T cell activating agent than if it had not been administered.

Embodiment 83. The method of embodiment 82, wherein the T cell activating agent is the tLNP encapsulating an RNA encoding the CAR, TCR, or TCE.

Embodiment 84. The method of embodiment 82, wherein the T cell activating agent is a tLNP encapsulating an RNA encoding a CAR, TCR, or TCE different than the therapeutic agent.

Embodiment 85. The method of embodiment 82 or 83, wherein the CAR, TCR, or TCE of the T cell activating agent binds to a B cell antigen

Embodiment 86. The method of embodiment 85, wherein the B cell antigen is CD19 or BCMA.

Embodiment 87. The method of embodiment 82, wherein the T cell activating agent is a conditioning agent.

Embodiment 88. The method of any one of embodiments 82-87, wherein the tLNP encapsulated RNA is an mRNA.

Embodiment 89. A method of increasing in vivo T cell reprogramming efficiency, comprising administering to a mammalian subject in a compact regimen at least one dose of a T cell-targeted lipid nanoparticle (tLNP) encapsulating an RNA encoding a first CAR, TCR, or TCE that binds an antigen having non-restricted expression, followed by administering within 1, 2, 3, 4, or 5 days at least one subsequent dose of a T cell-targeted lipid nanoparticle (tLNP) encapsulating an RNA encoding a second CAR, TCR, or TCE that binds an antigen having restricted expression, whereby more T cells express the second CAR, TCR, or TCE as a result of the at least one subsequent administration than if it had not been preceded by the at least one dose of the T cell-targeted lipid nanoparticle (tLNP) encapsulating an RNA encoding the first CAR, TCR, or TCE.

Embodiment 90. A method of depleting B cells in a mammalian subject, the method comprising administering in a compact regimen at least one dose of a T cell activating agent and subsequently administering within 1, 2, 3, 4, or 5 days at least one dose of a T cell-targeted lipid nanoparticle (tLNP) encapsulating an RNA encoding a CAR, TCR, or TCE that binds a B cell antigen, whereby more T cells express the CAR, TCR, or TCE that binds the B cell antigen as a result of a subsequent administration than if it had not been preceded by the at least one dose of the T cell activating agent.

Embodiment 91. A method of blunting induction of an anti-drug antibodies (ADA) reaction, comprising administering in a compact regimen at least one dose of a T cell activating agent and subsequently administering within 1, 2, 3, 4, or 5 days at least one dose of a T cell-targeted lipid nanoparticle (tLNP) encapsulating an RNA encoding a CAR, TCR, or TCE that binds a B cell antigen, whereby B cells are sufficiently depleted for an interval of time that administration of a immunogenic drug within that interval of time results in a diminished or absent ADA reaction.

Embodiment 92. The method of any one of clams 90 or 91, wherein the T cell activating agent is a conditioning agent or a T cell-targeted tLNP encapsulating an RNA encoding a conditioning agent.

Embodiment 93. The method of embodiment 10, 39, 41, 87, or 92 wherein the conditioning agent is a 7-chain receptor cytokine, a pan-activating cytokine, or an immune checkpoint inhibitor.

Embodiment 94. The method of embodiment 93, wherein the 7-chain receptor cytokine is IL-7.

Embodiment 95. The method of embodiment 93, wherein the pan-activating cytokine is IL-18.

Embodiment 96. The method of embodiment 93, wherein the immune checkpoint inhibitor is an antibody that binds PD-1 or PD-L1.

Embodiment 97. The method of any one of embodiments 90 or 91, wherein the activating agent is a T cell-targeted tLNP encapsulating an RNA encoding a CAR, TCR, or TCE that binds an antigen having non-restricted expression.

Embodiment 98. The method of embodiment 97, wherein the antigen having non-restricted expression is a B cell antigen.

Embodiment 99. The method of embodiment 98, wherein the B cell antigen is CD19, CD20, or BCMA.

Embodiment 100. The method of embodiment 91, wherein the immunogenic drug comprises a payload encapsulated in an LNP.

Embodiment 101. The method of embodiment 100, wherein the LNP is a tLNP.

Embodiment 102. The method of embodiment 101, wherein the tLNP is a T cell-targeted tLNP.

Embodiment 103. The method of any one of embodiments 100-102, wherein the payload comprises a nucleic acid.

Embodiment 104. The method of embodiment 103, wherein the nucleic acid is RNA.

Embodiment 10⁵. The method of embodiment 104, wherein the RNA is mRNA, circular RNA, or self-amplifying RNA.

Embodiment 106. The method of embodiment 10⁵, wherein the RNA is small interfering RNA (siRNA), microRNA (miRNA), or an antisense oligonucleotide (ASO).

Embodiment 107. The method of embodiment 89, wherein the antigen bound by the second CAR, TCR, or TCE is an antigen having non-restricted expression.

Embodiment 108. The method of embodiment 89, wherein the antigen bound by the second CAR, TCR, or TCE is an antigen having restricted expression.

Embodiment 109. The method of embodiment 108, wherein the antigen having restricted expression is FAP

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing descript tion, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

APPENDIX A

| | | Commercial Source(s) (if available) | | |
|---|---|---|---|---|
| | Antigen/ | | Supplier and | |
| Antibody | Target | Product Name | Catalog Number | Publication Information |
| 1.5.3 | CD20 | | | Gazit-Bornstein et al., 2010, |
| | | | | Invest. New Drugs 28: 561- |
| | | | | 574. |
| 1C5 | CD117 | c-Kit/CD117 Antibody (1C5) | ThermoFisher, | |
| | | | #MA5-15894 | |
| 1D1C2 | IL-21Rα | IL21R Monoclonal Antibody | ThermoFisher, | |
| | | (1D1C2) | #66319-1-IG | |
| 1F5 | CD20 | Anti-CD20 [1F5] | Absolute Antibody, | |
| | | | #Ab01133 | |
| 1mono2A6 (T113) | CD2 | Ellis Reinherz, Dana-Farber Cancer Institute, Boston, | | |
| | | MA | | |
| 1OLD2-4C1 (T112) | CD2 | Ellis Reinherz, Dana-Farber Cancer Institute, Boston, | | US 2022/0073639 |
| | | MA | | (SEQ ID NO. 51 & 52) |
| 11B1H1B4 | CD150 | CD150 Monoclonal Antibody | Fisher Scientific, | |
| | | (11B1H1B4), Invitrogen ™ | #16384654 | |
| 158-4D3 | CD45 | CD45 Monoclonal Antibody | ThermoFisher, | |
| | | (158-4D3) | #MA1-7636 | |
| 190-2F2.5 | CD45RO | CD45RO (T-Cell Marker) | ThermoFisher, | |
| | | Monoclonal Antibody (190- | #5788-MSM12-P0 | |
| | | 2F2.5) | | |
| 2.1.2 | CD20 | Anti-Human MS4A1 | Creative Biolabs, | US 2007/0014720 |
| | | Recombinant Antibody (2.1.2) | #TAB-1645CL | |
| 22HCLC | CD117 | Phospho-c-Kit (Tyr703) | Fisher Scientific, | |
| | | Recombinant Polyclonal | #PI710762 | |
| | | Antibody (22HCLC), | | |
| | | Invitrogen ™ | | |
| 28H1 | FAP | | | US 2012/0128591 |
| 2A9 | SSEA-3 | Anti-SSEA-3/GalGb4 Antibody | AntibodySystem, | |
| | | (2A9) | #RGK27501 | |
| 2A10D6 | FCRL5 | CD307e (FcRL5) Monoclonal | ThermoFisher, | |
| | | Antibody | #MA5-48789 | |
| 2A10H7 | FCRL5 | CD307e (FcRL5) Monoclonal | ThermoFisher, | |
| | | Antibody | #MA5-38483 | |
| 2A11D6 | CD43 | CD43 Monoclonal Antibody | ThermoFisher, | |
| | | (2A11D6) | #66224-1-IG | |
| 2B8 | CD117 | CD117 (c-Kit) Monoclonal | ThermoFisher, | |
| | | Antibody (2B8), eBioscience ™ | #14-1171-82 | |
| 2B8/BM | CD117 | CD117 (c-Kit) Monoclonal | ThermoFisher, | |
| | | Antibody (2B8/BM) | #MA5-28270 | |
| 2D4-C9-F1 | Sca-1 | Mouse anti-Casp3-1(138-157) | Biocat, #130-10623- | |
| | | (2D4-C9-F1) | 20-RB | |
| 2E10 | CD34 | available from multiple sources, including: | | |
| | | Anti-CD34 [2E10] | Absolute Antibody, | |
| | | | #Ab01129 | |
| | | Human Anti-CD34 Recombinant | Creative Biolabs, | |
| | | Antibody (clone 2E10) | #FAMAB-1049CQ | |
| 2ST8.5H7 | CD8ß | available from multiple sources, including: | | |
| | | CD8 Beta antibody \| 2ST8.5H7 | Bio-Rad, #MCA1723 | |
| | | CD8 Antibody (2ST8.5H7) | Novus Biologicals, | |
| | | | #NB100-65928 | |
| 307307 | FCRL5 | CD307e (FcRL5) Monoclonal | ThermoFisher, | |
| | | Antibody | #MA5-24020 | |
| 3A1E | CD7 | CD7 Chimeric Recombinant | ThermoFisher, | |
| | | Rabbit Monoclonal Antibody | #MHCD0800 | |
| | | (3A1E) | | |
| 3A1F | CD7 | Anti-CD7 [3A1F] | Absolute Antibody, | |
| | | | #Ab03052 | |
| 3B5 | CD8 | available from multiple sources, including: | | |
| | | CD8 Monoclonal Antibody | ThermoFisher, | |
| | | (3B5) | #MHCD0800 | |
| | | | Aviva Systems | |
| | | | Biology, | |
| | | | #OAAI00153 | |
| 3B8-2C2 | CD48 | CD48 Monoclonal Antibody | ThermoFisher, | |
| | | (3B8-2C2) | #H00000962-M01 | |
| 3F7 | CD73 | | | Qiao et al., 2019, Int. J. Mol. |
| | | | | Sci. 20 (5): 1057. |
| 3F7C6 | CD68 | CD68 monoclonal antibody, | Abnova, | |
| | | clone 3F7C6 | #MAB12263 | |
| 3F7D3 | CD68 | available from multiple sources, including: | | |
| | | CD68 monoclonal antibody, | Abnova, | |
| | | clone 3F7D3 | #MAB12260 | |
| | | Anti-CD68 antibody [3F7D3] | Abcam, #ab201973 | |
| 3F8BiAb | GD2 | | | Yankelevich et al., 2012, |
| | CD3 | | | Pediatr. Blood Cancer 59(7): |
| | | | | 1198-1205. |
| 3G9-2D2 | CD205 | | | Cheong et al., 2010, Blood |
| | | | | 116 (19): 3828-3838. |
| 3H420 | SSEA-3 | SSEA-3 Antibody (3H420) | Santa Cruz | |
| | | | Biotechnology, #sc- | |
| | | | 73066 | |
| 3T4-8B5 (T111) | CD2 | Ellis Reinherz, Dana-Farber Cancer Institute, Boston, | | |
| | | MA | | |
| 4C5 | Stro-4 | | | Thomaidou and Patsavoudi, |
| | | | | 1993, Neuroscience 53: 813- |
| | | | | 827. |
| 4G5 | FAP | | | WO 2021/061708; |
| | | | | WO 2021/061778 |
| 4G7 | CD19 | | | Meeker et al., 1984, |
| | | | | Hybridoma 3: 305-320. |
| 4KB5 | CD45RA | Anti-CD45RA antibody [4KB5] | Abcam, #ab755 | |
| 47G4 | CD19 | | | US 2010/0104509 |
| 51.1 | CD8 | Anti-CD8 [51.1] | Absolute Antibody, | |
| | | | #Ab04357 | |
| 53H8L29 | CD117 | Phospho-c-Kit (Tyr703) | ThermoFisher, | |
| | | Recombinant Rabbit Monoclonal | #700135 | |
| | | Antibody (53H8L29) | | |
| 5B4G1 | CD150 | SLAM/CD150 Monoclonal | ThermoFisher, | |
| | | Antibody (5B4G1) | #60043-1-IG | |
| 5B12 | CD34 | available from multiple sources, including: | | |
| | | Anti-CD34 [5B120 | Absolute Antibody, | |
| | | | #Ab00422 | |
| | | Human Anti-CD34 Recombinant | Creative Biolabs, | |
| | | Antibody (clone 5B12) | #FAMAB-1047CQ | |
| 5D7 | CD5 | CD5 Monoclonal Antibody | ThermoFisher, | |
| | | (CD5-5D7), APC | #MHCD0505 | |
| 5E3E1 | IL-15Rα | IL-15RA Monoclonal Antibody | ThermoFisher, | |
| | | (5E3E1) | #MHCD0505 | |
| 5E10 | CD90 | available from multiple sources, including: | | |
| | | CD90 (Thy-1) Monoclonal | ThermoFisher, #14- | |
| | | Antibody (eBio5E10 (5E10)), | 0909-82 | |
| | | eBioscience ™ | | |
| | | Anti-Human CD90 Antibody, | Stemcell | |
| | | Clone 5E10 | Technologies, | |
| | | | #60045 | |
| 509F6 | FCRL5 | CD307e (FcRL5) Monoclonal | ThermoFisher, #50- | |
| | | Antibody (509F6), eFluor ™ 660, | 3078-42 | |
| | | eBioscience ™ | | |
| 6D276 | CD29 | Anti-CD49b, CD29 Complex | VWR, #USBIC2404- | |
| | CD49b | Mouse Monoclonal Antibody | 07W | |
| | | [clone: 6D276] | | |
| 6D9 | GPRC5D | Anti-GPRC5D (6D9) h(41BB- | Creative Biolabs, | |
| | | CD3ζ) CAR, pCDCAR1 | #CAR-LY1362 | |
| 6F2 | CD117 | KIT Monoclonal Antibody (6F2) | ThermoFisher, | |
| | | | #H00003815-M02 | |
| 7D11 | FCRL5 | Anti-FcRH5 (clone 7D11)- | Creative Biolabs, | |
| | | SMCC-DM1 ADC | #ADC-161CL | |
| 7E1 | CD48 | CD48 Recombinant Rabbit | ThermoFisher, | |
| | | Monoclonal Antibody (7E1) | #MA5-38301 | |
| 8A7 | SSEA-3 | Anti-SSEA-3/GalGb4 Antibody | Antibody System, | |
| | | (8A7) | #RGK27504 | |
| 8D7 | CD117 | c-Kit Monoclonal Antibody | ThermoFisher, | |
| | | (8D7) | #MA5-15327 | |
| 9.1 | CD2 | Peter Linsley, Benaroya Research Institute, Seattle, WA | | Connelly et al., 1998, Int. |
| | | | | Immunol., 10 (12): 1863- |
| | | | | 1872. |
| 9.6 | CD2 | Peter Linsley, Benaroya Research Institute, Seattle, WA | | Immunol., 10 (12): 1863- |
| | | | | Connelly et al., 1998, Int. |
| | | | | 1872. |
| 9C5 | CD34 | | | U.S. Pat. No. 8,399,249 |
| 9D1 | CD150 | CD150 Monoclonal Antibody | ThermoFisher, | |
| | | (9D1), PE, eBioscience ™ | #12-1501-82 | |
| 10A8 | FCRL5 | Anti-FcRL5 (10A8)-SPDB-DM4 | Creative Biolabs, | |
| | | ADC | #ADC-117LCT | |
| 12G10 | CD29 | available from multiple sources, including: | | |
| | | CD29 (Stem Cell Marker) | ThermoFisher, | |
| | | Monoclonal Antibody (12G10) | #3688-MSM2-P0 | |
| | | CD29 antibody \| 12G10 | Bio-Rad, #MCA2028 | |
| 13G9 | FCRL5 | Mouse Anti-FCRL5 | Creative Biolabs, | |
| | | Recombinant Antibody | #MOB-057CQ | |
| 17D8 | CD8 | CD8 Monoclonal Antibody | ThermoFisher, | |
| | | (17D8) | #MA1-12027 | |
| 18A5 | IL-21Rα | Human Anti-IL21R | Creative Biolabs, | |
| | | Recombinant Antibody | #HPAB-0668-CN | |
| 84-3C1 | CD43 | CD43 Monoclonal Antibody | ThermoFisher, | |
| | | (PE, eBioscience ™) | #12-0439-42 | |
| 104D2 | CD117 | available from multiple sources, including: | | |
| | | Purified anti-human CD117 (c- | BioLegend, #313201 | |
| | | kit) Antibody | | |
| | | Anti-Human CD117 (c-Kit) | Stemcell Tech- | |
| | | Antibody, Clone 104D2 | nologies, #60087 | |
| | | MOUSE ANTI HUMAN CD117 | Bio-Rad, #MCA1841 | |
| | | c-Kit Monoclonal Antibody | ThermoFisher, | |
| | | (104D2) | #MA1-10072 | |
| 131-I-apamistamab | CD45 | | | Seropian et al., 2023, Blood |
| | | | | 142 (Supplement 1): 2159. |
| 131-I-omburtamab | B7H3 | | | Kramer et al., 2022, J. |
| (131-I-8H9) | | | | Hematol. Oncol. 15: 165. |
| 177-Lu-DTPA-omburtamab | B7H3 | | | Khatua et al., 2021, Ann. |
| | | | | Oncol. 32 (Suppl. 5): S528- |
| | | | | S529. |
| 156-4H9 | CD48 | CD48 Monoclonal Antibody | ThermoFisher, | |
| | | (eBio156-4H9 (156-4H9)), | #14-0489-82 | |
| | | eBioscience ™ | | |
| 293C3 | CD133 | available from multiple sources, including: | | |
| | | Anti-CD133 antibody [293C3] | Abcam, #ab252553 | |
| | | CD133/2 Antibody, anti-human, | Miltenyi Biotec, | |
| | | pure | #130-090-851 | |
| 341 | CD48 | CD48 Recombinant Rabbit | ThermoFisher, | |
| | | Monoclonal Antibody (341) | #MA5-29675 | |
| 514H12 | CD68 | CD68 Monoclonal Antibody | ThermoFisher, | |
| | | (514H12) | #MA1-80133 | |
| 1279B | SUSD2 | SUSD2 Antibody (1279B) | Novus Biologicals, | |
| | | | #MAB90564 | |
| 2639B | IL-15Rα | Human IL-15R alpha Antibody | R&D Systems, | |
| | | | #MAB10900 | |
| A7R34 | CD127 | CD127 Monoclonal Antibody | ThermoFisher, | |
| | | (A7R34), eBioscience ™ | #14-1271-82 | |
| A12 (7D4) | CD150 | CD150 Monoclonal Antibody | ThermoFisher, | |
| | | (A12 (7D4)), PE, eBioscience ™ | #12-1509-42 | |
| AB75 | CD2 | available from multiple sources, including: | | |
| | | CD2 Monoclonal Antibody | ThermoFisher, | |
| | | (AB75) | #MA5-11373 | |
| | | Anti-CD2 Mouse Monoclonal | Avantor (VWR), | |
| | | Antibody [clone: AB75] | #GTX75367 (89365- | |
| | | | 772) | |
| Ab85 | CD117 | | | WO 2019/084053 |
| ABL501 | LAG-3 | | | Park et al., 2021, Cancer Res. |
| | PD-L1 | | | 81 (13_Supplement): 1633. |
| ABM53F5 | CD68 | Monoclonal antibody to CD68 | Abeomics, #10-4171 | |
| | | (Clone: ABM53F5) | | |
| ABX-MA1 | CD146 | Anti-CD146 [ABX-MA1 | Absolute Antibody, | |
| | | (3.19.1)] | #Ab02820 | |
| AC133 | CD133 | CD133/1 Antibody, anti-human, | Miltenyi Biotec, | |
| | | pure | #130-090-422 | |
| acazicolcept | CD28 | Acazicolcept | MedChemExpress, | |
| | | | #HY-P99420 | |
| ACK2 | CD117 | CD117 (c-Kit) Monoclonal | ThermoFisher, | |
| | | Antibody (ACK2), APC, | #17-1172-82 | |
| | | eBioscience ™ | | |
| ACK4 | CD117 | c-Kit Monoclonal Antibody | ThermoFisher, | |
| | | (ACK4) | #MA1-70079 | |
| Adcitmer ® | CD56 | | | Esnault et al., 2022, Br. J. |
| | | | | Dermatol. 186 (2): 295-306. |
| ADG106 | CD137 | | | Ma et al., 2024, Cell Rep. |
| | | | | Med. 5 (2): 101414. |
| ADG116 | CTLA-4 | | | Park et al., 2022, J. |
| | | | | Immunother. Cancer 10 |
| | | | | (Suppl 2): A1-A1603. |
| ADG126 | CTLA-4 | | | Richardson et al., 2022, Ann. |
| | | | | Oncol. 33 (suppl 7): S882- |
| | | | | S883. |
| ADU-1604 | CTLA-4 | | | De Velasco Oria de Rueda et |
| | | | | al., 2023, J. Immunother. |
| | | | | Cancer 11 (Suppl 1): A1- |
| | | | | A1731. |
| AFM13 | CD16a | Anti-FcgR3a/CD16a (AFM13) | Selleckchem, | |
| | | | #A2750 | |
| AGEN1181 | CTLA-4 | | | El-Khoueiry, et al., 2021, J. |
| | | | | Immunother. Cancer 9 (Suppl |
| | | | | 2): A1-A1054. |
| AK112 | PD-1 | Research Grade Ivonescimab | AntibodySystem, | |
| | VEGF | | #DHD12605 | |
| AK119 | CD73 | Research Grade Dresbuxelimab | Cell Sciences, | |
| | | | #DHD46603A | |
| AK129 | LAG-3 | | | Huang et al., 2022, Cancer |
| | | | | Res. 82 (12_Supplement): |
| | | | | 5520. |
| alefacept | CD2 | Alefacept | MedChemExpress, | |
| | | | #HY-P99429 | |
| AlexaFluor647 anti-hIgG Fc | IgG | available from multiple sources, including: | | |
| detection antibody | | Alexa Fluor ® 647 AffiniPure ™ | Jackson | |
| | | Goat Anti-Human IgG, Fcγ | ImmunoResearch | |
| | | fragment specific | Laboratories, #109- | |
| | | | 605-098 | |
| | | Human IgG Alexa Fluor ® 647- | R&D Systems, | |
| | | conjugated Antibody | #FAB110R | |
| alirocumab | PCSK9 | available from multiple sources, including: | | |
| | | Alirocumab | MedChemExpress, | |
| | | | #HY-P9928 | |
| | | Alirocumab Biosimilar - Anti- | Proteogenix, #PX- | |
| | | PCSK9 mAb | TA1287 | |
| | | Alirocumab (anti-PCSK9) | SelleckChem, | |
| | | | #A2047 | |
| alnuctumab | BCMA | | | U.S. Pat. No. 10,683,369 |
| AMG-172 | CD70 | | | Massard et al., 2019, Cancer |
| | | | | Chemother. Pharmacol. |
| | | | | 83(6):1057-63. |
| AMG 224 | BCMA | | | U.S. Pat. No. 9,243,058 |
| AMG 228 | GITR | | | Tran et al., 2017, J. Clin. |
| | | | | Oncol. 35 (15_suppl): 2521. |
| AMG 404 | PD-1 | Zeluvalimab | MedChemExpress, | |
| | | | #HY-P99957 | |
| AMM22070N | Sca-1 | Caspase-3 Mouse mAb | Leading Biology, | |
| | | | #AMM22070N | |
| anti-BCMA CARs | BCMA | | | US 2020/0246381; |
| | | | | US 2020/0339699 |
| anti-CD3 FITC antibody | CD3 | BD Pharmingen ™ FITC mouse | BD Biosciences, | |
| conjugate | | anti-human CD3ε | #556611 | |
| anti-CD3 SP34-2 Alexa Fluor | CD3 | BD Pharmingen ™ Alexa Fluor ® | BD Biosciences, | |
| 700 conjugate antibody | | 700 mouse anti-human CD3 | #557917 | |
| anti-CD3 V450 | CD3 | available from multiple sources, including: | | |
| | | BD Horizon ™ V450 Mouse | BD Biosciences, | |
| | | Anti-Human CD3 | #560366 | |
| | | violetFluor ™ 450 Anti-Human | Cytek, #75-0038- | |
| | | CD3 (UCHT1) | T025 | |
| anti-CD4 (PerCP-Cy5.5) | CD4 | available from multiple sources, including: | | |
| | | BD Pharmingen ™ PerCP- | BD Biosciences, | |
| | | Cy ™5.5 Mouse Anti-Human | #560650 | |
| | | CD4 | | |
| | | CD4 Monoclonal Antibody | ThermoFisher, #45- | |
| | | (RM4-5), PerCP-Cyanine5.5, | 0042-82 | |
| | | eBioscience ™ | | |
| anti-CD4 BV 421 antibody | CD4 | Brilliant Violet 421 ™ anti- | BioLegend, #317434 | |
| conjugate | | human CD4 Antibody | | |
| anti-CD4 OKT4 BV650 | CD4 | Brilliant Violet 650 ™ anti- | BioLegend, #317436 | |
| conjugate antibody | | human CD4 Antibody | | |
| anti-CD8a (APC-H7) | CD8a | BD Pharmingen ™ APC-H7 | BD Biosciences, | |
| | | Mouse anti-Human CD8 | #560179 | |
| anti-claudin 18.2 antibodies | CLDN18.2 | | | WO 2013/167259, |
| | | | | WO 2021/032157, |
| | | | | WO 2021/254481, |
| | | | | WO 2022/007808, |
| | | | | WO 2021/008463, |
| | | | | WO 2022/111616, |
| | | | | WO 2018/006882, |
| | | | | WO 2020/147321, |
| | | | | WO 2019/219089, |
| | | | | US 2020/0040101, |
| | | | | WO 2020/025792, |
| | | | | WO 2020/139956, |
| | | | | WO 2020/135201, |
| | | | | US 2024/0228610, |
| | | | | WO 2021/218874, |
| | | | | WO 2021/027850, |
| | | | | WO 2021/129765, |
| | | | | WO 2022/068854, |
| | | | | WO 2021/111003 |
| anti-FAP CARS | FAP | | | WO 2021/061778 |
| anti-FCRL5 CARs and ADCs | FCRL5 | | | WO 2016/090337, WO |
| | | | | 2017/096120, WO |
| | | | | 2022/263855, WO |
| | | | | 2024/047558, Elkins et al., |
| | | | | Mol. Cancer Ther. 11(10): |
| | | | | 2222-2232 (2012) |
| anti-G4S PE antibody | G4S | G4S Linker (E702V) Rabbit | Cell Signaling | |
| | | mAb (PE Conjugate) | Technology, #38907 | |
| anti-human Fc secondary | Fc | Brilliant Violet 421 ™ anti- | BioLegend, #410704 | |
| antibody (conjugated to | | human IgG Fc Antibody | | |
| BV421 fluorophore) | | | | |
| anti-scFv linker PE antibody | scFv linker | MonoRab ™ Rabbit Anti-scFv | GenScript, #A02285 | |
| | | Cocktail [PE] | | |
| anti-siglec-15 antibody | Siglec-15 | | | U.S. Pat. No. 8,575,531 |
| anti-Whitlow linker PE | | Whitlow/218 Linker (E3U7Q) | Cell Signaling | |
| antibody | | Rabbit mAb (PE Conjugate) | Technology, #62405 | |
| ANV419 | CD122 | | | Joerger et al., 2023, J. |
| | | | | Immunother. Cancer 11 (11): |
| | | | | e007784. |
| ARC0672 | CD127 | CD127 Recombinant Rabbit | ThermoFisher, | |
| | | Monoclonal Antibody | #MA5-35152 | |
| | | (ARC0672) | | |
| ARX305 | CD70 | | | Skidmore et al., 2023, Ann. |
| | | | | Oncol. 34 (Suppl. 2): S208. |
| ASD141 | CD11b | | | Chang et al., 2021, J. |
| | | | | Immunother. Cancer 9 (Suppl. |
| | | | | 2): A1-A1054. |
| ASKB589 | CLDN18.2 | | | Zhang et al., 2023, J. Clin. |
| | | | | Oncol. 41 (4 suppl.), 397. |
| ASP1951 | GITR | | | Seidel-Dugan et al., 2018, J. |
| | | | | Immunother. Cancer 6 (115). |
| AT002 | CD166 | available from multiple sources, including: | | |
| | | Recombinant Anti-CD166 | Antibodies.com, | |
| | | antibody | #A323924 | |
| | | Research Grade Anti-Human | Antibody System, | |
| | | CD166/ALCAM | #DHG64002 | |
| atibuclimab | CD14 | available from multiple sources, including: | | |
| | | Atibuclimab | MedChemExpress, | |
| | | | #HY-P99008 | |
| | | Atibuclimab (Anti-CD14) | SelleckChem, | |
| | | | #A2566 | |
| ATL301 | GD2 | Research Grade Anti- | Antibody System, | |
| | | Ganglioside GD2 (ATL301) | #DHK07801 | |
| ATOR-1015 | OX40 | | | Veitonmäki et al., 2018, |
| | | | | Cancer Res. 78 |
| | | | | (13_Supplement): 3623. |
| ATOR-1144 | GITR | | | Fritzell et al., 2019, Cancer |
| | | | | Res. 79 (13_Supplement): |
| | | | | 4077. |
| axicabtagene ciloleucel | CD19 | | | Neelapu et al., 2017, N. Engl. |
| | | | | J. Med. 377: 2531-44. |
| AZD0901 | CLDN18.2 | | | Raufi et al., 2024, J. Clin. |
| | | | | Oncol. 42 (16 Suppl.), |
| | | | | TPS3163. |
| AZD7789 | TIM-3 | | | Clancy-Thompson et al., J. |
| | | | | Immunother. Cancer 10 |
| | | | | (Suppl. 2): A1-A1603. |
| AZN-L50 | CD166 | available from multiple sources, including: | | |
| | | CD166, Human, mAB AZN-L50 | Hycult Biotech, | |
| | | | #HM2002 | |
| | | CD166 antibody [AZN-L50] | GeneTex, | |
| | | | #GTX18159 | |
| B4 | CD19 | | | Freedman et al., 1987, Blood |
| | | | | 70: 418-427. |
| B4 HB12b | CD19 | | | Kansas & Tedder, 1991, J. |
| | | | | Immunol. 147: 4094-4102; |
| | | | | Yazawa et al., 2005, PNAS |
| | | | | 102: 15178-15183; |
| | | | | Herbst et al., 2010, J. |
| | | | | Pharmacol. Exp. Ther. 335: |
| | | | | 213-222. |
| B7V3V2 | CD200 | Anti-Human CD200 Antibody | AntibodySystem, | |
| | | (B7V3V2) | #FHE30310 | |
| B43 | CD19 | | | Bejcek et al., 1995, Cancer |
| | | | | Res. 55 (11): 2346-2351. |
| balstilimab | PD-1 | | | Moore et al., 2018, J. Clin. |
| | | | | Oncol. 36 (15 suppl.): 3086. |
| barzolvolimab | CD117 | CDX 0159 | MedChemExpress, | |
| | | | #HY-P99462 | |
| basiliximab (STI-003) | CD25 | Basiliximab Recombinant | ThermoFisher, | Onrust et al., 1999, Drugs 57, |
| | | Human Monoclonal Antibody | #MA5-41768 | 207-213. |
| BAT6026 | OX40 | | | Liang et al., 2023, Front. |
| | | | | Oncol., 13: 1211759. |
| bavunalimab | LAG-3 | available from multiple sources, including: | | |
| | | Bavunalimab | MedChemExpress, | |
| | | | #HY-P99354 | |
| | | Bavunalimab | ChemScene, #CS- | |
| | | | 0621488 | |
| BB2121 | BCMA | | | WO 2012/163805 |
| BC8-B10 | CD45 | | | Li et al., 2018, PLoS One |
| | | | | 13(10): e0205135. |
| BCD-145 | CTLA-4 | available from multiple sources, including: | | |
| | | Nurulimab | MedChemExpress, | |
| | | | #HY-P99760 | |
| | | Nurulimab | AbMole, #M24967 | |
| BCD-245 | GD2 | | | Clinical Trial ID: |
| | | | | NCT05782959 |
| belantamab | BCMA | | | U.S. Pat. No. 9,273,141 |
| BGB-A445 | OX40 | | | Jiang et al., 2023, Front. Med. |
| | | | | 17(6): 1170-1185. |
| bleselumab | CD40 | available from multiple sources, including: | | |
| | | Bleselumab | MedChemExpress, | |
| | | | #HY-P99257 | |
| | | Bleselumab | SelleckChem, | |
| | | | #A2634 | |
| BL-178-12C7 | CD45 | Anti-CD45 antibody | Abcam, #ab243869 | |
| BLY3 | CD19 | | | Bejcek et al., 1995, Cancer |
| | | | | Res. 55 (11): 2346-2351. |
| BMS-986156 | GITR | | | Siu et al., 2017, J. Clin. Oncol. |
| | | | | 35 (15_suppl): 104. |
| BMS-986178 | OX40 | | | Gutierrez et al., 2021, Clin. |
| | | | | Cancer Res. 27 (2): 460-472. |
| BMS-986179 | CD73 | | | Siu et al., 2018, Cancer Res. |
| | | | | 78(13_Suppl): CT180. |
| BMS-986218 | CTLA-4 | | | Engelhardt et al., 2020, Cancer |
| | | | | Res. 80 (16_Supplement): |
| | | | | 4552. |
| BMS-986249 | CTLA-4 | | | Gutierrez et al., 2020, J. Clin. |
| | | | | Oncol. 38 (15_suppl): 3058. |
| BMS-986258 | TIM-3 | | | Clinical Trial ID: |
| | | | | NCT03446040 |
| BMS-986393 | GPRC5D | | | Bal et al. (2023). Blood 142 |
| | | | | (Suppl. 1): 219. |
| bococizumab | PCSK9 | available from multiple sources, including: | | |
| | | Bococizumab | MedChemExpress, | |
| | | | #HY-P99187 | |
| | | Bococizumab (Anti-PCSK9) | SelleckChem, | |
| | | | #A2788 | |
| | | Bococizumab Recombinant | ThermoFisher, | |
| | | Human Monoclonal Antibody | #MA5-42019 | |
| bococizumab NEI | PCSK9 | | | Dyson et al. (2020). MAbs 12: |
| | | | | 1829335. |
| botensilimab | CTLA-4 | | | Wilky et al., 2022, J. |
| | | | | Immunother. Cancer, 10 |
| | | | | (Suppl. 2): A1-A1603. |
| BP-02489 | CD10 | Accession No. NITE BP-02489 (JP) | | WO 2018/235247 |
| brexucabtagene autoleucel | CD19 | | | Anderson et al., 2022, Ann. |
| | | | | Pharmacother. 56(5): 609-619. |
| briquilimab (JSP-191) | CD117 | available from multiple sources, including: | | |
| | | Briquilimab | MedChemExpress, | |
| | | | #HY-P99488 | |
| | | Research Grade Briquilimab | Cell Sciences, | |
| | | | #DHC83102A | |
| BTI-322 | CD2 | Anti-Human CD2 Recombinant | Creative Biolabs, | |
| | | Antibody (BTI-322) | #TAB-110CL | |
| BU12 | CD19 | | | Callard et al., 1992, J. |
| | | | | Immunology, 148(10): 2983- |
| | | | | 2987 |
| Bu88 | CD8 | Anti-CD8 alpha Antibody | Abcam, #ab316355 | |
| budigalimab | PD-1 | Budigalimab | MedChemExpress, | |
| | | | #HY-P99489 | |
| C2e10 | CD34 | available from multiple sources, including: | | |
| | | Mouse Anti-CD34 Recombinant | Creative Biolabs, | |
| | | Antibody (clone C2e10); scFv | #HPAB-1044-FY- | |
| | | Fragment | F(E) | |
| | | Anti-CD34 [2E10], Human | Absolute Antibody, | |
| | | IgG1, Kappa | #Ab01129 | |
| C5B12 | CD34 | available from multiple sources, including: | | |
| | | Human Anti-CD34 Recombinant | Creative Biolabs, | |
| | | Antibody (clone C5B12); Fab | #HPAB-1043-FY- | |
| | | Fragment | S(P) | |
| | | Anti-CD34 [5B12], Human | Absolute Antibody, | |
| | | IgG1, Kappa | #Ab00422 | |
| C8/144B | CD8α | available from multiple sources, including: | | |
| | | CD8 alpha Monoclonal Antibody | ThermoFisher, | |
| | | (C8/144B) | #MA5-13473 | |
| | | Anti-CD8 alpha antibody | Abcam, #ab17147 | |
| | | [C8/144B] | | |
| C11D5.3 | BCMA | | | WO 2010/104949 |
| C12A3.2 | | | | |
| C363.16A | CD45RB | CD45RB Monoclonal Antibody | ThermoFisher, | |
| | | (PE, eBioscience ™) | #12-0455-82 | |
| cadonilimab | PD-1 | | | Pang et al., 2023, MAbs 15(1): |
| | | | | 2180794. |
| camidanlumab tesirine | CD25 | | | Puzanov et al., 2020, Ann. |
| | | | | Oncol. 31(suppl. 4), S710- |
| | | | | S711. |
| camrelizumab | PD-1 | Camrelizumab (anti-PD-1) | SelleckChem, | |
| | | | #A2016 | |
| carotuximab | CD105 | available from multiple sources, including: | | |
| | | Carotuximab | MedChemExpress, | |
| | | | #HY-P99494 | |
| | | Carotuximab (Anti-Endoglin/ | SelleckChem, | |
| | | CD105) | #A2514 | |
| CBXS-1620 | SUSD2 | Rabbit Anti-SUSD2 | Creative Biolabs, | |
| | | Recombinant Antibody (CBXS- | #CBMAB-S4469-CQ | |
| | | 1620) | | |
| CBXS-1671 | SUSD2 | Rabbit Anti-SUSD2 | Creative Biolabs, | |
| | | Recombinant Antibody (CBXS- | #CBMAB-S0094-CQ | |
| | | 1671) | | |
| CBXS-1989 | SUSD2 | Mouse Anti-SUSD2 | Creative Biolabs, | |
| | | Recombinant Antibody (CBXS- | #CBMAB-S4758-CQ | |
| | | 1989) | | |
| CBXS-1990 | SUSD2 | Mouse Anti-SUSD2 | Creative Biolabs, | |
| | | Recombinant Antibody (CBXS- | #CBMAB-S0156-CQ | |
| | | 1990) | | |
| CBXS-3571 | SUSD2 | Human Anti-SUSD2 | Creative Biolabs, | |
| | | Recombinant Antibody (CBXS- | #CBMAB-S0386-CQ | |
| | | 3571) | | |
| CBXS-3676 | SUSD2 | Rabbit Anti-SUSD2 | Creative Biolabs, | |
| | | Recombinant Antibody (CBXS- | #CBMAB-S0214-CQ | |
| | | 3676) | | |
| CBYY-I0413 | IL-12R | Mouse Anti-IL12RB2 | Creative Biolabs, | |
| | | Recombinant Antibody (CBYY- | #CBMAB-I1506-YY | |
| | | I0683) | | |
| C-CAR066 Leu16 and 2.1.2 | CD20 | | | Liang et al., 2021, J. Clin. |
| | | | | Oncol. 39(15) suppl: 2508) |
| CD58 (LFA-3) | CD2 | Recombinant Human | R&D Systems, | |
| | | CD58/LFA-3 His-tag Protein | #1689-CD | |
| CDLA68-1 | CD68 | CD68 Antibody [clone CDLA68- | NSJ Bioreagents, | |
| | | 1] | #V7045 | |
| CDX-0158 | CD117 | Anti-SCFR/c-Kit/CD117 | BiOrbyt, | |
| | | Reference Antibody (CDX-0158) | #orb 1817461 | |
| cedelizumab | CD4 | available from multiple sources, including: | | |
| | | Cedelizumab | MedChemExpress, | |
| | | | #HY-P99495 | |
| | | Cedelizumab (CD4) | ProSci, #10-093 | |
| cemiplimab | PD-1 | | | Burova et al., 2017, Mol. |
| | | | | Cancer Ther. 16 (5), 861-870. |
| cetrelimab | PD-1 | Cetrelimab | MedChemExpress, | |
| | | | #HY-P99499 | |
| cetuximab | EGFR | available from multiple sources, including: | | |
| | | EGF R antibody \| C225 | BioRad, #MCA6102 | |
| | | Cetuximab | MedChemExpress, | |
| | | | #HY-P9905 | |
| | | Cetuximab (anti-EGFR) | SelleckChem, | |
| | | | #A2000 | |
| cevostamab | CD3 | Cevostamab | MedChemExpress, | |
| | | | #HY-P99601 | |
| ch28/11 | SSEA-4 | Chimeric (Mouse/Human) Anti- | Creative Biolabs, | |
| | | SSEA-4 Recombinant Antibody | #PABJ-0121 | |
| | | (clone ch28/11) | | |
| cifurtilimab | CD40 | Cifurtilimab; SEA-CD40 | MedChemExpress, | |
| | | | #HY-P99603 | |
| ciltacabtagene autoleucel | BCMA | | | Berdeja et al., 2021, Lancet |
| | | | | 398 (10297): 314-324. |
| CK6 | CD117 | | | Lebron et al., 2014, Cancer |
| | | | | Biol. Ther. 15 (9): 1208-1218. |
| CL1659 | CD117 | CD117/c-kit Antibody (CL1657) | Novus Biologicals, | |
| | | | #NBP2-52975 | |
| CLB-CD19 | CD19 | | | De Rie, 1989, Cell. Immunol. |
| | | | | 118: 368-381 |
| clenoliximab | CD4 | Clenoliximab | Novus Biologicals, | |
| | | | #NBP2-52681H | |
| Cmab-43 | CD133 | Recombinant Anti-CD133 | Abcam, #AB264538 | |
| | | antibody | | |
| CO.44B8 | CD43 | CD43 Monoclonal Antibody | ThermoFisher, | |
| | | (CO.44B8) | #MA5-28371 | |
| cobolimab | TIM-3 | Cobolimab | MedChemExpress, | |
| | | | #HY-P99827 | |
| CPP32 4-1-18 | Sca-1 | Caspase 3 Monoclonal Antibody | ThermoFisher, MA1- | |
| | | (CPP32 4-1-18) | 16843 | |
| crefmirlimab | CD8α | available from multiple sources, including: | | Farwell et al., J Nucl Med |
| | | Crefmirlimab | MedChemExpress, | 63(5): 720-726 (2021); |
| | | | #HY-P99834 | U.S. Pat. No. 10,414,820 |
| | | Crefmirlimab Biosimilar - Anti- | ProteGenix, #PX- | |
| | | CD8A mAb - Research Grade | TA1828-100 | |
| CS1002 | CTLA-4 | | | Bishnoi et al., 2021, Ann. |
| | | | | Oncol. 32, (Suppl. 5): S840. |
| CT041 | CLDN18.2 | | | Qi et al., 2022, Nat. Med. 28, |
| | | | | 1189-1198. |
| CT103A (CAR0085) | BCMA | | | U.S. Pat. No. 11,026,975 |
| cudarolimab | OX40 | Cudarolimab | MedChemExpress, | Kuang, Z et al. Cancer |
| | | | #HY-P99836 | Immunol Immunother. 2020 |
| | | | | June; 69(6):939-950. |
| cusatuzumab | CD70 | Cusatuzumab | MedChemExpress, | Riether, C et al. Nat Med. |
| | | | #HY-P99014 | 2020 September; 26(9): 1459-1467. |
| CYT-91000 | CD25 | Research Grade Anti-Human | AntibodySystem, | |
| | | CD25/IL2RA (CYT-91000) | #DHB95805 | |
| dacetuzumab | CD40 | Dacetuzumab | MedChemExpress, | Hussein, M et al. |
| | | | #HY-P99015 | Haematologica. 2010 |
| | | | | May; 95(5): 845-8. |
| daclizumab | CD25 | Daclizumab | MedChemExpress, | Cohan, S. L. et al. |
| | | | #HY-108738 | Biomedicines. 2019 Mar. |
| | | | | 11; 7(1):18. |
| daratumumab | CD38 | Daratumumab (anti-CD38) | SelleckChem, | |
| | | | #A2027 | |
| davoceticept | CD28 | Davoceticept | MedChemExpress, | Patnaik, A. et al. JCO 40, |
| | | | #HY-P99844 | TPS2683-TPS2683(2022). |
| DF-T1 | CD43 | CD43 Antibody (DF-T1) | Santa Cruz | |
| | | | Biotechnology, | |
| | | | #sc-6256 | |
| dinutuximab | GD2 | Dinutuximab | ThermoFisher, | Mohd, A. B. et al. J Res Med |
| | | | #MA5-42000 | Sci. 2023 Sep. 29; 28: 71. |
| dostarlimab | PD-1 | Dostarlimab | SelleckChem, | Mirza, M. R. et al. N Engl J |
| | | | #A2835 | Med. 2023 Jun. |
| | | | | 8; 388(23): 2145-2158. |
| DS-7300A | B7H3 | | | Yamato et al., 2022, Mol. |
| | | | | Cancer Ther. 21(4): 635-646. |
| EasySep ™ | | Easy Sep ™ Human T Cell | Stemcell | |
| | | Isolation Kit | Technologies, #100- | |
| | | | 0695 | |
| efungumab | Stro-4 | Efungumab | MedChemExpress, | Karwa, R. et al. Ann |
| | | | #HY-P9962 | Pharmacother. 2009 |
| | | | | November; 43(11): 1818-23. |
| ektomab | GD2 | | | Ruf, P. et al. J Transl Med. |
| | | | | 2012 Nov. 7; 10: 219. |
| ELISA MAX ™ Deluxe Set | TNF-α | ELISA MAX ™ Deluxe Set | BioLegend, #430204 | |
| Human TNF-α kit | | Human TNF-α kit | | |
| elranatamab | CD3 | Research Grade Elranatamab | Antibody System, | Lesokhin, A. M. et al. Nat |
| | | | #DHF92406 | Med. 2023 September; 29(9): 2259- |
| | | | | 2267. |
| EMB-02 | LAG-3 | | | Day et al., 2023, Ann. Oncol. |
| | PD-1 | | | 34 (Suppl. 2): S625. |
| EMB-06 | BCMA | | | US 2023/0002489 |
| EMB-09 | OX40 | | | Li et al., 2023, Antib. Ther. 6 |
| | PD-L1 | | | (Suppl 1): tbad014.008. |
| EMD 273063 | GD2 | Anti-GD2 (EMD 273063) | SelleckChem, | |
| | | | #A2916 | |
| encelimab | LAG-3 | Encelimab | MedChemExpress, | Perez-Santos, M. et al. Expert |
| | | | #HY-P99922 | Opin Drug Discov. 2022 |
| | | | | December; 17(12): 1341-1355. |
| enoblituzumab | B7H3 | Anti-Human CD276 | Creative Biolabs, | |
| | | Recombinant Antibody | #TAB-424CQ | |
| EOS-448 | TIGIT | | | Cuende et al., 2022, Cancer |
| | | | | Res. 82 (12_Supplement): |
| | | | | LB189. |
| EP322Y | CD45 | Anti-CD45 antibody [EP322Y] | Abcam, #ab40763 | |
| EPR8913(2) | SUSD2 | Anti-SUSD2 antibody | Abcam, #ab 182147 | |
| | | [EPR8913(2)] | | |
| EPR20545 | CD68 | Anti-CD68 antibody | Abcam, #ab213363 | |
| | | [EPR20545] | | |
| EPR26538-16 | CD8β | Recombinant Anti-CD8 beta | Abcam, #ab300067 | |
| | | antibody [EPR26538-16] | | |
| EPR26948-19 | FCRL5 | Anti-FCRL5 antibody | Abcam, #ab307569 | |
| | | [EPR26948-19] - BSA and Azide | | |
| | | free (Detector) | | |
| EPR26948-67 | FCRL5 | Anti-FCRL5 antibody | Abcam, #ab307568 | |
| | | [EPR26948-67] - BSA and Azide | | |
| | | free (Capture) | | |
| EPR27365-87 | | Anti-FCRL5 antibody | Abcam, #ab307730 | |
| | | [EPR27365-87] | | |
| epratuzumab | CD22 | Anti-Human CD22 Recombinant | Creative Biolabs, | |
| | | Antibody | #TAB-176 | |
| ET200-31 | FCRL5 | | | U.S. Pat. No. 10,913,796 |
| ET200-39 | | | | |
| ET200-69 | | | | |
| ET200-104 | | | | |
| ET200-105 | | | | |
| ET200-109 | | | | |
| ET200-117 | | | | |
| etigilimab | TIGIT | | | Mettu et al., 2022, Clin. |
| | | | | Cancer. Res. 28(5): 882-892. |
| ezabenlimab | PD-1 | Ezabenlimab | MedChemExpress, | Zettl, M. et al. |
| | | | #HY-P99610 | Oncoimmunology. 2022 Jun. |
| | | | | 16; 11(1): 2080328. |
| F10-89-4 | CD45 | Anti-CD45 antibody [F10-89-4] - | Abcam, #ab30470 | |
| | | Hematopoietic Stem Cell Marker | | |
| F19 | FAP | Mouse Anti-FAP Recombinant | Creative Biolabs, | |
| | | Antibody (clone F19) | #HPAB-A1368-YJ | |
| F119 | FCRL5 | | | Ise et al. (2005) Clin. Cancer |
| | | | | Res. 11(1): 87-96. |
| F25 | FCRL5 | Anti-FcRH5 Antibody, clone | Millipore Sigma, | |
| | | F25 | #MABF2103 | |
| F26 | FCRL5 | | | Franco et al. (2013) J. |
| | | | | Immunol. 190(11): 5739- |
| | | | | 5746. |
| FAP5 | FAP | Anti-FAP h(41BB-CD3ζ) CAR, | Creative Biolabs, | Ostermann et al., 2008, Clin. |
| | | pCDCAR1 vector | #CAR-ZP927 | Cancer. Res. 14: 4584-4592. |
| favezelimab | LAG-3 | Favezelimab | MedChemExpress, | Gareth, P. et al. Cancer Res1 |
| | | | #HY-P99613 | July 2019; 79 |
| | | | | (13_Supplement): CT106. |
| felzartamab | CD38 | Felzartamab | MedChemExpress, | |
| | | | #HY-P99616 | |
| FHVH33 | BCMA | | | WO 2019/006072 |
| fianlimab | LAG-3 | Fianlimab | MedChemExpress, | Omid, H. et al. JCO 39, 9515- |
| | | | #HY-P99617 | 9515 (2021). |
| FMC63 | CD19 | | | Nicholson et al., 1997, Mol. |
| | | | | Immun. 34(16-17): 1157-1165; |
| | | | | WO 2018/213337, WO |
| | | | | 2015/187528 |
| forimtamig | GPRC5D | | | Harrison et al. (2023). Clin. |
| | | | | Lymphoma Myeloma Leuk. |
| | | | | 23 (Suppl. 2): S3-4. |
| FR4D11 | CD10 | Anti-Human CD10 Antibody, | Stemcell | |
| | | Clone FR4D11 | Technologies, | |
| | | | #60149 | |
| FR104CD | CD28 | Recombinant Anti-CD28 | Antibodies.com, | |
| | | Antibody [FR104] | #A323936 | |
| FS118 | LAG-3 | | | Kraman et al., 2020, Clin. |
| | | | | Cancer Res. 26 (13): 3333- |
| | | | | 3344. |
| FS120 | OX40 | | | Gaspar et al., 2020, Cancer |
| | CD137 | | | Immunol. Res. 8 (6): 781-793. |
| G.813.2 | CD117 | Phospho-c-Kit (Tyr703) | ThermoFisher, | |
| | | Monoclonal Antibody (G.813.2) | #MA5-14830 | |
| ganglidiximab | GD2 | | | Eger et al., 2016, PLoS One |
| | | | | 11 (3): e0150479. |
| GEN-3014 | CD38 | | | Hiemstra et al., 2023, |
| | | | | EBioMedicine 93: 104663. |
| geptanolimab | PD-1 | Geptanolimab | Antibody System, | Shi, Y. et al. Clin Cancer Res. |
| | | | #DHH02224 | 2020 Dec. 15; 26(24): 6445- |
| | | | | 6452. doi: 10.1158/1078- |
| | | | | 0432.CCR-20-2819. Epub |
| | | | | 2020 Oct. 12. PMID: |
| | | | | 33046518. |
| GIGA-564 | CTLA-4 | | | Stone, E. et al. Journal for |
| | | | | ImmunoTherapy of Cancer, |
| | | | | 2022; 10. |
| givastomig | CLDN18.2 | | | Gao et al., 2023, J. |
| | | | | Immunother. Cancer 11(6): |
| | | | | e006704 |
| GN1412 | CD28 | Theralizumab | MedChemExpress, | Hussain, K. et al. Blood. 2015 |
| | | | #HY-P9975 | Jan. 1; 125(1): 102-10. |
| gresonitamab | CLDN18.2 | Gresonitamab | MedChemExpress, | |
| | | | #HY-P99350 | |
| grisnilimab | CD7 | Grisnilimab | MedChemExpress, | Scharnhorst, V, et al. Gene. |
| | | | #HY-P99650 | 2001 Aug. 8; 273(2): 141-61. |
| | | | | doi: 10.1016/s0378- |
| | | | | 1119(01)00593-5. PMID: |
| | | | | 11595161. |
| GSK2831781 | LAG-3 | | | Clinical Trial ID: |
| | | | | NCT03893565 |
| GSK3174998 | OX40 | | | Infante, J. R. et al. JCO 34, |
| | | | | TPS3107- TPS3107 (2016). |
| GWN323 | GITR | | | Piha-Paul, S. A. et al. Journal |
| | | | | for Immuno Therapy of Cancer |
| | | | | 2021; 9: e002863. |
| h4C8 | CD34 | Anti-Human CD34 Recombinant | Creative Biolabs, | |
| | | Antibody Fab Fragment (h4C8) | #TAB-149LC-F(E) | |
| H22 | CD64 | | | Hristodorov, D. et al. MAbs. |
| | | | | 2014; 6(5): 1283-9. doi: |
| | | | | 10.4161/mabs.32182. PMID: |
| | | | | 25517313; PMCID: |
| | | | | PMC4622438. |
| H44 | IL-18Rα | APC anti-human CD218a (IL- | Biolegend, #313814 | |
| | | 18Rα) Antibody | | |
| H65 | CD5 | CD5 Antibody | Novus Biologicals, | |
| | | | #NB100-2667 | |
| hB-F5 | CD4 | NeuroMab ™ Anti-CD4 BBB | Creative Biolabs, | |
| | | Shuttle Antibody, Clone hB-F5 | #NRZP-1022- | |
| | | | ZP3717 | |
| HBM1007 | CD73 | | | Gan et al., 2020, 2020 AACR |
| | | | | Poster #6056. |
| HBM4003 | CTLA-4 | | | Shanzhi, G. et al. JCO 40, |
| | | | | 2641-2641 (2022). |
| HC34LC14 | CD117 | c-Kit Recombinant Rabbit | ThermoFisher, | |
| | | Monoclonal Antibody | #701494 | |
| | | (HC34LC14) | | |
| HCD27.15 | CD27 | | | U.S. Pat. No. 9,527,916 |
| HD37 | CD19 | | | Pezutto et al., 1987, J. |
| | | | | Immunol. 138(9): 2793-2799 |
| HE3 | CD5 | | | U.S. Pat. No. 5,770,196 |
| HFB301001 | OX40 | | | Fulton, R. et al. Cancer Res 1 |
| | | | | July 2021; 81 |
| | | | | (13_Supplement): 1882. |
| HI30 | CD45 | Purified anti-human CD45 | BioLegend, #304001 | |
| | | Antibody | | |
| HIT8a | CD8α | Purified anti-human CD8a | BioLegend, #300901 | |
| | | Antibody | | |
| HLX26 | LAG-3 | | | Rujiao, L. et al. JCO 41, |
| | | | | e14671-e14671 (2023). |
| HLX51 | OX40 | | | Clinical Trial ID: |
| | | | | NCT05788107 |
| HM48-1 | CD48 | CD48 Monoclonal Antibody | ThermoFisher, | |
| | | (HM48-1), APC, eBioscience ™ | #17-0481-82 | |
| HPAB-0260-YJ | CD200 | Human Anti-CD200 | Creative Biolabs, | |
| | | Recombinant Antibody | #HPAB-0260-YJ- | |
| | | | F(E) | |
| HPAB-3334LY | PODXL | Human Anti-PODXL | Creative Biolabs, | |
| | | Recombinant Antibody | #HPAB-3334LY | |
| HPAB-MO612-YC | PODXL | Human Anti-LAM Recombinant | Creative Biolabs, | |
| | | Antibody | #HPAB-M0561-YC | |
| HPN217 | BCMA | | | U.S. Pat. No. 11,136,403 |
| Hu001-MMAE | CD73 | | | Jin et al., 2020, Mol. Cancer |
| | | | | Ther. 19 (11): 2340-2352. |
| hu3G8-5.1-N297Q | CD16a | | | U.S. Pat. No. 7,351,803 |
| hu14.18k322A | GD2 | Anti-Human GD2 Recombinant | Creative Biolabs, | |
| | | Antibody (hu14.18K322A) | #TAB-304CL | |
| Hu222 | OX40 | | | U.S. Pat. No. 9,006,399 |
| huAA98 | CD146 | Anti-Human MCAM | Creative Biolabs, | |
| | | Recombinant Antibody | #TAB-444MZ | |
| | | (huAA98) | | |
| Huly-m2 | CD7 | CD7 (T-Cell Leukemia Marker) | ThermoFisher, #924- | |
| | | Monoclonal Antibody (HuLy- | MSM2-P1ABX | |
| | | m2) | | |
| HuMab 611 | CD64 | | | WO 2006/002438 |
| Human IgG1 poly-specificity | IgG1 | Human IgG1 Poly-Specificity | Medna Scientific, | |
| control antibody | | Control Antibody | #H1308 | |
| Human IgG4 isotype control | IgG4 | Human IgG4 Isotype Control | Medna Scientific, | |
| antibody | | Antibody | #H1314 | |
| humanized AT-10 | CD32A | CD32 Antibody | Biorad, #MCA1075 | |
| humanized IV.3 | CD32A | Anti-Human CD32 Antibody, | StemCell | |
| | | Clone IV.3 | Technologies, | |
| | | | #60012 | |
| HuMax-TAC | CD25 | Camidanlumab | MedChemExpress, | |
| | | | #HY-P99233 | |
| huRH105 | CD105 | Anti-CD105 [huRH105-1] | Absolute Antibody, | |
| | | | #Ab04017 | |
| IAB22M | CD8 | | | U.S. Pat. No. 11,254,744 |
| IAb_CysDb3b_CD8 | CD8 | | | U.S. Pat. No. 10,414,820 |
| IAb_M1b_CD8 | CD8 | | | |
| IAb_M2b_CD8 | CD8 | | | |
| ibalizumab | CD4 | Ibalizumab | MedChemExpress, | |
| | | | #HY-P99028 | |
| IBI110 | LAG-3 | | | Xu et al., 2022, J. Clin. Oncol. |
| | | | | 40 (16_suppl): 2650. |
| IBI310 | CTLA-4 | | | Zhou et al., 2022, J. Clin. |
| | | | | Oncol. 40 (4_suppl): 421. |
| IBI318 | PD-1 | Reozalimab (LY3434172) | MedChemExpress, | |
| | | | #HY-P9996 | |
| IBI323 | LAG-3 | | | Ni et al., 2020, Cancer Res. 80 |
| | | | | (16_Supplement): 3270. |
| IBI325 | CD73 | | | Zhou et al., 2023, Int. J. Biol. |
| | | | | Macromol. 229: 158-167. |
| IBI343 | CLDN18.2 | | | Yu et al., 2024, J. Clin. Oncol. |
| | | | | 42 (16 Suppl.), 3037. |
| ibritumomab | CD20 | Ibritumomab Biosimilar - Anti- | ProteoGenix, #PX- | |
| | | MS4A1(CD20, MS4A-1) mAb - | TA1620 | |
| | | Research Grade | | |
| idecabtagene vicleucel | BCMA | | | Munshi et al., 2021, N. Engl. |
| | | | | J. Med. 384: 705-716. |
| ieramilimab | LAG-3 | Ieramilimab | MedChemExpress, | Schoffski, P et al. J |
| | | | #HY-P99027 | Immunother Cancer. 2022 |
| | | | | February; 10(2): e003776. |
| IL-A116 | CD45RO | CD45RO Monoclonal Antibody | ThermoFisher, | |
| | | (IL-A116) | #MA5-28403 | |
| imaprelimab | CD146 | Imaprelimab | MedChemExpress, | Pelletier, J. P. R. Et al. |
| | | | #HY-P99658 | Immunologic Concepts in |
| | | | | Transfusion Medicine, |
| | | | | Elsevier, 2020, 251-348. |
| INBRX-106 | OX40 | | | Clinical Trial ID: |
| | | | | NCT04198766 |
| INCA00186 | CD73 | | | Stewart, S. et al. Cancer Res 1 |
| | | | | July 2021; 81 |
| | | | | (13_Supplement): LB174. |
| INCA32459 | LAG-3 | | | Clinical Trial ID: |
| | | | | NCT05577182 |
| INCAGN1949 | OX40 | | | Davis, E. J. et al. J |
| | | | | Immunother Cancer. 2022 |
| | | | | October; 10(10): e004235. doi: |
| | | | | 10.1136/jitc-2021-004235. |
| | | | | PMID: 36316061; PMCID: |
| | | | | PMC9628691. |
| INCAGN2385 | LAG-3 | | | Clinical Trial ID: |
| | | | | NCT03538028 |
| INCAGN02390 | TIM-3 | | | Clinical Trial ID: |
| | | | | NCT03652077 |
| inezetamab | CD4 | Inezetamab | MedChemExpress, | |
| | | | #HY-P99663 | |
| inolimomab | CD25 | Research Grade Inolimomab | Antibody System, | |
| | | | #DHB95804 | |
| inotuzumab | CD22 | Humanized Anti-CD22 | Creative Biolabs, | |
| | | Recombinant Antibody (clone | #TAB-198 | |
| | | Inotuzumab) | | |
| IPH5301 | CD73 | | | Gonçalves et al., 2022, |
| | | | | Immuno-Oncology and |
| | | | | Technology 16 (S1): 199TiP. |
| ipilimumab | CTLA-4 | Ipilimumab | MedChemExpress, | |
| | | | #HY-P9901 | |
| IPO-3 | CD150 | Anti-SLAM/CD150 antibody | Abcam, #ab2604 | |
| | | [IPO-3] | | |
| isatuximab | CD38 | Isatuximab (anti-CD38) | SelleckChem, | |
| | | | #A2039 | |
| iscalimab | CD40 | Iscalimab | MedChemExpress, | |
| | | | #HY-P99670 | |
| ispectamab | BCMA | | | US 2021/0130483 |
| IT1208 | CD4 | | | Shitara, K et al. J Immunother |
| | | | | Cancer. 2019 Jul. 24; 7(1):195. |
| ivuxolimab | OX40 | Ivuxolimab | MedChemExpress | Diab, A et al. Clin Cancer Res. |
| | | | (MCE), #HY-P99159 | 2022 Jan. 1; 28(1): 71-83. |
| JAB-BX102 | CD73 | | | Clinical Trial ID: |
| | | | | NCT05174585 |
| JK08 | CTLA-4 | | | Kotecki, et al., 2023, Ann. |
| | | | | Oncol. 34(S2), pg. S635. |
| JM1-24-3 | CD146 | | | Feng et al., 2020, J. Exp. Clin. |
| | | | | Cancer Res. 39: 273. |
| JM7A4 | IL-15Rα | available from multiple sources, including: | | |
| | | Anti-IL-15RA antibody [JM7A4] | Abcam, #ab91270 | |
| | | IL-15R alpha Antibody (JM7A4) - | Novus Biologicals, | |
| | | BSA Free | #NBP1-43238 | |
| JMW-3B3 | CTLA-4 | Afuco ™ Anti-Human CTLA4 | Creative Biolabs, | |
| | | ADCC Recombinant Antibody | #AFC-185CL | |
| | | (JMW-3B3), ADCC Enhanced | | |
| JNJ-64164711 | GITR | Research Grade Anti-Human | Antibody System, | Holland et al., 2018, Cancer |
| | | CD357/TNFRSF18/GITR | #DHJ89808 | Res. 78 (13_Supplement): |
| | | | | 3813. |
| JS007 | CTLA-4 | | | Guan et al., 2023, MAbs |
| | | | | 15(1): 2153409. |
| JTX-2011 | ICOS | Vopratelimab | MedChemExpress, | |
| | | | #HY-P99382 | |
| K45 | CD117 | c-Kit Monoclonal Antibody | ThermoFisher, | |
| | | (K45) | #MA5-12944 | |
| K117 | CD90 | Anti-CD90 Antibody, Purified | RayBiotech, #136- | |
| | | | 00048 | |
| KD6001 | CTLA-4 | | | Chen et al., 2024, Cancer Res. |
| | | | | 84 (6_Suppl.): 1350. |
| keliximab | CD4 | available from multiple sources, including: | | |
| | | Keliximab | MedChemExpress, | |
| | | | #HY-P99680 | |
| | | Keliximab (CD4) - Research | ProSci, #10-178 | |
| | | Grade Biosimilar | | |
| Ki-M7 | CD68 | available from multiple sources, including: | | |
| | | CD68 antibody \| Ki-M7 | Bio-Rad, | |
| | | | #MCA2375F | |
| | | CD68 Monoclonal Antibody (Ki- | ThermoFisher, | |
| | | M7), FITC | #MA1-82715 | |
| kit2c75 | CD117 | CD117 (c-Kit) Monoclonal | ThermoFisher, | |
| | | Antibody (kit2c75), PE | #MA5-28805 | |
| KM666 | GD2 | | | Nakamura et al., 2001, Cancer |
| | | | | Immunol. Immunother. 50, |
| | | | | 275-284. |
| KN044 | CTLA-4 | | | Clinical Trial ID: |
| | | | | NCT04126590 |
| L17F12 | CD5 | available from multiple sources, including: | | |
| | | Purified anti-human CD5 | BioLegend, #364001 | |
| | | Antibody | | |
| | | CD5 Antibody (L17F12) | Santa Cruz | |
| | | | Biotechnology, #sc- | |
| | | | 18898 | |
| LAMP4-824 | CD68 | available from multiple sources, including: | | |
| | | CD68 Monoclonal Mouse | Biotium, #0824 | |
| | | Antibody (LAMP4/824) | | |
| | | CD68 Monoclonal Antibody | ThermoFisher, #968- | |
| | | (LAMP4, 824) | MSM4-P1 | |
| LB1410 | TIM-3 | | | Liu, 2023, J. Clin. Oncol. 41 |
| | | | | (16_suppl): TPS2663. |
| LCAR-B38M | BCMA | | | Zhao et al., 2018, J. Hematol. |
| | | | | Oncol. 11(1): 141; |
| | | | | WO 2018/028647 |
| LCAR-C18S (LB1908) | CLDN18.2 | | | Zhen et al., 2023, J. Clin. |
| | | | | Oncol. 41 (4 suppl.), TPS480. |
| Leu16 | CD20 | | | Wu et al., 2001, Protein |
| | | | | Engineering. 14(12): 1025- |
| | | | | 1033 |
| linvoseltamab | BCMA | | | U.S. Pat. No. 11,919,965 |
| lisocabtagene maraleucel | CD19 | | | Abramson et al., 2020, Lancet |
| | | | | 396: 839-852. |
| LM-302 | CLDN18.2 | | | Huang et al., 2022, Eur. J. |
| | | | | Cancer 174 (Suppl. 1): S41- |
| | | | | S42. |
| LOP628 | CD117 | Anti-cKit-maitansine ADC | Creative Biolabs, | |
| | | (LOP628) | #ADC-L039 | |
| lorigerlimab | PD-1 | | | Luke et al., J. Clin. Oncol. 41 |
| | | | | (6_suppl): 155. |
| lorvotuzumab | CD56 | available from multiple sources, including: | | |
| | | Lorvotuzumab | MedChemExpress, | |
| | | | #HY-P99372 | |
| | | Lorvotuzumab Recombinant | ThermoFisher, | |
| | | Human Monoclonal Antibody | #MA5-41708 | |
| LT-2 | CD2 | CD2 Monoclonal Antibody | ThermoFisher, | |
| | | (LT2) | #MA1-10131 | |
| LT-8 | CD8 | available from multiple sources, including: | | |
| | | Mouse monoclonal [LT8] | Antibodies.com, | |
| | | antibody to CD8 (PE) | #A242891 | |
| | | Monoclonal Anti-CD8 antibody | Millipore Sigma, | |
| | | | #SAB4701064 | |
| lucatumumab | CD40 | available from multiple sources, including: | | |
| | | Lucatumumab | MedChemExpress, | |
| | | | #HY-P99167 | |
| | | Anti-CD40 Recombinant | Creative Biolabs, | |
| | | Antibody (Lucatumumab) | #TAB-759 | |
| lulizumab | CD28 | available from multiple sources, including: | | |
| | | Lulizumab Recombinant Human | ThermoFisher, | |
| | | Monoclonal Antibody | #MA5-42032 | |
| | | Humanized Anti-CD28 | Creative Biolabs, | |
| | | Recombinant Antibody (clone | #TAB-H46 | |
| | | Lulizumab) | | |
| LVGN6051 | CD137 | | | Qi et al., 2019, Nat. Commun. |
| | | | | 10(1): 2141. |
| LY75_A1 | CD205 | LY75/DEC-205 Protein (AA 28- | Antibodies Online, | |
| | | 1667) (His tag) | #ABIN7275222 | |
| LY3321367 | TIM-3 | | | Harding et al., 2021, Clin. |
| | | | | Cancer Res. 27(8): 2168-2178. |
| LY3415244 | TIM-3 | | | Hellmann et al., 2021, Clin. |
| | | | | Cancer Res. 27(10): 2773- |
| | | | | 2781. |
| M108 (FG-M108) | CLDN18.2 | | | Jin et al., 2024, J. Clin. Oncol. |
| | | | | 42 (16 suppl.), 4142. |
| M971 | CD22 | Anti-Siglec-2/CD22 (NCI | SelleckChem, | |
| | | m971) | #A3057 | |
| mAb19 | CD73 | | | Wurm et al., 2021, Mol. |
| | | | | Cancer Ther. 20(11): 2250- |
| | | | | 2261 |
| MAB107 | CD11b | available from multiple sources, including: | | |
| | | Anti-CD11b/CD18 Antibody, | Millipore Sigma, | |
| | | clone mAb107 | #MABF2085 | |
| | | Anti-human IgE mAb (107), | Mabtech, #3810-3 | |
| | | unconjugated | | |
| MAB1472-100 | IL-15Rα | available from multiple sources, including: | | |
| | | Human IL-15R alpha Antibody | R&D Systems, | |
| | | | #MAB1472 | |
| | | IL-15R alpha Antibody | Novus Biologicals, | |
| | | (1020456) [Unconjugated] | #MAB1472 | |
| MAB5511 | IL-15Rα | Mouse IL-15R alpha Antibody | R&D Systems, | |
| | | | #MAB5511 | |
| MAI1738 | PODXL | Research Grade Anti-Human | Antibody System, | |
| | | PODXL (MAI1738) | #DHA16801 | |
| MAT304 | CD5 | Research Grade Anti-Human | AntibodySystem, | |
| | | CD5 (MAT 304) | #DHC18804 | |
| MAX.16H5 | CD4 | | | Guse et al., 1994, J. |
| | | | | Chromatogr. A 661: 13-23. |
| MB-106 | CD20 | Fred Hutchinson Cancer Research Center | | Shadman et al., 2019, Blood |
| | | | | 134(Suppl. 1): 3235 |
| MC631 | SSEA-3 | available from multiple sources, including: | | |
| | | Anti-SSEA3 antibody [MC631] | Abcam, #ab16286 | |
| | | Anti-Mouse SSEA-3 Antibody, | StemCell | |
| | | Clone MC-631 | Technologies, | |
| | | | #60061.1 | |
| MC-813-70 | SSEA-4 | available from multiple sources, including: | | |
| | | SSEA4 Monoclonal Antibody | ThermoFisher, | |
| | | (MC-813-70) | #MA1-021 | |
| | | Anti-Human SSEA-4 Antibody, | StemCell | |
| | | Clone MC-813-70 | Technologies, | |
| | | | #60062 | |
| MCARH109 | GPRC5D | | | Mailankody et al., N Engl J |
| | | | | Med. 387(13): 1196-1206 |
| | | | | (2022) |
| MDE-8 | CD32A | Anti-CD32 [MDE-8] | Absolute Antibodies, | |
| | | | #Ab02072 | |
| MDX-1203 | CD70 | | | Owonikoko et al., 2014, J. |
| | | | | Clin. Oncol. 32: 5s, (suppl; |
| | | | | abstr 2558). |
| MDX-1411 | CD70 | Anti-CD70 monoclonal antibody | Creative Biolabs, | |
| | | (MDX-1411) | #Gly-008CL | |
| MEDI0680 | PD-1 | | | Naing et al., 2019, J. |
| | | | | Immunother. Cancer 7: 225. |
| MEDI1873 | GITR | | | Tigue et al., 2017, |
| | | | | Oncoimmunology 6(3): |
| | | | | e1280645. |
| MEDI2228 | BCMA | | | U.S. Pat. No. 10,988,546 |
| MEDI5752 | PD-1 | | | Dovedi et al., 2021, Cancer |
| | | | | Discov. 11(5): 1100-1117. |
| MEDI6383 | OX40 | | | Hammond et al., 2015, J. Clin. |
| | | | | Oncol. 33 (15_suppl): 3056. |
| MEDI6469 | OX40 | | | Leidner et al., 2015, J. Clin. |
| | | | | Oncol. 33 (15_suppl): |
| | | | | TPS6083. |
| MEM-28 | CD45 | Anti-CD45 antibody [MEM-28] | Abcam, #ab8216 | |
| MEM-31 | CD8 | available from multiple sources, including: | | |
| | | CD8 Monoclonal Antibody | ThermoFisher, | |
| | | (MEM-31) | #MA1-19082 | |
| | | Monoclonal Anti-CD8 antibody | SigmaAldrich, | |
| | | produced in mouse | #SAB4700084 | |
| MEM-55 | CD45RB | Anti-CD45RB antibody [MEM- | Abcam, #ab8218 | |
| | | 55] | | |
| MEM-59 | CD43 | CD43 Monoclonal Antibody | ThermoFisher, | |
| | | (MEM-59) | #MA1-19009 | |
| MEM-65 | CD2 | available from multiple sources, including: | | |
| | | CD2 Monoclonal Antibody | ThermoFisher, | |
| | | (MEM-65) | #MA1-19070 | |
| | | Anti-CD2 Antibody [MEM-65] | Antibodies.com, | |
| | | | #A86387 | |
| MEM-87 | CD8 | available from multiple sources, including: | | |
| | | Anti-CD8 Antibody (MEM-87) | Antibodies.com, | |
| | | | #A85681 | |
| | | Monoclonal Anti-CD8 antibody | SigmaAldrich, | |
| | | produced in mouse | #SAB4700089 | |
| MEM-102 | CD48 | CD48 Monoclonal Antibody | ThermoFisher, | |
| | | (MEM-102) | #MA1-19119 | |
| mezagitamab | CD38 | Mezagitamab | MedChemExpress, | |
| | | | #HY-P99730 | |
| MGC018 | B7H3 | | | Scribner et al., 2020, Mol. |
| | | | | Cancer Ther. 19(11): 2235- |
| | | | | 2244. |
| MGTA-117 | CD117 | | | Westervelt et al., 2022, Blood |
| | | | | 140 (Supplement 1): 2117- |
| | | | | 2119. |
| MiK-Beta-1 | CD122 | CD122 Monoclonal Antibody | ThermoFisher, | |
| | | (MIK-Beta 1) | #MA1-35896 | |
| miptenalimab | LAG-3 | Miptenalimab | MedChemExpress, | |
| | | | #HY-P99736 | |
| mitazalimab | CD40 | | | Calvo et al., 2019, J. Clin. |
| | | | | Oncol. 37 (15_suppl): 2527. |
| MK-1248 | GITR | | | Geva et al., 2020, Cancer |
| | | | | 126(22): 4926-4935. |
| MK-4166 | GITR | | | Sukumar et al., 2017, Cancer |
| | | | | Res. 77 (16): 4378-4388. |
| MORAb-028 | GD2 | MORAb-028 Recombinant | ThermoFisher, | |
| | | Human Monoclonal Antibody | #MA5-41810 | |
| moxetumomab | CD22 | Anti-CD22 (Moxetumomab | Creative Biolabs, | |
| | | pasudotox)-SMCC-DM1 ADC | #ADC-W-800 | |
| mShad150 | CD150 | CD150 Monoclonal Antibody | ThermoFisher, | |
| | | (mShad150), PE, eBioscience ™ | #12-1502-82 | |
| M-T413 | CD4 | | | Rieber et al., 1992, PNAS |
| | | | | 89(22): 10792-6. |
| MT95-4 | CD13 | | | WO 2021/072312 |
| MRTX1011A | CD4 | | | Scheerens et al., 2011, |
| | | | | Arthritis Res. Ther. 13 (5): |
| | | | | R177. |
| MT807-R1 | CD8α | Anti-CD8 alpha (MT807R1) | NIH Nonhuman | Nakamura-Hoshi et al., 2024, |
| | | | Primate Reagent | Mol. Ther. 32 (7): 1-12. |
| | | | Resources, | |
| | | | #AB_2716320 | |
| mupadolimab | CD73 | Mupadolimab | MedChemExpress, | |
| | | | #HY-P99181 | |
| My10 | CD34 | Anti-CD34 antibody [My10] | Abcam, #ab245689 | |
| naxitamab | GD2 | Naxitamab | MedChemExpress, | |
| | | | #HY-P99206 | |
| Nb157 | CD13 | | | WO 2021/072312 |
| ND-742 | SSEA-3 | Anti-SSEA-3 monoclonal | Creative Diagnostics, | |
| | | antibody | #DMAB12907 | |
| nivatrotamab | GD2 | Nivatrotamab | MedChemExpress, | |
| | | | #HY-P99757 | |
| nivolumab | PD-1 | available from multiple sources, including: | | |
| | | Nivolumab (anti-PD-1) | SelleckChem, | |
| | | | #A2002 | |
| | | Nivolumab | Evidentic, #8006657 | |
| NN2101 | CD117 | | | Kim et al., 2020, Int. J. Biol. |
| | | | | Macromol. 159: 66-78. |
| nofazinlimab | PD-1 | Nofazinlimab | MedChemExpress, | |
| | | | #HY-P99758 | |
| nurulimab | CTLA-4 | available from multiple sources, including: | | |
| | | Nurulimab | MedChemExpress, | |
| | | | #HY-P99760 | |
| | | Nurulimab (Anti-CTLA-4/ | SelleckChem, | |
| | | CD152 | #A2859 | |
| NZV930 | CD73 | | | Fu et al., 2022, Cancer Res. |
| | | | | 82(12_Suppl), CT503. |
| obinutuzumab | CD20 | available from multiple sources, including: | | |
| | | Obinutuzumab | MedChemExpress, | |
| | | | #HY-P9910 | |
| | | Obinutuzumab (anti-CD20) | SelleckChem, | |
| | | | #A2023 | |
| | | Obinutuzumab Monoclonal | ThermoFisher, | |
| | | Antibody (16B7) | #A01946-40 | |
| ocrelizumab | CD20 | available from multiple sources, including: | | |
| | | Ocrelizumab | MedChemExpress, | |
| | | | #HY-P9960 | |
| | | Ocrelizumab (Anti-CD20) | SelleckChem, | |
| | | | #A2561 | |
| | | Ocrelizumab Recombinant | ThermoFisher, | |
| | | Human Monoclonal Antibody | #MA5-41829 | |
| odronextamab | CD20 | available from multiple sources, including: | | |
| | CD3 | Odronextamab | MedChemExpress, | |
| | | | #HY-P99038 | |
| | | Odronextamab Recombinant | ThermoFisher, | |
| | | Human Monoclonal Antibody | #MA5-42283 | |
| ofatumumab | CD20 | available from multiple sources, including: | | |
| | | Ofatumumab | MedChemExpress, | |
| | | | #HY-P9961 | |
| | | Ofatumumab Biosimilar - Anti- | ProteoGenix, #PX- | |
| | | MS4A1, CD20 mAb - Research | TA1146 | |
| | | Grade | | |
| | | Ofatumumab Recombinant | ThermoFisher, | |
| | | Human Monoclonal Antibody | #MA5-41813 | |
| OKT1 | CD5 | CD5 Unconjugated OKT1 IgG1k | Biohippo, | |
| | | (RUO) | #BHA19900487 | |
| OKT3 (muromonab-CD3) | CD3 | CD3 Monoclonal Antibody | ThermoFisher, #14- | |
| | | (OKT3), eBioscience ™ | 0037-82 | |
| OKT4 | CD4 | Brilliant Violet 650 ™ anti- | BioLegend, #317436 | |
| | | human CD4 Antibody | | |
| OKT8 | CD8α | CD8α Monoclonal Antibody | ThermoFisher, #14- | |
| | | (OKT8) | 0086-80 | |
| OKT11 | CD2 | available from multiple sources, including: | | U.S. Pat. No. 4,364,937 |
| | | OKT11 | ATCC, #CRL-8027 | |
| | | | MilliporeSigma, | |
| | | | #86050802 | |
| oleclumab | CD73 | Oleclumab | MedChemExpress, | |
| | | | #HY-P99039 | |
| ONC-392 | CTLA-4 | | | Rolfo et al., 2020, J. Clin. |
| | | | | Oncol. 38 (15_suppl): |
| | | | | TPS3159. |
| OriCAR-017 | GPRC5D | | | Rodriguez-Otero et al., Blood |
| | | | | Cancer J. 14(1): 24 (2024) |
| OS2966 | CD29 | | | Nigim et al., 2019, Target. |
| | | | | Oncol. 14: 479-489. |
| osemitamab | CLDN18.2 | | | Qian et al., 2023, Ann. Oncol. |
| | | | | 34 (Suppl. 2), S873, 1560P. |
| otelixizumab | CD3 | Otelixizumab | MedChemExpress, | |
| | | | #HY-P99211 | |
| OTI14B1 | CD117 | KIT Mouse anti-Human, Clone: | Fisher Scientific, | |
| | | OTI14B1, lyophilized, | #50-166-9129 | |
| | | TrueMAB ™ | | |
| OTI1B6 | | KIT Monoclonal Antibody | ThermoFisher, | |
| | | (OTI1B6), TrueMAB ™ | #CF808721 | |
| OTI1E2 | | CD117 Mouse Monoclonal | OriGene, #TA801037 | |
| | | Antibody [Clone ID: OTI1E2] | | |
| OTI1F6 | | KIT Monoclonal Antibody | ThermoFisher, | |
| | | (OTI1F6), TrueMAB ™ | #CF801131 | |
| OTI2B12 | | CD117 Mouse Monoclonal | OriGene, #CF808739 | |
| | | Antibody [Clone ID: OTI2B12] | | |
| OTI2B5 | | KIT Monoclonal Antibody | ThermoFisher, | |
| | | (OTI2B5), TrueMAB ™ | #TA801047 | |
| OTI2C1D5 | | KIT Monoclonal Antibody | ThermoFisher, | |
| | | (OTI2C1D5), TrueMAB ™ | #CF801043 | |
| OTI2C1H4 | | KIT Monoclonal Antibody | ThermoFisher, | |
| | | (OTI2C1H4), TrueMAB ™ | #CF805183 | |
| OTI2E3 | | Monoclonal Mouse anti-Human | LSBio, | |
| | | c-Kit/CD117 Antibody | #LS-C789071-100 | |
| | | (Carrier-free, aa546-976, IHC, | | |
| | | WB) | | |
| OTI3F9 | CD117 | KIT Monoclonal Antibody | ThermoFisher, | |
| | | (OTI3F9), TrueMAB ™ | #TA801045 | |
| OTI4E4 | CD2 | available from multiple sources, including: | | |
| | | CD2 Monoclonal Antibody | ThermoFisher, | |
| | | (OTI4E4) | #TA500404 | |
| | | CD2 Mouse Monoclonal | Cambridge | |
| | | Antibody [Clone ID: OTI4E4] | Bioscience, | |
| | | | #TA500404 | |
| OTI4G8 | CD43 | ASPDH Monoclonal Antibody | ThermoFisher, | |
| | | (OTI4G8) | #MA5-27041 | |
| OTI6A4 | CD43 | PRRX1 Monoclonal Antibody | ThermoFisher, | |
| | | (OTI6A4) | #MA5-26579 | |
| OTI6F8 | CD117 | KIT Monoclonal Antibody | ThermoFisher, | |
| | | (OTI6F8), TrueMAB ™ | #CF808746 | |
| OTI9A11 | CD117 | CD117 Mouse Monoclonal | OriGene, #CF801205 | |
| | | Antibody [Clone ID: OTI9A11] | | |
| OX7 | CD90 | available from multiple sources, including: | | |
| | | CD90 Monoclonal Antibody | ThermoFisher, | |
| | | (OX-7) | #MA1-81491 | |
| | | Thy-1/CD90 Antibody (OX7) | Santa Cruz | |
| | | | Biotechnology, #sc- | |
| | | | 53116 | |
| OX-45 | CD48 | CD48 Monoclonal Antibody | ThermoFisher, | |
| | | (OX-45), PE | #MA5-17528 | |
| OX-104 | CD200 | available from multiple sources, including: | | |
| | | CD200 Monoclonal Antibody | ThermoFisher, #14- | |
| | | (OX104), eBioscience ™ | 9200-82 | |
| | | CD200 antibody \| OX-104 | Bio-Rad, #MCA1960 | |
| pavurutamab | CD3 | Pavurutamab | MedChemExpress, | |
| | | | #HY-P99814 | |
| PD7/26 | CD45RB | CD45RB Monoclonal Antibody | ThermoFisher, | |
| | | (PD7/26), eBioscience ™ | #14-9458-82 | |
| pembrolizumab | PD-1 | available from multiple sources, including: | | |
| | | Pembrolizumab | MedChemExpress, | |
| | | | #HY-P9902 | |
| | | | Evidentic, | |
| | | | #6302603D04 | |
| penpulimab | PD-1 | available from multiple sources, including: | | |
| | | Penpulimab | AbMole, #M25087 | |
| | | Penpulimab (Anti-PDCD1/PD- | Selleckchem, | |
| | | 1/CD279 | #A3034 | |
| peresolimab | PD-1 | Peresolimab | MedChemExpress, | |
| | | | #HY-P9993 | |
| PF-03475952 | CD44 | | | Runnels et al., 2010, Adv. |
| | | | | Ther. 27(3): 168-180. |
| PG-M1 | CD68 | available from multiple sources, including: | | |
| | | Anti-CD68 antibody [PG-M1] | Abcam, #ab783 | |
| | | CD68 Monoclonal Antibody | ThermoFisher, | |
| | | (PG-M1) | #MA5-12407 | |
| PI37012 | TREM2 | | | WO 2019/118513 |
| pidilizumab | PD-1 | Anti-Human PD- | Creative Biolabs, | |
| | | 1/PDCD1/CD279 Recombinant | #TAB-900 | |
| | | Antibody (Pidilizumab) | | |
| pimivalimab | PD-1 | Pimivalimab | MedChemExpress, | |
| | | | #HY-P99887 | |
| pinatuzumab | CD22 | Pinatuzumab | MedChemExpress, | |
| | | | #HY-P99230 | |
| praluzatamab | CD166 | | | Boni et al., 2022, Clin. Cancer |
| | | | | Res. 28(10): 2020-2029. |
| prezalumab | CD28 | Prezalumab | MedChemExpress, | |
| | | | #HY-P99414 | |
| priliximab | CD4 | available from multiple sources, including: | | |
| | | Priliximab | MedChemExpress, | |
| | | | #HY-P99790 | |
| | | Anti-Human CD4 Recombinant | Creative Biolabs, | |
| | | Antibody (Priliximab) | #TAB-164 | |
| PRO1160 | CD70 | | | Jonasch et al., 2023, |
| | | | | Oncologist 28(Suppl 1): S14- |
| | | | | S15. |
| prolgolimab | PD-1 | Prolgolimab | MedChemExpress, | |
| | | | #HY-P99807 | |
| promiximab | CD56 | Human Anti-NCAM1 | Creative Biolabs, | |
| | | Recombinant Antibody | #HPAB-N0187-YC | |
| PRS-343 | CD137 | | | Hinner et al., 2016, Cancer |
| | | | | Immunol. Res. 4 |
| | | | | (11_Supplement): B016. |
| pucotenlimab | PD-1 | Pucotenlimab | MedChemExpress, | |
| | | | #HY-P99938 | |
| Q-1802 | CLDN18.2 | | | Yk et al., 2023, J. Clin. Oncol. |
| | | | | 41 (4 suppl.), 382. |
| QBEND/10 | CD34 | CD34 Monoclonal Antibody | ThermoFisher, | |
| | | (QBEND/10), PE | #MA1-10205 | |
| QL1706 | PD-1 | | | Zhang et al., 2021, Cancer |
| | CTLA-4 | | | Res. 81(13_Suppl): CT119. |
| quavonlimab | CTLA-4 | Quavonlimab | MedChemExpress, | |
| | | | #HY-P99809 | |
| r18D11 | CD14 | Anti-Human CD14 Recombinant | Creative Biolabs, | |
| | | Antibody (18D11) | #TAB-1583CL | |
| ragifilimab | GITR | Ragifilimab | MedChemExpress, | |
| | | | #HY-P99812 | |
| RAVB3 | CD8 | available from multiple sources, including: | | |
| | | CD8 alpha Monoclonal Antibody | ThermoFisher, | |
| | | (RAVB3) | #MA5-17048 | |
| | | CD8 (RAVB3) | Santa Cruz | |
| | | | Biotechnology, #sc- | |
| | | | 32812 | |
| RC118 | CLDN18.2 | | | Liu et al., 2024, Ann. Oncol. |
| | | | | 35 (Suppl. 2), S903, 1456P. |
| REA101 | SSEA-4 | SSEA-4 Antibody, anti-human, | Miltenyi Biotec, | |
| | | PE, REAfinity ™ | #130-122-914 | |
| REA157 | PODXL | TRA-1-60 Antibody, anti- | Miltenyi Biotec, | |
| | | human, PE, REAfinity ™ | #130-122-921 | |
| REA246 | PODXL | TRA-1-81 Antibody, anti- | Miltenyi Biotec, | |
| | | human, PE, REAfinity ™ | #130-123-293 | |
| REA233 | IL-21Rα | CD360 (IL-21R) Antibody, anti- | Miltenyi Biotec, | |
| | | human, PE, REAfinity ™ | #130-126-094 | |
| REA333 | IL-12R | IL-12R β2 Antibody, anti- | Miltenyi Biotec, | |
| | | human, PE, REAfinity ™ | #130-120-068 | |
| REA442 | CD166 | CD166 Antibody, anti-human, | Miltenyi Biotec, | |
| | | PE, REAfinity ™ | #130-118-349 | |
| REA795 | SUSD2 | SUSD2 Antibody, anti-human, | Miltenyi Biotec, | |
| | | PE, REAfinity ™ | #130-111-641 | |
| REA844 | CD271 | CD271 (LNGFR) Antibody, anti- | Miltenyi Biotec, | |
| | | human, PE, REAfinity ™ | #130-112-601 | |
| REA877 | CD10 | CD10 Antibody, anti-human, | Miltenyi Biotec, | |
| | | pure, REAfinity ™ | #130-124-312 | |
| REA897 | CD90 | CD90 Antibody, anti-human, PE, | Miltenyi Biotec, | |
| | | REAfinity ™ | #130-114-860 | |
| REA1060 | CD29 | CD29 Antibody, anti-human, PE, | Miltenyi Biotec, | |
| | | REAfinity ™ | #130-118-121 | |
| REA1067 | CD200 | CD200 Antibody, anti-human, | Miltenyi Biotec, | |
| | | PE, REAfinity ™ | #130-118-129 | |
| REA1164 | CD34 | CD34 Antibody, anti-human, PE, | Miltenyi Biotec, | |
| | | REAfinity ™ | #130-120-515 | |
| REAL219 | MSCA-1 | MSCA-1 Antibody, anti-human, | Miltenyi Biotec, | |
| | | PE, REAlease ® | #130-119-917 | |
| REAL709 | CD271 | CD271 (LNGFR) Antibody, anti- | Miltenyi Biotec, | |
| | | human, PE, REAlease ® | #130-125-053 | |
| REGN5459 | BCMA | | | U.S. Pat. No. 11,384,153 |
| REGN4659 | CTLA-4 | | | Burova et al., 2018, Cancer |
| | | | | Res. 78 (13_Supplement): |
| | | | | 3824 |
| REGN6569 | GITR | | | Lakhani et al., 2022, IOTECH |
| | | | | 16 (S1): 196TiP. |
| REGN7257 | CD132 | | | Le Floch-Ramondou et al., |
| | | | | 2022, Blood 140 (Supplement |
| | | | | 1): 473-474. |
| relatlimab | LAG-3 | Relatlimab | MedChemExpress, | |
| | | | #HY-P99156 | |
| retifanlimab | PD-1 | Retifanlimab | MedChemExpress, | |
| | | | #HY-P99941 | |
| RG7356 | CD44 | Anti-Human CD44 Recombinant | Creative BIolabs, | |
| | | Antibody (RG7356) | #TAB-128CL | |
| rituximab | CD20 | available from multiple sources, including: | | |
| | | BD Pharmingen ™ Purified | BD Biosciences, | |
| | | NA/LE Anti-Human CD20 | #569496 | |
| | | Rituximab Biosimilar | | |
| | | HUMAN ANTI CD20 | BioRad, #MCA6091 | |
| | | (RITUXIMAB BIOSIMILAR) | | |
| | | Rituximab | MedChemExpress, | |
| | | | #HY-P9913 | |
| RIV-11 | CD8 | available from multiple sources, including: | | |
| | | Anit-CD8 Antibody (RIV-11) | Antibodies.com, | |
| | | | #A115691 | |
| | | CD8 Recombinant | Aviva Systems | |
| | | Antibody(RIV-11) | Biology, | |
| | | | #OAAV00971 | |
| RM2051 | CD45 | Anti-CD45 antibody [RM2051] | Abcam, #ab318154 | |
| RO5429083 | CD44 | | | Perez et al., 2012, Cancer Res. |
| | | | | 72 (8_Suppl.): 2521. |
| RO7121661 | PD-1 | | | Clinical Trial ID: |
| | TIM-3 | | | NCT04785820 |
| RO7247669 | PD-1 | | | Clinical Trial ID: |
| | LAG-3 | | | NCT04785820 |
| RO7296682 | CD25 | Research Grade Anti-Human | Antibody System, | |
| | | CD25/IL2RA (RO7296682) | #DHB95808 | |
| rocatinlimab | OX40 | Rocatinlimab | MedChemExpress, | |
| | | | #HY-P99955 | |
| RPA-2.10 | CD2 | available from multiple sources, including: | | |
| | | Purified anti-human CD2 | BioLegend, #300202 | |
| | | antibody | | |
| | | CD2 Monoclonal Antibody | ThermoFisher, #14- | |
| | | (RPA-2.10) | 0029-82 | |
| | | Anti-Human CD2 Antibody, | Stemcell | |
| | | Clone RPA-2.10 | Technologies, | |
| | | | #60007 | |
| RPA-T8 (CT8) | CD8α | available from multiple sources, including: | | |
| | | MOUSE ANTI HUMAN CD8 | Bio-Rad, | |
| | | | #MCA4609T | |
| | | CD8a Monoclonal Antibody | ThermoFisher, #17- | |
| | | (RPA-T8), APC | 0088-42 | |
| RTX-003 | CD25 | | | “Nature-inspired, antibody |
| | | | | discovery driven by AI.” |
| | | | | Nature, December 2020, B44. |
| rulonilimab | PD-1 | Rulonilimab | MedChemExpress, | |
| | | | #HY-P99895 | |
| RW03 | CD133 | | | Vora et al., 2020, Cell Stem |
| | | | | Cell 26(6): 832-844.e6 |
| sabatolimab | TIM-3 | Sabatolimab | MedChemExpress, | |
| | | | #HY-P99044 | |
| samalizumab | CD200 | Samalizumab | MedChemExpress, | |
| | | | #HY-P99400 | |
| SAR445514 | BCMA | | | US 2024/0034816 |
| sasanlimab | PD-1 | Sasanlimab (Anti-PDCD1/PD-1/ | Selleckchem, | |
| | | CD279) | #A3038 | |
| sdA1 | CD16a | | | WO 2018/039626 |
| sdA2 | CD16a | | | WO 2018/039626 |
| SEA-BCMA | BCMA | | | U.S. Pat. No. 11,078,291 |
| selicrelumab | CD40 | Selicrelumab | MedChemExpress, | |
| | | | #HY-P99046 | |
| semzuvolimab | CD4 | Semzuvolimab | MedChemExpress, | |
| | | | #HY-P9998 | |
| serplulimab | PD-1 | Serplulimab (Anti-PDCD1/PD- | Selleckchem, | |
| | | 1/CD279) | #A2381 | |
| SG001 | PD-1 | | | Clinical Trial ID: |
| | | | | NCT04886700 |
| SGN-CD70A | CD70 | | | Sandall et al., 2014, Cancer |
| | | | | Res. 74 (19_Supplement): |
| | | | | 2647. |
| SHR-1702 | TIM-3 | | | Clinical Trial ID: |
| | | | | NCT03871855 |
| SHR-1802 | LAG-3 | | | Deng et al., 2023, Ther. Adv. |
| | | | | Med. Oncol. 15: |
| | | | | 17588359231186025. |
| sibrotuzumab | FAP | Anti-Human FAP Recombinant | Creative Biolabs, | |
| | | Antibody | #TAB-211 | |
| SIDI8BEE | CD8B | available from multiple sources, including: | | |
| | | CD8b Monoclonal Antibody | ThermoFisher, # 14- | |
| | | (SIDI8BEE) | 5273-82 | |
| | | Mouse Anti-CD8B Recombinant | CreativeBiolabs, | |
| | | Antibody (SIDI8BEE) | #CBMAB-C10732- | |
| | | | LY | |
| sindelizumab | PD-1 | | | Li et al., 2022, Front. |
| | | | | Pharmacol. 13: 918709. |
| sintilimab | PD-1 | Sintilimab | MedChemExpress, | |
| | | | #HY-P99048 | |
| siplizumab | CD2 | available from multiple sources, including: | | |
| | | Siplizumab | Medchem Express, | |
| | | | #HY-P99904 | |
| | | Anti-Human CD2 Recombinant | Creative Biolabs, | |
| | | Antibody | #TAB-104 | |
| SJ25C1 | CD19 | | | Bejcek et al., 1995, Cancer |
| | | | | Res. 55:2346-2351 |
| SK1 | CD8α | available from multiple sources, including: | | |
| | | Purified Anti-human CD8 | AAT Bioquest, | |
| | | Antibody *SK1* | #10081000 | |
| | | Anti-CD8 Mouse Monoclonal | Avantor (VWR), | |
| | | Antibody [clone: SK1] | #344702-BL | |
| SKB315 | CLDN18.2 | | | Clinical Trial ID: |
| | | | | NCT05367635 |
| SLAM.4 | CD150 | CD150 Monoclonal Antibody | ThermoFisher, | |
| | | (SLAM.4) | #MA5-46198 | |
| SM03 | CD22 | | | Wong et al., 2022, J. |
| | | | | Immunol. 208(12): 2726- |
| | | | | 2737. |
| SOT102 | CLDN18.2 | | | Spisek, 2023, ESMO Open |
| | | | | 8(1), Suppl. 2, 101196, 2P. |
| sotigalimab | CD40 | Sotigalimab | MedChemExpress, | |
| | | | #HY-P99049 | |
| SP-16 | CD8 | available from multiple sources, including: | | |
| | | Anti-CD8 monoclonal antibody | ThermoFisher, | |
| | | (SP-16) | #MA5-14548 | |
| | | | Novus Biologicals, | |
| | | | #NBP2-26484 | |
| SP34-2 | CD3 | BD Pharmingen ™ APC-Cy ™7 | BD Biosciences, | |
| | | Mouse Anti-Human CD3 | #557757 | |
| SP55 | CD43 | CD43 Monoclonal Antibody | ThermoFisher, | |
| | | (SP55) | #MA5-16339 | |
| spartalizumab | PD-1 | Spartalizumab | MedChemExpress, | |
| | | | #HY-P9972 | |
| SPM504 | CD45RA | Anti-CD45RA antibody | Abcam, #ab231426 | |
| | | [SPM504] | | |
| SPX-101 | CLDN18.2 | | | Zhu et al., 2020, Cancer Res. |
| | | | | 80 (16_Supplement): 3361. |
| SR1 | CD117 | Anti-Human KIT Recombinant | Creative Biolabs, | |
| | | Antibody (SR1) | #TAB-467LC | |
| ST04-99 | CD117 | CD117/c-kit Rabbit anti-Human, | Fisher Scientific, | |
| | | Clone: ST04-99, Novus | #NBP267531 | |
| | | Biologicals ™ | | |
| STRO-1 | Stro-1 | STRO-1 Monoclonal Antibody | ThermoFisher, #39- | |
| | | (STRO-1) | 8401 | |
| STRO-4 | Hsp90 | | | Gronthos et al., 2009, Stem |
| | | | | Cells Dev. 18(9): 1253-62. |
| surzebiclimab | TIM-3 | Surzebiclimab | MedChemExpress, | |
| | | | #HY-P99962 | |
| Sym022 | LAG-3 | Research Grade Anti-Human | Antibody System, | |
| | | CD223/LAG3 (Sym022) | #DHD30423 | |
| Sym023 | TIM-3 | | | Lindsted et al., 2018, Cancer |
| | | | | Res. 78 (13_Supplement): |
| | | | | 5629. |
| Sym024 | CD73 | | | Jakobsen et al., 2021, Cancer |
| | | | | Res. 81 (13_Supplement): |
| | | | | 1797. |
| SYSA180 | CLDN18.2 | | | Wang et al., 2023, J. Clin. |
| | | | | Oncol. 41 (16 suppl.), 3016. |
| T200/797 | CD45RO | Anti-CD45RO antibody | Abcam, #ab216024 | |
| | | [T200/797] | | |
| T3-3A1 | CD7 | CD7 (T-Cell Leukemia Marker) | ThermoFisher, #924- | |
| | | Monoclonal Antibody (T3-3A1) | MSM5-P0 | |
| T6.3 | CD2 | available from multiple sources, including: | | |
| | | Mouse Anti-CD2 Recombinant | Creative Biolabs, | |
| | | Antibody (clone T6.3) | #ZG-0903F | |
| | | Monoclonal Mouse anti-Human | LS Bio, | |
| | | CD2 Antibody (clone T6.3) | #LS-C140259 | |
| T595 | CD117 | CD117 Mouse Monoclonal | OriGene, #TA355261 | |
| | | Antibody [Clone ID: T595] | | |
| TAB-071CL | BMPR2 | Anti-Human BMPR | Creative Biolabs, | |
| | | Recombinant Antibody | #TAB-071CL | |
| talquetamab | GPRC5D | | | Pillarisetti et al., Blood |
| | | | | 135:1232-43 (2020) |
| tavolimab | OX40 | Tavolimab Recombinant Human | ThermoFisher, | |
| | | Monoclonal Antibody | #MA5-42300 | |
| TB19 | CD73 | In VivoMAb Anti-Human | AntibodySystem, | |
| | | CD73/NT5E Antibody (TB19) | #VHD46601 | |
| TB38 | CD73 | In VivoMAb Anti-Human | Antibody System, | |
| | | CD73/NT5E Antibody (TB38) | #VHD46602 | |
| tebotelimab | LAG-3 | Tebotelimab | MedChemExpress, | |
| | PD-1 | | #HY-P99573 | |
| teclistamab | CD3 | Teclistamab | MedChemExpress, | |
| | | | #HY-P99392 | |
| telazorlimab | OX40 | Telazorlimab | MedChemExpress, | |
| | | | #HY-P99570 | |
| teplizumab | CD3 | Teplizumab | MedChemExpress, | |
| | | | #HY-P99222 | |
| TG6050 | CTLA-4 | | | Marchand et al., 2023, Cancer |
| | | | | Res. 83 (7_Supplement): 694. |
| TH-69 | CD7 | Anti-CD7 [TH69] | Absolute Antibody, | |
| | | | #Ab03054 | |
| theralizumab | CD28 | Theralizumab | MedChemExpress, | |
| | | | #HY-P9975 | |
| tifcemalimab | BTLA | | | Song et al., 2023, Blood 142 |
| | | | | (Supplement 1): 4458. |
| tiragolumab | TIGIT | | | Kim et al., 2023, JAMA |
| | | | | Oncol. 9(11): 1574-1582. |
| tisagenlecleucel | CD19 | | | Mueller et al., 2018, Clin. |
| | | | | Cancer Res. 24 (24): 6175- |
| | | | | 6184. |
| tislelizumab | PD-1 | Tislelizumab | MedChemExpress, | |
| | | | #HY-P99052 | |
| TNB-383B | BCMA | | | U.S. Pat. No. 11,505,606 |
| tocilizumab | IL-6R | available from multiple sources, including: | | |
| | | IL6R antibody \| rhPM-1 | BioRad, #MCA6106 | |
| | | Tocilizumab | MedChemExpress, | |
| | | | #HY-P9917 | |
| | | Tocilizumab Recombinant | ThermoFisher, | |
| | | Human Monoclonal Antibody | #MA5-48061 | |
| | | (rhPM-1 (Tocilizumab)) | | |
| toripalimab | PD-1 | Toripalimab | MedChemExpress, | |
| | | | #HY-P9978 | |
| TORL-2-307-ADC | CLDN18.2 | | | Clinical Trial ID: |
| | | | | NCT05156866 |
| tositumomab | CD20 | available from multiple sources, including: | | |
| | | Human CD20 (Research Grade | R&D Systems, | |
| | | Tositumomab Biosimilar) Alexa | #FAB10440R | |
| | | Fluor ® 647-conjugated Antibody | | |
| | | Tositumomab Recombinant | ThermoFisher, | |
| | | Mouse Monoclonal Antibody | #MA5-41763 | |
| TQB2618 | TIM-3 | Research Grade Anti-Human | AntibodySystem, | |
| | | CD366/HAVCR2/TIM-3 | #DHJ28406 | |
| | | (TQB2618) | | |
| TQB2934 | BCMA | | | US 2023/0193292 |
| TRC205 | CD105 | | | U.S. Pat. No. 9,926,375 |
| tregalizumab | CD4 | Tregalizumab | MedChemExpress, | |
| | | | #HY-P99327 | |
| tremelimumab | CTLA-4 | Tremelimumab | MedChemExpress, | |
| | | | #HY-P9918 | |
| TRX1 | CD4 | Anti-Human CD4 Recombinant | Creative Biolabs, | |
| | | Antibody (TRX1) | #TAB-168LC | |
| TRX2 | CD8 | Oxford Therapeutic Antibody Centre, Oxford | | |
| | | University, Oxford, United Kingdom | | |
| TRX518 | GITR | Research Grade Anti-Human | AntibodySystem, | |
| | | CD357/TNFRSF18/GITR | #DHJ89807 | |
| | | (TRX518) | | |
| TS1/8 | CD2 | available from multiple sources, including: | | |
| | | CD2 Monoclonal Antibody | ThermoFisher, | |
| | | (TS1/8) | #MA5-44080 | |
| | | Purified anti-human CD2 | BioLegend, #309202 | |
| | | Antibody | | |
| TS2/18 | CD2 | available from multiple sources, including: | | |
| | | CD2 Monoclonal Antibody | ThermoFisher, | |
| | | (TS2/18) | #MA0200 | |
| | | CD2 Antibody (TS2/18.1.1) | Santa Cruz Bio, #sc- | |
| | | | 19640 | |
| TTI-CD200 | CD200 | | | Rastogi et al., 2021, Br. J. |
| | | | | Haematol. 193, 155-159. |
| TUSP-2 | Stro-1 | Anti-STRO-1 monoclonal | Creative Diagnostics, | |
| | | antibody [Alexa Fluor ® 647] | #DMAB18656 | |
| UB-421 | CD4 | Research Grade Semzuvolimab | AntibodySystem, | |
| | | | #DHB95908 | |
| ublituximab | CD20 | available from multiple sources, including: | | |
| | | Ublituximab | MedChemExpress, | |
| | | | #HY-P99538 | |
| | | Ublituximab (Anti-CD20) | SelleckChem, | |
| | | | #A2807 | |
| | | Ublituximab Recombinant | ThermoFisher, | |
| | | Human Monoclonal Antibody | #MA5-41938 | |
| UCART20 | CD20 | | | Kang et al., 2024, J. Hem. |
| | | | | Oncol. 17: 29. |
| UCHL1 | CD45RO | Anti-CD45RO antibody [UCH- | Abcam, #ab23 | |
| | | L1] | | |
| UCHT2 | CD5 | CD5 Monoclonal Antibody | ThermoFisher, #14- | |
| | | (UCHT2), eBioscience ™ | 0059-82 | |
| UCHT4 | CD8 | available from multiple sources, including: | | |
| | | CD8 Monoclonal Antibody | ThermoFisher, # | |
| | | (UCHT4) | 65204-1-IG | |
| | | Anti-CD8 [UCHT4] | Absolute Antibody, | |
| | | | #Ab00694-2.0 | |
| uliledlimab | CD73 | Uliledlimab | MedChemExpress, | |
| | | | #HY-P99169 | |
| UMAB216 | CD117 | KIT Monoclonal Antibody | ThermoFisher, | |
| | | (UMAB216), UltraMAB ™ | #UM800108CF | |
| UMCD2 | CD2 | available from multiple sources, including: | | |
| | | anti-human CD2 | Biohippo, | |
| | | | #BHA14700014 | |
| | | CD2 Monoclonal Mouse | Biotium, #1235 | |
| | | Antibody (UMCD2) | | |
| urelumab (BMS-663513) | CD137 | Urelumab | MedChemExpress, | |
| | | | #HY-P99055 | |
| ustekinumab | IL-12 | available from multiple sources, including: | | |
| | IL-23 | Ustekinumab Biosimilar - Anti- | Proteogenix, #PX- | |
| | | Human IL-12 IL-23 mAb - | TA1011 | |
| | | Research Grade | | |
| | | Ustekinumab (anti-IL-12/IL-23) | SelleckChem, | |
| | | | #A2024 | |
| utomilumab (PF-05082566) | CD137 | Utomilumab | MedChemExpress, | |
| | | | #HY-P99056 | |
| varlilumab | CD27 | Varlilumab Recombinant Human | ThermoFisher, | |
| | | Monoclonal Antibody | #MA5-42030 | |
| VB6-008 | CD44 | | | U.S. Pat. No. 8,383,117 |
| veltuzumab | CD20 | available from multiple sources, including: | | |
| | | Veltuzumab | MedChemExpress, | |
| | | | #HY-P99224 | |
| | | Veltuzumab Biosimilar - Anti- | Proteogenix, #PX- | |
| | | CD20 receptors mAb - Research | TA1616 | |
| | | Grade | | |
| | | Veltuzumab Recombinant | ThermoFisher, | |
| | | Human Monoclonal Antibody | #MA5-42301 | |
| VHH-B1, VHH-B2 | FAP | | | WO 2022/053651 |
| VIB9600 | CD32A | | | Chen et al., 2019, Ann. |
| | | | | Rheum. Dis. 78(2): 228-237. |
| vibecotamab | CD3 | Vibecotamab Recombinant | Fisher Scientific, | |
| | | Human Monoclonal Antibody, | #PIMA542248 | |
| | | Invitrogen ™ | | |
| vibostolimab (MK-7684) | TIGIT | | | Niu et al., 2022, Ann. Oncol. |
| | | | | 33(2): 169-180. |
| visilizumab | CD3 | Anti-CD3E Recombinant | Creative Biolabs, | |
| | | Antibody (Visilizumab) | #TAB-714 | |
| vonlerizumab | OX40 | Vonlerizumab (Anti-TNFRSF4/ | SelleckChem, | |
| | | OX40/ | #A2325 | |
| | | CD134) | | |
| vorsetuzumab | CD27 | Vorsetuzumab | MedChemExpress, | |
| | CD70 | | #HY-P99273 | |
| vudalimab | PD-1 | Research Grade Vudalimab | Antibody System, | |
| | | | #DHH02222 | |
| vunakizumab | CD27 | Anti-Human IL17A | Creative Biolabs, | |
| | | Recombinant Antibody | #TAB-458CQ | |
| | | (Vunakizumab) | | |
| W3/13HLK | CD43 | CD43 Monoclonal Antibody | ThermoFisher, | |
| | | (W3/13HLK), PE | #MA5-17386 | |
| W5C5 | SUSD2 | Purified anti-human SUSD2 | BioLegend, #327401 | |
| | | Antibody | | |
| W8B2 | MSCA-1 | MSCA-1 Antibody, anti-human | Miltenyi Biotec, | |
| | | | #130-093-595 | |
| WT1 | CD7 | Mouse Anti-CD7 Recombinant | Creative Biolabs, | |
| | | Antibody (WT1) | #CBMAB-C10581- | |
| | | | LY | |
| WV078 | BCMA | | | U.S. Pat. No. 11,492,409 |
| X9C3 | MSCA-1 | Mouse anti-Human MSCA-1 | Creative Diagnostics, | |
| | | monoclonal antibody, PE | #CABT-B12374 | |
| XTX101 | CTLA-4 | | | Jenkins et al., 2023, J. |
| | | | | Immunother. Cancer 11(12): |
| | | | | e007785. |
| Y1/82A | CD68 | CD68 antibody \| Y1/82A | Bio-Rad, | |
| | | | #MCA6014GA | |
| YB5.B8 | CD117 | CD117 (c-Kit) Monoclonal | ThermoFisher, | |
| | | Antibody (YB5.B8), | #14-1179-82 | |
| | | eBioscience ™ | | |
| YH001 | CTLA-4 | | | Ganju et al., 2021, J. Clin. |
| | | | | Oncol. 39 (15_suppl): 2577. |
| YTC182.20 | CD8 | Anti-CD8 alpha antibody | Abcam, #ab60076 | |
| | | [YTC182.20] | | |
| YTH3.2.6 | CD7 | Anti-CD7 [YTH3.2.6] | Absolute Antibody, | |
| | | | #Ab01019 | |
| zalifrelimab | CTLA-4 | Zalifrelimab | MedChemExpress, | |
| | | | #HY-P99514 | |
| zanolimumab | CD4 | Zanolimumab Recombinant | ThermoFisher, | |
| | | Human Monoclonal Antibody | #MA5-41819 | |
| zimberelimab | PD-1 | Zimberelimab | MedChemExpress, | |
| | | | #HY-P99109 | |
| zolbetuximab (IMAB362) | CLDN18.2 | | | Sahin et al., 2018, Eur. J. |
| | | | | Cancer 100, 17-26. |

We claim:

1. A method of increasing in vivo transfection efficiency of T cells, comprising administering to a mammalian subject in a compact regimen multiple doses of a T cell-targeted lipid nanoparticle (tLNP) encapsulating an RNA encoding a T cell activating agent, wherein the tLNP delivers the RNA encoding the T cell activating agent to the targeted T cells in the subject and the targeted T cells express the T cell activating agent, wherein the compact dose regimen comprises administering a second dose after an initial dose within 1 to 5 days, whereby more T cells express the T cell activating agent as a result of a subsequent administration than as a result of the initial administration, and wherein any subsequent dose is administered within 1 to 5 days of the immediately preceding dose.

2. The method of claim 1, wherein the second dose is administered 2 days, 3 days, or 4 days after the initial dose.

3. The method of claim 1, wherein the compact dose regimen comprises 2 or 3 doses.

4. The method of claim 3, wherein the 2 to 3 doses are administered at 72-hour intervals (2xQ72h or 3xQ72h).

5. The method of claim 1, wherein the tLNP dosage for each administration ranges from about 0.03to about 1.5 mg RNA/kg.

6. The method of claim 1, wherein (a) the initial tLNP dose is the same as each subsequent dose, (b) the initial tLNP dose is lower than each subsequent dose, or (c) the initial tLNP dose is higher than each subsequent dose.

7. The method of claim 1, wherein the tLNP encapsulated RNA is mRNA.

8. The method of claim 1, wherein the encoded T cell activating agent is a chimeric antigen receptor (CAR), a T cell receptor (TCR), a T cell engager (TCE), a conditioning agent, or any combination thereof.

9. The method of claim 8, wherein the encoded T cell activating agent is a CAR, wherein the CAR comprises a binding moiety specific for a B cell lineage antigen.

10-11. (canceled)

12. The method of claim 1, wherein the tLNP comprises an ionizable cationic lipid of:
i) Formula 1 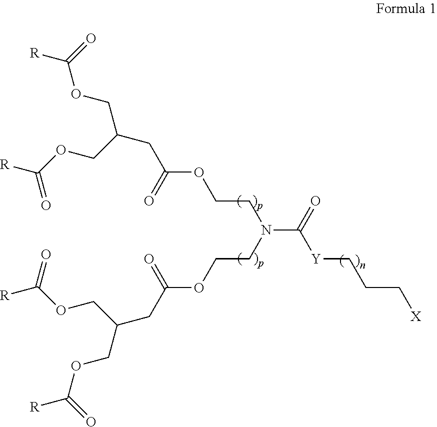
wherein
Y is O, NH, N—CH₃, or CH₂,
n is an integer from 0 to 4,
X is 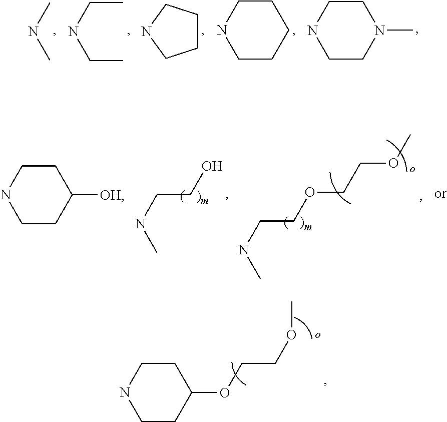
m is an integer from 1 to 3,
is an integer from 1 to 4, and
p is an integer from 1 to 4,
wherein when p=1:
each R is independently C₆ to C₁₆ straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain;
wherein when p=2:
each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or C₈ to C₁₆ aryl-alkyl in which the aryl is phenyl or naphthalenyl and is positioned at either end or within the alkyl chain;
wherein when p=3:
each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain; and
wherein when p=4:
each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or C₈ to C₁₂ aryl-alky in which the aryl is phenyl or naphthalenyl and is positioned at the either end or within the alkyl chain
ii) Formula M5: 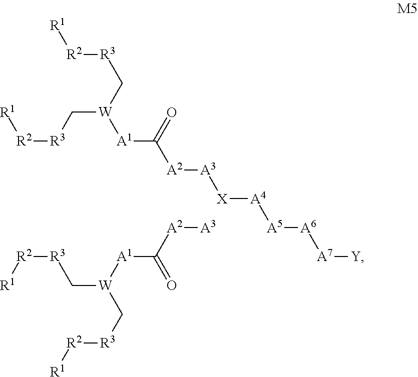
wherein
each R¹ is independently selected from a C₇-C₁₁ alkyl or a C₇-C₁₁ alkenyl,
A¹ is CH₂i-2,
A² is O,
A³ is (CH₂)₁₋₅, wherein A³ is not CH₂ if X is N,
X is N, CH, or C—CH₃,
A⁴ is CH₂, C═O, NH, NCH₃, or O,
A⁵ is absent, O, S, NH, or NCH₃ if A⁴ is C═O, or A⁵ is C═O if A⁴ is not C═O,
A⁶ is O, S, NH, NCH₃ or (CH₂)₀₋₂,
A⁷ is (CH₂)₀₋₆, wherein if A⁶ is O, S, NH, NCH₃, A⁷ is (CH₂)₂₋₄,
Y is 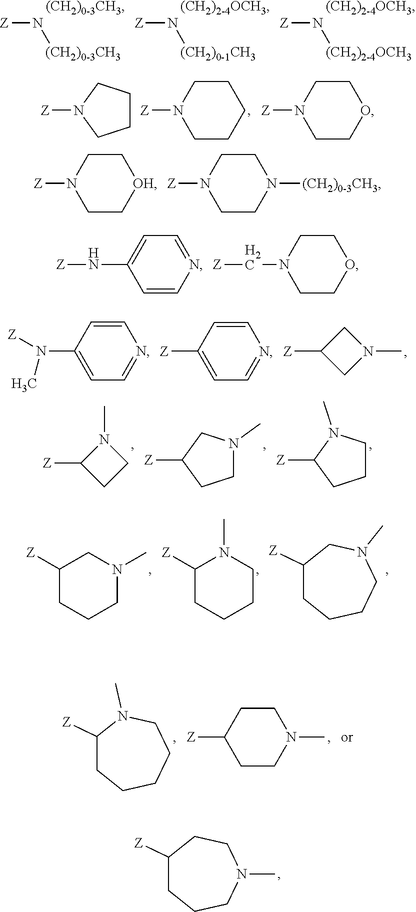
wherein Z is a bond; and
R² is O, R³ is C═O and W is CH or N, or R² is C═O, R³ is O and W is CH:
wherein A⁶ and A⁷ are not both (CH₂)₀ unless A⁵ is C═O:
wherein
a) A¹ is CH₂, A³ is (CH₂)₂₋₅, X is N, A⁴ is C═O, A⁵ is O, S, NH, NCH₃, A⁶ is (CH₂)₁₋₂, A⁷ is (CH₂)₁₋₄, or
b) A¹ is CH₂, A³ is (CH₂)₁₋₄, X is CH, A⁴ is CH₂, NH, NCH₃, O, A⁵ is C═O, A⁶ is O, NH, NCH₃, A⁷ is (CH)₂₋₆, or
c) A¹ is (CH₂)₂, A³ is (CH₂)₁₋₄, X is C—CH₃, A⁴ is C═O, A⁵ is O, NH, NCH₃, A⁶ is (CH₂)₁₋₂, A⁷ is (CH₂)₁₋₄, or
d) A¹ is CH₂, A³ is (CH₂)₂₋₅, X is N, A⁴ is C═O, A⁵ is absent, A⁶ is (CH₂)₀, A⁷ is (CH₂)₀, and Y is 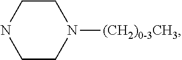
or
e) A¹ is CH₂, A³ is (CH₂)₁₋₅, X is CH, A⁴ is CH₂, NH, NCH₃ or O, A⁵ is C═O, A⁶ is (CH₂)₀, A⁷ is (CH₂)₀, and Y is 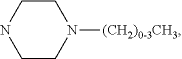
or
f) A¹ is (CH₂)₂, A³ is (CH₂)₁₋₅, X is CCH₃, A⁴ is C═O, A⁵ is absent, A⁶ is (CH₂)₀, A⁷ is (CH₂)₀, and Y is 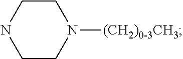
wherein
the number of contiguous atoms present in a span: 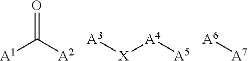
is in the range from 7-17; or
iii) Formula M6: 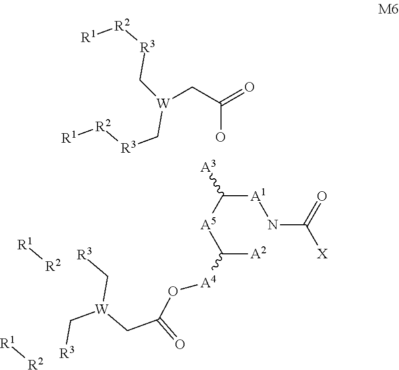
wherein X is 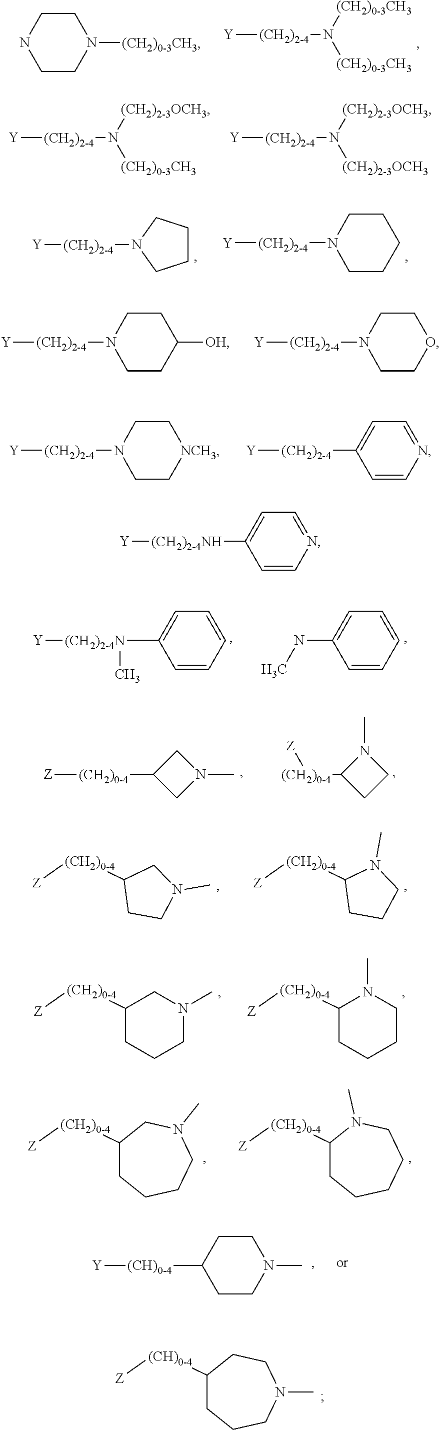
and
Y is O, S, NH, or NCH₃:
Z is O, NH, or NCH₃:
R² is O, R³ is C═O and W is CH or N, or R² is C═O, R³ is O and W is CH; and
each R¹ is independently selected from a C₇-C₁₁ alkyl or a C₇-C₁₁ alkenyl;
each A¹, A², A³, and A⁴ is independently selected from (CH₂)₀ and (CH₂)₁,
A⁵ is selected from (CH₂)₀₋₄, CH═CH, and CH₂—CH═CH—CH₂; and
a wavy bond indicates that any relative or absolute stereo-configuration of the corresponding ring atom, or a mixture of stereo-configurations.

13-14. (canceled)

15. The method of claim 12, wherein the ionizable cationic lipid comprises

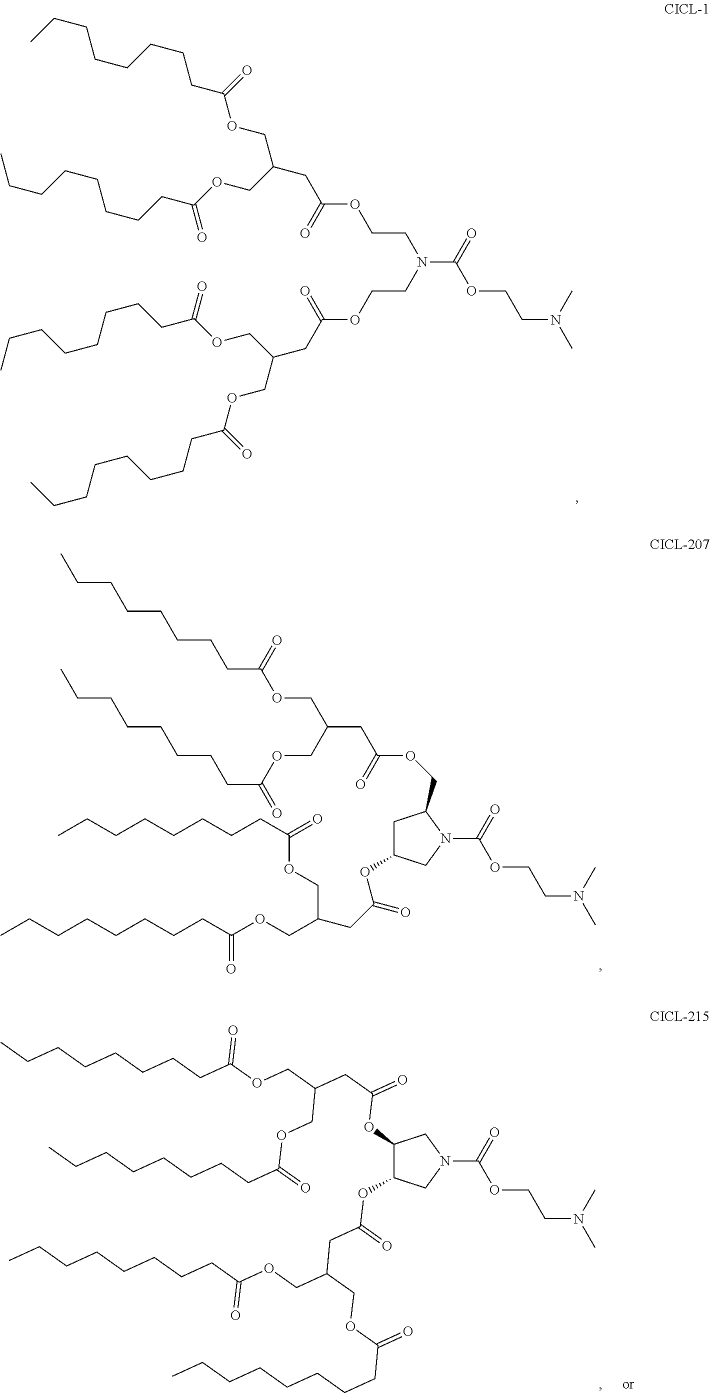

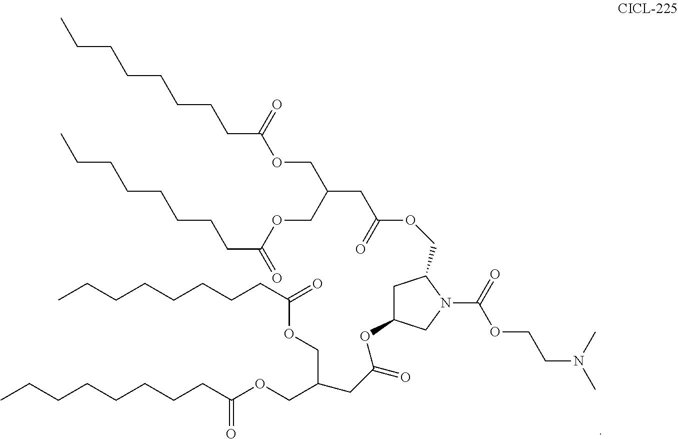 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OCCN(C)C]

16. (canceled)

17. The method of claim 1, wherein the tLNP comprises about 35 to about 65 mol % ionizable cationic lipid, about 0.5 to about 3 mol % PEG-lipid comprising functionalized PEG-lipid and non-functionalized PEG-lipid, about 7 to about 13 mol % phospholipid, and about 27 to about 50 mol % sterol.

18. The method of claim 17, wherein the tLNP comprises about 58% ionizable cationic lipid, about 30.5 mol % cholesterol, about 10 mol % distearoylphosphatidylcholine (DSPC), about 1.4 mol % distearoylglycerol-polyethylene glycol, and about 0.1 mol % distearoylphosphatidylethanolamine-polyethylene glycol (DSPE-PEG).

19. The method of claim 18, wherein the DSPE-PEG is conjugated to a targeting moiety comprising an antibody or antigen binding portion thereof.

20. The method of claim 19, wherein the antibody or antigen binding portion thereof comprises a F(ab′) analog.

21. The method of claim 19, wherein the antibody or antigen binding portion thereof is specific for CD8, CD7, CD5, or CD2.

22. The method of claim 1, wherein the targeted T cell is a CD8+ T cell.

23. The method of claim 1, wherein a low dose corticosteroid is administered about 1 hour before the first dose or last dose of tLNP.

24-26. (canceled)

27. The method of claim 1, wherein the T cell activating agent of each of the multiple doses is a CAR, TCR, or TCE.

28-33. (canceled)

34. The method of claim 1, wherein the T cell activating agent of the first or first and second of the multiple doses is a conditioning agent and the T cell activating agent of the subsequent doses is a CAR, TCR, or TCE.

35. The method of claim 1, wherein administering comprises intravenous infusion.

36. A method of treating a disease or disorder associated with a pathogenic cell comprising administering to a subject in need thereof in a compact regimen multiple doses of a T cell-targeted tLNP encapsulating an RNA encoding a T cell activating agent, wherein the tLNP delivers the RNA encoding the T cell activating agent to the targeted T cells in the subject and the targeted T cells express the T cell activating agent, wherein the compact dose regimen comprises administering each dose within 1 to 5 days of the immediately preceding dose, wherein the T cell activating agent of the initial dose, or the initial and second dose, is a conditioning agent, a CAR, a TCR, or a TCE and wherein the T cell activating agent of each dose subsequent to the initial dose, or the initial and second dose is a CAR, a TCR, or a TCE that is specific for an antigen expressed by the pathogenic cell.

37-53. (canceled)

54. The method of claim 1, wherein the compact regimen comprises administering each subsequent dose of the multiple doses within 2 to 5 days of the immediately preceding previous dose.

55. The method of claim 54, wherein each subsequent dose of the multiple doses is within 2 to 3 days of the immediately preceding previous dose.

56. The method of claim 55, wherein a dose is administered every 3^{rd} day.

57. The method of claim 56, wherein a total of 2-6 doses, 2-4 doses, 3 doses, or 2 doses are administered in a cycle of treatment.

58-59. (canceled)

60. The method of claim 54, wherein the cumulative dosage is ≤3 mg RNA/kg/6 days.

61-63. (canceled)

64. A pharmaceutical composition comprising a T cell-targeted tLNP encapsulating an mRNA encoding a T cell-activating agent suitable for administration at a dosage of at least 0.03 mg RNA/kg or in a range of about 0.03 to about 1.0 mg/kg in a compact regimen.

65-74. (canceled)

75. A method of increasing in vivo transfection efficiency of T cells for introducing a therapeutic agent into the T cells comprising administering to a mammalian subject in a compact regimen, at least one dose of a T cell activating agent and subsequently administering within 1, 2, 3, 4, or 5 days at least one dose of a therapeutic agent wherein the therapeutic agent comprises a T cell-targeted lipid nanoparticle (tLNP) encapsulating an RNA encoding a CAR, TCR, or TCE, wherein a population of cells in the subject expresses an antigen recognized by the CAR, TCR, or TCE, whereby more T cells express the CAR, TCR, or TCE as a result of the initial administration of the T cell activating agent than if it had not been administered.

76-80. (canceled)

81. A method of increasing in vivo T cell reprogramming efficiency, comprising administering to a mammalian subject in a compact regimen at least one dose of a T cell-targeted lipid nanoparticle (tLNP) encapsulating an RNA encoding a first CAR, TCR, or TCE that binds an antigen having non-restricted expression, followed by administering within 1, 2, 3, 4, or 5 days at least one subsequent dose of a T cell-targeted lipid nanoparticle (tLNP) encapsulating an RNA encoding a second CAR, TCR, or TCE that binds an antigen having restricted expression, whereby more T cells express the second CAR, TCR, or TCE as a result of the at least one subsequent administration than if it had not been preceded by the at least one dose of the T cell-targeted lipid nanoparticle (tLNP) encapsulating an RNA encoding the first CAR, TCR, or TCE.

82. A method of depleting B cells in a mammalian subject, the method comprising administering in a compact regimen at least one dose of a T cell activating agent and subsequently administering within 1, 2, 3, 4, or 5 days at least one dose of a T cell-targeted lipid nanoparticle (tLNP) encapsulating an RNA encoding a CAR, TCR, or TCE that binds a B cell antigen, whereby more T cells express the CAR, TCR, or TCE that binds the B cell antigen as a result of a subsequent administration than if it had not been preceded by the at least one dose of the T cell activating agent.

83. A method of blunting induction of an anti-drug antibodies (ADA) reaction, comprising administering in a compact regimen at least one dose of a T cell activating agent and subsequently administering within 1, 2, 3, 4, or 5 days at least one dose of a T cell-targeted lipid nanoparticle (tLNP) encapsulating an RNA encoding a CAR, TCR, or TCE that binds a B cell antigen, whereby B cells are sufficiently depleted for an interval of time that administration of a immunogenic drug within that interval of time results in a diminished or absent ADA reaction.

84-94. (canceled)
